SEQUENCE LISTING (140 sequences)

SEQ ID NO: 1 - protein, 9 aa, synthetic construct
FTFSSYAMS

SEQ ID NO: 2 - protein, 17 aa, synthetic construct
AISASGGSTYYADSVKG

SEQ ID NO: 3 - protein, 21 aa, synthetic construct
ARGSPIGYSYARVSYYYYMDV

SEQ ID NO: 4 - protein, 11 aa, synthetic construct
RASQGISRWLA

SEQ ID NO: 5 - protein, 7 aa, synthetic construct
AASSLQS

SEQ ID NO: 6 - protein, 9 aa, synthetic construct
QQGNSFPLT

SEQ ID NO: 7 - protein, 9 aa, synthetic construct
FTFGDYAMW

SEQ ID NO: 8 - protein, 17 aa, synthetic construct
AISASGDVTYYADSVKG

SEQ ID NO: 9 - protein, 21 aa, synthetic construct
ARGSLIGYSYARVSYYYYSDV

SEQ ID NO: 10 - protein, 9 aa, synthetic construct
QQGWSFPLT

SEQ ID NO: 11 - protein, 9 aa, synthetic construct
GSISSYYWS

SEQ ID NO: 12 - protein, 16 aa, synthetic construct
RIYTSGSTNYNPSLKS

SEQ ID NO: 13 - protein, 20 aa, synthetic construct
ARDLTNYGSGKSIRFYGMDV

SEQ ID NO: 14 - protein, 11 aa, synthetic construct
QASQDISNYLN

SEQ ID NO: 15 - protein, 7 aa, synthetic construct
DASNLET

SEQ ID NO: 16 - protein, 8 aa, synthetic construct
QQDDNFIT

SEQ ID NO: 17 - protein, 9 aa, synthetic construct
GSISSNYWH

SEQ ID NO: 18 - protein, 16 aa, synthetic construct
RIHTSGSTFYNPSLKS

SEQ ID NO: 19 - protein, 20 aa, synthetic construct
ARDITNYGSGKSIRFYPMDV

SEQ ID NO: 20 - protein, 8 aa, synthetic construct
QQDDTFIT

SEQ ID NO: 21 - protein, 9 aa, synthetic construct
GSISSHYWH

SEQ ID NO: 22 - protein, 16 aa, synthetic construct
RIATSGSTYYNPSLKS

SEQ ID NO: 23 - protein, 20 aa, synthetic construct
ARDLTNYGITKSIRFYGMDV

SEQ ID NO: 24 - protein, 8 aa, synthetic construct
QQVDNFIT

SEQ ID NO: 25 - protein, 20 aa, synthetic construct
ARDLTNYGWSKSIRFYGMDV

SEQ ID NO: 26 - protein, 8 aa, synthetic construct
QQTDNFIT

SEQ ID NO: 27 - protein, 9 aa, synthetic construct
YTFTGYYMH

SEQ ID NO: 28 - protein, 17 aa, synthetic construct
SINPSSGGTNYAQKFQG

SEQ ID NO: 29 - protein, 20 aa, synthetic construct
ARVGGAAAAGKAYTASAFDI

SEQ ID NO: 30 - protein, 11 aa, synthetic construct
RASQSVGSNLA

SEQ ID NO: 31 - protein, 7 aa, synthetic construct
GASTRAT

SEQ ID NO: 32 - protein, 10 aa, synthetic construct
QQFHNWPPLT

SEQ ID NO: 33 - protein, 9 aa, synthetic construct
YTFTDHYMH

SEQ ID NO: 34 - protein, 17 aa, synthetic construct
AINPSSGGTAYAQKFQG

SEQ ID NO: 35 - protein, 20 aa, synthetic construct
ARVGGAARVGKAYTASAFDI

SEQ ID NO: 36 - protein, 9 aa, synthetic construct
YTFTSYGIS

SEQ ID NO: 37 - protein, 17 aa, synthetic construct
WISAYSGDTNYAQKLQG

SEQ ID NO: 38 - protein, 18 aa, synthetic construct
ARGDEVVGKMTYYYGMDV

SEQ ID NO: 39 - protein, 9 aa, synthetic construct
QQANSFPIT

SEQ ID NO: 40 - protein, 9 aa, synthetic construct
YTFTDYAIS

SEQ ID NO: 41 - protein, 17 aa, synthetic construct
WISASTADTNYAQKLQG

SEQ ID NO: 42 - protein, 18 aa, synthetic construct
ARGDEVVGKRTYYIGMDV

SEQ ID NO: 43 - protein, 18 aa, synthetic construct
ARGTEVVGKMTVYLGMDV

SEQ ID NO: 44 - protein, 18 aa, synthetic construct
ARQDEVVGKMTYYIGTDV

SEQ ID NO: 45 - protein, 9 aa, synthetic construct
QQANSFPLT

SEQ ID NO: 46 - protein, 17 aa, synthetic construct
WISAGTADTNYAQKLQG

SEQ ID NO: 47 - protein, 18 aa, synthetic construct
ARGDEVVGKMTYYYDMDV

SEQ ID NO: 48 - protein, 9 aa, synthetic construct
QQASSFPIT

SEQ ID NO: 49 - protein, 9 aa, synthetic construct
YTFDDYGIS

SEQ ID NO: 50 - protein, 17 aa, synthetic construct
WISATVADTNYAQKLQG

SEQ ID NO: 51 - protein, 18 aa, synthetic construct
ARGDEPVGKMTYYYGMDV

SEQ ID NO: 52 - protein, 9 aa, synthetic construct
QQANSLPIT

SEQ ID NO: 53 - protein, 21 aa, synthetic construct
ARDPVLLLRQQRILHESAFDI

SEQ ID NO: 54 - protein, 9 aa, synthetic construct
YTFEGHYMH

SEQ ID NO: 55 - protein, 17 aa, synthetic construct
AINPYSGQTNYAQKFQG

SEQ ID NO: 56 - protein, 21 aa, synthetic construct
ARDFVLLLRQSRILHESAFDI

SEQ ID NO: 57 - protein, 128 aa, synthetic construct
EVQLLESGGGLVQPGGSLRLSCAASGFTFSSYAMSWVRQAPGKGLEWVSAISASGGSTYYADSVKGRFTISRDNSKNTLYLQMNSLRAEDTAVYYCARGSPIGYSYARVSYYYYMDVWGKGTTVTVSS

SEQ ID NO: 58 - protein, 107 aa, synthetic construct
DIQMTQSPSSVSASVGDRVTITCRASQGISRWLAWYQQKPGKAPKLLIYAASSLQSGVPSRFSGSGSGTDFTLTISSLQPEDFATYYCQQGNSFPLTFGGGTKVEIK

SEQ ID NO: 59 - protein, 128 aa, synthetic construct
EVQLLESGGGLVQPGGSLRLSCAASGFTFGDYAMWWVRQAPGKGLEWVSAISASGDVTYYADSVKGRFTISRDNSKNTLYLQMNSLRAEDTAVYYCARGSLIGYSYARVSYYYYSDVWGKGTTVTVSS

SEQ ID NO: 60 - protein, 107 aa, synthetic construct
DIQMTQSPSSVSASVGDRVTITCRASQGISRWLAWYQQKPGKAPKLLIYAASSLQSGVPSRFSGSGSGTDFTLTISSLQPEDFATYYCQQGWSFPLTFGGGTKVEIK

SEQ ID NO: 61 - protein, 126 aa, synthetic construct
QVQLQESGPGLVKPSETLSLTCTVSGGSISSYYWSWIRQPAGKGLEWIGRIYTSGSTNYNPSLKSRVTMSVDTSKNQFSLKLSSVTAADTAVYYCARDLTNYGSGKSIRFYGMDVWGQGTTVTVSS

SEQ ID NO: 62 - protein, 106 aa, synthetic construct
DIQMTQSPSSLSASVGDRVTITCQASQDISNYLNWYQQKPGKAPKLLIYDASNLETGVPSRFSGSGSGTDFTFTISSLQPEDIATYYCQQDDNFITFGGGTKVEIK

SEQ ID NO: 63 - protein, 126 aa, synthetic construct
QVQLQESGPGLVKPSETLSLTCTVSGGSISSNYWHWIRQPAGKGLEWIGRIHTSGSTFYNPSLKSRVTMSVDTSKNQFSLKLSSVTAADTAVYYCARDITNYGSGKSIRFYPMDVWGQGTTVTVSS

SEQ ID NO: 64 - protein, 106 aa, synthetic construct
DIQMTQSPSSLSASVGDRVTITCQASQDISNYLNWYQQKPGKAPKLLIYDASNLETGVPSRFSGSGSGTDFTFTISSLQPEDIATYYCQQDDTFITFGGGTKVEIK

SEQ ID NO: 65 - protein, 126 aa, synthetic construct
QVQLQESGPGLVKPSETLSLTCTVSGGSISSHYWHWIRQPAGKGLEWIGRIATSGSTYYNPSLKSRVTMSVDTSKNQFSLKLSSVTAADTAVYYCARDLTNYGITKSIRFYGMDVWGQGTTVTVSS

SEQ ID NO: 66 - protein, 106 aa, synthetic construct
DIQMTQSPSSLSASVGDRVTITCQASQDISNYLNWYQQKPGKAPKLLIYDASNLETGVPSRFSGSGSGTDFTFTISSLQPEDIATYYCQQVDNFITFGGGTKVEIK

SEQ ID NO: 67 - protein, 126 aa, synthetic construct
QVQLQESGPGLVKPSETLSLTCTVSGGSISSHYWHWIRQPAGKGLEWIGRIHTSGSTFYNPSLKSRVTMSVDTSKNQFSLKLSSVTAADTAVYYCARDLTNYGWSKSIRFYGMDVWGQGTTVTVSS

SEQ ID NO: 68 - protein, 106 aa, synthetic construct
DIQMTQSPSSLSASVGDRVTITCQASQDISNYLNWYQQKPGKAPKLLIYDASNLETGVPSRFSGSGSGTDFTFTISSLQPEDIATYYCQQTDNFITFGGGTKVEIK

SEQ ID NO: 69 - protein, 127 aa, synthetic construct
QVQLVQSGAEVKKPGASVKVSCKASGYTFTGYYMHWVRQAPGQGLEWMGSINPSSGGTNYAQKFQGRVTMTRDTSISTAYMELSRLRSDDTAVYYCARVGGAAAAGKAYTASAFDIWGQGTMVTVSS

SEQ ID NO: 70 - protein, 108 aa, synthetic construct
EIVMTQSPATLSVSPGERATLSCRASQSVGSNLAWYQQKPGQAPRLLIYGASTRATGIPARFSGSGSGTEFTLTISSLQSEDFAVYYCQQFHNWPPLTFGGGTKVEIK

SEQ ID NO: 71 - protein, 127 aa, synthetic construct
QVQLVQSGAEVKKPGASVKVSCKASGYTFTDHYMHWVRQAPGQGLEWMGAINPSSGGTAYAQKFQGRVTMTRDTSISTAYMELSRLRSDDTAVYYCARVGGAARVGKAYTASAFDIWGQGTMVTVSS

SEQ ID NO: 72 - protein, 125 aa, synthetic construct
QVQLVQSGAEVKKPGASVKVSCKASGYTFTSYGISWVRQAPGQGLEWMGWISAYSGDTNYAQKLQGRVTMTTDTSTSTAYMELRSLRSDDTAVYYCARGDEVVGKMTYYYGMDVWGQGTTVTVSS

SEQ ID NO: 73 - protein, 107 aa, synthetic construct
DIQMTQSPSSVSASVGDRVTITCRASQGISRWLAWYQQKPGKAPKLLIYAASSLQSGVPSRFSGSGSGTDFTLTISSLQPEDFATYYCQQANSFPITFGGGTKVEIK

SEQ ID NO: 74 - protein, 125 aa, synthetic construct
QVQLVQSGAEVKKPGASVKVSCKASGYTFTDYAISWVRQAPGQGLEWMGWISASTADTNYAQKLQGRVTMTTDTSTSTAYMELRSLRSDDTAVYYCARGDEVVGKMTYYYGMDVWGQGTTVTVSS

SEQ ID NO: 75 - protein, 125 aa, synthetic construct
QVQLVQSGAEVKKPGASVKVSCKASGYTFTDYAISWVRQAPGQGLEWMGWISASTADTNYAQKLQGRVTMTTDTSTSTAYMELRSLRSDDTAVYYCARGDEVVGKRTYYIGMDVWGQGTTVTVSS

SEQ ID NO: 76 - protein, 125 aa, synthetic construct
QVQLVQSGAEVKKPGASVKVSCKASGYTFTDYAISWVRQAPGQGLEWMGWISASTADTNYAQKLQGRVTMTTDTSTSTAYMELRSLRSDDTAVYYCARGTEVVGKMTVYLGMDVWGQGTTVTVSS

SEQ ID NO: 77 - protein, 125 aa, synthetic construct
QVQLVQSGAEVKKPGASVKVSCKASGYTFTDYAISWVRQAPGQGLEWMGWISASTADTNYAQKLQGRVTMTTDTSTSTAYMELRSLRSDDTAVYYCARQDEVVGKMTYYIGTDVWGQGTTVTVSS

SEQ ID NO: 78-81 - skipped (4 entries)

SEQ ID NO: 82 - protein, 107 aa, synthetic construct
DIQMTQSPSSVSASVGDRVTITCRASQGISRWLAWYQQKPGKAPKLLIYAASSLQSGVPSRFSGSGSGTDFTLTISSLQPEDFATYYCQQANSLPITFGGGTKVEIK

SEQ ID NO: 83 - protein, 128 aa, synthetic construct
QVQLVQSGAEVKKPGASVKVSCKASGYTFTGYYMHWVRQAPGQGLEWMGSINPSSGGTNYAQKFQGRVTMTRDTSISTAYMELSRLRSDDTAVYYCARDPVLLLRQQRILHESAFDIWGQGTTVTVSS

SEQ ID NO: 84 - protein, 128 aa, synthetic construct
QVQLVQSGAEVKKPGASVKVSCKASGYTFEGHYMHWVRQAPGQGLEWMGAINPYSGQTNYAQKFQGRVTMTRDTSISTAYMELSRLRSDDTAVYYCARDFVLLLRQSRILHESAFDIWGQGTTVTVSS

SEQ ID NO: 85 - protein, 9 aa, synthetic construct
  VARIANT 4: note = X is S or G
  VARIANT 5: note = X is S or D
  VARIANT 9: note = X is S or W
FTFXXYAMX

SEQ ID NO: 86 - protein, 17 aa, synthetic construct
  VARIANT 7: note = X is G or D
  VARIANT 8: note = X is S or V
AISASGXXTYYADSVKG

SEQ ID NO: 87 - protein, 21 aa, synthetic construct
  VARIANT 5: note = X is P or L
  VARIANT 19: note = X is M or S
ARGSXIGYSYARVSYYYYXDV

SEQ ID NO: 88 - protein, 9 aa, synthetic construct
  VARIANT 4: note = X is N or W
QQGXSFPLT

SEQ ID NO: 89 - protein, 9 aa, synthetic construct
  VARIANT 6: note = X is Y, N, or H
  VARIANT 9: note = X is S or H
GSISSXYWX

SEQ ID NO: 90 - protein, 16 aa, synthetic construct
  VARIANT 3: note = X is Y, H, or A
  VARIANT 9: note = X is N, F, or Y
RIXTSGSTXYNPSLKS

SEQ ID NO: 91 - protein, 20 aa, synthetic construct
  VARIANT 4: note = X is L or I
  VARIANT 9: note = X is S, I, or W
  VARIANT 10: note = X is G, T, or S
  VARIANT 17: note = X is G or P
ARDXTNYGXXKSIRFYXMDV

SEQ ID NO: 92 - protein, 8 aa, synthetic construct
  VARIANT 3: note = X is D,V, or T
  VARIANT 5: note = X is N or T
QQXDXFIT

SEQ ID NO: 93 - protein, 9 aa, synthetic construct
  VARIANT 5: note = X is G or D
  VARIANT 6: note = X is Y or H
YTFTXXYMH

SEQ ID NO: 94 - protein, 17 aa, synthetic construct
  VARIANT 1: note = X is S or A
  VARIANT 10: note = X is N or A
XINPSSGGTXYAQKFQG

SEQ ID NO: 95 - protein, 20 aa, synthetic construct
  VARIANT 8: note = X is A or R
  VARIANT 9: note = X is A or V
ARVGGAAXXGKAYTASAFDI

SEQ ID NO: 96 - protein, 9 aa, synthetic construct
  VARIANT 4: note = X is T or D
  VARIANT 5: note = X is S or D
  VARIANT 7: note = X is G or A
YTFXXYXIS

SEQ ID NO: 97 - protein, 17 aa, synthetic construct
  VARIANT 5: note = X is Y, S, G or T
  VARIANT 6: note = X is S, T, or V
  VARIANT 7: note = X is G or A
WISAXXXDTNYAQKLQG

SEQ ID NO: 98 - protein, 18 aa, synthetic construct
  VARIANT 3: note = X is G or Q
  VARIANT 4: note = X is D or T
  VARIANT 6: note = X is V or P
  VARIANT 10: note = X is M or R
  VARIANT 12: note = X is Y or V
  VARIANT 14: note = X is Y, I, or L
  VARIANT 15: note = X is G or D
  VARIANT 16: note = X is M or T
ARXXEXVGKXTXYXXXDV

SEQ ID NO: 99 - protein, 9 aa, synthetic construct
  VARIANT 4: note = X is N or S
  VARIANT 6: note = X us F or L
  VARIANT 8: note = X is I or L
QQAXSXPXT

SEQ ID NO: 100 - protein, 9 aa, synthetic construct
  VARIANT 4: note = X is T or E
  VARIANT 6: note = X is Y or H
YTFXGXYMH

SEQ ID NO: 101 - protein, 17 aa, synthetic construct
  VARIANT 1: note = X is S or A
  VARIANT 5: note = X is S or Y
  VARIANT 8: note = X is G or Q
XINPXSGXTNYAQKFQG

SEQ ID NO: 102 - protein, 21 aa, synthetic construct
  VARIANT 4: note = X is P or F
  VARIANT 11: note = X is Q or S
ARDXVLLLRQXRILHESAFDI

SEQ ID NO: 103 - protein, 230 aa, Homo sapiens
IVGGYNCEENSVPYQVSLNSGYHFCGGSLINEQWVVSAGHCYKSRIQVRLGEHNIEVLEGNEQFINAAKIIRHPQYDRKTLNNDIMLIKLSSRAVINARVSTISLPTAPPATGTKCLISGWGNTASSGADYPDELQCLDAPVLSQAKCEASYPGKITSNMFCVGFLEGGKDSCQGDAGGPVVCNGQLQGVVSWGDGCAQKNKPGVYTKVYNYVKWIKNTIAANSHHHHHH

SEQ ID NO: 104 - protein, 229 aa, Homo sapiens
VGGYICEENSVPYQVSLNSGYHFCGGSLISEQWVVSAGHCYKSRIQVRLGEHNIEVLEGNEQFINAAKIIRHPKYNSRTLDNDILLIKLSSPAVINSRVSAISLPTAPPAAGTESLISGWGNTLSSGADYPDELQCLDAPVLSQAECEASYPGKITNNMFCVGFLEGGKDSCQGDAGGPVVSNGELQGIVSWGYGCAQKNRPGVYTKVYNYVDWIKDTIAANSHHHHHH

SEQ ID NO: 105 - DNA, 384 bp, synthetic construct
  source 1..384: mol_type = other DNA
gaggtgcagctgctggaaagcggaggaggcctggtgcaacccggcggttcgctgaggctgagctgtgccgcctctggtttcaccttttcaagctacgcgatgagctgggtcagacaggccccgggaaagggattggaatgggtgtccgctatctccgcttccggagggtcaacctactacgccgactccgtgaagggacggttcaccatctcgcgcgacaactccaagaacaccctttacctccaaatgaactccctgagggccgaggacactgcagtctactactgcgcgagaggctcacctattggctactcctatgcccgcgtgtcgtactactactacatggatgtctggggaaaggggactaccgtgactgtctcctcg

SEQ ID NO: 106 - DNA, 321 bp, synthetic construct
  source 1..321: mol_type = other DNA
gacatccaaatgactcagtccccgtcctccgtgtctgcctccgtgggagatagagtgaccatcacttgtcgggcctcccaaggcatttcacgctggctggcctggtaccagcagaaaccagggaaggcacccaagctcctgatctacgccgcgtcgtcactgcaatccggagtcccttcgcggttctccggttcgggatccggaaccgatttcacccttaccattagcagcctgcagcctgaggacttcgctacctactactgccagcaggggaacagctttccgctcaccttcggcggaggcactaaggtcgaaatcaag

SEQ ID NO: 107 - DNA, 384 bp, synthetic construct
  source 1..384: mol_type = other DNA
gaggtgcagctgctggaatccgggggcggactggtgcagcccggcggctccctgaggctttcatgtgccgcctccgggtttaccttcggcgactacgccatgtggtgggtcagacaggcgccgggaaagggactggaatgggtgtccgccatttccgcctcgggcgacgtgacctactacgcggattccgtgaagggtcgcttcactatctctcgggacaacagcaaaaacaccctctacttgcaaatgaactcactgcgcgctgaggacaccgcagtgtattactgcgcccggggaagcctcatcggatacagctatgcccgggtgtcgtactactactactccgatgtctggggaaagggtaccactgtcactgtgtcgagc

SEQ ID NO: 108 - DNA, 321 bp, synthetic construct
  source 1..321: mol_type = other DNA
gacatccaaatgacccagtcgccctcatccgtgagcgcctctgtgggcgacagagtgaccatcacgtgtcgggccagccagggcatttcgcggtggctcgcatggtatcagcagaagcctggaaaagccccaaagctgctgatctacgccgctagcagccttcagtccggagtgccgtcccgcttctccgggtcgggatccgggactgatttcaccctgaccatttcctcattgcaacccgaggacttcgcgacctactactgccaacagggctggtccttcccgctgacttttggtggcggaactaaggtcgaaatcaag

SEQ ID NO: 109 - DNA, 378 bp, synthetic construct
  source 1..378: mol_type = other DNA
caagtgcagctgcaggaatcaggtcctgggctggtcaagcccagcgagactctgtctctcacttgcacggtgtccggtggcagcatctcgtcctactattggtcgtggattaggcaaccagccggaaagggactggaatggatcggccggatctacacttcggggagcaccaactacaacccgagcctcaagtcacgcgtcaccatgtccgtggacacctccaagaaccagttctccctgaaactttcgtccgtgactgccgctgataccgcggtgtactactgcgcacgggacctgaccaactacggctccggaaagtccattagattctacggaatggacgtctggggccagggaaccactgtgaccgtgtccagc

SEQ ID NO: 110 - DNA, 318 bp, synthetic construct
  source 1..318: mol_type = other DNA
gatattcaaatgacccagtcaccgtcctccctgtccgcaagcgtcggtgaccgcgtgactattacgtgtcaagccagccaggacatctccaactacctgaactggtatcagcagaagcctggaaaggcgccaaagctgctcatctacgatgcctctaatctggaaaccggagtgccctcgagattcagcggatcaggatccgggaccgactttaccttcaccatctcgtccctgcaaccggaagatattgctacttactactgccagcaggacgacaacttcatcactttcggcggcggcaccaaagtggagatcaag

SEQ ID NO: 111 - DNA, 378 bp, synthetic construct
  source 1..378: mol_type = other DNA
caagtgcagcttcaggagtccggcccaggactggtcaagccgagcgaaactttgtccctgacttgcacggtgtccggcggatcgatcagcagcaattactggcattggattcggcagccggccgggaagggactcgaatggatcggcaggattcacacctccggctccaccttttacaacccttcactgaaatcgcgcgtgaccatgtccgtggatacctccaagaaccagttcagcctgaagctctcctcggtgaccgctgccgacactgcggtgtactattgcgcaagagacatcaccaactacggttcgggaaagtcaatccggttctaccccatggacgtctggggtcaagggactacagtgaccgtgtcctct

SEQ ID NO: 112 - DNA, 318 bp, synthetic construct
  source 1..318: mol_type = other DNA
gatatccaaatgacccagtcgccctcatccctgtccgcctccgtgggtgatcgcgtgaccattacttgccaagccagccaggacatttcgaactacctgaactggtatcagcagaagcctggcaaagccccgaagcttctgatctacgacgcttcaaacctggaaaccggagtgccatcccggttctccggatccgggtcgggaaccgacttcaccttcacaatcagcagcctccagccggaagatatcgcgacttactactgccaacaagacgacacgtttatcaccttcgggggcggcactaaggtcgagattaag

SEQ ID NO: 113 - DNA, 378 bp, synthetic construct
  source 1..378: mol_type = other DNA
caagtgcagctgcaagagagcggtccggggctggtcaagccctcggaaactctctcactgacctgtaccgtgtccggcggctcgatttcctcccactattggcattggatcaggcagccagccgggaagggcttggaatggatcggacggattgctaccagcggttcaacgtactacaaccctagcctgaagtccagagtgacaatgtccgtggacacctccaagaaccagttctctctgaaactctcctccgtgaccgcagccgatactgcggtgtactactgcgcccgggaccttaccaactacggcatcactaagagcatccgcttctacggaatggacgtctggggacagggaaccaccgtgactgtgtcgtcc

SEQ ID NO: 114 - DNA, 318 bp, synthetic construct
  source 1..318: mol_type = other DNA
gacatccaaatgactcagtccccatcaagcctgagcgcctccgtgggggatcgcgtgaccattacgtgtcaggcgtcccaggacattagcaactacctgaactggtaccagcagaagcctgggaaggccccgaagctccttatctacgatgcctcgaatctggaaactggagtgccgtcacggttctcgggatccggctccggcaccgacttcacctttaccatttcgtccctgcaacccgaggacatcgctacctactattgccaacaagtggacaacttcatcaccttcggtggcggaactaaggtcgaaatcaaa

SEQ ID NO: 115 - DNA, 378 bp, synthetic construct
  source 1..378: mol_type = other DNA
caagtgcaactgcaggaatccggaccggggcttgtgaagccctccgagactctgtcactgacttgcactgtctcgggtggcagcatctcctcccactactggcattggatcagacagccagccggaaagggactggaatggattggcaggatccacacctcgggttccaccttctacaaccctagcctcaagtcccgcgtgacgatgagcgtggacacctctaaaaaccagttcagcctcaagttgtcctccgtgaccgctgcggatactgccgtgtactattgtgcccgggacctgaccaactacggctggtccaagtcgattcggttttacggaatggacgtctggggccaggggacaaccgtgaccgtgtcatcg

SEQ ID NO: 116 - DNA, 318 bp, synthetic construct
  source 1..318: mol_type = other DNA
gacattcaaatgacccagagcccatcctcactgtcggcgtccgtgggggatcgggtcactatcacctgtcaagccagccaggacatcagcaactacctcaactggtaccagcagaagcccgggaaggctcctaagctgcttatctatgacgcctccaatctggaaaccggagtgccgtcacgcttctcgggatccggctccggtactgactttaccttcaccatctcgtccctgcaaccggaagatattgccacctactactgccagcagaccgacaacttcattactttcggcggcggaacgaaagtggagatcaag

SEQ ID NO: 117 - DNA, 381 bp, synthetic construct
  source 1..381: mol_type = other DNA
caagtgcagctggtgcagtccggtgccgaagtcaagaagccaggagccagcgtgaaagtgtcctgcaaggcctccggctataccttcactgggtactacatgcactgggtccgccaagcacctggccagggactcgaatggatgggctccattaacccgtcgagcggaggaaccaactacgcgcagaagttccagggcagagtgaccatgactagggacacctcgatctccacggcctacatggagctgtcacggctgcggtcggatgacactgccgtgtactactgcgcgagagtgggaggagctgctgccgctggaaaggcatacaccgcctccgcgttcgatatctggggacaggggaccatggtcactgtgtccagc

SEQ ID NO: 118 - DNA, 324 bp, synthetic construct
  source 1..324: mol_type = other DNA
gaaatcgtgatgacccaatccccggccactctgtcggtgtcaccaggagagcgggccaccttgagctgccgcgctagccagtccgtgggctccaacctcgcgtggtaccagcagaagcccggacaggcacctcgcctcctgatctacggtgcctccactcgggctaccggaatccctgcgagattctcagggtccggttccggcaccgaattcaccctgacgatctcgagcctgcagtccgaggacttcgccgtgtactactgtcaacagttccacaactggcccccgctgacttttggaggagggaccaaggtcgagattaag

SEQ ID NO: 119 - DNA, 381 bp, synthetic construct
  source 1..381: mol_type = other DNA
caagtgcagctcgtgcagtccggggcagaagtcaagaagccgggtgccagcgtgaaagtgtcctgcaaagccagcggttacactttcaccgaccactacatgcattgggtccgccaggcgcccggacagggactggagtggatgggcgctattaacccttcatccggcggaacggcctatgcccaaaagttccaggggagagtgaccatgacccgggacacctcaatctccaccgcctacatggaactgtcgaggctgcggagcgacgatactgcggtgtactactgtgcacgcgtgggcggcgctgcccgggtcggaaaggcctacaccgcctcggcgtttgatatctggggacagggaactatggtcaccgtgtcctcc

SEQ ID NO: 120 - DNA, 324 bp, synthetic construct; identical to SEQ ID NO: 118
  source 1..324: mol_type = other DNA

SEQ ID NO: 121 - DNA, 375 bp, synthetic construct
  source 1..375: mol_type = other DNA
caagtgcagctggtgcaatccggcgccgaggtcaagaagcctggagcctccgtgaaagtgtcctgcaaggcctcgggttacactttcacctcatacgggatttcatgggtcagacaggcaccgggacagggactcgaatggatgggctggatcagcgcctactcgggcgacaccaactacgcccagaagctgcagggtcgggtcacaatgaccaccgacaccagcacgtccaccgcgtacatggaacttaggtccctgcggtccgatgacactgctgtgtactactgtgcgcgcggagatgaggtggtcggaaagatgacctactattacgggatggacgtctggggccagggaactaccgtgactgtgtcgagc

SEQ ID NO: 122 - DNA, 321 bp, synthetic construct
  source 1..321: mol_type = other DNA
gacattcaaatgactcagtccccgtcctccgtgtcggcttccgtgggcgacagggtcacgatcacctgtcgcgcctcccaagggatcagcagatggctggcctggtaccagcagaagccagggaaagcccccaagctgcttatctatgccgcgagctcactgcagtccggagtgcctagccggttctccggctccggatcaggaaccgactttaccctcaccatctcgagcctgcagcccgaagatttcgccacttactactgccaacaagcaaactcgttcccgatcactttcggtggaggcaccaaggtcgagattaag

SEQ ID NO: 123 - DNA, 375 bp, synthetic construct
  source 1..375: mol_type = other DNA
caagtgcaactggtgcagtccggtgccgaggtcaagaagcccggagcgtccgtgaaagtgtcctgcaaggcctcgggatacactttcaccgactacgcgattagctgggtcagacaggccccgggacagggcctggagtggatgggctggatctccgcatcaaccgccgacactaactacgcccagaagctccagggtcgcgtgactatgaccaccgacacaagcacttccaccgcctacatggaactgcggtcccttcggagcgacgataccgctgtgtactattgtgcccgcggcgacgaagtggtcggaaagatgacctactactacgggatggatgtctggggccaggggaccaccgtgacggtgtcatcg

SEQ ID NO: 124 - DNA, 321 bp, synthetic construct; identical to SEQ ID NO: 122
  source 1..321: mol_type = other DNA

SEQ ID NO: 125 - DNA, 375 bp, synthetic construct
  source 1..375: mol_type = other DNA
caagtgcagctcgtgcagtcgggcgctgaagtgaaaaagcccggagcctccgtgaaagtgtcctgcaaggcctccgggtacactttcaccgactacgcaatcagctgggtcagacaggccccgggccagggtcttgagtggatgggttggatctccgcctcaaccgccgacaccaactacgcgcaaaagctgcagggaagggtcaccatgaccaccgacacgtccacatcaactgcgtacatggagctgcgctcgctgcggagcgatgacaccgccgtgtactattgtgcccggggcgatgaagtggtcggaaagcgcacttactacattggcatggacgtctggggacaggggactaccgtgaccgtcagctcc

SEQ ID NO: 126 - DNA, 321 bp, synthetic construct; identical to SEQ ID NO: 122
  source 1..321: mol_type = other DNA

SEQ ID NO: 127 - DNA, 375 bp, synthetic construct
  source 1..375: mol_type = other DNA
caagtgcagctcgtgcagtccggcgccgaagtcaagaagcccggtgcatcagtgaaagtcagctgcaaggcctcagggtacacattcacggactacgcgattagctgggtcagacaggctccgggccaaggtcttgagtggatgggctggatctcggcctccaccgccgataccaactacgcccagaagctgcagggccgcgtgaccatgactaccgatacctccacctcgactgcctacatggagctgcggtccctgcggagcgacgacactgccgtgtattactgtgcgaggggaaccgaagtcgtgggaaagatgaccgtgtacctggggatggacgtctggggacagggaaccactgtgactgtgtcctcc

SEQ ID NO: 128 - DNA, 321 bp, synthetic construct; identical to SEQ ID NO: 122
  source 1..321: mol_type = other DNA

SEQ ID NO: 129 - DNA, 375 bp, synthetic construct
  source 1..375: mol_type = other DNA
caagtgcaactcgtgcagtccggagcagaagtcaagaagcccggggcttccgtgaaagtgtcctgcaaggcctccggctacaccttcactgactacgcgatttcctgggtccgccaggccccgggccagggtctggagtggatgggctggatcagcgcctcgaccgccgacactaactatgcccaaaagctgcagggtcgcgtgacaatgaccactgacacctccacgagcaccgcctacatggagcttaggtcactgcggagcgatgacaccgccgtgtactactgtgcgagacaggatgaagtcgtggggaagatgacctactacatcggaaccgacgtctggggacagggaactactgtgaccgtgtcgtca

SEQ ID NO: 130 - DNA, 321 bp, synthetic construct
  source 1..321: mol_type = other DNA
gacattcaaatgacccagagcccctcgtccgtgtctgcctcggtcggcgaccgcgtgactatcacttgccgggctagccagggaatctccagatggcttgcctggtatcagcagaagcctgggaaggccccaaaactgttgatctacgccgcctcatccctccaatccggagtgccgtcaaggttctccggaagcggaagcggcactgattttacgctgaccatttcctccctgcaacccgaggacttcgcgacctactactgtcagcaagcaaactcgttcccgctgaccttcggtggcgggaccaaggtcgaaatcaag

SEQ ID NO: 131 - DNA, 375 bp, synthetic construct
  source 1..375: mol_type = other DNA
caagtgcaactggtgcagtcgggtgccgaagtcaagaagcccggggcgtccgtgaaagtgtcctgcaaggcctccggctacaccttcaccgattacgccattagctgggtccgccaggccccgggacagggcctggagtggatgggatggatctcggctggaacggccgacactaactacgcccagaagctccagggcagagtgaccatgaccaccgacacttccacttcaaccgcctacatggaacttcggtccctgaggtccgacgatacagcggtgtactactgtgcacggggcgacgaggtcgtggggaagatgacctactactatgatatggacgtctggggacagggtaccaccgtgactgtgtccagc

SEQ ID NO: 132 - DNA, 321 bp, synthetic construct
  source 1..321: mol_type = other DNA
gacattcaaatgatccagtcgcctagctccgtgtccgcctccgtgggggaccgcgtgactattacctgtcgggcctcacaaggcatctccagatggcttgcctggtaccagcagaagcccggaaaggccccgaaactcctgatctatgcggctagcagcctgcagtccggagtgccctcccggttctccggctcgggatccgggaccgatttcaccctgactatttcgagcctgcagccagaggacttcgcaacctactactgccaacaggcctcatctttcccgatcacctttggtggcggaactaaggtcgaaatcaag

SEQ ID NO: 133 - DNA, 375 bp, synthetic construct
  source 1..375: mol_type = other DNA
caagtgcaactggtgcagtccggcgcagaggtcaagaagcccggagcctccgtgaaagtgtcgtgcaaagccagcggatacactttcgacgactacgggatctcatgggtcagacaggcgccgggacagggcctggagtggatgggttggattagcgccaccgtggctgacactaactacgcccagaagctccagggccgcgtgaccatgacgaccgataccagcacttccaccgcctacatggaacttaggtccctgcggtcggatgatacagccgtgtactactgtgcgcggggcgacgaacctgtcgggaagatgacctactactatggaatggacgtctggggacagggtaccaccgtgactgtgtcctcc

SEQ ID NO: 134 - DNA, 321 bp, synthetic construct
  source 1..321: mol_type = other DNA
gatatccaaatgacccagtccccaagctccgtgtcggcctccgtgggagacagagtgaccatcacttgtcgggccagccagggcatttcccgctggttggcttggtatcagcagaagcccgggaaggccccgaagctgctcatctacgcggccagctcactgcagtcgggagtcccgtcaaggttctctggatccggatcgggcactgacttcaccctgacgatttcctccctgcaacccgaggactttgcaacttactactgccaacaggccaacagccttcctatcaccttcggtggcgggaccaaagtggaaatcaag

SEQ ID NO: 135 - DNA, 384 bp, synthetic construct
  source 1..384: mol_type = other DNA
caagtgcagctggtgcagtcaggggcagaagtcaagaagcctggagccagcgtgaaagtgtcctgcaaggcctcgggttatactttcaccgggtactacatgcattgggtcagacaggccccaggacagggactggagtggatgggctcgatcaacccgagctcaggaggcaccaactacgcgcagaagtttcagggccgcgtcaccatgacgcgcgacacctccatctccactgcctacatggagctctcccggctgagaagcgacgatactgcggtgtactactgcgctagggaccccgtgctgctgcttcggcaacagcggattctccacgaatcggccttcgatatctggggacaaggcaccactgtgaccgtgtcctcc

SEQ ID NO: 136 - DNA, 324 bp, synthetic construct; identical to SEQ ID NO: 118
  source 1..324: mol_type = other DNA

SEQ ID NO: 137 - DNA, 384 bp, synthetic construct
  source 1..384: mol_type = other DNA
caggtccaactcgtgcagtcgggagccgaagtcaagaagcccggagcatccgtgaaagtgtcctgcaaggcgtccggctacacctttgaggggcactatatgcactgggtcagacaggccccaggtcaaggcctcgaatggatgggggccatcaacccgtactcaggacagaccaactacgcgcagaagttccagggtcgcgtgacgatgacccgggacactagcatctcgaccgcctacatggaactgtccagattgcgctccgatgacactgccgtgtactactgtgctcgggattttgtgcttctgctgaggcagagccggatcctgcatgagagcgccttcgacatttggggccagggaactaccgtgaccgtgtccagc

SEQ ID NO: 138 - DNA, 324 bp, synthetic construct; identical to SEQ ID NO: 118
  source 1..324: mol_type = other DNA

SEQ ID NO: 139 - protein, 7 aa, synthetic construct
RPKPVGX

SEQ ID NO: 140 - protein, 4 aa, synthetic construct
AAPV

(19) United States
(12) Patent Application Publication
(10) Pub. No.: US 2026/0274991 A1
(43) Pub. Date: Sep. 17, 2026

(54) DUAL INHIBITOR TRYPSIN ANTIBODIES AND USES THEREOF

(71) Applicant: Triveni Bio, Inc., Watertown, MA (US)

(72) Inventors: Jennifer L. Dovey, Auburndale, MA (US); Allan Capili, Somerville, MA (US); Brantley Herrin, Watertown, MA (US); Jason Hon, Watertown, MA (US); Ye Tang, Winchester, MA (US); Sonya Entova, Arlington, MA (US); Todd Borland, Mansfield, MA (US); Eva Asp, Watertown, MA (US); Jia Li, Watertown, MA (US); Libin Lui, Watertown, MA (US); Leonard Zon, Brookline, MA (US); Bradley Lunde, Lebanon, NH (US)

(73) Assignee: Triveni Bio, Inc., Watertown, MA (US)

(21) Appl. No.: 19/474,490

(22) PCT Filed: Apr. 12, 2024
(86) PCT No.: PCT/US2024/024370

§ 371 Date: Oct. 10, 2025

Related U.S. Application Data
(60) Provisional application No. 63/459,593, filed on Apr. 14, 2023.

(51) Int. Cl. C07K 16/40; A61K 39/00; A61P 1/18; C12N 9/76

(52) U.S. Cl. CPC C07K 16/40; A61P 1/18; C12N 9/6427; A61K 2039/505; C07K 2317/14; C07K 2317/31; C07K 2317/565; C07K 2317/76; C07K 2317/92; C07K 2319/21

(57) ABSTRACT

Aspects of the application provide dual inhibitor anti-trypsin 1/trypsin 2 antibodies and methods of using the same for treating pancreatitis.

### RELATED APPLICATIONS

This application claims priority under 35 U.S.C. § 119(e) to U.S. Provisional Application, U.S. Ser. No. 63/459,593, filed Apr. 14, 2023, the entire contents of which are incorporated herein by reference.

### REFERENCE TO AN ELECTRONIC SEQUENCE LISTING

The contents of the electronic sequence listing (A140770002W000-SEQ-LJG.xml; Size: 152,469 bytes; and Date of Creation: Apr. 12, 2024) is herein incorporated by reference in its entirety.

### BACKGROUND

Pancreatitis is an inflammatory disease of the pancreas, which can be an acute or chronic condition. In certain cases, pancreatitis can be associated with genetic abnormalities that may be inherited or spontaneously arising. However, there are other causes, including heavy alcohol use, gallstones, treatment with certain medicines, and still others. Effective treatment options remain limited, and consequently, pancreatitis can result in significant morbidity and mortality.

### SUMMARY

Certain aspects of the disclosure relate to a recognition that activation of trypsin (e.g., trypsin 1 and trypsin 2) in the pancreas is associated with the development of pancreatitis. In some embodiments, methods and related compositions are provided that are useful for inhibition of trypsin 1 and trypsin 2 for purposes of treating pancreatitis. In particular, aspects of the disclosure provide dual inhibitor antibodies targeting trypsin 1 and trypsin 2 (referred to as anti-trypsin 1/trypsin 2 antibodies) that have high binding affinity and specificity to both trypsin 1 and trypsin 2 via a common distinct antigen-specific binding site. Accordingly, in some embodiments, the disclosure provides methods and related antibody compositions for treating conditions associated with trypsin 1 and trypsin 2 dysregulation, such as hereditary pancreatitis, acute pancreatitis, recurrent acute pancreatitis, or chronic pancreatitis.

### BRIEF DESCRIPTION OF THE DRAWINGS

The accompanying drawings, which are incorporated in and constitute a part of this specification, illustrate certain embodiments, and together with the written description, serve to provide non-limiting examples of certain aspects of the compositions and methods disclosed herein.

FIG. 1 is a graph showing that anti-trypsin 1/trypsin 2 antibodies R1, R2, and R3 reduced acinar cell degeneration compared to IgG control in rats having caerulein-induced pancreatitis.

FIG. 2 is a graph showing that anti-trypsin 1/trypsin 2 antibodies R1, R2, and R3 reduced edema compared to IgG control in rats having caerulein-induced pancreatitis.

### DETAILED DESCRIPTION

As discussed herein, activation of trypsin (e.g., trypsin 1 and trypsin 2) in the pancreas is associated with the development of pancreatitis. Trypsin enzymes are synthesized as trypsinogen, an inactive precursor, in the rough endoplasmic reticulum and transported to the Golgi apparatus for sorting in the pancreas. Trypsinogen is co-synthesized and packed with a pancreatic secretory trypsin inhibitor (PSTI) that inhibits its premature activation. These zymogen granules are released into the lumen of pancreatic duct, which carries the digestive enzymes into the duodenum. Once in duodenum, enteropeptidase activates trypsinogen into active trypsin. Trypsin 1 is the most abundant trypsin enzyme secreted by the pancreas followed by trypsin 2. Accordingly, the present disclosure, at least in part, is based on the development of dual inhibitor antibodies and variants thereof targeting trypsin 1 and trypsin 2. These dual inhibitor antibodies target trypsin 1 and trypsin 2 via a common distinct antigen-specific binding site. Such dual inhibitor antibodies have high binding affinity and specificity to trypsin 1 and trypsin 2 (anti-trypsin 1/trypsin 2 antibodies). Also provided are methods of using the anti-trypsin 1/trypsin 2 antibodies and their variants in research, diagnostic/detection, and therapeutic applications.

### I. Definitions

Administering: As used herein, the terms “administering” or “administration” mean to provide an antibody or a composition thereof to a subject in a manner that is physiologically and/or pharmacologically useful (e.g., to treat a condition in the subject).

Affinity Matured Antibody: The term “Affinity Matured Antibody” is used herein to refer to an antibody with one or more alterations in one or more CDRs, which result in an improvement in the affinity (e.g., KD, kd or ka) of the antibody for a target antigen compared to a parent antibody, which does not possess the alteration(s). Exemplary affinity matured antibodies may have nanomolar or even picomolar affinities for the target antigen in some embodiments. A variety of procedures for producing affinity matured antibodies are available, including the screening of a combinatory antibody library that has been prepared using bio-display. For example, Marks et al., BioTechnology, 10: 779-783 (1992) describes affinity maturation by VH and VL domain shuffling. Random mutagenesis of CDR and/or framework residues is described by Barbas et al., Proc. Nat. Acad. Sci. USA, 91: 3809-3813 (1994); Schier et al., Gene, 169: 147-155 (1995); Yelton et al., J. Immunol., 155: 1994-2004 (1995); Jackson et al., J. Immunol., 154(7): 3310-3319 (1995); and Hawkins et al, J. Mol. Biol., 226: 889-896 (1992). Selective mutation at selective mutagenesis positions and at contact or hypermutation positions with an activity-enhancing amino acid residue is described in U.S. Pat. No. 6,914,128 Bi.

Antibody: As used herein, the term “antibody” refers to a polypeptide that comprises at least one immunoglobulin variable domain, which comprises at least one distinct antigen-specific binding site, or a portion of an immunoglobulin variable domain (such as a paratope or portion thereof) that comprises at least one distinct antigen-specific binding site. In some embodiments, an antibody is a full-length antibody. In some embodiments, an antibody is a chimeric antibody. In some embodiments, an antibody is a humanized antibody. However, in some embodiments, an antibody is a Fab fragment, a F(ab′)2 fragment, a Fv fragment or a scFv fragment. In some embodiments, an antibody is a multi-specific antibody (e.g., a bispecific antibody). In some embodiments, an antibody is a nanobody derived from a camelid antibody or a nanobody derived from shark antibody. In some embodiments, an antibody is a diabody. In some embodiments, an antibody comprises a framework having a human germline sequence. In another embodiment, an antibody comprises a heavy chain constant domain selected from the group consisting of IgG, IgG1, IgG2, IgG2A, IgG2B, IgG2C, IgG3, IgG4, IgA1, IgA2, IgD, IgM, and IgE constant domains. In some embodiments, an antibody comprises a heavy (H) chain variable region (abbreviated herein as VH), and/or a light (L) chain variable region (abbreviated herein as VL). In some embodiments, an antibody comprises a constant domain, e.g., an Fc region. An immunoglobulin constant domain refers to a heavy or light chain constant domain. Human IgG heavy chain and light chain constant domain amino acid sequences and their functional variations are known. With respect to the heavy chain, in some embodiments, the heavy chain of an antibody described herein can be an alpha (α), delta (Δ), epsilon (ε), gamma (γ) or mu (μ) heavy chain. In some embodiments, the heavy chain of an antibody described herein can comprise a human alpha (α), delta (Δ), epsilon (c), gamma (γ) or mu (μ) heavy chain. In a particular embodiment, an antibody described herein comprises a human gamma 1 CH1, CH2, and/or CH3 domain. In some embodiments, the amino acid sequence of the VH domain comprises the amino acid sequence of a human gamma (γ) heavy chain constant region, such as any known in the art. Non-limiting examples of human constant region sequences have been described in the art, e.g., see U.S. Pat. No. 5,693,780 and Kabat E A et al., (1991) supra. In some embodiments, the VH domain comprises an amino acid sequence that is at least 70%, 75%, 80%, 85%, 90%, 95%, 98%, or at least 99% identical to any of the variable chain constant regions provided herein. In some embodiments, an antibody is modified, e.g., modified via glycosylation, phosphorylation, sumoylation, and/or methylation. In some embodiments, an antibody is a glycosylated antibody, which is conjugated to one or more sugar or carbohydrate molecules. In some embodiments, the one or more sugar or carbohydrate molecule are conjugated to the antibody via N-glycosylation, O-glycosylation, C-glycosylation, glypiation (GPI anchor attachment), and/or phosphoglycosylation. In some embodiments, the one or more sugar or carbohydrate molecule are monosaccharides, disaccharides, oligosaccharides, or glycans. In some embodiments, the one or more sugar or carbohydrate molecule is a branched oligosaccharide or a branched glycan. In some embodiments, the one or more sugar or carbohydrate molecule includes a mannose unit, a glucose unit, an N-acetylglucosamine unit, or a phospholipid unit. In some embodiments, an antibody is a construct that comprises a polypeptide comprising one or more antigen binding fragments of the disclosure linked to a linker polypeptide or an immunoglobulin constant domain. Linker polypeptides comprise two or more amino acid residues joined by peptide bonds and are used to link one or more antigen binding portions. Examples of linker polypeptides have been reported (see e.g., Holliger, P., et al. (1993) Proc. Natl. Acad. Sci. USA 90:6444-6448; Poljak, R. J., et al. (1994) Structure 2:1121-1123). Still further, an antibody may be part of a larger immunoadhesion molecule, formed by covalent or noncovalent association of the antibody or antibody portion with one or more other proteins or peptides. Examples of such immunoadhesion molecules include use of the streptavidin core region to make a tetrameric scFv molecule (Kipriyanov, S. M., et al. (1995) Human Antibodies and Hybridomas 6:93-101) and use of a cysteine residue, a marker peptide and a C-terminal polyhistidine tag to make bivalent and biotinylated scFv molecules (Kipriyanov, S. M., et al. (1994) Mol. Immunol. 31:1047-1058).

Approximately: As used herein, the term “approximately” or “about,” as applied to one or more values of interest, refers to a value that is similar to a stated reference value. In certain embodiments, the term “approximately” or “about” refers to a range of values that fall within 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Bispecific Antibody: As used herein, the term “bispecific antibody” refers to an antibody that comprises two distinct antigen-specific binding sites or two linked (covalently or non-covalently) antibodies that, combined, comprise two distinct antigen-specific binding sites. Non-limiting examples of bispecific antibody formats or architectures are provided in Labrijn, A F, et al., Bispecific antibodies: a mechanistic review of the pipeline, Nature Reviews Drug Discovery volume 18, pages 585-608 (2019) and Brinkmann U and Kontermann E E, The making of bispecific antibodies, MAbs. 2017 February/March; 9(2):182-212, the entire contents of each of which are incorporated herein by reference in their entireties.

CDR: As used herein, the term “CDR” refers to the complementarity determining region within antibody variable sequences. A typical antibody molecule comprises a heavy chain variable region (VH) and a light chain variable region (VL), which are usually involved in antigen binding. The VH and VL regions can be further subdivided into regions of hypervariability, also known as “complementarity determining regions” (“CDR”), interspersed with regions that are more conserved, which are known as “framework regions” (“FR”). Each VH and VL is typically composed of three CDRs and four FRs, arranged from amino-terminus to carboxy-terminus in the following order: FR1, CDR1, FR2, CDR2, FR3, CDR3, FR4. The extent of the framework region and CDRs can be precisely identified using methodology known in the art, for example, by the Kabat definition, the IMGT definition, the Chothia definition, the AbM definition, and/or the contact definition, all of which are well known in the art. See, e.g., Kabat, E. A., et al. (1991) Sequences of Proteins of Immunological Interest, Fifth Edition, U.S. Department of Health and Human Services, NIH Publication No. 91-3242; IMGT®, the international ImMunoGeneTics information System®, Lefranc, M.-P. et al., Nucleic Acids Res., 27:209-212 (1999); Ruiz, M. et al., Nucleic Acids Res., 28:219-221 (2000); Lefranc, M.-P., Nucleic Acids Res., 29:207-209 (2001); Lefranc, M.-P., Nucleic Acids Res., 31:307-310 (2003); Lefranc, M.-P. et al., In Silico Biol., 5, 0006 (2004) Epub, 5:45-60 (2005); Lefranc, M.-P. et al., Nucleic Acids Res., 33:D593-597 (2005); Lefranc, M.-P. et al., Nucleic Acids Res., 37:D1006-1012 (2009); Lefranc, M.-P. et al., Nucleic Acids Res., 43:D413-422 (2015); Chothia et al., (1989) Nature 342:877; Chothia, C. et al. (1987) J. Mol. Biol. 196:901-917, Al-lazikani et al (1997) J. Molec. Biol. 273:927-948; and Almagro, J. Mol. Recognit. 17:132-143 (2004). ee also hgmp.mrc.ac.uk and bioinf.org.uk/abs. As used herein, a CDR may refer to the CDR defined by any method known in the art. Two antibodies having the same CDR means that the two antibodies have the same amino acid sequence of that CDR as determined by the same method, for example, the IMGT definition.

In certain embodiments, there are three CDRs in each of the variable regions of a heavy chain and a light chain, which are designated CDR1, CDR2 and CDR3, for each of the variable regions. The term “CDR set” as used herein refers to a group of three CDRs that occur in a single variable region capable of binding the antigen. The exact boundaries of these CDRs have been defined differently according to different systems. The system described by Kabat (Kabat et al., Sequences of Proteins of Immunological Interest (National Institutes of Health, Bethesda, Md. (1987) and (1991)) not only provides an unambiguous residue numbering system applicable to any variable region of an antibody, but also provides precise residue boundaries defining the three CDRs. These CDRs may be referred to as Kabat CDRs. Sub-portions of CDRs may be designated as L1, L2 and L3 or H1, H2 and H3 where the “L” and the “H” designates the light chain and the heavy chains regions, respectively. These regions may be referred to as Chothia CDRs, which have boundaries that overlap with Kabat CDRs. Other boundaries defining CDRs overlapping with the Kabat CDRs have been described by Padlan (FASEB J. 9:133-139 (1995)) and MacCallum (J Mol Biol 262(5):732-45 (1996)). Still other CDR boundary definitions may not strictly follow one of the above systems, but will nonetheless overlap with the Kabat CDRs, although they may be shortened or lengthened in light of prediction or experimental findings that particular residues or groups of residues or even entire CDRs do not significantly impact antigen binding. The methods used herein may utilize CDRs defined according to any of these systems, although preferred embodiments use Kabat or Chothia defined CDRs.

In certain embodiments, the CDRs of an antibody may have different amino acid sequences when different definition systems are used (e.g., the IMGT definition, the Kabat definition, the Chothia definition, or any other known definition). A definition system annotates each amino acid in a given antibody sequence (e.g., VH or VL sequence) with a number, and numbers corresponding to the heavy chain and light chain CDRs are provided in Table 2. One skilled in the art is able to derive the CDR sequences using the different numbering systems for the anti-trypsin 1/trypsin 2 antibodies provided in Tables 1a and 1b.

TABLE 2
CDR Definitions

| | IMGT¹ | Kabat² | Chothia³ |
|---|---|---|---|
| HC CDR1 | 27-38 | 31-35 | 26-32 |
| HC CDR2 | 56-65 | 50-65 | 53-55 |
| HC CDR3 | 105-116/117 | 95-102 | 96-101 |
| LC CDR1 | 27-38 | 24-34 | 26-32 |
| LC CDR2 | 56-65 | 50-56 | 50-52 |
| LC CDR3 | 105-116/117 | 89-97 | 91-96 |

¹IMGT ®, the international ImMunoGeneTics information system ®, imgt.org, Lefranc, M.-P. et al., Nucleic Acids Res., 27: 209-212 (1999)
²Kabat et al. (1991) Sequences of Proteins of Immunological Interest, Fifth Edition, U.S. Department of Health and Human Services, NIH Publication No. 91-3242
³Chothia et al., J. Mol. Biol. 196: 901-917 (1987))

CDR-grafted antibody: As used herein, the term “CDR-grafted antibody” refers to antibodies which comprise heavy and light chain variable region sequences from one species but in which the sequences of one or more of the CDR regions of VH and/or VL are replaced with CDR sequences of another species, such as antibodies having murine heavy and light chain variable regions in which one or more of the murine CDRs (e.g., CDR3) has been replaced with human CDR sequences.

Chimeric antibody: As used herein, the term “chimeric antibody” refers to antibodies which comprise heavy and light chain variable region sequences from one species and constant region sequences from another species, such as antibodies having murine heavy and light chain variable regions linked to human constant regions.

Complementary: As used herein, the term “complementary” refers to the capacity for precise pairing between two nucleotides or two sets of nucleotides. In particular, complementary is a term that characterizes an extent of hydrogen bond pairing that brings about binding between two nucleotides or two sets of nucleotides. For example, if a base at one position of an oligonucleotide is capable of hydrogen bonding with a base at the corresponding position of a target nucleic acid (e.g., an mRNA), then the bases are considered to be complementary to each other at that position. Base pairings may include both canonical Watson-Crick base pairing and non-Watson-Crick base pairing (e.g., Wobble base pairing and Hoogsteen base pairing). For example, in some embodiments, for complementary base pairings, adenosine-type bases (A) are complementary to thymidine-type bases (T) or uracil-type bases (U), that cytosine-type bases (C) are complementary to guanosine-type bases (G), and that universal bases such as 3-nitropyrrole or 5-nitroindole can hybridize to and are considered complementary to any A, C, U, or T. Inosine (I) has also been considered in the art to be a universal base and is considered complementary to any A, C, U or T.

Conservative amino acid substitution: As used herein, a “conservative amino acid substitution” refers to an amino acid substitution that does not alter the relative charge or size characteristics of the protein in which the amino acid substitution is made. Variants can be prepared according to methods for altering polypeptide sequence known to one of ordinary skill in the art such as are found in references which compile such methods, e.g. Molecular Cloning: A Laboratory Manual, J. Sambrook, et al., eds., Fourth Edition, Cold Spring Harbor Laboratory Press, Cold Spring Harbor, New York, 2012, or Current Protocols in Molecular Biology, F. M. Ausubel, et al., eds., John Wiley & Sons, Inc., New York. Conservative substitutions of amino acids include substitutions made amongst amino acids within the following groups: (a) M, I, L, V; (b) F, Y, W; (c) K, R, H; (d) A, G; (e) S, T; (f) Q, N; and (g) E, D.

Cross-reactive: As used herein, the term “cross-reactive,” refers to a property of the agent being capable of specifically binding to more than one antigen of a similar type or class (e.g., antigens of multiple homologs, paralogs, or orthologs) with similar affinity or avidity. For example, in some embodiments, an antibody that is cross-reactive against human and non-human primate antigens of a similar type or class (e.g., a human trypsin 1 and non-human primate trypsin 1, a human trypsin 2 and non-human primate trypsin 2) is capable of binding to the human antigen and non-human primate antigens with a similar affinity or avidity. In some embodiments, an antibody is cross-reactive against a human antigen and a rodent antigen of a similar type or class. In some embodiments, an antibody is cross-reactive against a rodent antigen and a non-human primate antigen of a similar type or class. In some embodiments, an antibody is cross-reactive against a human antigen, a non-human primate antigen, and a rodent antigen of a similar type or class.

Dual Inhibitor Antibody: As used herein, the term “dual inhibitor antibody” refers to an antibody that targets at least two (e.g., two, three) different antigens via a common distinct antigen-specific binding site and inhibits activity of those antigens. In some embodiments, a dual inhibitor antibody targets at least two different proteins (e.g., expressed from two different genes (e.g., endogenous genes, e.g., homologues, paralogues) via a common distinct antigen-specific binding site and inhibits activity of the at least two different proteins (e.g., enzymes, such as proteases). In some embodiments, a dual inhibitor antibody targets at least two different proteases (e.g., expressed by two different endogenous genes, e.g., trypsin 1 and trypsin 2) via a common distinct antigen-specific binding site and inhibits activity of the at least two different proteases. In some embodiments, the common distinct antigen-specific binding site binds to a similar (e.g., homologous) domain shared between or among the at least two different antigens. For example, in some embodiments, the common distinct antigen-specific binding site binds to a similar (e.g., homologous) catalytic domain or substrate binding site shared between or among the at least two different enzymes, e.g., proteases. In some embodiments, the common distinct antigen-specific binding site of a dual inhibitor antibody comprises amino acids of one or more complementarity determining regions of the antibody. In some embodiments, the common distinct antigen-specific binding site of a dual inhibitor antibody is within a heavy chain variable region and/or a light chain variable region of the antibody. In some embodiments, the common distinct antigen-specific binding site of a dual inhibitor antibody comprises one or more complementarity determining regions of a heavy chain variable region and/or a light chain variable region of the antibody. In some embodiments, the common distinct antigen-specific binding site of a dual inhibitor antibody comprises HC CDR1, HC CDR2, HC CDR3, LC CDR1, LC CDR2, and LC CDR3 of a heavy chain variable region and a light chain variable region of the antibody.

Effective Amount: As used herein, “an effective amount” refers to the amount of each active agent (e.g., anti-trypsin 1/trypsin 2 antibody) required to confer a desired effect (e.g., a therapeutic effect on the subject), either alone or in combination with one or more other active agents. In some embodiments, the therapeutic effect is reduced trypsin 1 and/or trypsin 2 activity and/or alleviated disease (e.g., pancreatitis) or related symptoms, e.g., improved barrier function.

Framework: As used herein, the term “framework” or “framework sequence” refers to the remaining sequences of a variable region minus the CDRs. Because the exact definition of a CDR sequence can be determined by different systems, the meaning of a framework sequence is subject to correspondingly different interpretations. The six CDRs (CDR-L1, CDR-L2, and CDR-L3 of light chain and CDR-H1, CDR-H2, and CDR-H3 of heavy chain) also divide the framework regions on the light chain and the heavy chain into four sub-regions (FR1, FR2, FR3 and FR4) on each chain, in which CDR1 is positioned between FR1 and FR2, CDR2 between FR2 and FR3, and CDR3 between FR3 and FR4. Without specifying the particular sub-regions as FR1, FR2, FR3 or FR4, a framework region, as referred by others, represents the combined FRs within the variable region of a single, naturally occurring immunoglobulin chain. As used herein, a FR represents one of the four sub-regions, and FRs represents two or more of the four sub-regions constituting a framework region. Human heavy chain and light chain acceptor sequences are known in the art. In one embodiment, the acceptor sequences known in the art may be used in the antibodies disclosed herein.

Human antibody: The term “human antibody”, as used herein, is intended to include antibodies having variable and constant regions derived from human germline immunoglobulin sequences. The human antibodies of the disclosure may include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody”, as used herein, is not intended to include antibodies in which CDR sequences derived from the germline of another mammalian species, such as a mouse, have been grafted onto human framework sequences.

Humanized antibody: As used herein, the term “humanized antibody” refers to antibodies which comprise heavy and light chain variable region sequences from a non-human species (e.g., a mouse) but in which at least a portion of the VH and/or VL sequence has been altered to be more “human-like”, i.e., more similar to human germline variable sequences. One type of humanized antibody is a CDR-grafted antibody, in which human CDR sequences are introduced into non-human VH and VL sequences to replace the corresponding nonhuman CDR sequences. In one embodiment, humanized antibodies are provided. Such antibodies may be generated by obtaining murine monoclonal antibodies using traditional hybridoma technology followed by humanization using in vitro genetic engineering, such as those disclosed in Kasaian et al PCT publication No. WO 2005/123126 A2.

Humanized antibodies are human immunoglobulins (recipient antibody) in which residues from a complementary determining region (CDR) of the recipient are replaced by residues from a CDR of a non-human species (donor antibody) such as mouse, rat, or rabbit having the desired specificity, affinity, and capacity. In some embodiments, Fv framework region (FR) residues of the human immunoglobulin are replaced by corresponding non-human residues. Furthermore, the humanized antibody may comprise residues that are found neither in the recipient antibody nor in the imported CDR or framework sequences, but are included to further refine and optimize antibody performance. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region or domain (Fc), typically that of a human immunoglobulin. Antibodies may have Fc regions modified as described in WO 99/58572. Other forms of humanized antibodies have one or more CDRs (one, two, three, four, five, six) which are altered with respect to the original antibody, which are also termed one or more CDRs derived from one or more CDRs from the original antibody. Humanized antibodies may also involve affinity maturation.

In some embodiments, humanization is achieved by grafting the CDRs (e.g., as shown in Tables 1a, or 1b) into the human variable domains (e.g., IGKV1-NL1*01 and IGHV1-3*01 human variable domain). In some embodiments, an antibody of the present disclosure is a humanized variant comprising one or more amino acid substitutions (e.g., in the VH framework region) as compared with any one of the VHs listed in Tables 1a, or 1b and/or one or more amino acid substitutions (e.g., in the VL framework region) as compared with any one of the VLs listed in Tables 1a, or 1b.

Isolated antibody: An “isolated antibody”, as used herein, is intended to refer to an antibody that is substantially free of other antibodies having different antigenic specificities (e.g., an isolated dual inhibitor antibody that specifically binds trypsin 1 and trypsin 2 is substantially free of antibodies that specifically bind antigens other than trypsin 1 and trypsin 2). An isolated antibody may, however, have cross-reactivity to other antigens, in some embodiments. Moreover, an isolated antibody may be substantially free of other cellular material and/or chemicals.

Kabat numbering: As used herein, the terms “Kabat numbering”, “Kabat definitions and “Kabat labeling” are used interchangeably herein. These terms, which are recognized in the art, refer to a system of numbering amino acid residues which are more variable (i.e. hypervariable) than other amino acid residues in the heavy and light chain variable regions of an antibody, or an antigen binding portion thereof (Kabat et al. (1971) Ann. NY Acad, Sci. 190:382-391 and, Kabat, E. A., et al. (1991) Sequences of Proteins of Immunological Interest, Fifth Edition, U.S. Department of Health and Human Services, NIH Publication No. 91-3242). For the heavy chain variable region, the hypervariable region ranges from amino acid positions 31 to 35 for CDR1, amino acid positions 50 to 65 for CDR2, and amino acid positions 95 to 102 for CDR3. For the light chain variable region, the hypervariable region ranges from amino acid positions 24 to 34 for CDR1, amino acid positions 50 to 56 for CDR2, and amino acid positions 89 to 97 for CDR3.

Multi-Specific Antibody: As used herein, the term “multi-specific antibody” refers to an antibody that comprises at least two distinct antigen-specific binding sites or at least two linked (covalently or non-covalently) antibodies that, combined, comprise at least two distinct antigen-specific binding sites. In some embodiments, a multi-specific antibody is a bispecific antibody. Non-limiting examples of multi-specific specific antibody formats or architectures are provided in Sawant M S, et al., Toward Drug-Like Multispecific Antibodies by Design, Int J Mol Sci. 2020 Oct. 12; 21(20):7496; Klein C, et al., The use of CrossMAb technology for the generation of bi-and multispecific antibodies, MAbs 2016 August-September; 8(6):1010-20; and Brinkmann U and Kontermann E E, The making of bispecific antibodies, MAbs. 2017 February/March; 9(2):182-212, the entire contents of each of which are incorporated herein by reference in their entireties.

Recombinant antibody: As used herein, the term “recombinant antibody” is intended to include all antibodies that are prepared, expressed, created or isolated by recombinant means, such as antibodies expressed using a recombinant expression vector transfected into a host cell (described in more details in this disclosure), including, for example, antibodies isolated from a recombinant, combinatorial human antibody library (Hoogenboom H. R., (1997) TIB Tech. 15:62-70; Azzazy H., and Highsmith W. E., (2002) Clin. Biochem. 35:425-445; Gavilondo J. V., and Larrick J. W. (2002) BioTechniques 29:128-145; Hoogenboom H., and Chames P. (2000) Immunology Today 21:371-378), antibodies isolated from an animal (e.g., a mouse) that is transgenic for human immunoglobulin genes (see e.g., Taylor, L. D., et al. (1992) Nucl. Acids Res. 20:6287-6295; Kellermann S-A., and Green L. L. (2002) Current Opinion in Biotechnology 13:593-597; Little M. et al (2000) Immunology Today 21:364-370) or antibodies prepared, expressed, created or isolated by any other means that involves splicing of human immunoglobulin gene sequences to other DNA sequences. In some embodiments, recombinant human antibodies are provided herein. In certain embodiments, such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo. One embodiment of the disclosure provides fully human antibodies, e.g., human antibody VH domain and a human antibody VL domain, which can be generated using appropriate techniques, such as, but not limited to, using human Ig phage libraries such as those disclosed in Jermutus et al., PCT publication No. WO 2005/007699 A2.

Selective: As used herein, the term “selective” or “selectively” refers to the ability of a molecule to produce an effect (e.g., inhibit, antagonize, agonize, etc) in relation to its target molecule compared to a reference molecule. For example, a molecule that selectively inhibits its target molecule means that this molecule is capable of inhibiting its target molecule with a degree that is distinguishable from a reference molecule in an inhibition assay or other inhibitory context. For example, with respect to an inhibitor, the term, “selectively inhibits”, refers to the ability of the inhibitor to inhibit its target molecule with a degree that is distinguishable from a reference molecule that is not substantially inhibited in an inhibition assay, e.g., to an extent that permit selective inhibition of the target molecule, as described herein. Once the reaction is terminated, the signal produced by inhibiting the target molecule can be measured. The half maximal inhibitor concentration for the target molecule and the reference molecule can be calculated.

Specifically binds: As used herein, the term “specifically binds” refers to the ability of a molecule to bind to a binding partner with a degree of affinity or avidity that enables the molecule to be used to distinguish the binding partner from an appropriate control in a binding assay or other binding context. With respect to an antibody, the term, “specifically binds”, refers to the ability of the antibody to bind to a specific antigen with a degree of affinity or avidity, compared with an appropriate reference antigen or antigens, that enables the antibody to be used to distinguish the specific antigen from others, as described herein. In some embodiments, an antibody specifically binds to a target if the antibody has a K_{D} for binding the target of at least about 10⁻⁴ M, 10⁻⁵ M, 10⁻⁶ M, 10⁻⁷ M, 10⁻⁸ M, 10⁻⁹ M, 10⁻¹⁰ M, 10⁻¹¹ M, 10⁻¹² M, 10⁻¹³ M, or less. In some embodiments, an antibody specifically binds trypsin 1 or trypsin 2.

Subject: As used herein, the term “subject” refers to a mammal. In some embodiments, a subject is a non-human primate, or a rodent. In some embodiments, a subject is a human. In some embodiments, a subject is a patient, e.g., a human patient that has or is suspected of having a disease.

Treatment: As used herein, the term “treating” or “treatment” refers to the application or administration of a composition including one or more active agents (e.g., anti-trypsin 1/trypsin 2 antibodies) to a subject, who has a target disease or disorder, a symptom of the disease/disorder, or a predisposition toward the disease/disorder, with the purpose to cure, heal, alleviate, relieve, alter, remedy, ameliorate, improve, or affect the disorder, the symptom of the disease, or the predisposition toward the disease or disorder. Alleviating a target disease/disorder includes delaying or preventing the development or progression of the disease, or reducing disease severity.

### II. Dual Inhibitor Antibodies Targeting Trypsin 1 and Trypsin 2

In some embodiments, an anti-trypsin 1/trypsin 2 antibody is an antibody specific for both trypsin 1 and trypsin 2 via a common, specific antigen binding site. Provided herein, in some aspects, are antibodies that bind to trypsin-1 (e.g., human trypsin 1, or non-human primate trypsin 1) and trypsin 2 (e.g., human trypsin 2, or non-human primate trypsin 2) with high specificity and affinity via a common antigen binding site. In some embodiments, the anti-trypsin 1/trypsin 2 antibody described herein specifically binds to an epitope of trypsin 1 that is exposed or becomes exposed to an antibody, and an epitope of trypsin 2 that is exposed or becomes exposed to an antibody. In some embodiments, anti-Trypsin 1/trypsin 2 antibodies provided herein bind specifically to trypsin 1 from human, non-human primates, etc. In some embodiments, anti-trypsin 1 antibodies provided herein specifically bind to human trypsin 1. In some embodiments, anti-trypsin 1 antibodies provided herein specifically bind to mouse trypsin 1.

Trypsin 1, also known as cationic trypsinogen, is a protease encoded by the PRSS1 gene on chromosome 7. Trypsin-1 is the main isoform of trypsinogen secreted by pancreas. The PRSS1 gene encodes a trypsinogen, which is a member of the trypsin family of serine proteases. This enzyme is secreted by the pancreas and cleaved to its active form in the small intestine. It is active on peptide linkages involving the carboxyl group of lysine or arginine. Certain mutations in the PRSS1 gene are autosome dominant mutations that lead to overactivation of trypsin 1, which is associated with hereditary pancreatitis. Further, the activity of trypsin 1 (e.g., with or without PRSS1 mutation) has been associated with the occurrence of pancreatitis (see, e.g., Gui et al., Trypsin activity governs increased susceptibility to pancreatitis in mice expressing human PRSS1^{R122H}, J Clin Invest. 2020; 130(1):189-202; Sendler et al., The Complex Role of Trypsin in Pancreatitis, Gastroenterology, EDITORIAL VOLUME 158, ISSUE 4, P822-826, MARCH 2020; Logsdon, Phosphatidylinositol 3-kinase and trypsin activation in pancreatitis, J Clin Invest. 2001 Nov. 1; 108(9): 1267-1268).

Trypsin 2, also known as anionic trypsinogen, is a protease encoded by the PRSS2 gene on chromosome 7. Trypsin 2 is the second most abundant trypsin secreted by the pancreas. Trypsin 2 has also been shown to be associated with pancreatitis (see, e.g., Dixit, Role of trypsinogen activation in genesis of pancreatitis, Pancreapedia: Exocrine Pancreas Knowledge Base, DOI: 10.3998/panc.2016.25; Kemppainen et al., Increased serum trypsinogen 2 and trypsin 2-al-antitrypsin complex values identify endoscopic retrograde cholangiopancreatography induced pancreatitis with high accuracy, Gut 1997; 41:690-695; Andersen et al., The ratio of trypsin-2-alpha(1)-antitrypsin to trypsinogen-1 discriminates biliary and alcohol-induced acute pancreatitis, Clinical Chemistry, 1 Feb. 2001, 47(2):231-236; Yu et al., Altered Gene Expression in Caerulein-Stimulated Pancreatic Acinar Cells: Pathologic Mechanism of Acute Pancreatitis, Korean J Physiol Pharmacol 2009; 13(6):409-416).

In some embodiments, an anti-trypsin 1/trypsin 2 antibody described herein specifically binds to an epitope on human trypsin 1. Exemplary amino acid sequences of human trypsin 1 are set forth in NCBI Accession Numbers NP_002760.1, and UniProt Accession Numbers: P07477, or P00760, the entire sequences of which are incorporated herein by reference.

In some embodiments, an anti-trypsin 1/trypsin 2 antibody described herein specifically binds to an epitope on human trypsin 1. In some embodiments, the serine in the catalytic domain of human trypsin 1 is mutated to alanine to raise the antibodies described herein. An exemplary amino acid sequence of human trypsin 1 with serine to alanine mutation is set forth in SEQ ID NO: 103 (including a His tag at C terminus):

IVGGYNCEENSVPYQVSLNSGYHFCGGSLINEQWVVSAGHCYKSRIQVR
LGEHNIEVLEGNEQFINAAKIIRHPQYDRKTLNNDIMLIKLSSRAVINA
RVSTISLPTAPPATGTKCLISGWGNTASSGADYPDELQCLDAPVLSQAK
CEASYPGKITSNMFCVGFLEGGKDSCQGDAGGPVVCNGQLQGVVSWGDG
CAQKNKPGVYTKVYNYVKWIKNTIAANSHHHHHH.

In some embodiments, an anti-trypsin 1/trypsin 2 antibody described herein specifically binds to an epitope on human trypsin 2. Exemplary amino acid sequences of human trypsin 2 are set forth in NCBI Accession Numbers NP_001290343.1, or NP_002761.1, and UniProt Accession Numbers: P07478, the entire sequences of which are incorporated herein by reference. In some embodiments,

In some embodiments, an anti-trypsin 1/trypsin 2 antibody described herein specifically binds to an epitope on human trypsin 2. In some embodiments, the serine in the catalytic domain of human trypsin 2 is mutated to alanine to raise the antibodies described herein. An exemplary amino acid sequence of human trypsin 2 with serine to alanine mutation is set forth in SEQ ID NO: 104 (including a His ta at C terminus):

IVGGYICEENSVPYQVSLNSGYHFCGGSLISEQWVVSAGHCYKSRIQVR
LGEHNIEVLEGNEQFINAAKIIRHPKYNSRTLDNDILLIKLSSPAVINS
RVSAISLPTAPPAAGTESLISGWGNTLSSGADYPDELQCLDAPVLSQAE
CEASYPGKITNNMFCVGFLEGGKDSCQGDAGGPVVSNGELQGIVSWGYG
CAQKNRPGVYTKVYNYVDWIKDTIAANSHHHHHH.

In some embodiments, an anti-trypsin 1/trypsin 2 antibody described herein specifically binds to an epitope on trypsin 1 (e.g., the catalytic domain/pocket of human trypsin 1 or non-human primate trypsin 1) and an epitope on trypsin 2 (e.g., the catalytic domain/pocket of human trypsin 2 or non-human primate trypsin 2). In some embodiments, an anti-trypsin 1/trypsin 2 antibody described herein prevents trypsin 1 (e.g., the catalytic domain/pocket of human trypsin 1 or non-human primate trypsin 1) and trypsin 2 (e.g., the catalytic domain/pocket of human trypsin 2 or non-human primate trypsin 2) from cleaving its substrates. In some embodiments, an anti-trypsin 1/trypsin 2 antibody described herein bind to a fragment of trypsin 1 (e.g., the catalytic domain/pocket of human trypsin 1 or non-human primate trypsin 1) and a fragment of trypsin 2 (e.g., the catalytic domain/pocket of human trypsin 2 or non-human primate trypsin 2). The fragment of trypsin 1 and/or trypsin 2 (e.g., human or non-human primate) may be between about 5 and about 425 amino acids, between about 10 and about 400 amino acids, between about 50 and about 350 amino acids, between about 100 and about 300 amino acids, between about 150 and about 250 amino acids, between about 200 and about 300 amino acids, between about 75 and about 150 amino acids, between about 25 and about 100 amino acids, between about 10 and about 30 amino acids in length. Not wishing to be bound to any particular theory, and in some embodiments, a heavy chain (HC) complementarity-determining region 3 (CDR3) of any one of the anti-trypsin 1/trypsin 2 antibodies described herein inhibits trypsin 1 (e.g., human or non-human primate trypsin 1) and trypsin 2 (e.g., human or non-human primate trypsin 2) by binding to the catalytic domain/pocket of trypsin 1 and trypsin 2 respectively.

In some embodiments, antibodies described herein are optimized versions (e.g., affinity matured) of the parental antibody. In some embodiments, an antibody described herein specifically binds trypsin 1 (e.g., a human or non-human primate trypsin 1) and trypsin 2 (e.g., a human or non-human primate trypsin 2) with binding affinity (e.g., as indicated by K_{D}) of at least about 10⁻⁴ M, 10⁻⁵ M, 10⁻⁶ M, 10⁻⁷ M, 10⁻⁸ M, 10⁻⁹ M, 10⁻¹⁰ M, 10⁻¹¹ M, 10⁻¹² M, 10⁻¹³ M, or less. In some embodiments, an antibody described herein specifically binds trypsin 1 (e.g., a human or non-human primate trypsin 1) and trypsin 2 (e.g., a human or non-human primate trypsin 2) with binding affinity (e.g., as indicated by K_{D}) of between 1×10⁻¹⁰ M and 5×10⁻⁹ M, between 1×10⁻¹⁰ M and 1×10⁻⁹ M, between 5×10⁻¹⁰ and 1×10⁻⁹ M, between 5×10⁻¹¹ and 1×10⁻¹⁰ M, between 1×10⁻¹¹ and 5×10⁻¹⁰ M, or between 5×10⁻¹³ and 1×10⁻¹² M. For example, an antibody of the present disclosure can bind to a trypsin 1 protein (e.g., a human or non-human primate trypsin 1) and a trypsin 2 protein (e.g., a human or non-human primate trypsin 2) with an affinity between 1 pM and 500 nM, e.g., between 50 pM and 100 nM, between 500 pM and 50 nM, between 1 pM and 100 pM, between 10 pM and 100 pM, between 50 pM and 100 pM, between 100 pM and 500 pM, between 500 pM and 1 nM, between 1 nM and 5 nM, between 1 nM and 10 nM, between 5 nM and 25 nM, between 10 nM and 50 nM between 50 nM and 100 nM, between 100 nM and 500 nM. The disclosure also includes antibodies that compete with any of the antibodies described herein for binding to a trypsin 1 protein (e.g., a human or non-human primate trypsin 1) and a trypsin 2 protein (e.g., a human or non-human primate trypsin 2) and that have an affinity of 100 nM or lower (e.g., 80 nM or lower, 50 nM or lower, 20 nM or lower, 10 nM or lower, 1 nM or lower, 500 pM or lower, 50 pM or lower, 10 pM or lower, or 5 pM or lower). The affinity and binding kinetics of an antibody can be tested using any suitable method including but not limited to biosensor technology (e.g., OCTET or BIACORE). In some embodiments, antibodies described herein binds to trypsin 1 and trypsin 2 with a K_{D} of sub-nanomolar range.

Binding affinity (or binding specificity) can be determined by a variety of methods including equilibrium dialysis, equilibrium binding, gel filtration, ELISA, surface plasmon resonance (SPR), florescent activated cell sorting (FACS) or spectroscopy (e.g., using a fluorescence assay). Exemplary conditions for evaluating binding affinity are in HBS-P buffer (10 mM HEPES pH7.4, 150 mM NaCl, 0.005% (v/v) surfactant P20) and PBS buffer (10 mM P04-3, 137 mM NaCl, and 2.7 mM KCl). These techniques can be used to measure the concentration of bound proteins as a function of target protein concentration. The concentration of bound protein ([[Bound]]) is generally related to the concentration of free target protein ([[Free]]) by the following equation:

$$[ [ Bound ] ] = [ [ Free ] ] / ( Kd + [ [ Free ] ] )$$

It is not always necessary to make an exact determination of K_{A}, though, since sometimes it is sufficient to obtain a quantitative measurement of affinity, e.g., determined using a method such as ELISA or FACS analysis, is proportional to K_{A}, and thus can be used for comparisons, such as determining whether a higher affinity is, e.g., 2-fold higher, to obtain a qualitative measurement of affinity, or to obtain an inference of affinity, e.g., by activity in a functional assay, e.g., an in vitro or in vivo assay.

Exemplary anti-trypsin 1/trypsin 2 antibody sequences (e.g., heavy chain variable domain (VH) and light chain variable domain (VL), CDR sequences are provided in Tables 1a and 1b.

TABLE la
Exemplary Anti-trypsin 1/trypsin 2 Antibodies

| Trypsin 1/Trypsin 2 | | | SEQ |
|---|---|---|---|
| Antibody | | Sequences | ID NO |
| Trypsin | HC CDR1 | FTFSSYAMS | 1 |
| 1/Trypsin 2- | HC CDR2 | AISASGGSTYYADSVKG | 2 |
| Dual-L1- | HC CDR3 | ARGSPIGYSYARVSYYYYMDV | 3 |
| Ab1 | LC CDR1 | RASQGISRWLA | 4 |
| | LC CDR2 | AASSLQS | 5 |
| | LC CDR3 | QQGNSFPLT | 6 |
| | VH | EVQLLESGGGLVQPGGSLRLSCAASGFTFSSYAMSWVRQAPG | 57 |
| | | KGLEWVSAISASGGSTYYADSVKGRFTISRDNSKNTLYLQMN | |
| | | SLRAEDTAVYYCARGSPIGYSYARVSYYYYMDVWGKGTTVTV | |
| | | SS | |
| | VL | DIQMTQSPSSVSASVGDRVTITCRASQGISRWLAWYQQKPGK | 58 |
| | | APKLLIYAASSLQSGVPSRFSGSGSGTDFTLTISSLQPEDFA | |
| | | TYYCQQGNSFPLTFGGGTKVEIK | |
| Trypsin | HC CDR1 | FTFGDYAMW | 7 |
| 1/Trypsin 2- | HC CDR2 | AISASGDVTYYADSVKG | 8 |
| Dual-L1- | HC CDR3 | ARGSLIGYSYARVSYYYYSDV | 9 |
| Ab2 | LC CDR1 | RASQGISRWLA | 4 |
| | LC CDR2 | AASSLQS | 5 |
| | LC CDR3 | QQGWSFPLT | 10 |
| | VH | EVQLLESGGGLVQPGGSLRLSCAASGFTFGDYAMWWVRQAPG | 59 |
| | | KGLEWVSAISASGDVTYYADSVKGRFTISRDNSKNTLYLQMN | |
| | | SLRAEDTAVYYCARGSLIGYSYARVSYYYYSDVWGKGTTVTV | |
| | | SS | |
| | VL | DIQMTQSPSSVSASVGDRVTITCRASQGISRWLAWYQQKPGK | 60 |
| | | APKLLIYAASSLQSGVPSRFSGSGSGTDFTLTISSLQPEDFA | |
| | | TYYCQQGWSFPLTFGGGTKVEIK | |
| Trypsin | HC CDR1 | GSISSYYWS | 11 |
| 1/Trypsin 2- | HC CDR2 | RIYTSGSTNYNPSLKS | 12 |
| Dual-L2- | HC CDR3 | ARDLTNYGSGKSIRFYGMDV | 13 |
| Ab1 | LC CDR1 | QASQDISNYLN | 14 |
| | LC CDR2 | DASNLET | 15 |
| | LC CDR3 | QQDDNFIT | 16 |
| | VH | QVQLQESGPGLVKPSETLSLTCTVSGGSISSYYWSWIRQPAG | 61 |
| | | KGLEWIGRIYTSGSTNYNPSLKSRVTMSVDTSKNQFSLKLSS | |
| | | VTAADTAVYYCARDLTNYGSGKSIRFYGMDVWGQGTTVTVSS | |
| | VL | DIQMTQSPSSLSASVGDRVTITCQASQDISNYLNWYQQKPGK | 62 |
| | | APKLLIYDASNLETGVPSRFSGSGSGTDFTFTISSLQPEDIA | |
| | | TYYCQQDDNFITFGGGTKVEIK | |
| Trypsin | HC CDR1 | GSISSNYWH | 17 |
| 1/Trypsin 2- | HC CDR2 | RIHTSGSTFYNPSLKS | 18 |
| Dual-L2- | HC CDR3 | ARDITNYGSGKSIRFYPMDV | 19 |
| Ab2 | LC CDR1 | QASQDISNYLN | 14 |
| | LC CDR2 | DASNLET | 15 |
| | LC CDR3 | QQDDTFIT | 20 |
| | VH | QVQLQESGPGLVKPSETLSLTCTVSGGSISSNYWHWIRQPAG | 63 |
| | | KGLEWIGRIHTSGSTFYNPSLKSRVTMSVDTSKNQFSLKLSS | |
| | | VTAADTAVYYCARDITNYGSGKSIRFYPMDVWGQGTTVTVSS | |
| | VL | DIQMTQSPSSLSASVGDRVTITCQASQDISNYLNWYQQKPGK | 64 |
| | | APKLLIYDASNLETGVPSRFSGSGSGTDFTFTISSLQPEDIA | |
| | | TYYCQQDDTFITFGGGTKVEIK | |
| Trypsin | HC CDR1 | GSISSHYWH | 21 |
| 1/Trypsin 2- | HC CDR2 | RIATSGSTYYNPSLKS | 22 |
| Dual-L2- | HC CDR3 | ARDLINYGITKSIRFYGMDV | 23 |
| Ab3 | LC CDR1 | QASQDISNYLN | 14 |
| | LC CDR2 | DASNLET | 15 |
| | LC CDR3 | QQVDNFIT | 24 |
| | VH | QVQLQESGPGLVKPSETLSLTCTVSGGSISSHYWHWIRQPAG | 65 |
| | | KGLEWIGRIATSGSTYYNPSLKSRVTMSVDTSKNQFSLKLSS | |
| | | VTAADTAVYYCARDLTNYGITKSIRFYGMDVWGQGTTVTVSS | |
| | VL | DIQMTQSPSSLSASVGDRVTITCQASQDISNYLNWYQQKPGK | 66 |
| | | APKLLIYDASNLETGVPSRFSGSGSGTDFTFTISSLQPEDIA | |
| | | TYYCQQVDNFITFGGGTKVEIK | |
| Trypsin | HC CDR1 | GSISSHYWH | 21 |
| 1/Trypsin 2- | HC CDR2 | RIHTSGSTFYNPSLKS | 18 |
| Dual-L2- | HC CDR3 | ARDLTNYGWSKSIRFYGMDV | 25 |
| Ab4 | LC CDR1 | QASQDISNYLN | 14 |
| | LC CDR2 | DASNLET | 15 |
| | LC CDR3 | QQTDNFIT | 26 |
| | VH | QVQLQESGPGLVKPSETLSLTCTVSGGSISSHYWHWIRQPAG | 67 |
| | | KGLEWIGRIHTSGSTFYNPSLKSRVTMSVDTSKNQFSLKLSS | |
| | | VTAADTAVYYCARDLTNYGWSKSIRFYGMDVWGQGTTVTVSS | |
| | VL | DIQMTQSPSSLSASVGDRVTITCQASQDISNYLNWYQQKPGK | 68 |
| | | APKLLIYDASNLETGVPSRFSGSGSGTDFTFTISSLQPEDIA | |
| | | TYYCQQTDNFITFGGGTKVEIK | |
| Trypsin | HC CDR1 | YTFTGYYMH | 27 |
| 1/Trypsin 2- | HC CDR2 | SINPSSGGTNYAQKFQG | 28 |
| Dual-L3- | HC CDR3 | ARVGGAAAAGKAYTASAFDI | 29 |
| Ab1 | LC CDR1 | RASQSVGSNLA | 30 |
| | LC CDR2 | GASTRAT | 31 |
| | LC CDR3 | QQFHNWPPLT | 32 |
| | VH | QVQLVQSGAEVKKPGASVKVSCKASGYTFTGYYMHWVRQAPG | 69 |
| | | QGLEWMGSINPSSGGTNYAQKFQGRVTMTRDTSISTAYMELS | |
| | | RLRSDDTAVYYCARVGGAAAAGKAYTASAFDIWGQGTMVTVS | |
| | | S | |
| | VL | EIVMTQSPATLSVSPGERATLSCRASQSVGSNLAWYQQKPGQ | 70 |
| | | APRLLIYGASTRATGIPARFSGSGSGTEFTLTISSLQSEDFA | |
| | | VYYCQQFHNWPPLTFGGGTKVEIK | |
| Trypsin | HC CDR1 | YTFTDHYMH | 33 |
| 1/Trypsin 2- | HC CDR2 | AINPSSGGTAYAQKFQG | 34 |
| Dual-L3- | HC CDR3 | ARVGGAARVGKAYTASAFDI | 35 |
| Ab2 | LC CDR1 | RASQSVGSNLA | 30 |
| | LC CDR2 | GASTRAT | 31 |
| | LC CDR3 | QQFHNWPPLT | 32 |
| | VH | QVQLVQSGAEVKKPGASVKVSCKASGYTFTDHYMHWVRQAPG | 71 |
| | | QGLEWMGAINPSSGGTAYAQKFQGRVTMTRDTSISTAYMELS | |
| | | RLRSDDTAVYYCARVGGAARVGKAYTASAFDIWGQGTMVTVS | |
| | | S | |
| | VL | EIVMTQSPATLSVSPGERATLSCRASQSVGSNLAWYQQKPGQ | 70 |
| | | APRLLIYGASTRATGIPARFSGSGSGTEFTLTISSLQSEDFA | |
| | | VYYCQQFHNWPPLTFGGGTKVEIK | |
| Trypsin | HC CDR1 | YTFTSYGIS | 36 |
| 1/Trypsin 2- | HC CDR2 | WISAYSGDTNYAQKLQG | 37 |
| Dual-L4- | HC CDR3 | ARGDEVVGKMTYYYGMDV | 38 |
| Ab1 | LC CDR1 | RASQGISRWLA | 4 |
| | LC CDR2 | AASSLQS | 5 |
| | LC CDR3 | QQANSFPIT | 39 |
| | VH | QVQLVQSGAEVKKPGASVKVSCKASGYTFTSYGISWVRQAPG | 72 |
| | | QGLEWMGWISAYSGDTNYAQKLQGRVTMTTDTSTSTAYMELR | |
| | | SLRSDDTAVYYCARGDEVVGKMTYYYGMDVWGQGTTVTVSS | |
| | VL | DIQMTQSPSSVSASVGDRVTITCRASQGISRWLAWYQQKPGK | 73 |
| | | APKLLIYAASSLQSGVPSRFSGSGSGTDFTLTISSLQPEDFA | |
| | | TYYCQQANSFPITFGGGTKVEIK | |
| Trypsin | HC CDR1 | YTFTDYAIS | 40 |
| 1/Trypsin 2- | HC CDR2 | WISASTADTNYAQKLQG | 41 |
| Dual-L4- | HC CDR3 | ARGDEVVGKMTYYYGMDV | 38 |
| Ab2 | LC CDR1 | RASQGISRWLA | 4 |
| | LC CDR2 | AASSLQS | 5 |
| | LC CDR3 | QQANSFPIT | 39 |
| | VH | QVQLVQSGAEVKKPGASVKVSCKASGYTFTDYAISWVRQAPG | 74 |
| | | QGLEWMGWISASTADTNYAQKLQGRVTMTTDTSTSTAYMELR | |
| | | SLRSDDTAVYYCARGDEVVGKMTYYYGMDVWGQGTTVTVSS | |
| | VL | DIQMTQSPSSVSASVGDRVTITCRASQGISRWLAWYQQKPGK | 73 |
| | | APKLLIYAASSLQSGVPSRFSGSGSGTDFTLTISSLQPEDFA | |
| | | TYYCQQANSFPITFGGGTKVEIK | |
| Trypsin | HC CDR1 | YTFTDYAIS | 40 |
| 1/Trypsin 2- | HC CDR2 | WISASTADTNYAQKLQG | 41 |
| Dual-L4- | HC CDR3 | ARGDEVVGKRTYYIGMDV | 42 |
| Ab3 | LC CDR1 | RASQGISRWLA | 4 |
| | LC CDR2 | AASSLQS | 5 |
| | LC CDR3 | QQANSFPIT | 39 |
| | VH | QVQLVQSGAEVKKPGASVKVSCKASGYTFTDYAISWVRQAPG | 75 |
| | | QGLEWMGWISASTADTNYAQKLQGRVTMTTDTSTSTAYMELR | |
| | | SLRSDDTAVYYCARGDEVVGKRTYYIGMDVWGQGTTVTVSS | |
| | VL | DIQMTQSPSSVSASVGDRVTITCRASQGISRWLAWYQQKPGK | 73 |
| | | APKLLIYAASSLQSGVPSRFSGSGSGTDFTLTISSLQPEDFA | |
| | | TYYCQQANSFPITFGGGTKVEIK | |
| Trypsin | HC CDR1 | YTFTDYAIS | 40 |
| 1/Trypsin 2- | HC CDR2 | WISASTADTNYAQKLQG | 41 |
| Dual-L4- | HC CDR3 | ARGTEVVGKMTVYLGMDV | 43 |
| Ab4 | LC CDR1 | RASQGISRWLA | 4 |
| | LC CDR2 | AASSLQS | 5 |
| | LC CDR3 | QQANSFPIT | 39 |
| | VH | QVQLVQSGAEVKKPGASVKVSCKASGYTFTDYAISWVRQAPG | 76 |
| | | QGLEWMGWISASTADTNYAQKLQGRVTMTTDTSTSTAYMELR | |
| | | SLRSDDTAVYYCARGTEVVGKMTVYLGMDVWGQGTTVTVSS | |
| | VL | DIQMTQSPSSVSASVGDRVTITCRASQGISRWLAWYQQKPGK | 73 |
| | | APKLLIYAASSLQSGVPSRFSGSGSGTDFTLTISSLQPEDFA | |
| | | TYYCQQANSFPITFGGGTKVEIK | |
| Trypsin | HC CDR1 | YTFTDYAIS | 40 |
| 1/Trypsin 2- | HC CDR2 | WISASTADTNYAQKLQG | 41 |
| Dual-L4- | HC CDR3 | ARQDEVVGKMTYYIGTDV | 44 |
| Ab5 | LC CDR1 | RASQGISRWLA | 4 |
| | LC CDR2 | AASSLQS | 5 |
| | LC CDR3 | QQANSFPLT | 45 |
| | VH | QVQLVQSGAEVKKPGASVKVSCKASGYTFTDYAISWVRQAPG | 77 |
| | | QGLEWMGWISASTADTNYAQKLQGRVTMTTDTSTSTAYMELR | |
| | | SLRSDDTAVYYCARQDEVVGKMTYYIGTDVWGQGTTVTVSS | |
| | VL | DIQMTQSPSSVSASVGDRVTITCRASQGISRWLAWYQQKPGK | 78 |
| | | APKLLIYAASSLQSGVPSRFSGSGSGTDFTLTISSLQPEDFA | |
| | | TYYCQQANSFPLTFGGGTKVEIK | |
| Trypsin | HC CDR1 | YTFTDYAIS | 40 |
| 1/Trypsin 2- | HC CDR2 | WISAGTADTNYAQKLQG | 46 |
| Dual-L4- | HC CDR3 | ARGDEVVGKMTYYYDMDV | 47 |
| Ab6 | LC CDR1 | RASQGISRWLA | 4 |
| | LC CDR2 | AASSLQS | 5 |
| | LC CDR3 | QQASSFPIT | 48 |
| | VH | QVQLVQSGAEVKKPGASVKVSCKASGYTFTDYAISWVRQAPG | 79 |
| | | QGLEWMGWISAGTADTNYAQKLQGRVTMTTDTSTSTAYMELR | |
| | | SLRSDDTAVYYCARGDEVVGKMTYYYDMDVWGQGTTVTVSS | |
| | VL | DIQMIQSPSSVSASVGDRVTITCRASQGISRWLAWYQQKPGK | 80 |
| | | APKLLIYAASSLQSGVPSRFSGSGSGTDFTLTISSLQPEDFA | |
| | | TYYCQQASSFPITFGGGTKVEIK | |
| Trypsin | HC CDR1 | YTFDDYGIS | 49 |
| 1/Trypsin 2- | HC CDR2 | WISATVADTNYAQKLQG | 50 |
| Dual-L4- | HC CDR3 | ARGDEPVGKMTYYYGMDV | 51 |
| Ab7 | LC CDR1 | RASQGISRWLA | 4 |
| | LC CDR2 | AASSLQS | 5 |
| | LC CDR3 | QQANSLPIT | 52 |
| | VH | QVQLVQSGAEVKKPGASVKVSCKASGYTFDDYGISWVRQAPG | 81 |
| | | QGLEWMGWISATVADTNYAQKLQGRVTMTTDTSTSTAYMELR | |
| | | SLRSDDTAVYYCARGDEPVGKMTYYYGMDVWGQGTTVTVSS | |
| | VL | DIQMTQSPSSVSASVGDRVTITCRASQGISRWLAWYQQKPGK | 82 |
| | | APKLLIYAASSLQSGVPSRFSGSGSGTDFTLTISSLQPEDFA | |
| | | TYYCQQANSLPITFGGGTKVEIK | |
| Trypsin | HC CDR1 | YTFTGYYMH | 27 |
| 1/Trypsin 2- | HC CDR2 | SINPSSGGTNYAQKFQG | 28 |
| Dual-L5- | HC CDR3 | ARDPVLLLRQQRILHESAFDI | 53 |
| Ab1 | LC CDR1 | RASQSVGSNLA | 30 |
| | LC CDR2 | GASTRAT | 31 |
| | LC CDR3 | QQFHNWPPLT | 32 |
| | VH | QVQLVQSGAEVKKPGASVKVSCKASGYTFTGYYMHWVRQAPG | 83 |
| | | QGLEWMGSINPSSGGTNYAQKFQGRVTMTRDTSISTAYMELS | |
| | | RLRSDDTAVYYCARDPVLLLRQQRILHESAFDIWGQGTTVTV | |
| | | SS | |
| | VL | EIVMTQSPATLSVSPGERATLSCRASQSVGSNLAWYQQKPGQ | 70 |
| | | APRLLIYGASTRATGIPARFSGSGSGTEFTLTISSLQSEDFA | |
| | | VYYCQQFHNWPPLTFGGGTKVEIK | |
| Trypsin | HC CDR1 | YTFEGHYMH | 54 |
| 1/Trypsin 2- | HC CDR2 | AINPYSGQTNYAQKFQG | 55 |
| Dual-L5- | HC CDR3 | ARDFVLLLRQSRILHESAFDI | 56 |
| Ab2 | LC CDR1 | RASQSVGSNLA | 30 |
| | LC CDR2 | GASTRAT | 31 |
| | LC CDR3 | QQFHNWPPLT | 32 |
| | VH | QVQLVQSGAEVKKPGASVKVSCKASGYTFEGHYMHWVRQAPG | 84 |
| | | QGLEWMGAINPYSGQTNYAQKFQGRVTMTRDTSISTAYMELS | |
| | | RLRSDDTAVYYCARDFVLLLRQSRILHESAFDIWGQGTTVTV | |
| | | SS | |
| | VL | EIVMTQSPATLSVSPGERATLSCRASQSVGSNLAWYQQKPGQ | 70 |
| | | APRLLIYGASTRATGIPARFSGSGSGTEFTLTISSLQSEDFA | |
| | | VYYCQQFHNWPPLTFGGGTKVEIK | |

In some embodiments, certain amino acid positions in an antibody described herein (e.g., amino acids in the VH/VL regions and/or CDR regions) are substitutable and the substitution results in an antibody with substantially similar binding and biological activities (e.g., substantially similar binding affinity, binding specificity, protease activity inhibitory activity, anti-inflammatory activity, or a combination thereof) as the reference antibody. In order to identify a substitutable position of an antibody, the amino acid sequence of that antibody is compared to the sequences of other antibodies belonging to the same group as that antibody. If the identity of that amino acid varies between the different related antibodies of a group at any particular position, that position is a substitutable position of the antibody. In other words, a substitutable position is a position in which the identity of the amino acid varies between the related antibodies. Positions that contain a constant amino acid are not substitutable positions.

In some embodiments, the above method may be employed to provide a consensus antibody sequence. In such a consensus sequence, a non-substitutable position is indicated by the amino acid present at that position, and a substitutable position is indicated as an “X”.

Depending on how the antibodies are to be employed, X may be a) any amino acid, b) any amino acid present at that position in any of the related antibodies in the group or a conservatively substituted variant thereof or c) any amino acid present at that position in any of the related antibodies in the group. Any antibody having a sequence that is encompassed by the consensus should bind to the same antigen as any of the related antibodies.

In some embodiments, the method described above may be employed in methods of designing and making a variant of a parental antibody that at least maintains (e.g., maintains or increases) the antigen binding activity of the parental antibody. Because antibodies containing substitutions at substitutable positions have already been produced and tested, substitutions at those positions can be made in the knowledge that they should not significantly decrease the binding activity of the antibody. In general, an antibody variant of a parental antibody has an antigen binding affinity that is at least 10%, at least 20%, at least 30%, at least 40%, at least 50%, at least 60%, at least 70%, at least 80%, at least 90% or at least 100% (e.g., at least 150%, at least 200%, at least 500%, at least 1000%, usually up to at least 10,000%) of the binding affinity of the parental antibody to a particular antigen.

In some embodiments, a substitutable position of a parental antibody may be substituted by a) any of the 20 naturally occurring amino acids to produce random substitutions, b) an amino acid having biochemical properties similar to the amino acid already present at the substitutable position to produce conservative substitutions, c) an amino acid that is present at the same position in a related antibody to produce a directed substitution, or d) an amino acid that is present at the same position in a similar human antibody to produce a humanizing substitution. A substitution may be made at any part of an antibody variable region, including any framework region or CDR. In certain embodiments, a single substitutable amino acid may be substituted. However, in other embodiments, a plurality of substitutable amino acids (e.g., up to about 5 or 10 or more) may be substituted. In particular embodiments, the type of substitution that can be made at each substitutable position may be indicated by the types of amino acids present at that position in the related antibodies. For example, if unrelated amino acids (e.g., Ala, Gly, Cys, Glu and Thr) are present at a certain position of a group of related antibodies, then any amino acid could be substituted at that position without significantly reducing binding activity of the antibody.

Exemplary amino acids substitutions of an anti-trypsin 1/trypsin 2 antibody described herein are set forth in Table 1b:

TABLE 1b
Exemplary Anti-Trypsin 1/Trypsin 2 Antibody Amino Acid Substitutions

| Trypsin 1/Trypsin 2 | | Sequences | SEQ ID NO |
|---|---|---|---|
| Trypsin | HC CDR1 | FTFX₁X₂YAMX₃ | 85 |
| 1/Trypsin 2- | | X₁ is S or G | |
| Dual-L1- | | X₂ is S or D | |
| Ab0 | | X₃ is S or W | |
| | HC CDR2 | AISASGX₄X₅TYYADSVKG | 86 |
| | | X₄ is G or D | |
| | | X₅ is S or V | |
| | HC CDR3 | ARGSX₆IGYSYARVSYYYYX₇DV | 87 |
| | | X₆ is P or L | |
| | | X₇ is M or S | |
| | LC CDR1 | RASQGISRWLA | 4 |
| | LC CDR2 | AASSLQS | 5 |
| | LC CDR3 | QQGX₈SFPLT | 88 |
| | | X₈ is N or W | |
| Trypsin | HC CDR1 | GSISSX₁YWX₂ | 89 |
| 1/Trypsin 2- | | X₁ is Y, N, or H | |
| Dual-L2- | | X₂ is S or H | |
| Ab0 | HC CDR2 | RIX₃TSGSTX₄YNPSLKS | 90 |
| | | X₃ is Y, H, or A | |
| | | X₄ is N, F, or Y | |
| | HC CDR3 | ARDX₅TNYGX₆X₇KSIRFYX₈MDV | 91 |
| | | X₅ is L or I | |
| | | X₆ is S, I, or W | |
| | | X₇ is G, T, or S | |
| | | X₈ is G or P | |
| | LC CDR1 | QASQDISNYLN | 14 |
| | LC CDR2 | DASNLET | 15 |
| | LC CDR3 | QQX₉DX₁₀FIT | 92 |
| | | X₉ is D, V, or T | |
| | | X₁₀ is N or T | |
| Trypsin | HC CDR1 | YTFTX₁X₂YMH | 93 |
| 1/Trypsin 2- | | X₁ is G or D | |
| Dual-L3- | | X₂ is Y or H | |
| Ab0 | HC CDR2 | X₃INPSSGGTX₄YAQKFQG | 94 |
| | | X₃ is S or A | |
| | | X₄ is N or A | |
| | HC CDR3 | ARVGGAAX₅X₆GKAYTASAFDI | 95 |
| | | X₅ is A or R | |
| | | X₆ is A or V | |
| | LC CDR1 | RASQSVGSNLA | 30 |
| | LC CDR2 | GASTRAT | 31 |
| | LC CDR3 | QQFHNWPPLT | 32 |
| Trypsin | HC CDR1 | YTFX₁X₂YX₃IS | 96 |
| 1/Trypsin 2- | | X₁ T or D | |
| Dual-L4- | | X₂ is S or D | |
| | | X₃ is G or A | |
| Ab0 | HC CDR2 | WISAX₄X₅X₆DTNYAQKLQG | 97 |
| | | X₄ is Y, S, G, or T | |
| | | X₅ is S, T, or V | |
| | | X₆ is G or A | |
| | HC CDR3 | ARX₇X₈EX₉VGKX₁₀TX₁₁YX₁₂X₁₃X₁₄DV | 98 |
| | | X₇ is G or Q | |
| | | X₈ is D or T | |
| | | X₉ is V or P | |
| | | X₁₀ is M or R | |
| | | X₁₁ is Y or V | |
| | | X₁₂ is Y, I, or L | |
| | | X₁₃ is G or D | |
| | | X₁₄ is M or T | |
| | LC CDR1 | RASQGISRWLA | 4 |
| | LC CDR2 | AASSLQS | 5 |
| | LC CDR3 | QQAX₁₅SX₁₆PX₁₇T | 99 |
| | | X₁₅ is N or S | |
| | | X₁₆ is F or L | |
| | | X₁₇ is I or L | |
| Trypsin | HC CDR1 | YTFX₁GX₂YMH | 100 |
| 1/Trypsin 2- | | X₁ is T or E | |
| Dual-L5- | | X₂ is Y or H | |
| Ab0 | HC CDR2 | X₃INPX4SGX₅TNYAQKFQG | 101 |
| | | X₃ is S or A | |
| | | X₄ is S or Y | |
| | | X₅ is G or Q | |
| | HC CDR3 | ARDX₆VLLLRQX₇RILHESAFDI | 102 |
| | | X₆ is P or F | |
| | | X₇ is Q or S | |
| | LC CDR1 | RASQSVGSNLA | 30 |
| | LC CDR2 | GASTRAT | 31 |
| | LC CDR3 | QQFHNWPPLT | 32 |

In some embodiments, an antibody of the present disclosure comprises a HC CDR1 comprising the amino acid sequence of FTFX₁X₂YAMX₃ (SEQ ID NO: 85), in which X₁ is S or G, X₂ is S or D, or X₃ is S or W; a HC CDR2 comprising the amino acid sequence of AISASGX₄X₅TYYADSVKG (SEQ ID NO: 86), in which X₄ is G or D, or X₅ is S or V; a HC CDR3 comprising the amino acid sequence of ARGSX₆IGYSYARVSYYYYX₇DV (SEQ ID NO: 87), in which X₆ is P or L, X₇ is M or S; a LC CDR1 comprising the amino acid sequence of SEQ ID NO: 4; a LC CDR2 comprising the amino acid sequence of SEQ ID NO: 5; and/or a LC CDR3 comprising the amino acid sequence of QQGX₈SFPLT (SEQ ID NO: 88), in which X₈ is N or W.

In some embodiments, an antibody of the present disclosure comprises a HC CDR1 comprising the amino acid sequence of GSISSX₁YWX₂ (SEQ ID NO: 89), in which X₁ is Y, N, or H, or X₂ is S or H; a HC CDR2 comprising the amino acid sequence of RIX₃TSGSTX₄YNPSLKS (SEQ ID NO: 90), in which X₃ is Y, H, or A, or X₄ is N, F, or Y; a HC CDR3 comprising the amino acid sequence of ARDX₅TNYGX₆X₇KSIRFYX₈MDV (SEQ ID NO: 91), in which X₅ is L or I, X₆ is S, I, or W, X₇ is G, T, or S, or X₈ is G or P; a LC CDR1 comprising the amino acid sequence of SEQ ID NO: 14; a LC CDR2 comprising the amino acid sequence of SEQ ID NO: 15; and/or a LC CDR3 comprising the amino acid sequence of QQX₉DX₁₀FIT (SEQ ID NO: 92), in which X₉ is D, V, or T, or X₁₀ is N or T.

In some embodiments, an antibody of the present disclosure comprises a HC CDR1 comprising the amino acid sequence of YTFTX₁X₂YMH (SEQ ID NO: 93), in which X₁ is G or D, or X₂ is Y or H; a HC CDR2 comprising the amino acid sequence of X₃INPSSGGTX₄YAQKFQG (SEQ ID NO: 94), in which X₃ is S or A, or X₄ is N or A; a HC CDR3 comprising the amino acid sequence of ARVGGAAX₅X₆GKAYTASAFDI (SEQ ID NO: 95), in which X₅ is A or R, or X₆ is A or V; a LC CDR1 comprising the amino acid sequence of SEQ ID NO: 30; a LC CDR2 comprising the amino acid sequence of SEQ ID NO: 31; and/or a LC CDR3 comprising the amino acid sequence of SEQ ID NO: 32.

In some embodiments, an antibody of the present disclosure comprises a HC CDR1 comprising the amino acid sequence of YTFX₁X₂YX₃IS (SEQ ID NO: 96), in which X₁ is T or D, X₂ is S or D, or X₂ is G or A; a HC CDR2 comprising the amino acid sequence of WISAX₄X₅X₆DTNYAQKLQG (SEQ ID NO: 97), in which X₄ is Y, S, G, or T, X₅ is S, T, or V, or X₆ is G or A; a HC CDR3 comprising the amino acid sequence of ARX₇X₈EX₉VGKX₁₀TX₁₁YX₁₂X₁₃X₁₄DV (SEQ ID NO: 98), in which X₇ is G or Q, X₈ is D or T, X₉ is V or P, X₁₀ is M or R, X₁₁ is Y or V, X₁₂ is Y, I, or L, X₁₃ is G or D, or X₁₄ is M or T; a LC CDR1 comprising the amino acid sequence of SEQ ID NO: 4; a LC CDR2 comprising the amino acid sequence of SEQ ID NO: 5; and/or a LC CDR3 comprising the amino acid sequence of QQAX₁₅SX₁₆PX₁₇T (SEQ ID NO: 99), in which X₁ₛ is N or S, X₁₆ is F or L, or X₁₇ is I or L.

In some embodiments, an antibody of the present disclosure comprises a HC CDR1 comprising the amino acid sequence of YTFX₁GX₂YMH (SEQ ID NO: 100), in which X₁ is T or E, or X₂ is Y or H; a HC CDR2 comprising the amino acid sequence of X₃INPX₄SGX₅TNYAQKFQG (SEQ ID NO: 101), in which X₃ is S or A, X₄ is S or Y, or X₅ is G or Q; a HC CDR3 comprising the amino acid sequence of ARDX6VLLLRQX7RILHESAFDI (SEQ ID NO: 102), in which X₆ is P or F, X₇ is Q or S; a LC CDR1 comprising the amino acid sequence of SEQ ID NO: 30; a LC CDR2 comprising the amino acid sequence of SEQ ID NO: 31; and/or a LC CDR3 comprising the amino acid sequence of SEQ ID NO: 32.

In some embodiments, an antibody of the present disclosure comprises one or more of the HC CDRs (e.g., HC CDR1, HC CDR2, or HC CDR3) amino acid sequences from any one of the anti-trypsin 1/trypsin 2 antibodies selected from Tables 1a and 1b. In some embodiments, an antibody of the present disclosure comprises the HC CDR3 amino acid sequences from any one of the anti-trypsin 1/trypsin 2 antibodies selected from Tables 1a and 1b. In some embodiments, an antibody of the present disclosure comprise the HC CDR1, HC CDR2, and HC CDR3 as provided for any one of the anti-trypsin 1/trypsin 2 antibodies elected from Tables 1a and 1b. In some embodiments, an antibody of the present disclosure comprises the LC CDR3 amino acid sequences from any one of the anti-trypsin 1/trypsin 2 antibodies selected from Tables 1a and 1b. In some embodiments, an antibody of the present disclosure comprises one or more of the LC CDRs (e.g., LC CDR1, LC CDR2, or LC CDR3) amino acid sequences from any one of the anti-trypsin 1/trypsin 2 antibodies selected from Tables 1a and 1b. In some embodiments, an antibody of the present disclosure comprise the LC CDR1, LC CDR2, and LC CDR3 s provided for any one of the anti-trypsin 1/trypsin 2 antibodies selected from Tables 1a and 1b.

In some embodiments, an antibody of the present disclosure comprises the HC CDR1, HC CDR2, HC CDR3, LC CDR1, LC CDR2, and LC CDR3 as provided for any one of the anti-trypsin 1/trypsin 2 antibodies selected from Tables 1a and 1b. In some embodiments, antibody heavy and/or light chain CDR3 domains may play a particularly important role in the binding specificity/affinity of an antibody for an antigen. Accordingly, an antibody of the disclosure may include at least the heavy and/or light chain CDR3s of any one of the anti-trypsin 1/trypsin 2 antibodies selected from Tables 1a and 1b.

Also within the scope of the present disclosure are variants of any of the exemplary anti-trypsin 1/trypsin 2 antibodies as disclosed herein. A variant may contain one or more amino acid residue variations in the VH and/or VL, or in one or more of the HC CDRs and/or one or more of the LC CDRs as relative to the reference antibody, while retaining substantially similar binding and biological activities (e.g., substantially similar binding affinity, binding specificity, protease activity inhibitory activity, anti-inflammatory activity, or a combination thereof) as the reference antibody.

In some embodiments, an antibody of the disclosure have one or more CDRs (e.g., HC CDR or LC CDR) sequences substantially similar to any of the HC CDR1, HC CDR2, HC CDR3, LC CDR1, LC CDR2, and/or LC CDR3 sequences from one of the anti-trypsin 1/trypsin 2 antibodies selected from Tables 1a and 1b. In some embodiments, the position of one or more CDRs along the VH (e.g., HC CDR1, HC CDR2, or HC CDR3) and/or VL (e.g., LC CDR1, LC CDR2, or LC CDR3) region of an antibody described herein can vary by one, two, three, four, five, or six amino acid positions so long as specific binding to trypsin 1 (e.g., human or non-human primate trypsin 1) and trypsin 2 (e.g., human or non-human primate trypsin 2) is maintained (e.g., substantially maintained, for example, at least 50%, at least 60%, at least 70%, at least 80%, at least 90%, at least 95% of the binding of the original antibody from which it is derived). For example, in some embodiments, the position defining a CDR of any antibody described herein can vary by shifting the N-terminal and/or C-terminal boundary of the CDR by one, two, three, four, five, or six amino acids, relative to the CDR position of any one of the antibodies described herein, so long as specific binding to trypsin 1 (e.g., human or non-human primate trypsin 1) and trypsin 2 (e.g., human or non-human primate trypsin 2) is maintained (e.g., substantially maintained, for example, at least 50%, at least 60%, at least 70%, at least 80%, at least 90%, at least 95% of the binding of the original antibody from which it is derived). In another embodiment, the length of one or more CDRs along the VH (e.g., HC CDR1, HC CDR2, or HC CDR3) and/or VL (e.g., LC CDR1, LC CDR2, or LC CDR3) region of an antibody described herein can vary (e.g., be shorter or longer) by one, two, three, four, five, or more amino acids, so long as immunospecific binding to trypsin 1 (e.g., human or non-human primate trypsin 1) and trypsin 2 (e.g., human or non-human primate trypsin 2) is maintained (e.g., substantially maintained, for example, at least 50%, at least 60%, at least 70%, at least 80%, at least 90%, at least 95% of the binding of the original antibody from which it is derived).

Accordingly, in some embodiments, a HC CDR1, HC CDR2, HC CDR3, LC CDR1, LC CDR2, and/or LC CDR3 described herein may be one, two, three, four, five or more amino acids shorter than one or more of the CDRs described herein (e.g., CDRS from any of the anti-trypsin 1/trypsin 2 antibodies selected from Tables 1a and 1b) so long as specific binding to trypsin 1 (e.g., human or non-human primate trypsin 1) and trypsin 2 (e.g., human or non-human primate trypsin 2) is maintained (e.g., substantially maintained, for example, at least 50%, at least 60%, at least 70%, at least 80%, at least 90%, at least 95% relative to the binding of the original antibody from which it is derived). In some embodiments, a HC CDR1, HC CDR2, HC CDR3, LC CDR1, LC CDR2, and/or LC CDR3 described herein may be one, two, three, four, five or more amino acids longer than one or more of the CDRs described herein (e.g., CDRs from any of the anti-trypsin 1/trypsin 2 antibodies selected from Tables 1a and 1b) so long as specific binding to trypsin 1 (e.g., human or non-human primate trypsin 1) and trypsin 2 (e.g., human or non-human primate trypsin 2) is maintained (e.g., substantially maintained, for example, at least 50%, at least 60%, at least 70%, at least 80%, at least 90%, at least 95% relative to the binding of the original antibody from which it is derived). In some embodiments, the amino portion of a HC CDR1, HC CDR2, HC CDR3, LC CDR1, LC CDR2, and/or LC CDR3 described herein can be extended by one, two, three, four, five or more amino acids compared to one or more of the CDRs described herein (e.g., CDRs from any of the anti-trypsin 1/trypsin 2 antibodies selected from Tables 1a and 1b) so long as specific binding to trypsin 1 (e.g., human or non-human primate trypsin 1) and trypsin 2 (e.g., human or non-human primate trypsin 2) is maintained (e.g., substantially maintained, for example, at least 50%, at least 60%, at least 70%, at least 80%, at least 90%, at least 95% relative to the binding of the original antibody from which it is derived). In some embodiments, the carboxy portion of a HC CDR1, HC CDR2, HC CDR3, LC CDR1, LC CDR2, and/or LC CDR3 described herein can be extended by one, two, three, four, five or more amino acids compared to one or more of the CDRs described herein (e.g., CDRs from any of the anti-trypsin 1/trypsin 2 antibodies selected from Tables 1a and 1b) so long as specific binding to trypsin 1 (e.g., human or non-human primate trypsin 1) and trypsin 2 (e.g., human or non-human primate trypsin 2) is maintained (e.g., substantially maintained, for example, at least 50%, at least 60%, at least 70%, at least 80%, at least 90%, at least 95% relative to the binding of the original antibody from which it is derived). In some embodiments, the amino portion of a HC CDR1, HC CDR2, HC CDR3, LC CDR1, LC CDR2, and/or LC CDR3 described herein can be shortened by one, two, three, four, five or more amino acids compared to one or more of the CDRs described herein (e.g., CDRs from any of the anti-trypsin 1/trypsin 2 antibodies selected from Tables 1a and 1b) so long as specific binding to trypsin 1 (e.g., human or non-human primate trypsin 1) and trypsin 2 (e.g., human or non-human primate trypsin 2) is maintained (e.g., substantially maintained, for example, at least 50%, at least 60%, at least 70%, at least 80%, at least 90%, at least 95% relative to the binding of the original antibody from which it is derived). In some embodiments, the carboxy portion of a HC CDR1, HC CDR2, HC CDR3, LC CDR1, LC CDR2, and/or LC CDR3 described herein can be shortened by one, two, three, four, five or more amino acids compared to one or more of the CDRs described herein (e.g., CDRs from any of the anti-trypsin 1/trypsin 2 antibodies selected from Tables 1a and 1b) so long as specific binding to trypsin 1 (e.g., human or non-human primate trypsin 1) and trypsin 2 (e.g., human or non-human primate trypsin 2) is maintained (e.g., substantially maintained, for example, at least 50%, at least 60%, at least 70%, at least 80%, at least 90%, at least 95% relative to the binding of the original antibody from which it is derived). Any method can be used to ascertain whether specific binding to trypsin 1 (e.g., human or non-human primate trypsin 1) and trypsin 2 (e.g., human or non-human primate trypsin 2) is maintained, for example, using binding assays and conditions described in the art.

In some examples, an antibody of the disclosure have one or more CDR (e.g., HC CDR or LC CDR) sequences substantially similar to any one of the anti-trypsin 1/trypsin 2 antibodies selected from Tables 1a and 1b. For example, an antibody described herein may include one or more CDR sequence(s) from any of the anti-trypsin 1/trypsin 2 antibodies selected from Tables 1a and 1b containing up to 5, 4, 3, 2, or 1 amino acid residue variations as compared to the corresponding CDR region in any one of the CDRs provided herein (e.g., CDRs from any of the anti-trypsin 1/trypsin 2 antibodies selected from Tables 1a and 1b) so long as specific binding to trypsin 1 (e.g., human or non-human primate trypsin 1) and trypsin 2 (e.g., human or non-human primate trypsin 2) is maintained (e.g., substantially maintained, for example, at least 50%, at least 60%, at least 70%, at least 80%, at least 90%, at least 95% relative to the binding of the original antibody from which it is derived). In some embodiments, any of the amino acid variations in any of the CDRs provided herein may be conservative variations. Conservative variations can be introduced into the CDRs at positions where the residues are not likely to be involved in interacting with a trypsin 1 (e.g., human or non-human primate trypsin 1) and/or a trypsin 2 (e.g., human or non-human primate trypsin 2), for example, as determined based on a crystal structure. Some aspects of the disclosure provide antibodies that comprise one or more of the heavy chain variable (VH) and/or light chain variable (VL) domains provided herein. In some embodiments, any of the VH domains provided herein include one or more of the HC CDR sequences (e.g., HC CDR1, HC CDR2, and HC CDR3) provided herein, for example, any of the HC CDR sequences provided in any one of the anti-trypsin 1/trypsin 2 selected from Tables 1a and 1b. In some embodiments, any of the VL domains provided herein include one or more of the LC CDR sequences (e.g., LC CDR1, LC CDR2, and LC CDR3) provided herein, for example, any of the LC CDR sequences provided in any one of the anti-trypsin 1/trypsin 2 antibodies selected from Tables 1a and 1b.

In some embodiments, an antibody of the disclosure include any antibody that includes a heavy chain variable domain and/or a light chain variable domain of any one of the anti-trypsin 1/trypsin 2 antibodies selected from Tables 1a and 1b, and variants thereof. In some embodiments, an antibody of the disclosure include any antibody that includes the heavy chain variable and light chain variable pairs of any anti-trypsin 1/trypsin 2 antibodies selected from Tables 1a and 1b.

Aspects of the disclosure provide antibodies having a heavy chain variable (VH) and/or a light chain variable (VL) domain amino acid sequence homologous to any of those described herein. In some embodiments, an antibody comprises a heavy chain variable sequence or a light chain variable sequence that is at least 75% (e.g., at least 80%, at least 85%, at least 90%, at least 91%, at least 92%, at least 93%, at least 94%, at least 95%, at least 96%, at least 97%, at least 98%, or at least 99%) identical to the heavy chain variable sequence and/or any light chain variable sequence of any one of the anti-trypsin 1/trypsin 2 antibodies selected from Tables 1a and 1b. In some embodiments, the homologous heavy chain variable and/or a light chain variable amino acid sequences do not vary within any of the CDR sequences provided herein. For example, in some embodiments, the degree of sequence variation ((e.g., at least 80%, at least 85%, at least 90%, at least 91%, at least 92%, at least 93%, at least 94%, at least 95%, at least 96%, at least 97%, at least 98%, or at least 99%) may occur within a heavy chain variable and/or a light chain variable sequence excluding any of the CDR sequences provided herein. In some embodiments, an antibody provided herein comprise a heavy chain variable sequence and a light chain variable sequence that comprises a framework sequence that is at least 80%, at least 85%, at least 90%, at least 91%, at least 92%, at least 93%, at least 94%, at least 95%, at least 96%, at least 97%, at least 98%, or at least 99% identical to the framework sequence of any anti-trypsin 1/trypsin 2 antibodies selected from Tables 1a and 1b.

In some embodiments, an anti-trypsin 1/trypsin 2 antibody of the present disclosure is an antibody comprising a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH of any of the anti-trypsin 1/trypsin 2 antibodies listed in Tables 1a and 1b. Alternatively or in addition, the anti-trypsin 1/trypsin 2 antibody of the present disclosure is an antibody comprising a VL containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the VL of any one of the anti-trypsin 1/trypsin 2 antibodies listed in Tables 1a and 1b. In some embodiments, a heavy chain variable and/or a light chain variable amino acid sequences do not vary within any of the CDR sequences provided herein. For example, in some embodiments, the number of amino acid variations (e.g., no more than 20, 19, 18, 17, 16, 15, 14, 13, 12, 11, 10, 9, 8, 7, 6, 5, 4, 3, 2, or 1 amino acid variation) may occur within a heavy chain variable and/or a light chain variable sequence excluding any of the CDR sequences provided herein. In some embodiments, any of the anti-trypsin 1/trypsin 2 antibodies provided herein comprise a heavy chain variable sequence that comprises a framework sequence that that contains no more than 20, 19, 18, 17, 16, 15, 14, 13, 12, 11, 10, 9, 8, 7, 6, 5, 4, 3, 2, or 1 amino acid variation to the framework sequence of a VH of any one of anti-trypsin 1/trypsin 2 antibodies selected from Tables 1a and 1b and/or a light chain variable sequence that comprises a framework sequence that that contains no more than 20, 19, 18, 17, 16, 15, 14, 13, 12, 11, 10, 9, 8, 7, 6, 5, 4, 3, 2, or 1 amino acid variation to the framework sequence of a VL of any one of anti-trypsin 1/trypsin 2 antibodies selected from Tables 1a and 1b.

In some embodiments, an anti-trypsin 1/trypsin 2 antibody of the present disclosure comprises a HC CDR1, HC CDR2 and HC CDR3 of a heavy chain variable domain having the amino acid sequence of SEQ ID NO: 57. Alternatively or in addition, the anti-trypsin 1/trypsin 2 antibody of the present disclosure comprises a LC CDR1, LC CDR2 and LC CDR3 of a light chain variable domain having the amino acid sequence of SEQ ID NO: 58.

In some embodiments, an anti-trypsin 1/trypsin 2 antibody of the present disclosure comprises a HC CDR1 having the amino acid sequence of SEQ ID NO: 1, a HC CDR2 having the amino acid sequence of SEQ ID NO: 2, a HC CDR3 having the amino acid sequence of SEQ ID NO: 3, a LC CDR1 having the amino acid sequence of SEQ ID NO: 4, a LC CDR2 having the amino acid sequence of SEQ ID NO: 5, and a LC CDR3 having the amino acid sequence of SEQ ID NO: 6.

In some embodiments, an anti-trypsin 1/trypsin 2 antibody of the present disclosure comprises a HC CDR1, a HC CDR2, and a HC CDR3, which collectively contains no more than 5 amino acid variations (e.g., no more than 5, 4, 3, 2, or I amino acid variation) as compared with the HC CDR1 having the amino acid sequence of SEQ ID NO: 1, HC CDR2 having the amino acid sequence of SEQ ID NO: 2, and HC CDR3 having the amino acid sequence of SEQ ID NO: 3. “Collectively,” as used anywhere in the present disclosure, means that the total number of amino acid variations in all of the three heavy chain CDRs is within the defined range. Alternatively or in addition, the anti-trypsin 1/trypsin 2 antibody of the present disclosure comprises a LC CDR1, a LC CDR2, and a LC CDR3, which collectively contains no more than 5 amino acid variations (e.g., no more than 5, 4, 3, 2 or I amino acid variation) as compared with the LC CDR1 having the amino acid sequence of SEQ ID NO: 4, LC CDR2 having the amino acid sequence of SEQ ID NO: 5, and LC CDR3 having the amino acid sequence of SEQ ID NO: 6.

In some embodiments, an anti-trypsin 1/trypsin 2 antibody of the present disclosure comprises a HC CDR1, a HC CDR2, and a HC CDR3 that collectively are at least 80% (e.g., at least 80%, at least 85%, at least 90%, at least 91%, at least 92%, at least 93%, at least 94%, at least 95%, at least 96%, at least 97%, at least 98%, or at least 99%) identical to the HC CDR1 having the amino acid sequence of SEQ ID NO: 1, HC CDR2 having the amino acid sequence of SEQ ID NO: 2, and HC CDR3 having the amino acid sequence of SEQ ID NO: 3. Alternatively or in addition, the anti-trypsin 1/trypsin 2 antibody of the present disclosure comprises a LC CDR1, a LC CDR2, and a LC CDR3 that collectively are at least 80% (e.g., at least 80%, at least 85%, at least 90%, at least 91%, at least 92%, at least 93%, at least 94%, at least 95%, at least 96%, at least 97%, at least 98%, or at least 99%) identical to the to the LC CDR1 having the amino acid sequence of SEQ ID NO: 4, LC CDR2 having the amino acid sequence of SEQ ID NO: 5, and LC CDR3 having the amino acid sequence of SEQ ID NO: 6.

In some embodiments, an anti-trypsin 1/trypsin 2 antibody of the present disclosure comprises: a HC CDR1 having no more than 3 amino acid variations (e.g., no more than 3, 2, or 1 amino acid variation) as compared with the HC CDR1 having the amino acid sequence of SEQ ID NO: 1; a HC CDR2 having no more than 3 amino acid variations (e.g., no more than 3, 2, or 1 amino acid variation) as compared with the HC CDR2 having the amino acid sequence of SEQ ID NO: 2; and/or a HC CDR3 having no more than 3 amino acid variations (e.g., no more than 3, 2, or 1 amino acid variation) as compared with the HC CDR3 having the amino acid sequence of SEQ ID NO: 3. Alternatively or in addition, the anti-trypsin 1/trypsin 2 antibody of the present disclosure comprises: a LC CDR1 having no more than 3 amino acid variations (e.g., no more than 3, 2, or I amino acid variation) as compared with the LC CDR1 having the amino acid sequence of SEQ ID NO: 4; a LC CDR2 having no more than 3 amino acid variations (e.g., no more than 3, 2, or 1 amino acid variation) as compared with the LC CDR2 having the amino acid sequence of SEQ ID NO: 5; and/or a LC CDR3 having no more than 3 amino acid variations (e.g., no more than 3, 2, or 1 amino acid variation) as compared with the LC CDR3 having the amino acid sequence of SEQ ID NO: 6.

In some embodiments, an anti-trypsin 1/trypsin 2 antibody of the present disclosure comprises: a HC CDR1 that is at least 80% (e.g., at least 80%, at least 85%, at least 90%, at least 91%, at least 92%, at least 93%, at least 94%, at least 95%, at least 96%, at least 97%, at least 98%, or at least 99%) identical to the HC CDR1 having the amino acid sequence of SEQ ID NO: 1; a HC CDR2 that is at least 80% (e.g., at least 80%, at least 85%, at least 90%, at least 91%, at least 92%, at least 93%, at least 94%, at least 95%, at least 96%, at least 97%, at least 98%, or at least 99%) identical to the HC CDR2 having the amino acid sequence SEQ ID NO: 2; and/or a HC CDR3 that is at least 80% (e.g., at least 80%, at least 85%, at least 90%, at least 91%, at least 92%, at least 93%, at least 94%, at least 95%, at least 96%, at least 97%, at least 98%, or at least 99%) identical to the HC CDR3 having the amino acid sequence of SEQ ID NO: 3. Alternatively or in addition, the anti-trypsin 1/trypsin 2 antibody of the present disclosure comprises: a LC CDR1 that is at least 80% (e.g., at least 80%, at least 85%, at least 90%, at least 91%, at least 92%, at least 93%, at least 94%, at least 95%, at least 96%, at least 97%, at least 98%, or at least 99%) identical to the LC CDR1 having the amino acid sequence of SEQ ID NO: 4; a LC CDR2 that is at least 80% (e.g., at least 80%, at least 85%, at least 90%, at least 91%, at least 92%, at least 93%, at least 94%, at least 95%, at least 96%, at least 97%, at least 98%, or at least 99%) identical to the LC CDR2 having the amino acid sequence of SEQ ID NO: 5; and/or a LC CDR3 that is at least 80% (e.g., at least 80%, at least 85%, at least 90%, at least 91%, at least 92%, at least 93%, at least 94%, at least 95%, at least 96%, at least 97%, at least 98%, or at least 99%) identical to the LC CDR3 having the amino acid sequence of SEQ ID NO: 6.

In some embodiments, an anti-trypsin 1/trypsin 2 antibody of the present disclosure comprises a VH comprising the amino acid sequence of SEQ ID NO: 57. Alternatively or in addition, the anti-trypsin 1/trypsin 2 antibody of the present disclosure comprises a VL comprising the amino acid sequence of SEQ ID NO: 58.

In some embodiments, an anti-trypsin 1/trypsin 2 antibody of the present disclosure comprises a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 57. Alternatively or in addition, the anti-trypsin 1/trypsin 2 antibody of the present disclosure comprises a VL containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the VL as set forth in SEQ ID NO: 58. In some embodiments, the number of amino acid variations (e.g., no more than 20, 19, 18, 17, 16, 15, 14, 13, 12, 11, 10, 9, 8, 7, 6, 5, 4, 3, 2, or 1 amino acid variation) may occur within a VH of SEQ ID NO: 57 and/or a VL of SEQ ID NO: 58 excluding any of the CDR sequences therein. In some embodiments, an anti-trypsin 1/trypsin 2 antibodies provided herein comprise a heavy chain variable sequence that comprises a framework sequence that that contains no more than 20, 19, 18, 17, 16, 15, 14, 13, 12, 11, 10, 9, 8, 7, 6, 5, 4, 3, 2, or 1 amino acid variation to the framework sequence of a VH of SEQ ID NO: 57, and/or a light chain variable sequence that comprises a framework sequence that that contains no more than 20, 19, 18, 17, 16, 15, 14, 13, 12, 11, 10, 9, 8, 7, 6, 5, 4, 3, 2, or 1 amino acid variation to the framework sequence of a VL of SEQ ID NO: 58.

In some embodiments, an anti-trypsin 1/trypsin 2 antibody of the present disclosure comprises a VH comprising an amino acid sequence that is at least 80% (e.g., at least 80%, at least 85%, at least 90%, at least 91%, at least 92%, at least 93%, at least 94%, at least 95%, at least 96%, at least 97%, at least 98%, or at least 99%) identical to the VH as set forth in SEQ ID NO: 57. Alternatively or in addition, the anti-trypsin 1/trypsin 2 antibody of the present disclosure comprises a VL comprising an amino acid sequence that is at least 80% (e.g., at least 80%, at least 85%, at least 90%, at least 91%, at least 92%, at least 93%, at least 94%, at least 95%, at least 96%, at least 97%, at least 98%, or at least 99%) identical to the VL as set forth in SEQ ID NO: 58. In some embodiments, the degree of sequence variation (e.g., at least 75%, at least 80%, at least 85%, at least 90%, at least 91%, at least 92%, at least 93%, at least 94%, at least 95%, at least 96%, at least 97%, at least 98%, or at least 99%) may occur within a VH of SEQ ID NO: 57, and/or a VL of SEQ ID NO: 58 excluding any of the CDR sequences therein. In some embodiments, an anti-trypsin 1/trypsin 2 antibody provided herein comprise a heavy chain variable sequence that is at least 75%, at least 80%, at least 85%, at least 90%, at least 91%, at least 92%, at least 93%, at least 94%, at least 95%, at least 96%, at least 97%, at least 98%, or at least 99% identical to the framework sequence of a VH of SEQ ID NO: 57, and/or a light chain variable sequence that comprises a framework sequence that is at least 75%, at least 80%, at least 85%, at least 90%, at least 91%, at least 92%, at least 93%, at least 94%, at least 95%, at least 96%, at least 97%, at least 98%, or at least 99% identical to the framework sequence of a VL of SEQ ID NO: 58.

In some embodiments, an anti-trypsin 1/trypsin 2 antibody of the present disclosure comprises a HC CDR1, HC CDR2 and HC CDR3 of a heavy chain variable domain having the amino acid sequence of SEQ ID NO: 59. Alternatively or in addition, the anti-anti-trypsin 1/trypsin 2 antibody of the present disclosure comprises a LC CDR1, LC CDR2 and LC CDR3 of a light chain variable domain having the amino acid sequence of SEQ ID NO: 60.

In some embodiments, an anti-trypsin 1/trypsin 2 antibody of the present disclosure comprises a HC CDR1 having the amino acid sequence of SEQ ID NO: 7, a HC CDR2 having the amino acid sequence of SEQ ID NO: 8, a HC CDR3 having the amino acid sequence of SEQ ID NO: 9, a LC CDR1 having the amino acid sequence of SEQ ID NO: 4, a LC CDR2 having the amino acid sequence of SEQ ID NO: 5, and a LC CDR3 having the amino acid sequence of SEQ ID NO: 10.

In some embodiments, an anti-trypsin 1/trypsin 2 antibody of the present disclosure comprises a HC CDR1, a HC CDR2, and a HC CDR3, which collectively contains no more than 5 amino acid variations (e.g., no more than 5, 4, 3, 2, or 1 amino acid variation) as compared with the HC CDR1 having the amino acid sequence of SEQ ID NO: 7, HC CDR2 having the amino acid sequence of SEQ ID NO: 8, and HC CDR3 having the amino acid sequence of SEQ ID NO: 9. Alternatively or in addition, the anti-trypsin 1/trypsin 2 antibody of the present disclosure comprises a LC CDR1, a LC CDR2, and a LC CDR3, which collectively contains no more than 5 amino acid variations (e.g., no more than 5, 4, 3, 2 or 1 amino acid variation) as compared with the LC CDR1 having the amino acid sequence of SEQ ID NO: 4, LC CDR2 having the amino acid sequence of SEQ ID NO: 5, and LC CDR3 having the amino acid sequence of SEQ ID NO: 10.

In some embodiments, an anti-trypsin 1/trypsin 2 antibody of the present disclosure comprises a HC CDR1, a HC CDR2, and a HC CDR3 that collectively are at least 80% (e.g., at least 80%, at least 85%, at least 90%, at least 91%, at least 92%, at least 93%, at least 94%, at least 95%, at least 96%, at least 97%, at least 98%, or at least 99%) identical to the HC CDR1 having the amino acid sequence of SEQ ID NO: 7, HC CDR2 having the amino acid sequence of SEQ ID NO: 8, and HC CDR3 having the amino acid sequence of SEQ ID NO: 9. Alternatively or in addition, the anti-trypsin 1/trypsin 2 antibody of the present disclosure comprises a LC CDR1, a LC CDR2, and a LC CDR3 that collectively are at least 80% (e.g., at least 80%, at least 85%, at least 90%, at least 91%, at least 92%, at least 93%, at least 94%, at least 95%, at least 96%, at least 97%, at least 98%, or at least 99%) identical to the to the LC CDR1 having the amino acid sequence of SEQ ID NO: 4, LC CDR2 having the amino acid sequence of SEQ ID NO: 5, and LC CDR3 having the amino acid sequence of SEQ ID NO: 10.

In some embodiments, an anti-trypsin 1/trypsin 2 antibody of the present disclosure comprises: a HC CDR1 having no more than 3 amino acid variations (e.g., no more than 3, 2, or 1 amino acid variation) as compared with the HC CDR1 having the amino acid sequence of SEQ ID NO: 7; a HC CDR2 having no more than 3 amino acid variations (e.g., no more than 3, 2, or 1 amino acid variation) as compared with the HC CDR2 having the amino acid sequence of SEQ ID NO: 8; and/or a HC CDR3 having no more than 3 amino acid variations (e.g., no more than 3, 2, or I amino acid variation) as compared with the HC CDR3 having the amino acid sequence of SEQ ID NO: 9. Alternatively or in addition, the anti-trypsin 1/trypsin 2 antibody of the present disclosure comprises: a LC CDR1 having no more than 3 amino acid variations (e.g., no more than 3, 2, or I amino acid variation) as compared with the LC CDR1 having the amino acid sequence of SEQ ID NO: 4; a LC CDR2 having no more than 3 amino acid variations (e.g., no more than 3, 2, or 1 amino acid variation) as compared with the LC CDR2 having the amino acid sequence of SEQ ID NO: 5; and/or a LC CDR3 having no more than 3 amino acid variations (e.g., no more than 3, 2, or I amino acid variation) as compared with the LC CDR3 having the amino acid sequence of SEQ ID NO: 10.

In some embodiments, an anti-trypsin 1/trypsin 2 antibody of the present disclosure comprises: a HC CDR1 that is at least 80% (e.g., at least 80%, at least 85%, at least 90%, at least 91% e, at least 92%, at least 93%, at least 94%, at least 95%, at least 96%, at least 97%, at least 98%, or at least 99%) identical to the HC CDR1 having the amino acid sequence of SEQ ID NO: 7; a HC CDR2 that is at least 80% (e.g., at least 80%, at least 85%, at least 90%, at least 91%, at least 92%, at least 93%, at least 94%, at least 95%, at least 96%, at least 97%, at least 98%, or at least 99%) identical to the HC CDR2 having the amino acid sequence SEQ ID NO: 8; and/or a HC CDR3 that is at least 80% (e.g., at least 80%, at least 85%, at least 90%, at least 91%, at least 92%, at least 93%, at least 94%, at least 95%, at least 96%, at least 97%, at least 98%, or at least 99%) identical to the HC CDR3 having the amino acid sequence of SEQ ID NO: 9. Alternatively or in addition, the anti-trypsin 1/trypsin 2 antibody of the present disclosure comprises: a LC CDR1 that is at least 80% (e.g., at least 80%, at least 85%, at least 90%, at least 91%, at least 92%, at least 93%, at least 94%, at least 95%, at least 96%, at least 97%, at least 98%, or at least 99%) identical to the LC CDR1 having the amino acid sequence of SEQ ID NO: 4; a LC CDR2 that is at least 80% (e.g., at least 80%, at least 85%, at least 90%, at least 91%, at least 92%, at least 93%, at least 94%, at least 95%, at least 96%, at least 97%, at least 98%, or at least 99%) identical to the LC CDR2 having the amino acid sequence of SEQ ID NO: 5; and/or a LC CDR3 that is at least 80% (e.g., at least 80%, at least 85%, at least 90%, at least 91%, at least 92%, at least 93%, at least 94%, at least 95%, at least 96%, at least 97%, at least 98%, or at least 99%) identical to the LC CDR3 having the amino acid sequence of SEQ ID NO: 10.

In some embodiments, an anti-trypsin 1/trypsin 2 antibody of the present disclosure comprises a VH comprising the amino acid sequence of SEQ ID NO: 59. Alternatively or in addition, the anti-trypsin 1/trypsin 2 antibody of the present disclosure comprises a VL comprising the amino acid sequence of SEQ ID NO: 60.

In some embodiments, an anti-trypsin 1/trypsin 2 antibody of the present disclosure comprises a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 59. Alternatively or in addition, the anti-trypsin 1/trypsin 2 antibody of the present disclosure comprises a VL containing no more than 20 amino acid variations (e.g., no more than 20, 19, 18, 17, 16, 15, 14, 13, 12, 11, 9, 8, 7, 6, 5, 4, 3, 2, or I amino acid variation) as compared with the VL as set forth in SEQ ID NO: 60. In some embodiments, the number of amino acid variations (e.g., no more than 20, 19, 18, 17, 16, 15, 14, 13, 12, 11, 10, 9, 8, 7, 6, 5, 4, 3, 2, or I amino acid variation) may occur within a VH of SEQ ID NO: 59 and/or a VL of SEQ ID NO: 60 excluding any of the CDR sequences therein. In some embodiments, an anti-trypsin 1/trypsin 2 antibodies provided herein comprise a heavy chain variable sequence that comprises a framework sequence that that contains no more than 20, 19, 18, 17, 16, 15, 14, 13, 12, 11, 10, 9, 8, 7, 6, 5, 4, 3, 2, or I amino acid variation to the framework sequence of a VH of SEQ ID NO: 59, and/or a light chain variable sequence that comprises a framework sequence that that contains no more than 20, 19, 18, 17, 16, 15, 14, 13, 12, 11, 10, 9, 8, 7, 6, 5, 4, 3, 2, or 1 amino acid variation to the framework sequence of a VL of SEQ ID NO: 60.

In some embodiments, an anti-trypsin 1/trypsin 2 antibody of the present disclosure comprises a VH comprising an amino acid sequence that is at least 80% (e.g., at least 80%, at least 85%, at least 90%, at least 91%, at least 92%, at least 93%, at least 94%, at least 95%, at least 96%, at least 97%, at least 98%, or at least 99%) identical to the VH as set forth in SEQ ID NO: 59. Alternatively or in addition, the anti-trypsin 1/trypsin 2 antibody of the present disclosure comprises a VL comprising an amino acid sequence that is at least 80% (e.g., at least 80%, at least 85%, at least 90%, at least 91%, at least 92%, at least 93%, at least 94%, at least 95%, at least 96%, at least 97%, at least 98%, or at least 99%) identical to the VL as set forth in SEQ ID NO: 60. In some embodiments, the degree of sequence variation (e.g., at least 75%, at least 80%, at least 85%, at least 90%, at least 91%, at least 92%, at least 93%, at least 94%, at least 95%, at least 96%, at least 97%, at least 98%, or at least 99%) may occur within a VH of SEQ ID NO: 59, and/or a VL of SEQ ID NO: 60 excluding any of the CDR sequences therein. In some embodiments, an anti-trypsin 1/trypsin 2 antibody provided herein comprise a heavy chain variable sequence that is at least 75%, at least 80%, at least 85%, at least 90%, at least 91%, at least 92%, at least 93%, at least 94%, at least 95%, at least 96%, at least 97%, at least 98%, or at least 99% identical to the framework sequence of a VH of SEQ ID NO: 59, and/or a light chain variable sequence that comprises a framework sequence that is at least 75%, at least 80%, at least 85%, at least 90%, at least 91%, at least 92%, at least 93%, at least 94%, at least 95%, at least 96%, at least 97%, at least 98%, or at least 99% identical to the framework sequence of a VL of SEQ ID NO: 60.

In some embodiments, an anti-trypsin 1/trypsin 2 antibody of the present disclosure comprises a HC CDR1, HC CDR2 and HC CDR3 of a heavy chain variable domain having the amino acid sequence of SEQ ID NO: 61. Alternatively or in addition, the anti-anti-trypsin 1/trypsin 2 antibody of the present disclosure comprises a LC CDR1, LC CDR2 and LC CDR3 of a light chain variable domain having the amino acid sequence of SEQ ID NO: 62.

In some embodiments, an anti-trypsin 1/trypsin 2 antibody of the present disclosure comprises a HC CDR1 having the amino acid sequence of SEQ ID NO: II, a HC CDR2 having the amino acid sequence of SEQ ID NO: 12, a HC CDR3 having the amino acid sequence of SEQ ID NO: 13, a LC CDR1 having the amino acid sequence of SEQ ID NO: 14, a LC CDR2 having the amino acid sequence of SEQ ID NO: 15, and a LC CDR3 having the amino acid sequence of SEQ ID NO: 16.

In some embodiments, an anti-trypsin 1/trypsin 2 antibody of the present disclosure comprises a HC CDR1, a HC CDR2, and a HC CDR3, which collectively contains no more than 5 amino acid variations (e.g., no more than 5, 4, 3, 2, or I amino acid variation) as compared with the HC CDR1 having the amino acid sequence of SEQ ID NO: 11, HC CDR2 having the amino acid sequence of SEQ ID NO: 12, and HC CDR3 having the amino acid sequence of SEQ ID NO: 13. Alternatively or in addition, the anti-trypsin 1/trypsin 2 antibody of the present disclosure comprises a LC CDR1, a LC CDR2, and a LC CDR3, which collectively contains no more than 5 amino acid variations (e.g., no more than 5, 4, 3, 2 or 1 amino acid variation) as compared with the LC CDR1 having the amino acid sequence of SEQ ID NO: 14, LC CDR2 having the amino acid sequence of SEQ ID NO: 15, and LC CDR3 having the amino acid sequence of SEQ ID NO: 16.

In some embodiments, an anti-trypsin 1/trypsin 2 antibody of the present disclosure comprises a HC CDR1, a HC CDR2, and a HC CDR3 that collectively are at least 80% (e.g., at least 80%, at least 85%, at least 90%, at least 91%, at least 92%, at least 93%, at least 94%, at least 95%, at least 96%, at least 97%, at least 98%, or at least 99%) identical to the HC CDR1 having the amino acid sequence of SEQ ID NO: 11, HC CDR2 having the amino acid sequence of SEQ ID NO: 12, and HC CDR3 having the amino acid sequence of SEQ ID NO: 13. Alternatively or in addition, the anti-trypsin 1/trypsin 2 antibody of the present disclosure comprises a LC CDR1, a LC CDR2, and a LC CDR3 that collectively are at least 80% (e.g., at least 80%, at least 85%, at least 90%, at least 91%, at least 92%, at least 93%, at least 94%, at least 95%, at least 96%, at least 97%, at least 98%, or at least 99%) identical to the to the LC CDR1 having the amino acid sequence of SEQ ID NO: 14, LC CDR2 having the amino acid sequence of SEQ ID NO: 15, and LC CDR3 having the amino acid sequence of SEQ ID NO: 16.

In some embodiments, an anti-trypsin 1/trypsin 2 antibody of the present disclosure comprises: a HC CDR1 having no more than 3 amino acid variations (e.g., no more than 3, 2, or 1 amino acid variation) as compared with the HC CDR1 having the amino acid sequence of SEQ ID NO: 11; a HC CDR2 having no more than 3 amino acid variations (e.g., no more than 3, 2, or 1 amino acid variation) as compared with the HC CDR2 having the amino acid sequence of SEQ ID NO: 12; and/or a HC CDR3 having no more than 3 amino acid variations (e.g., no more than 3, 2, or 1 amino acid variation) as compared with the HC CDR3 having the amino acid sequence of SEQ ID NO: 13. Alternatively or in addition, the anti-trypsin 1/trypsin 2 antibody of the present disclosure comprises: a LC CDR1 having no more than 3 amino acid variations (e.g., no more than 3, 2, or 1 amino acid variation) as compared with the LC CDR1 having the amino acid sequence of SEQ ID NO: 14; a LC CDR2 having no more than 3 amino acid variations (e.g., no more than 3, 2, or 1 amino acid variation) as compared with the LC CDR2 having the amino acid sequence of SEQ ID NO: 15; and/or a LC CDR3 having no more than 3 amino acid variations (e.g., no more than 3, 2, or 1 amino acid variation) as compared with the LC CDR3 having the amino acid sequence of SEQ ID NO: 16.

In some embodiments, an anti-trypsin 1/trypsin 2 antibody of the present disclosure comprises: a HC CDR1 that is at least 80% (e.g., at least 80%, at least 85%, at least 90%, at least 91%, at least 92%, at least 93%, at least 94%, at least 95%, at least 96%, at least 97%, at least 98%, or at least 99%) identical to the HC CDR1 having the amino acid sequence of SEQ ID NO: 11; a HC CDR2 that is at least 80% (e.g., at least 80%, at least 85%, at least 90%, at least 91%, at least 92%, at least 93%, at least 94%, at least 95%, at least 96%, at least 97%, at least 98%, or at least 99%) identical to the HC CDR2 having the amino acid sequence SEQ ID NO: 12; and/or a HC CDR3 that is at least 80% (e.g., at least 80%, at least 85%, at least 90%, at least 91%, at least 92%, at least 93%, at least 94%, at least 95%, at least 96%, at least 97%, at least 98%, or at least 99%) identical to the HC CDR3 having the amino acid sequence of SEQ ID NO: 13. Alternatively or in addition, the anti-trypsin 1/trypsin 2 antibody of the present disclosure comprises: a LC CDR1 that is at least 80% (e.g., at least 80%, at least 85%, at least 90%, at least 91%, at least 92%, at least 93%, at least 94%, at least 95%, at least 96%, at least 97%, at least 98%, or at least 99%) identical to the LC CDR1 having the amino acid sequence of SEQ ID NO: 14; a LC CDR2 that is at least 80% (e.g., at least 80%, at least 85%, at least 90%, at least 91%, at least 92%, at least 93%, at least 94%, at least 95%, at least 96%, at least 97%, at least 98%, or at least 99%) identical to the LC CDR2 having the amino acid sequence of SEQ ID NO: 15; and/or a LC CDR3 that is at least 80% (e.g., at least 80%, at least 85%, at least 90%, at least 91%, at least 92%, at least 93%, at least 94%, at least 95%, at least 96%, at least 97%, at least 98%, or at least 99%) identical to the LC CDR3 having the amino acid sequence of SEQ ID NO: 16.

In some embodiments, an anti-trypsin 1/trypsin 2 antibody of the present disclosure comprises a VH comprising the amino acid sequence of SEQ ID NO: 61. Alternatively or in addition, the anti-trypsin 1/trypsin 2 antibody of the present disclosure comprises a VL comprising the amino acid sequence of SEQ ID NO: 62.

In some embodiments, an anti-trypsin 1/trypsin 2 antibody of the present disclosure comprises a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 61. Alternatively or in addition, the anti-trypsin 1/trypsin 2 antibody of the present disclosure comprises a VL containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the VL as set forth in SEQ ID NO: 62. In some embodiments, the number of amino acid variations (e.g., no more than 20, 19, 18, 17, 16, 15, 14, 13, 12, 11, 10, 9, 8, 7, 6, 5, 4, 3, 2, or I amino acid variation) may occur within a VH of SEQ ID NO: 61 and/or a VL of SEQ ID NO: 62 excluding any of the CDR sequences therein. In some embodiments, an anti-trypsin 1/trypsin 2 antibodies provided herein comprise a heavy chain variable sequence that comprises a framework sequence that that contains no more than 20, 19, 18, 17, 16, 15, 14, 13, 12, 11, 10, 9, 8, 7, 6, 5, 4, 3, 2, or 1 amino acid variation to the framework sequence of a VH of SEQ ID NO: 61, and/or a light chain variable sequence that comprises a framework sequence that that contains no more than 20, 19, 18, 17, 16, 15, 14, 13, 12, 11, 10, 9, 8, 7, 6, 5, 4, 3, 2, or 1 amino acid variation to the framework sequence of a VL of SEQ ID NO: 62.

In some embodiments, an anti-trypsin 1/trypsin 2 antibody of the present disclosure comprises a VH comprising an amino acid sequence that is at least 80% (e.g., at least 80%, at least 85%, at least 90%, at least 91%, at least 92%, at least 93%, at least 94%, at least 95%, at least 96%, at least 97%, at least 98%, or at least 99%) identical to the VH as set forth in SEQ ID NO: 61. Alternatively or in addition, the anti-trypsin 1/trypsin 2 antibody of the present disclosure comprises a VL comprising an amino acid sequence that is at least 80% (e.g., at least 80%, at least 85%, at least 90%, at least 91%, at least 92%, at least 93%, at least 94%, at least 95%, at least 96%, at least 97%, at least 98%, or at least 99%) identical to the VL as set forth in SEQ ID NO: 62. In some embodiments, the degree of sequence variation (e.g., at least 75%, at least 80%, at least 85%, at least 90%, at least 91%, at least 92%, at least 93%, at least 94%, at least 95%, at least 96%, at least 97%, at least 98%, or at least 99%) may occur within a VH of SEQ ID NO: 61, and/or a VL of SEQ ID NO: 62 excluding any of the CDR sequences therein. In some embodiments, an anti-trypsin 1/trypsin 2 antibody provided herein comprise a heavy chain variable sequence that is at least 75%, at least 80%, at least 85%, at least 90%, at least 91%, at least 92%, at least 93%, at least 94%, at least 95%, at least 96%, at least 97%, at least 98%, or at least 99% identical to the framework sequence of a VH of SEQ ID NO: 61, and/or a light chain variable sequence that comprises a framework sequence that is at least 75%, at least 80%, at least 85%, at least 90%, at least 91%, at least 92%, at least 93%, at least 94%, at least 95%, at least 96%, at least 97%, at least 98%, or at least 99% identical to the framework sequence of a V L of SEQ ID NO: 62.

In some embodiments, an anti-trypsin 1/trypsin 2 antibody of the present disclosure comprises a HC CDR1, HC CDR2 and HC CDR3 of a heavy chain variable domain having the amino acid sequence of SEQ ID NO: 63. Alternatively or in addition, the anti-anti-trypsin 1/trypsin 2 antibody of the present disclosure comprises a LC CDR1, LC CDR2 and LC CDR3 of a light chain variable domain having the amino acid sequence of SEQ ID NO: 64.

In some embodiments, an anti-trypsin 1/trypsin 2 antibody of the present disclosure comprises a HC CDR1 having the amino acid sequence of SEQ ID NO: 17, a HC CDR2 having the amino acid sequence of SEQ ID NO: 18, a HC CDR3 having the amino acid sequence of SEQ ID NO: 19, a LC CDR1 having the amino acid sequence of SEQ ID NO: 14, a LC CDR2 having the amino acid sequence of SEQ ID NO: 15, and a LC CDR3 having the amino acid sequence of SEQ ID NO: 20.

In some embodiments, an anti-trypsin 1/trypsin 2 antibody of the present disclosure comprises a HC CDR1, a HC CDR2, and a HC CDR3, which collectively contains no more than 5 amino acid variations (e.g., no more than 5, 4, 3, 2, or I amino acid variation) as compared with the HC CDR1 having the amino acid sequence of SEQ ID NO: 17, HC CDR2 having the amino acid sequence of SEQ ID NO: 18, and HC CDR3 having the amino acid sequence of SEQ ID NO: 19. Alternatively or in addition, the anti-trypsin 1/trypsin 2 antibody of the present disclosure comprises a LC CDR1, a LC CDR2, and a LC CDR3, which collectively contains no more than 5 amino acid variations (e.g., no more than 5, 4, 3, 2 or 1 amino acid variation) as compared with the LC CDR1 having the amino acid sequence of SEQ ID NO: 14, LC CDR2 having the amino acid sequence of SEQ ID NO: 15, and LC CDR3 having the amino acid sequence of SEQ ID NO: 20.

In some embodiments, an anti-trypsin 1/trypsin 2 antibody of the present disclosure comprises a HC CDR1, a HC CDR2, and a HC CDR3 that collectively are at least 80% (e.g., at least 80%, at least 85%, at least 90%, at least 91%, at least 92%, at least 93%, at least 94%, at least 95%, at least 96%, at least 97%, at least 98%, or at least 99%) identical to the HC CDR1 having the amino acid sequence of SEQ ID NO: 17, NC CDR2 having the amino acid sequence of SEQ ID NO: 18, and HC CDR3 having the amino acid sequence of SEQ ID NO: 19. Alternatively or in addition, the anti-trypsin 1/trypsin 2 antibody of the present disclosure comprises a LC CDR1, a LC CDR2, and a LC CDR3 that collectively are at least 80% (e.g., at least 80%, at least 85%, at least 90%, at least 91%, at least 92%, at least 93%, at least 94%, at least 95%, at least 96%, at least 97%, at least 98%, or at least 99%) identical to the to the LC CDR1 having the amino acid sequence of SEQ ID NO: 14, LC CDR2 having the amino acid sequence of SEQ ID NO: 15, and LC CDR3 having the amino acid sequence of SEQ ID NO: 20.

In some embodiments, an anti-trypsin 1/trypsin 2 antibody of the present disclosure comprises: a HC CDR1 having no more than 3 amino acid variations (e.g., no more than 3, 2, or 1 amino acid variation) as compared with the HC CDR1 having the amino acid sequence of SEQ ID NO: 17; a HC CDR2 having no more than 3 amino acid variations (e.g., no more than 3, 2, or 1 amino acid variation) as compared with the HC CDR2 having the amino acid sequence of SEQ ID NO: 18; and/or a HC CDR3 having no more than 3 amino acid variations (e.g., no more than 3, 2, or 1 amino acid variation) as compared with the HC CDR3 having the amino acid sequence of SEQ ID NO: 19. Alternatively or in addition, the anti-trypsin 1/trypsin 2 antibody of the present disclosure comprises: a LC CDR1 having no more than 3 amino acid variations (e.g., no more than 3, 2, or 1 amino acid variation) as compared with the LC CDR1 having the amino acid sequence of SEQ ID NO: 14; a LC CDR2 having no more than 3 amino acid variations (e.g., no more than 3, 2, or I amino acid variation) as compared with the LC CDR2 having the amino acid sequence of SEQ ID NO: 15; and/or a LC CDR3 having no more than 3 amino acid variations (e.g., no more than 3, 2, or 1 amino acid variation) as compared with the LC CDR3 having the amino acid sequence of SEQ ID NO: 20.

In some embodiments, an anti-trypsin 1/trypsin 2 antibody of the present disclosure comprises: a HC CDR1 that is at least 80% (e.g., at least 80%, at least 85%, at least 90%, at least 91%, at least 92%, at least 93%, at least 94%, at least 95%, at least 96%, at least 97%, at least 98%, or at least 99%) identical to the HC CDR1 having the amino acid sequence of SEQ ID NO: 17; a HC CDR2 that is at least 80% (e.g., at least 80%, at least 85%, at least 90%, at least 91%, at least 92%, at least 93%, at least 94%, at least 95%, at least 96%, at least 97%, at least 98%, or at least 99%) identical to the HC CDR2 having the amino acid sequence SEQ ID NO: 18; and/or a HC CDR3 that is at least 80% (e.g., at least 80%, at least 85%, at least 90%, at least 91%, at least 92%, at least 93%, at least 94%, at least 95%, at least 96%, at least 97%, at least 98%, or at least 99%) identical to the HC CDR3 having the amino acid sequence of SEQ ID NO: 19. Alternatively or in addition, the anti-trypsin 1/trypsin 2 antibody of the present disclosure comprises: a LC CDR1 that is at least 80% (e.g., at least 80%, at least 85%, at least 90%, at least 91%, at least 92%, at least 93%, at least 94%, at least 95%, at least 96%, at least 97%, at least 98%, or at least 99%) identical to the LC CDR1 having the amino acid sequence of SEQ ID NO: 14; a LC CDR2 that is at least 80% (e.g., at least 80%, at least 85%, at least 90%, at least 91%, at least 92%, at least 93%, at least 94%, at least 95%, at least 96%, at least 97%, at least 98%, or at least 99%) identical to the LC CDR2 having the amino acid sequence of SEQ ID NO: 15; and/or a LC CDR3 that is at least 80% (e.g., at least 80%, at least 85%, at least 90%, at least 91%, at least 92%, at least 93%, at least 94%, at least 95%, at least 96%, at least 97%, at least 98%, or at least 99%) identical to the LC CDR3 having the amino acid sequence of SEQ ID NO: 20.

In some embodiments, an anti-trypsin 1/trypsin 2 antibody of the present disclosure comprises a VH comprising the amino acid sequence of SEQ ID NO: 63. Alternatively or in addition, the anti-trypsin 1/trypsin 2 antibody of the present disclosure comprises a VL comprising the amino acid sequence of SEQ ID NO: 64.

In some embodiments, an anti-trypsin 1/trypsin 2 antibody of the present disclosure comprises a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 63. Alternatively or in addition, the anti-trypsin 1/trypsin 2 antibody of the present disclosure comprises a VL containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the VL as set forth in SEQ ID NO: 64. In some embodiments, the number of amino acid variations (e.g., no more than 20, 19, 18, 17, 16, 15, 14, 13, 12, 11, 10, 9, 8, 7, 6, 5, 4, 3, 2, or I amino acid variation) may occur within a VH of SEQ ID NO: 63 and/or a VL of SEQ ID NO: 64 excluding any of the CDR sequences therein. In some embodiments, an anti-trypsin 1/trypsin 2 antibodies provided herein comprise a heavy chain variable sequence that comprises a framework sequence that that contains no more than 20, 19, 18, 17, 16, 15, 14, 13, 12, 11, 10, 9, 8, 7, 6, 5, 4, 3, 2, or I amino acid variation to the framework sequence of a VH of SEQ ID NO: 63, and/or a light chain variable sequence that comprises a framework sequence that that contains no more than 20, 19, 18, 17, 16, 15, 14, 13, 12, 11, 10, 9, 8, 7, 6, 5, 4, 3, 2, or 1 amino acid variation to the framework sequence of a VL of SEQ ID NO: 64.

In some embodiments, an anti-trypsin 1/trypsin 2 antibody of the present disclosure comprises a VH comprising an amino acid sequence that is at least 80% (e.g., at least 80%, at least 85%, at least 90%, at least 91%, at least 92%, at least 93%, at least 94%, at least 95%, at least 96%, at least 97%, at least 98%, or at least 99%) identical to the VH as set forth in SEQ ID NO: 63. Alternatively or in addition, the anti-trypsin 1/trypsin 2 antibody of the present disclosure comprises a VL comprising an amino acid sequence that is at least 80% (e.g., at least 80%, at least 85%, at least 90%, at least 91%, at least 92%, at least 93%, at least 94%, at least 95%, at least 96%, at least 97%, at least 98%, or at least 99%) identical to the VL as set forth in SEQ ID NO: 64. In some embodiments, the degree of sequence variation (e.g., at least 75%, at least 80%, at least 85%, at least 90%, at least 91%, at least 92%, at least 93%, at least 94%, at least 95%, at least 96%, at least 97%, at least 98%, or at least 99%) may occur within a VH of SEQ ID NO: 63, and/or a VL of SEQ ID NO: 64 excluding any of the CDR sequences therein. In some embodiments, an anti-trypsin 1/trypsin 2 antibody provided herein comprise a heavy chain variable sequence that is at least 75%, at least 80%, at least 85%, at least 90%, at least 91%, at least 92%, at least 93%, at least 94%, at least 95%, at least 96%, at least 97%, at least 98%, or at least 99% identical to the framework sequence of a VH of SEQ ID NO: 63, and/or a light chain variable sequence that comprises a framework sequence that is at least 75%, at least 80%, at least 85%, at least 90%, at least 91%, at least 92%, at least 93%, at least 94%, at least 95%, at least 96%, at least 97%, at least 98%, or at least 99% identical to the framework sequence of a VL of SEQ ID NO: 64.

In some embodiments, an anti-trypsin 1/trypsin 2 antibody of the present disclosure comprises a HC CDR1, HC CDR2 and HC CDR3 of a heavy chain variable domain having the amino acid sequence of SEQ ID NO: 65. Alternatively or in addition, the anti-anti-trypsin 1/trypsin 2 antibody of the present disclosure comprises a LC CDR1, LC CDR2 and LC CDR3 of a light chain variable domain having the amino acid sequence of SEQ ID NO: 66.

In some embodiments, an anti-trypsin 1/trypsin 2 antibody of the present disclosure comprises a HC CDR1 having the amino acid sequence of SEQ ID NO: 21, a HC CDR2 having the amino acid sequence of SEQ ID NO: 22, a HC CDR3 having the amino acid sequence of SEQ ID NO: 23, a LC CDR1 having the amino acid sequence of SEQ ID NO: 14, a LC CDR2 having the amino acid sequence of SEQ ID NO: 15, and a LC CDR3 having the amino acid sequence of SEQ ID NO: 24.

In some embodiments, an anti-trypsin 1/trypsin 2 antibody of the present disclosure comprises a HC CDR1, a HC CDR2, and a HC CDR3, which collectively contains no more than 5 amino acid variations (e.g., no more than 5, 4, 3, 2, or 1 amino acid variation) as compared with the HC CDR1 having the amino acid sequence of SEQ ID NO: 21, HC CDR2 having the amino acid sequence of SEQ ID NO: 22, and HC CDR3 having the amino acid sequence of SEQ ID NO: 23. Alternatively or in addition, the anti-trypsin 1/trypsin 2 antibody of the present disclosure comprises a LC CDR1, a LC CDR2, and a LC CDR3, which collectively contains no more than 5 amino acid variations (e.g., no more than 5, 4, 3, 2 or 1 amino acid variation) as compared with the LC CDR1 having the amino acid sequence of SEQ ID NO: 14, LC CDR2 having the amino acid sequence of SEQ ID NO: 15, and LC CDR3 having the amino acid sequence of SEQ ID NO: 24.

In some embodiments, an anti-trypsin 1/trypsin 2 antibody of the present disclosure comprises a HC CDR1, a HC CDR2, and a HC CDR3 that collectively are at least 80% (e.g., at least 80%, at least 85%, at least 90%, at least 91%, at least 92%, at least 93%, at least 94%, at least 95%, at least 96%, at least 97%, at least 98%, or at least 99%) identical to the HC CDR1 having the amino acid sequence of SEQ ID NO: 21, HC CDR2 having the amino acid sequence of SEQ ID NO: 22, and HC CDR3 having the amino acid sequence of SEQ ID NO: 23. Alternatively or in addition, the anti-trypsin 1/trypsin 2 antibody of the present disclosure comprises a LC CDR1, a LC CDR2, and a LC CDR3 that collectively are at least 80% (e.g., at least 80%, at least 85%, at least 90%, at least 91%, at least 92%, at least 93%, at least 94%, at least 95%, at least 96%, at least 97%, at least 98%, or at least 99%) identical to the to the LC CDR1 having the amino acid sequence of SEQ ID NO: 14, LC CDR2 having the amino acid sequence of SEQ ID NO: 15, and LC CDR3 having the amino acid sequence of SEQ ID NO: 24.

In some embodiments, an anti-trypsin 1/trypsin 2 antibody of the present disclosure comprises: a HC CDR1 having no more than 3 amino acid variations (e.g., no more than 3, 2, or 1 amino acid variation) as compared with the HC CDR1 having the amino acid sequence of SEQ ID NO: 21; a HC CDR2 having no more than 3 amino acid variations (e.g., no more than 3, 2, or I amino acid variation) as compared with the HC CDR2 having the amino acid sequence of SEQ ID NO: 22; and/or a HC CDR3 having no more than 3 amino acid variations (e.g., no more than 3, 2, or 1 amino acid variation) as compared with the HC CDR3 having the amino acid sequence of SEQ ID NO: 23. Alternatively or in addition, the anti-trypsin 1/trypsin 2 antibody of the present disclosure comprises: a LC CDR1 having no more than 3 amino acid variations (e.g., no more than 3, 2, or 1 amino acid variation) as compared with the LC CDR1 having the amino acid sequence of SEQ ID NO: 14; a LC CDR2 having no more than 3 amino acid variations (e.g., no more than 3, 2, or 1 amino acid variation) as compared with the LC CDR2 having the amino acid sequence of SEQ ID NO: 15; and/or a LC CDR3 having no more than 3 amino acid variations (e.g., no more than 3, 2, or 1 amino acid variation) as compared with the LC CDR3 having the amino acid sequence of SEQ ID NO: 24.

In some embodiments, an anti-trypsin 1/trypsin 2 antibody of the present disclosure comprises: a HC CDR1 that is at least 80% (e.g., at least 80%, at least 85%, at least 90%, at least 91%, at least 92%, at least 93%, at least 94%, at least 95%, at least 96%, at least 97%, at least 98%, or at least 99%) identical to the HC CDR1 having the amino acid sequence of SEQ ID NO: 21; a HC CDR2 that is at least 80% (e.g., at least 80%, at least 85%, at least 90%, at least 91%, at least 92%, at least 93%, at least 94%, at least 95%, at least 96%, at least 97%, at least 98%, or at least 99%) identical to the HC CDR2 having the amino acid sequence SEQ ID NO: 22; and/or a HC CDR3 that is at least 80% (e.g., at least 80%, at least 85%, at least 90%, at least 91%, at least 92%, at least 93%, at least 94%, at least 95%, at least 96%, at least 97%, at least 98%, or at least 99%) identical to the HC CDR3 having the amino acid sequence of SEQ ID NO: 23. Alternatively or in addition, the anti-trypsin 1/trypsin 2 antibody of the present disclosure comprises: a LC CDR1 that is at least 80% (e.g., at least 80%, at least 85%, at least 90%, at least 91%, at least 92%, at least 93%, at least 94%, at least 95%, at least 96%, at least 97%, at least 98%, or at least 99%) identical to the LC CDR1 having the amino acid sequence of SEQ ID NO: 14; a LC CDR2 that is at least 80% (e.g., at least 80%, at least 85%, at least 90%, at least 91%, at least 92%, at least 93%, at least 94%, at least 95%, at least 96%, at least 97%, at least 98%, or at least 99%) identical to the LC CDR2 having the amino acid sequence of SEQ ID NO: 15; and/or a LC CDR3 that is at least 80% (e.g., at least 80%, at least 85%, at least 90%, at least 91%, at least 92%, at least 93%, at least 94%, at least 95%, at least 96%, at least 97%, at least 98%, or at least 99%) identical to the LC CDR3 having the amino acid sequence of SEQ ID NO: 24.

In some embodiments, an anti-trypsin 1/trypsin 2 antibody of the present disclosure comprises a VH comprising the amino acid sequence of SEQ ID NO: 65. Alternatively or in addition, the anti-trypsin 1/trypsin 2 antibody of the present disclosure comprises a VL comprising the amino acid sequence of SEQ ID NO: 66.

In some embodiments, an anti-trypsin 1/trypsin 2 antibody of the present disclosure comprises a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 65. Alternatively or in addition, the anti-trypsin 1/trypsin 2 antibody of the present disclosure comprises a VL containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the VL as set forth in SEQ ID NO: 66. In some embodiments, the number of amino acid variations (e.g., no more than 20, 19, 18, 17, 16, 15, 14, 13, 12, 11, 10, 9, 8, 7, 6, 5, 4, 3, 2, or I amino acid variation) may occur within a VH of SEQ ID NO: 65 and/or a VL of SEQ ID NO: 66 excluding any of the CDR sequences therein. In some embodiments, an anti-trypsin 1/trypsin 2 antibodies provided herein comprise a heavy chain variable sequence that comprises a framework sequence that that contains no more than 20, 19, 18, 17, 16, 15, 14, 13, 12, 11, 10, 9, 8, 7, 6, 5, 4, 3, 2, or 1 amino acid variation to the framework sequence of a VH of SEQ ID NO: 65, and/or a light chain variable sequence that comprises a framework sequence that that contains no more than 20, 19, 18, 17, 16, 15, 14, 13, 12, 11, 10, 9, 8, 7, 6, 5, 4, 3, 2, or 1 amino acid variation to the framework sequence of a VL of SEQ ID NO: 66.

In some embodiments, an anti-trypsin 1/trypsin 2 antibody of the present disclosure comprises a VH comprising an amino acid sequence that is at least 80% (e.g., at least 80%, at least 85%, at least 90%, at least 91%, at least 92%, at least 93%, at least 94%, at least 95%, at least 96%, at least 97%, at least 98%, or at least 99%) identical to the VH as set forth in SEQ ID NO: 65. Alternatively or in addition, the anti-trypsin 1/trypsin 2 antibody of the present disclosure comprises a VL comprising an amino acid sequence that is at least 80% (e.g., at least 80%, at least 85%, at least 90%, at least 91%, at least 92%, at least 93%, at least 94%, at least 95%, at least 96%, at least 97%, at least 98%, or at least 99%) identical to the VL as set forth in SEQ ID NO: 66. In some embodiments, the degree of sequence variation (e.g., at least 75%, at least 80%, at least 85%, at least 90%, at least 91%, at least 92%, at least 93%, at least 94%, at least 95%, at least 96%, at least 97%, at least 98%, or at least 99%) may occur within a VH of SEQ ID NO: 65, and/or a VL of SEQ ID NO: 66 excluding any of the CDR sequences therein. In some embodiments, an anti-trypsin 1/trypsin 2 antibody provided herein comprise a heavy chain variable sequence that is at least 75%, at least 80%, at least 85%, at least 90%, at least 91%, at least 92%, at least 93%, at least 94%, at least 95%, at least 96%, at least 97%, at least 98%, or at least 99% identical to the framework sequence of a VH of SEQ ID NO: 65, and/or a light chain variable sequence that comprises a framework sequence that is at least 75%, at least 80%, at least 85%, at least 90%, at least 91%, at least 92%, at least 93%, at least 94%, at least 95%, at least 96%, at least 97%, at least 98%, or at least 99% identical to the framework sequence of a V L of SEQ ID NO: 66.

In some embodiments, an anti-trypsin 1/trypsin 2 antibody of the present disclosure comprises a HC CDR1, HC CDR2 and HC CDR3 of a heavy chain variable domain having the amino acid sequence of SEQ ID NO: 67. Alternatively or in addition, the anti-anti-trypsin 1/trypsin 2 antibody of the present disclosure comprises a LC CDR1, LC CDR2 and LC CDR3 of a light chain variable domain having the amino acid sequence of SEQ ID NO: 68.

In some embodiments, an anti-trypsin 1/trypsin 2 antibody of the present disclosure comprises a HC CDR1 having the amino acid sequence of SEQ ID NO: 21, a HC CDR2 having the amino acid sequence of SEQ ID NO: 18, a HC CDR3 having the amino acid sequence of SEQ ID NO: 25, a LC CDR1 having the amino acid sequence of SEQ ID NO: 14, a LC CDR2 having the amino acid sequence of SEQ ID NO: 15, and a LC CDR3 having the amino acid sequence of SEQ ID NO: 26.

In some embodiments, an anti-trypsin 1/trypsin 2 antibody of the present disclosure comprises a HC CDR1, a HC CDR2, and a HC CDR3, which collectively contains no more than 5 amino acid variations (e.g., no more than 5, 4, 3, 2, or I amino acid variation) as compared with the HC CDR1 having the amino acid sequence of SEQ ID NO: 21, HC CDR2 having the amino acid sequence of SEQ ID NO: 18, and HC CDR3 having the amino acid sequence of SEQ ID NO: 25. Alternatively or in addition, the anti-trypsin 1/trypsin 2 antibody of the present disclosure comprises a LC CDR1, a LC CDR2, and a LC CDR3, which collectively contains no more than 5 amino acid variations (e.g., no more than 5, 4, 3, 2 or 1 amino acid variation) as compared with the LC CDR1 having the amino acid sequence of SEQ ID NO: 14, LC CDR2 having the amino acid sequence of SEQ ID NO: 15, and LC CDR3 having the amino acid sequence of SEQ ID NO: 26.

In some embodiments, an anti-trypsin 1/trypsin 2 antibody of the present disclosure comprises a HC CDR1, a HC CDR2, and a HC CDR3 that collectively are at least 80% (e.g., at least 80%, at least 85%, at least 90%, at least 91%, at least 92%, at least 93%, at least 94%, at least 95%, at least 96%, at least 97%, at least 98%, or at least 99%) identical to the HC CDR1 having the amino acid sequence of SEQ ID NO: 21, HC CDR2 having the amino acid sequence of SEQ ID NO: 18, and HC CDR3 having the amino acid sequence of SEQ ID NO: 25. Alternatively or in addition, the anti-trypsin 1/trypsin 2 antibody of the present disclosure comprises a LC CDR1, a LC CDR2, and a LC CDR3 that collectively are at least 80% (e.g., at least 80%, at least 85%, at least 90%, at least 91%, at least 92%, at least 93%, at least 94%, at least 95%, at least 96%, at least 97%, at least 98%, or at least 99%) identical to the to the LC CDR1 having the amino acid sequence of SEQ ID NO: 14, LC CDR2 having the amino acid sequence of SEQ ID NO: 15, and LC CDR3 having the amino acid sequence of SEQ ID NO: 26.

In some embodiments, an anti-trypsin 1/trypsin 2 antibody of the present disclosure comprises: a HC CDR1 having no more than 3 amino acid variations (e.g., no more than 3, 2, or 1 amino acid variation) as compared with the HC CDR1 having the amino acid sequence of SEQ ID NO: 21; a HC CDR2 having no more than 3 amino acid variations (e.g., no more than 3, 2, or I amino acid variation) as compared with the HC CDR2 having the amino acid sequence of SEQ ID NO: 18; and/or a HC CDR3 having no more than 3 amino acid variations (e.g., no more than 3, 2, or 1 amino acid variation) as compared with the HC CDR3 having the amino acid sequence of SEQ ID NO: 25. Alternatively or in addition, the anti-trypsin 1/trypsin 2 antibody of the present disclosure comprises: a LC CDR1 having no more than 3 amino acid variations (e.g., no more than 3, 2, or I amino acid variation) as compared with the LC CDR1 having the amino acid sequence of SEQ ID NO: 14; a LC CDR2 having no more than 3 amino acid variations (e.g., no more than 3, 2, or I amino acid variation) as compared with the LC CDR2 having the amino acid sequence of SEQ ID NO: 15; and/or a LC CDR3 having no more than 3 amino acid variations (e.g., no more than 3, 2, or 1 amino acid variation) as compared with the LC CDR3 having the amino acid sequence of SEQ ID NO: 26.

In some embodiments, an anti-trypsin 1/trypsin 2 antibody of the present disclosure comprises: a HC CDR1 that is at least 80% (e.g., at least 80%, at least 85%, at least 90%, at least 91%, at least 92%, at least 93%, at least 94%, at least 95%, at least 96%, at least 97%, at least 98%, or at least 99%) identical to the HC CDR1 having the amino acid sequence of SEQ ID NO: 21; a HC CDR2 that is at least 80% (e.g., at least 80%, at least 85%, at least 90%, at least 91%, at least 92%, at least 93%, at least 94%, at least 95%, at least 96%, at least 97%, at least 98%, or at least 99%) identical to the HC CDR2 having the amino acid sequence SEQ ID NO: 18; and/or a HC CDR3 that is at least 80% (e.g., at least 80%, at least 85%, at least 90%, at least 91%, at least 92%, at least 93%, at least 94%, at least 95%, at least 96%, at least 97%, at least 98%, or at least 99%) identical to the HC CDR3 having the amino acid sequence of SEQ ID NO: 25. Alternatively or in addition, the anti-trypsin 1/trypsin 2 antibody of the present disclosure comprises: a LC CDR1 that is at least 80% (e.g., at least 80%, at least 85%, at least 90%, at least 91%, at least 92%, at least 93%, at least 94%, at least 95%, at least 96%, at least 97%, at least 98%, or at least 99%) identical to the LC CDR1 having the amino acid sequence of SEQ ID NO: 14; a LC CDR2 that is at least 80% (e.g., at least 80%, at least 85%, at least 90%, at least 91%, at least 92%, at least 93%, at least 94%, at least 95%, at least 96%, at least 97%, at least 98%, or at least 99%) identical to the LC CDR2 having the amino acid sequence of SEQ ID NO: 15; and/or a LC CDR3 that is at least 80% (e.g., at least 80%, at least 85%, at least 90%, at least 91%, at least 92%, at least 93%, at least 94%, at least 95%, at least 96%, at least 97%, at least 98%, or at least 99%) identical to the LC CDR3 having the amino acid sequence of SEQ ID NO: 26.

In some embodiments, an anti-trypsin 1/trypsin 2 antibody of the present disclosure comprises a VH comprising the amino acid sequence of SEQ ID NO: 67. Alternatively or in addition, the anti-trypsin 1/trypsin 2 antibody of the present disclosure comprises a VL comprising the amino acid sequence of SEQ ID NO: 68.

In some embodiments, an anti-trypsin 1/trypsin 2 antibody of the present disclosure comprises a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 67. Alternatively or in addition, the anti-trypsin 1/trypsin 2 antibody of the present disclosure comprises a VL containing no more than 20 amino acid variations (e.g., no more than 20, 19, 18, 17, 16, 15, 14, 13, 12, 11, 9, 8, 7, 6, 5, 4, 3, 2, or I amino acid variation) as compared with the V L as set forth in SEQ ID NO: 68. In some embodiments, the number of amino acid variations (e.g., no more than 20, 19, 18, 17, 16, 15, 14, 13, 12, 11, 10, 9, 8, 7, 6, 5, 4, 3, 2, or I amino acid variation) may occur within a VH of SEQ ID NO: 67 and/or a VL of SEQ ID NO: 68 excluding any of the CDR sequences therein. In some embodiments, an anti-trypsin 1/trypsin 2 antibodies provided herein comprise a heavy chain variable sequence that comprises a framework sequence that that contains no more than 20, 19, 18, 17, 16, 15, 14, 13, 12, 11, 10, 9, 8, 7, 6, 5, 4, 3, 2, or I amino acid variation to the framework sequence of a VH of SEQ ID NO: 67, and/or a light chain variable sequence that comprises a framework sequence that that contains no more than 20, 19, 18, 17, 16, 15, 14, 13, 12, 11, 10, 9, 8, 7, 6, 5, 4, 3, 2, or 1 amino acid variation to the framework sequence of a VL of SEQ ID NO: 68.

In some embodiments, an anti-trypsin 1/trypsin 2 antibody of the present disclosure comprises a VH comprising an amino acid sequence that is at least 80% (e.g., at least 80%, at least 85%, at least 90%, at least 91%, at least 92%, at least 93%, at least 94%, at least 95%, at least 96%, at least 97%, at least 98%, or at least 99%) identical to the VH as set forth in SEQ ID NO: 67. Alternatively or in addition, the anti-trypsin 1/trypsin 2 antibody of the present disclosure comprises a VL comprising an amino acid sequence that is at least 80% (e.g., at least 80%, at least 85%, at least 90%, at least 91%, at least 92%, at least 93%, at least 94%, at least 95%, at least 96%, at least 97%, at least 98%, or at least 99%) identical to the VL as set forth in SEQ ID NO: 68. In some embodiments, the degree of sequence variation (e.g., at least 75%, at least 80%, at least 85%, at least 90%, at least 91%, at least 92%, at least 93%, at least 94%, at least 95%, at least 96%, at least 97%, at least 98%, or at least 99%) may occur within a VH of SEQ ID NO: 67, and/or a VL of SEQ ID NO: 68 excluding any of the CDR sequences therein. In some embodiments, an anti-trypsin 1/trypsin 2 antibody provided herein comprise a heavy chain variable sequence that is at least 75%, at least 80%, at least 85%, at least 90%, at least 91%, at least 92%, at least 93%, at least 94%, at least 95%, at least 96%, at least 97%, at least 98%, or at least 99% identical to the framework sequence of a VH of SEQ ID NO: 67, and/or a light chain variable sequence that comprises a framework sequence that is at least 75%, at least 80%, at least 85%, at least 90%, at least 91%, at least 92%, at least 93%, at least 94%, at least 95%, at least 96%, at least 97%, at least 98%, or at least 99% identical to the framework sequence of a VL of SEQ ID NO: 68.

In some embodiments, an anti-trypsin 1/trypsin 2 antibody of the present disclosure comprises a HC CDR1, HC CDR2 and HC CDR3 of a heavy chain variable domain having the amino acid sequence of SEQ ID NO: 69. Alternatively or in addition, the anti-anti-trypsin 1/trypsin 2 antibody of the present disclosure comprises a LC CDR1, LC CDR2 and LC CDR3 of a light chain variable domain having the amino acid sequence of SEQ ID NO: 70.

In some embodiments, an anti-trypsin 1/trypsin 2 antibody of the present disclosure comprises a HC CDR1 having the amino acid sequence of SEQ ID NO: 27, a HC CDR2 having the amino acid sequence of SEQ ID NO: 28, a HC CDR3 having the amino acid sequence of SEQ ID NO: 29, a LC CDR1 having the amino acid sequence of SEQ ID NO: 30, a LC CDR2 having the amino acid sequence of SEQ ID NO: 31, and a LC CDR3 having the amino acid sequence of SEQ ID NO: 32.

In some embodiments, an anti-trypsin 1/trypsin 2 antibody of the present disclosure comprises a HC CDR1, a HC CDR2, and a HC CDR3, which collectively contains no more than 5 amino acid variations (e.g., no more than 5, 4, 3, 2, or 1 amino acid variation) as compared with the HC CDR1 having the amino acid sequence of SEQ ID NO: 27, HC CDR2 having the amino acid sequence of SEQ ID NO: 28, and HC CDR3 having the amino acid sequence of SEQ ID NO: 29. Alternatively or in addition, the anti-trypsin 1/trypsin 2 antibody of the present disclosure comprises a LC CDR1, a LC CDR2, and a LC CDR3, which collectively contains no more than 5 amino acid variations (e.g., no more than 5, 4, 3, 2 or 1 amino acid variation) as compared with the LC CDR1 having the amino acid sequence of SEQ ID NO: 30, LC CDR2 having the amino acid sequence of SEQ ID NO: 31, and LC CDR3 having the amino acid sequence of SEQ ID NO: 32.

In some embodiments, an anti-trypsin 1/trypsin 2 antibody of the present disclosure comprises a HC CDR1, a HC CDR2, and a HC CDR3 that collectively are at least 80% (e.g., at least 80%, at least 85%, at least 90%, at least 91%, at least 92%, at least 93%, at least 94%, at least 95%, at least 96%, at least 97%, at least 98%, or at least 99%) identical to the HC CDR1 having the amino acid sequence of SEQ ID NO: 27, HC CDR2 having the amino acid sequence of SEQ ID NO: 28, and HC CDR3 having the amino acid sequence of SEQ ID NO: 29. Alternatively or in addition, the anti-trypsin 1/trypsin 2 antibody of the present disclosure comprises a LC CDR1, a LC CDR2, and a LC CDR3 that collectively are at least 80% (e.g., at least 80%, at least 85%, at least 90%, at least 91%, at least 92%, at least 93%, at least 94%, at least 95%, at least 96%, at least 97%, at least 98%, or at least 99%) identical to the to the LC CDR1 having the amino acid sequence of SEQ ID NO: 30, LC CDR2 having the amino acid sequence of SEQ ID NO: 31, and LC CDR3 having the amino acid sequence of SEQ ID NO: 32.

In some embodiments, an anti-trypsin 1/trypsin 2 antibody of the present disclosure comprises: a HC CDR1 having no more than 3 amino acid variations (e.g., no more than 3, 2, or 1 amino acid variation) as compared with the HC CDR1 having the amino acid sequence of SEQ ID NO: 27; a HC CDR2 having no more than 3 amino acid variations (e.g., no more than 3, 2, or I amino acid variation) as compared with the HC CDR2 having the amino acid sequence of SEQ ID NO: 28; and/or a HC CDR3 having no more than 3 amino acid variations (e.g., no more than 3, 2, or 1 amino acid variation) as compared with the HC CDR3 having the amino acid sequence of SEQ ID NO: 29. Alternatively or in addition, the anti-trypsin 1/trypsin 2 antibody of the present disclosure comprises: a LC CDR1 having no more than 3 amino acid variations (e.g., no more than 3, 2, or 1 amino acid variation) as compared with the LC CDR1 having the amino acid sequence of SEQ ID NO: 30; a LC CDR2 having no more than 3 amino acid variations (e.g., no more than 3, 2, or 1 amino acid variation) as compared with the LC CDR2 having the amino acid sequence of SEQ ID NO: 31; and/or a LC CDR3 having no more than 3 amino acid variations (e.g., no more than 3, 2, or 1 amino acid variation) as compared with the LC CDR3 having the amino acid sequence of SEQ ID NO: 32.

In some embodiments, an anti-trypsin 1/trypsin 2 antibody of the present disclosure comprises: a HC CDR1 that is at least 80% (e.g., at least 80%, at least 85%, at least 90%, at least 91%, at least 92%, at least 93%, at least 94%, at least 95%, at least 96%, at least 97%, at least 98%, or at least 99%) identical to the HC CDR1 having the amino acid sequence of SEQ ID NO: 27; a HC CDR2 that is at least 80% (e.g., at least 80%, at least 85%, at least 90%, at least 91%, at least 92%, at least 93%, at least 94%, at least 95%, at least 96%, at least 97%, at least 98%, or at least 99%) identical to the HC CDR2 having the amino acid sequence SEQ ID NO: 28; and/or a HC CDR3 that is at least 80% (e.g., at least 80%, at least 85%, at least 90%, at least 91%, at least 92%, at least 93%, at least 94%, at least 95%, at least 96%, at least 97%, at least 98%, or at least 99%) identical to the HC CDR3 having the amino acid sequence of SEQ ID NO: 29. Alternatively or in addition, the anti-trypsin 1/trypsin 2 antibody of the present disclosure comprises: a LC CDR1 that is at least 80% (e.g., at least 80%, at least 85%, at least 90%, at least 91%, at least 92%, at least 93%, at least 94%, at least 95%, at least 96%, at least 97%, at least 98%, or at least 99%) identical to the LC CDR1 having the amino acid sequence of SEQ ID NO: 30; a LC CDR2 that is at least 80% (e.g., at least 80%, at least 85%, at least 90%, at least 91%, at least 92%, at least 93%, at least 94%, at least 95%, at least 96%, at least 97%, at least 98%, or at least 99%) identical to the LC CDR2 having the amino acid sequence of SEQ ID NO: 31; and/or a LC CDR3 that is at least 80% (e.g., at least 80%, at least 85%, at least 90%, at least 91%, at least 92%, at least 93%, at least 94%, at least 95%, at least 96%, at least 97%, at least 98%, or at least 99%) identical to the LC CDR3 having the amino acid sequence of SEQ ID NO: 32.

In some embodiments, an anti-trypsin 1/trypsin 2 antibody of the present disclosure comprises a VHI comprising the amino acid sequence of SEQ ID NO: 69. Alternatively or in addition, the anti-trypsin 1/trypsin 2 antibody of the present disclosure comprises a VL comprising the amino acid sequence of SEQ ID NO: 70.

In some embodiments, an anti-trypsin 1/trypsin 2 antibody of the present disclosure comprises a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 69. Alternatively or in addition, the anti-trypsin 1/trypsin 2 antibody of the present disclosure comprises a VL containing no more than 20 amino acid variations (e.g., no more than 20, 19, 18, 17, 16, 15, 14, 13, 12, 11, 9, 8, 7, 6, 5, 4, 3, 2, or I amino acid variation) as compared with the VL as set forth in SEQ ID NO: 70. In some embodiments, the number of amino acid variations (e.g., no more than 20, 19, 18, 17, 16, 15, 14, 13, 12, 11, 10, 9, 8, 7, 6, 5, 4, 3, 2, or I amino acid variation) may occur within a VH of SEQ ID NO: 69 and/or a VL of SEQ ID NO: 70 excluding any of the CDR sequences therein, In some embodiments, an anti-trypsin 1/trypsin 2 antibodies provided herein comprise a heavy chain variable sequence that comprises a framework sequence that that contains no more than 20, 19, 18, 17, 16, 15, 14, 13, 12, 11, 10, 9, 8, 7, 6, 5, 4, 3, 2, or I amino acid variation to the framework sequence of a VH of SEQ ID NO: 69, and/or a light chain variable sequence that comprises a framework sequence that that contains no more than 20, 19, 18, 17, 16, 15, 14, 13, 12, 11, 10, 9, 8, 7, 6, 5, 4, 3, 2, or 1 amino acid variation to the framework sequence of a VL of SEQ ID NO: 70.

In some embodiments, an anti-trypsin 1/trypsin 2 antibody of the present disclosure comprises a VH comprising an amino acid sequence that is at least 80% (e.g., at least 80%, at least 85%, at least 90%, at least 91%, at least 92%, at least 93%, at least 94%, at least 95%, at least 96%, at least 97%, at least 98%, or at least 99%) identical to the VH as set forth in SEQ ID NO: 69, Alternatively or in addition, the anti-trypsin 1/trypsin 2 antibody of the present disclosure comprises a VL comprising an amino acid sequence that is at least 80% (e.g., at least 80%, at least 85%, at least 90%, at least 91%, at least 92%, at least 93%, at least 94%, at least 95%, at least 96%, at least 97%, at least 98%, or at least 99%) identical to the VL as set forth in SEQ ID NO: 70. In some embodiments, the degree of sequence variation (e.g., at least 75%, at least 80%, at least 85%, at least 90%, at least 91%, at least 92%, at least 93%, at least 94%, at least 95%, at least 96%, at least 97%, at least 98%, or at least 99%) may occur within a VH of SEQ ID NO: 69, and/or a VL of SEQ ID NO: 70 excluding any of the CDR sequences therein. In some embodiments, an anti-trypsin 1/trypsin 2 antibody provided herein comprise a heavy chain variable sequence that is at least 75%, at least 80%, at least 85%, at least 90%, at least 91%, at least 92%, at least 93%, at least 94%, at least 95%, at least 96%, at least 97%, at least 98%, or at least 99% identical to the framework sequence of a VH of SEQ ID NO: 69, and/or a light chain variable sequence that comprises a framework sequence that is at least 75%, at least 80%, at least 85%, at least 90%, at least 91%, at least 92%, at least 93%, at least 94%, at least 95%, at least 96%, at least 97%, at least 98%, or at least 99% identical to the framework sequence of a VL of SEQ ID NO: 70.

In some embodiments, an anti-trypsin 1/trypsin 2 antibody of the present disclosure comprises a HC CDR1, HC CDR2 and HC CDR3 of a heavy chain variable domain having the amino acid sequence of SEQ ID NO: 71. Alternatively or in addition, the anti-trypsin 1/trypsin 2 antibody of the present disclosure comprises a LC CDR1, LC CDR2 and LC CDR3 of a light chain variable domain having the amino acid sequence of SEQ ID NO: 70.

In some embodiments, an anti-trypsin 1/trypsin 2 antibody of the present disclosure comprises a HC CDR1 having the amino acid sequence of SEQ ID NO: 33, a HC CDR2 having the amino acid sequence of SEQ ID NO: 34, a HC CDR3 having the amino acid sequence of SEQ ID NO: 35, a LC CDR1 having the amino acid sequence of SEQ ID NO: 30, a LC CDR2 having the amino acid sequence of SEQ ID NO: 31, and a LC CDR3 having the amino acid sequence of SEQ ID NO: 32.

In some embodiments, an anti-trypsin 1/trypsin 2 antibody of the present disclosure comprises a HC CDR1, a HC CDR2, and a HC CDR3, which collectively contains no more than 5 amino acid variations (e.g., no more than 5, 4, 3, 2, or I amino acid variation) as compared with the HC CDR1 having the amino acid sequence of SEQ ID NO: 33, HC CDR2 having the amino acid sequence of SEQ ID NO: 34, and HC CDR3 having the amino acid sequence of SEQ ID NO: 35. Alternatively or in addition, the anti-trypsin 1/trypsin 2 antibody of the present disclosure comprises a LC CDR1, a LC CDR2, and a LC CDR3, which collectively contains no more than 5 amino acid variations (e.g., no more than 5, 4, 3, 2 or 1 amino acid variation) as compared with the LC CDR1 having the amino acid sequence of SEQ ID NO: 30, LC CDR2 having the amino acid sequence of SEQ ID NO: 31, and LC CDR3 having the amino acid sequence of SEQ ID NO: 32.

In some embodiments, an anti-trypsin 1/trypsin 2 antibody of the present disclosure comprises a HC CDR1, a HC CDR2, and a HC CDR3 that collectively are at least 80% (e.g., at least 80%, at least 85%, at least 90%, at least 91%, at least 92%, at least 93%, at least 94%, at least 95%, at least 96%, at least 97%, at least 98%, or at least 99%) identical to the HC CDR1 having the amino acid sequence of SEQ ID NO: 33, HC CDR2 having the amino acid sequence of SEQ ID NO: 34, and HC CDR3 having the amino acid sequence of SEQ ID NO: 35. Alternatively or in addition, the anti-trypsin 1/trypsin 2 antibody of the present disclosure comprises a LC CDR1, a LC CDR2, and a LC CDR3 that collectively are at least 80% (e.g., at least 80%, at least 85%, at least 90%, at least 91%, at least 92%, at least 93%, at least 94%, at least 95%, at least 96%, at least 97%, at least 98%, or at least 99%) identical to the to the LC CDR1 having the amino acid sequence of SEQ ID NO: 30, LC CDR2 having the amino acid sequence of SEQ ID NO: 31, and LC CDR3 having the amino acid sequence of SEQ ID NO: 32.

In some embodiments, an anti-trypsin 1/trypsin 2 antibody of the present disclosure comprises: a HC CDR1 having no more than 3 amino acid variations (e.g., no more than 3, 2, or 1 amino acid variation) as compared with the HC CDR1 having the amino acid sequence of SEQ ID NO: 33; a HC CDR2 having no more than 3 amino acid variations (e.g., no more than 3, 2, or I amino acid variation) as compared with the HC CDR2 having the amino acid sequence of SEQ ID NO: 34; and/or a HC CDR3 having no more than 3 amino acid variations (e.g., no more than 3, 2, or 1 amino acid variation) as compared with the HC CDR3 having the amino acid sequence of SEQ ID NO: 35. Alternatively or in addition, the anti-trypsin 1/trypsin 2 antibody of the present disclosure comprises: a LC CDR1 having no more than 3 amino acid variations (e.g., no more than 3, 2, or I amino acid variation) as compared with the LC CDR1 having the amino acid sequence of SEQ ID NO: 30; a LC CDR2 having no more than 3 amino acid variations (e.g., no more than 3, 2, or I amino acid variation) as compared with the LC CDR2 having the amino acid sequence of SEQ ID NO: 31; and/or a LC CDR3 having no more than 3 amino acid variations (e.g., no more than 3, 2, or 1 amino acid variation) as compared with the LC CDR3 having the amino acid sequence of SEQ ID NO: 32.

In some embodiments, an anti-trypsin 1/trypsin 2 antibody of the present disclosure comprises: a HC CDR1 that is at least 80% (e.g., at least 80%, at least 85%, at least 90%, at least 91%, at least 92%, at least 93%, at least 94%, at least 95%, at least 96%, at least 97%, at least 98%, or at least 99%) identical to the HC CDR1 having the amino acid sequence of SEQ ID NO: 33; a HC CDR2 that is at least 80% (e.g., at least 80%, at least 85%, at least 90%, at least 91%, at least 92%, at least 93%, at least 94%, at least 95%, at least 96%, at least 97%, at least 98%, or at least 99%) identical to the HC CDR2 having the amino acid sequence SEQ ID NO: 34; and/or a HC CDR3 that is at least 80% (e.g., at least 80%, at least 85%, at least 90%, at least 91%, at least 92%, at least 93%, at least 94%, at least 95%, at least 96%, at least 97%, at least 98%, or at least 99%) identical to the HC CDR3 having the amino acid sequence of SEQ ID NO: 35. Alternatively or in addition, the anti-trypsin 1/trypsin 2 antibody of the present disclosure comprises: a LC CDR1 that is at least 80% (e.g., at least 80%, at least 85%, at least 90%, at least 91%, at least 92%, at least 93%, at least 94%, at least 95%, at least 96%, at least 97%, at least 98%, or at least 99%) identical to the LC CDR1 having the amino acid sequence of SEQ ID NO: 30; a LC CDR2 that is at least 80% (e.g., at least 80%, at least 85%, at least 90%, at least 91%, at least 92%, at least 93%, at least 94%, at least 95%, at least 96%, at least 97%, at least 98%, or at least 99%) identical to the LC CDR2 having the amino acid sequence of SEQ ID NO: 31; and/or a LC CDR3 that is at least 80% (e.g., at least 80%, at least 85%, at least 90%, at least 91%, at least 92%, at least 93%, at least 94%, at least 95%, at least 96%, at least 97%, at least 98%, or at least 99%) identical to the LC CDR3 having the amino acid sequence of SEQ ID NO: 32.

In some embodiments, an anti-trypsin 1/trypsin 2 antibody of the present disclosure comprises a VH comprising the amino acid sequence of SEQ ID NO: 71. Alternatively or in addition, the anti-trypsin 1/trypsin 2 antibody of the present disclosure comprises a VL comprising the amino acid sequence of SEQ ID NO: 70.

In some embodiments, an anti-trypsin 1/trypsin 2 antibody of the present disclosure comprises a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 71. Alternatively or in addition, the anti-trypsin 1/trypsin 2 antibody of the present disclosure comprises a VL containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the VL as set forth in SEQ ID NO: 70. In some embodiments, the number of amino acid variations (e.g., no more than 20, 19, 18, 17, 16, 15, 14, 13, 12, 11, 10, 9, 8, 7, 6, 5, 4, 3, 2, or 1 amino acid variation) may occur within a VH of SEQ ID NO: 71 and/or a VL of SEQ ID NO: 70 excluding any of the CDR sequences therein. In some embodiments, an anti-trypsin 1/trypsin 2 antibodies provided herein comprise a heavy chain variable sequence that comprises a framework sequence that that contains no more than 20, 19, 18, 17, 16, 15, 14, 13, 12, 11, 10, 9, 8, 7, 6, 5, 4, 3, 2, or 1 amino acid variation to the framework sequence of a VH of SEQ ID NO: 71, and/or a light chain variable sequence that comprises a framework sequence that that contains no more than 20, 19, 18, 17, 16, 15, 14, 13, 12, 11, 10, 9, 8, 7, 6, 5, 4, 3, 2, or 1 amino acid variation to the framework sequence of a VL of SEQ ID NO: 70.

In some embodiments, an anti-trypsin 1/trypsin 2 antibody of the present disclosure comprises a VH comprising an amino acid sequence that is at least 80% (e.g., at least 80%, at least 85%, at least 90%, at least 91%, at least 92%, at least 93%, at least 94%, at least 95%, at least 96%, at least 97%, at least 98%, or at least 99%) identical to the VH as set forth in SEQ ID NO: 71. Alternatively or in addition, the anti-trypsin 1/trypsin 2 antibody of the present disclosure comprises a VL comprising an amino acid sequence that is at least 80% (e.g., at least 80%, at least 85%, at least 90%, at least 91%, at least 92%, at least 93%, at least 94%, at least 95%, at least 96%, at least 97%, at least 98%, or at least 99%) identical to the VL as set forth in SEQ ID NO: 70. In some embodiments, the degree of sequence variation (e.g., at least 75%, at least 80%, at least 85%, at least 90%, at least 91%, at least 92%, at least 93%, at least 94%, at least 95%, at least 96%, at least 97%, at least 98%, or at least 99%) may occur within a VH of SEQ ID NO: 71, and/or a VL of SEQ ID NO: 70 excluding any of the CDR sequences therein. In some embodiments, an anti-trypsin 1/trypsin 2 antibody provided herein comprise a heavy chain variable sequence that is at least 75%, at least 80%, at least 85%, at least 90%, at least 91%, at least 92%, at least 93%, at least 94%, at least 95%, at least 96%, at least 97%, at least 98%, or at least 99% identical to the framework sequence of a VH of SEQ ID NO: 71, and/or a light chain variable sequence that comprises a framework sequence that is at least 75%, at least 80%, at least 85%, at least 90%, at least 91%, at least 92%, at least 93%, at least 94%, at least 95%, at least 96%, at least 97%, at least 98%, or at least 99% identical to the framework sequence of a VL of SEQ ID NO: 70.

In some embodiments, an anti-trypsin 1/trypsin 2 antibody of the present disclosure comprises a HC CDR1, HC CDR2 and HC CDR3 of a heavy chain variable domain having the amino acid sequence of SEQ ID NO: 72. Alternatively or in addition, the anti-trypsin 1/trypsin 2 antibody of the present disclosure comprises a LC CDR1, LC CDR2 and LC CDR3 of a light chain variable domain having the amino acid sequence of SEQ ID NO: 73.

In some embodiments, an anti-trypsin 1/trypsin 2 antibody of the present disclosure comprises a HC CDR1 having the amino acid sequence of SEQ ID NO: 36, a HC CDR2 having the amino acid sequence of SEQ ID NO: 37, a HC CDR3 having the amino acid sequence of SEQ ID NO: 38, a LC CDR1 having the amino acid sequence of SEQ ID NO: 4, a LC CDR2 having the amino acid sequence of SEQ ID NO: 5, and a LC CDR3 having the amino acid sequence of SEQ ID NO: 39.

In some embodiments, an anti-trypsin 1/trypsin 2 antibody of the present disclosure comprises a HC CDR1, a HC CDR2, and a HC CDR3, which collectively contains no more than 5 amino acid variations (e.g., no more than 5, 4, 3, 2, or I amino acid variation) as compared with the HC CDR1 having the amino acid sequence of SEQ ID NO: 36, HC CDR2 having the amino acid sequence of SEQ ID NO: 37, and HC CDR3 having the amino acid sequence of SEQ ID NO: 38. Alternatively or in addition, the anti-trypsin 1/trypsin 2 antibody of the present disclosure comprises a LC CDR1, a LC CDR2, and a LC CDR3, which collectively contains no more than 5 amino acid variations (e.g., no more than 5, 4, 3, 2 or 1 amino acid variation) as compared with the LC CDR1 having the amino acid sequence of SEQ ID NO: 4, LC CDR2 having the amino acid sequence of SEQ ID NO: 5, and LC CDR3 having the amino acid sequence of SEQ ID NO: 39.

In some embodiments, an anti-trypsin 1/trypsin 2 antibody of the present disclosure comprises a HC CDR1, a HC CDR2, and a HC CDR3 that collectively are at least 80% (e.g., at least 80%, at least 85%, at least 90%, at least 91%, at least 92%, at least 93%, at least 94%, at least 95%, at least 96%, at least 97%, at least 98%, or at least 99%) identical to the HC CDR1 having the amino acid sequence of SEQ ID NO: 36, HC CDR2 having the amino acid sequence of SEQ ID NO: 37, and HC CDR3 having the amino acid sequence of SEQ ID NO: 38. Alternatively or in addition, the anti-trypsin 1/trypsin 2 antibody of the present disclosure comprises a LC CDR1, a LC CDR2, and a LC CDR3 that collectively are at least 80% (e.g., at least 80%, at least 85%, at least 90%, at least 91%, at least 92%, at least 93%, at least 94%, at least 95%, at least 96%, at least 97%, at least 98%, or at least 99%) identical to the to the LC CDR1 having the amino acid sequence of SEQ ID NO: 4, LC CDR2 having the amino acid sequence of SEQ ID NO: 5, and LC CDR3 having the amino acid sequence of SEQ ID NO: 39.

In some embodiments, an anti-trypsin 1/trypsin 2 antibody of the present disclosure comprises: a HC CDR1 having no more than 3 amino acid variations (e.g., no more than 3, 2, or 1 amino acid variation) as compared with the HC CDR1 having the amino acid sequence of SEQ ID NO: 36; a HC CDR2 having no more than 3 amino acid variations (e.g., no more than 3, 2, or I amino acid variation) as compared with the HC CDR2 having the amino acid sequence of SEQ ID NO: 37; and/or a HC CDR3 having no more than 3 amino acid variations (e.g., no more than 3, 2, or 1 amino acid variation) as compared with the HC CDR3 having the amino acid sequence of SEQ ID NO: 38. Alternatively or in addition, the anti-trypsin 1/trypsin 2 antibody of the present disclosure comprises: a LC CDR1 having no more than 3 amino acid variations (e.g., no more than 3, 2, or 1 amino acid variation) as compared with the LC CDR1 having the amino acid sequence of SEQ ID NO: 4; a LC CDR2 having no more than 3 amino acid variations (e.g., no more than 3, 2, or 1 amino acid variation) as compared with the LC CDR2 having the amino acid sequence of SEQ ID NO: 5; and/or a LC CDR3 having no more than 3 amino acid variations (e.g., no more than 3, 2, or I amino acid variation) as compared with the LC CDR3 having the amino acid sequence of SEQ ID NO: 39.

In some embodiments, an anti-trypsin 1/trypsin 2 antibody of the present disclosure comprises: a HC CDR1 that is at least 80% (e.g., at least 80%, at least 85%, at least 90%, at least 91%, at least 92%, at least 93%, at least 94%, at least 95%, at least 96%, at least 97%, at least 98%, or at least 99%) identical to the HC CDR1 having the amino acid sequence of SEQ ID NO: 36; a HC CDR2 that is at least 80% (e.g., at least 80%, at least 85%, at least 90%, at least 91%, at least 92%, at least 93%, at least 94%, at least 95%, at least 96%, at least 97%, at least 98%, or at least 99%) identical to the HC CDR2 having the amino acid sequence SEQ ID NO: 37; and/or a HC CDR3 that is at least 80% (e.g., at least 80%, at least 85%, at least 90%, at least 91%, at least 92%, at least 93%, at least 94%, at least 95%, at least 96%, at least 97%, at least 98%, or at least 99%) identical to the HC CDR3 having the amino acid sequence of SEQ ID NO: 38. Alternatively or in addition, the anti-trypsin 1/trypsin 2 antibody of the present disclosure comprises: a LC CDR1 that is at least 80% (e.g., at least 80%, at least 85%, at least 90%, at least 91%, at least 92%, at least 93%, at least 94%, at least 95%, at least 96%, at least 97%, at least 98%, or at least 99%) identical to the LC CDR1 having the amino acid sequence of SEQ ID NO: 4; a LC CDR2 that is at least 80% (e.g., at least 80%, at least 85%, at least 90%, at least 91%, at least 92%, at least 93%, at least 94%, at least 95%, at least 96%, at least 97%, at least 98%, or at least 99%) identical to the LC CDR2 having the amino acid sequence of SEQ ID NO: 5; and/or a LC CDR3 that is at least 80% (e.g., at least 80%, at least 85%, at least 90%, at least 91%, at least 92%, at least 93%, at least 94%, at least 95%, at least 96%, at least 97%, at least 98%, or at least 99%) identical to the LC CDR3 having the amino acid sequence of SEQ ID NO: 39.

In some embodiments, an anti-trypsin 1/trypsin 2 antibody of the present disclosure comprises a VH comprising the amino acid sequence of SEQ ID NO: 72. Alternatively or in addition, the anti-trypsin 1/trypsin 2 antibody of the present disclosure comprises a VL comprising the amino acid sequence of SEQ ID NO: 73.

In some embodiments, an anti-trypsin 1/trypsin 2 antibody of the present disclosure comprises a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 72. Alternatively or in addition, the anti-trypsin 1/trypsin 2 antibody of the present disclosure comprises a VL containing no more than 20 amino acid variations (e.g., no more than 20, 19, 18, 17, 16, 15, 14, 13, 12, 11, 9, 8, 7, 6, 5, 4, 3, 2, or I amino acid variation) as compared with the VL as set forth in SEQ ID NO: 73. In some embodiments, the number of amino acid variations (e.g., no more than 20, 19, 18, 17, 16, 15, 14, 13, 12, 11, 10, 9, 8, 7, 6, 5, 4, 3, 2, or I amino acid variation) may occur within a VH of SEQ ID NO: 72 and/or a VL of SEQ ID NO: 73 excluding any of the CDR sequences therein. In some embodiments, an anti-trypsin 1/trypsin 2 antibodies provided herein comprise a heavy chain variable sequence that comprises a framework sequence that that contains no more than 20, 19, 18, 17, 16, 15, 14, 13, 12, 11, 10, 9, 8, 7, 6, 5, 4, 3, 2, or I amino acid variation to the framework sequence of a VH of SEQ ID NO: 72, and/or a light chain variable sequence that comprises a framework sequence that that contains no more than 20, 19, 18, 17, 16, 15, 14, 13, 12, 11, 10, 9, 8, 7, 6, 5, 4, 3, 2, or 1 amino acid variation to the framework sequence of a VL of SEQ ID NO: 73.

In some embodiments, an anti-trypsin 1/trypsin 2 antibody of the present disclosure comprises a VH comprising an amino acid sequence that is at least 80% (e.g., at least 80%, at least 85%, at least 90%, at least 91%, at least 92%, at least 93%, at least 94%, at least 95%, at least 96%, at least 97%, at least 98%, or at least 99%) identical to the VH as set forth in SEQ ID NO: 72. Alternatively or in addition, the anti-trypsin 1/trypsin 2 antibody of the present disclosure comprises a VL comprising an amino acid sequence that is at least 80% (e.g., at least 80%, at least 85%, at least 90%, at least 91%, at least 92%, at least 93%, at least 94%, at least 95%, at least 96%, at least 97%, at least 98%, or at least 99%) identical to the VL as set forth in SEQ ID NO: 73. In some embodiments, the degree of sequence variation (e.g., at least 75%, at least 80%, at least 85%, at least 90%, at least 91%, at least 92%, at least 93%, at least 94%, at least 95%, at least 96%, at least 97%, at least 98%, or at least 99%) may occur within a VH of SEQ ID NO: 72, and/or a VL of SEQ ID NO: 73 excluding any of the CDR sequences therein. In some embodiments, an anti-trypsin 1/trypsin 2 antibody provided herein comprise a heavy chain variable sequence that is at least 75%, at least 80%, at least 85%, at least 90%, at least 91%, at least 92%, at least 93%, at least 94%, at least 95%, at least 96%, at least 97%, at least 98%, or at least 99% identical to the framework sequence of a VH of SEQ ID NO: 72, and/or a light chain variable sequence that comprises a framework sequence that is at least 75%, at least 80%, at least 85%, at least 90%, at least 91%, at least 92%, at least 93%, at least 94%, at least 95%, at least 96%, at least 97%, at least 98%, or at least 99% identical to the framework sequence of a VL of SEQ ID NO: 73.

In some embodiments, an anti-trypsin 1/trypsin 2 antibody of the present disclosure comprises a HC CDR1, HC CDR2 and HC CDR3 of a heavy chain variable domain having the amino acid sequence of SEQ ID NO: 74. Alternatively or in addition, the anti-trypsin 1/trypsin 2 antibody of the present disclosure comprises a LC CDR1, LC CDR2 and LC CDR3 of a light chain variable domain having the amino acid sequence of SEQ ID NO: 73.

In some embodiments, an anti-trypsin 1/trypsin 2 antibody of the present disclosure comprises a HC CDR1 having the amino acid sequence of SEQ ID NO: 40, a HBC CDR2 having the amino acid sequence of SEQ ID NO: 41, a HC CDR3 having the amino acid sequence of SEQ ID NO: 38, a LC CDR1 having the amino acid sequence of SEQ ID NO: 4, a LC CDR2 having the amino acid sequence of SEQ ID NO: 5, and a LC CDR3 having the amino acid sequence of SEQ ID NO: 39.

In some embodiments, an anti-trypsin 1/trypsin 2 antibody of the present disclosure comprises a HC CDR1, a HC CDR2, and a HC CDR3, which collectively contains no more than 5 amino acid variations (e.g., no more than 5, 4, 3, 2, or I amino acid variation) as compared with the HC CDR1 having the amino acid sequence of SEQ ID NO: 40, HC CDR2 having the amino acid sequence of SEQ ID NO: 41, and HC CDR3 having the amino acid sequence of SEQ ID NO: 38. Alternatively or in addition, the anti-trypsin 1/trypsin 2 antibody of the present disclosure comprises a LC CDR1, a LC CDR2, and a LC CDR3, which collectively contains no more than 5 amino acid variations (e.g., no more than 5, 4, 3, 2 or 1 amino acid variation) as compared with the LC CDR1 having the amino acid sequence of SEQ ID NO: 4, LC CDR2 having the amino acid sequence of SEQ ID NO: 5, and LC CDR3 having the amino acid sequence of SEQ ID NO: 39.

In some embodiments, an anti-trypsin 1/trypsin 2 antibody of the present disclosure comprises a HC CDR1, a HC CDR2, and a HC CDR3 that collectively are at least 80% (e.g., at least 80%, at least 85%, at least 90%, at least 91%, at least 92%, at least 93%, at least 94%, at least 95%, at least 96%, at least 97%, at least 98%, or at least 99%) identical to the HC CDR1 having the amino acid sequence of SEQ ID NO: 40, HC CDR2 having the amino acid sequence of SEQ ID NO: 41, and HC CDR3 having the amino acid sequence of SEQ ID NO: 38. Alternatively or in addition, the anti-trypsin 1/trypsin 2 antibody of the present disclosure comprises a LC CDR1, a LC CDR2, and a LC CDR3 that collectively are at least 80% (e.g., at least 80%, at least 85%, at least 90%, at least 91%, at least 92%, at least 93%, at least 94%, at least 95%, at least 96%, at least 97%, at least 98%, or at least 99%) identical to the to the LC CDR1 having the amino acid sequence of SEQ ID NO: 4, LC CDR2 having the amino acid sequence of SEQ ID NO: 5, and LC CDR3 having the amino acid sequence of SEQ ID NO: 39.

In some embodiments, an anti-trypsin 1/trypsin 2 antibody of the present disclosure comprises: a HC CDR1 having no more than 3 amino acid variations (e.g., no more than 3, 2, or 1 amino acid variation) as compared with the HC CDR1 having the amino acid sequence of SEQ ID NO: 40; a HC CDR2 having no more than 3 amino acid variations (e.g., no more than 3, 2, or I amino acid variation) as compared with the HC CDR2 having the amino acid sequence of SEQ ID NO: 41; and/or a HC CDR3 having no more than 3 amino acid variations (e.g., no more than 3, 2, or 1 amino acid variation) as compared with the HC CDR3 having the amino acid sequence of SEQ ID NO: 38. Alternatively or in addition, the anti-trypsin 1/trypsin 2 antibody of the present disclosure comprises: a LC CDR1 having no more than 3 amino acid variations (e.g., no more than 3, 2, or 1 amino acid variation) as compared with the LC CDR1 having the amino acid sequence of SEQ ID NO: 4; a LC CDR2 having no more than 3 amino acid variations (e.g., no more than 3, 2, or 1 amino acid variation) as compared with the LC CDR2 having the amino acid sequence of SEQ ID NO: 5; and/or a LC CDR3 having no more than 3 amino acid variations (e.g., no more than 3, 2, or 1 amino acid variation) as compared with the LC CDR3 having the amino acid sequence of SEQ ID NO: 39.

In some embodiments, an anti-trypsin 1/trypsin 2 antibody of the present disclosure comprises: a HC CDR1 that is at least 80% (e.g., at least 80%, at least 85%, at least 90%, at least 91%, at least 92%, at least 93%, at least 94%, at least 95%, at least 96%, at least 97%, at least 98%, or at least 99%) identical to the HC CDR1 having the amino acid sequence of SEQ ID NO: 40; a HC CDR2 that is at least 80% (e.g., at least 80%, at least 85%, at least 90%, at least 91%, at least 92%, at least 93%, at least 94%, at least 95%, at least 96%, at least 97%, at least 98%, or at least 99%) identical to the HC CDR2 having the amino acid sequence SEQ ID NO: 41; and/or a HC CDR3 that is at least 80% (e.g., at least 80%, at least 85%, at least 90%, at least 91%, at least 92%, at least 93%, at least 94%, at least 95%, at least 96%, at least 97%, at least 98%, or at least 99%) identical to the HC CDR3 having the amino acid sequence of SEQ ID NO: 38. Alternatively or in addition, the anti-trypsin 1/trypsin 2 antibody of the present disclosure comprises: a LC CDR1 that is at least 80% (e.g., at least 80%, at least 85%, at least 90%, at least 91%, at least 92%, at least 93%, at least 94%, at least 95%, at least 96%, at least 97%, at least 98%, or at least 99%) identical to the LC CDR1 having the amino acid sequence of SEQ ID NO: 4; a LC CDR2 that is at least 80% (e.g., at least 80%, at least 85%, at least 90%, at least 91%, at least 92%, at least 93%, at least 94%, at least 95%, at least 96%, at least 97%, at least 98%, or at least 99%) identical to the LC CDR2 having the amino acid sequence of SEQ ID NO: 5; and/or a LC CDR3 that is at least 80% (e.g., at least 80%, at least 85%, at least 90%, at least 91%, at least 92%, at least 93%, at least 94%, at least 95%, at least 96%, at least 97%, at least 98%, or at least 99%) identical to the LC CDR3 having the amino acid sequence of SEQ ID NO: 39.

In some embodiments, an anti-trypsin 1/trypsin 2 antibody of the present disclosure comprises a VH comprising the amino acid sequence of SEQ ID NO: 74. Alternatively or in addition, the anti-trypsin 1/trypsin 2 antibody of the present disclosure comprises a VL comprising the amino acid sequence of SEQ ID NO: 73.

In some embodiments, an anti-trypsin 1/trypsin 2 antibody of the present disclosure comprises a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 74. Alternatively or in addition, the anti-trypsin 1/trypsin 2 antibody of the present disclosure comprises a VL containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the VL as set forth in SEQ ID NO: 73. In some embodiments, the number of amino acid variations (e.g., no more than 20, 19, 18, 17, 16, 15, 14, 13, 12, 11, 10, 9, 8, 7, 6, 5, 4, 3, 2, or 1 amino acid variation) may occur within a VH of SEQ ID NO: 74 and/or a VL of SEQ ID NO: 73 excluding any of the CDR sequences therein. In some embodiments, an anti-trypsin 1/trypsin 2 antibodies provided herein comprise a heavy chain variable sequence that comprises a framework sequence that that contains no more than 20, 19, 18, 17, 16, 15, 14, 13, 12, 11, 10, 9, 8, 7, 6, 5, 4, 3, 2, or 1 amino acid variation to the framework sequence of a VH of SEQ ID NO: 74, and/or a light chain variable sequence that comprises a framework sequence that that contains no more than 20, 19, 18, 17, 16, 15, 14, 13, 12, 11, 10, 9, 8, 7, 6, 5, 4, 3, 2, or 1 amino acid variation to the framework sequence of a VL of SEQ ID NO: 73.

In some embodiments, an anti-trypsin 1/trypsin 2 antibody of the present disclosure comprises a VH comprising an amino acid sequence that is at least 80% (e.g., at least 80%, at least 85%, at least 90%, at least 91%, at least 92%, at least 93%, at least 94%, at least 95%, at least 96%, at least 97%, at least 98%, or at least 99%) identical to the VH as set forth in SEQ ID NO: 74. Alternatively or in addition, the anti-trypsin 1/trypsin 2 antibody of the present disclosure comprises a VL comprising an amino acid sequence that is at least 80% (e.g., at least 80%, at least 85%, at least 90%, at least 91%, at least 92%, at least 93%, at least 94%, at least 95%, at least 96%, at least 97%, at least 98%, or at least 99%) identical to the VL as set forth in SEQ ID NO: 73. In some embodiments, the degree of sequence variation (e.g., at least 75%, at least 80%, at least 85%, at least 90%, at least 91%, at least 92%, at least 93%, at least 94%, at least 95%, at least 96%, at least 97%, at least 98%, or at least 99%) may occur within a VH of SEQ ID NO: 74, and/or a VL of SEQ ID NO: 73 excluding any of the CDR sequences therein. In some embodiments, an anti-trypsin 1/trypsin 2 antibody provided herein comprise a heavy chain variable sequence that is at least 75%, at least 80%, at least 85%, at least 90%, at least 91%, at least 92%, at least 93%, at least 94%, at least 95%, at least 96%, at least 97%, at least 98%, or at least 99% identical to the framework sequence of a VH of SEQ ID NO: 74, and/or a light chain variable sequence that comprises a framework sequence that is at least 75%, at least 80%, at least 85%, at least 90%, at least 91%, at least 92%, at least 93%, at least 94%, at least 95%, at least 96%, at least 97%, at least 98%, or at least 99% identical to the framework sequence of a VL of SEQ ID NO: 73.

In some embodiments, an anti-trypsin 1/trypsin 2 antibody of the present disclosure comprises a HC CDR1, HC CDR2 and HC CDR3 of a heavy chain variable domain having the amino acid sequence of SEQ ID NO: 75. Alternatively or in addition, the anti-anti-trypsin 1/trypsin 2 antibody of the present disclosure comprises a LC CDR1, LC CDR2 and LC CDR3 of a light chain variable domain having the amino acid sequence of SEQ ID NO: 73.

In some embodiments, an anti-trypsin 1/trypsin 2 antibody of the present disclosure comprises a HC CDR1 having the amino acid sequence of SEQ ID NO: 40, a HC CDR2 having the amino acid sequence of SEQ ID NO: 41, a HC CDR3 having the amino acid sequence of SEQ ID NO: 42, a LC CDR1 having the amino acid sequence of SEQ ID NO: 4, a LC CDR2 having the amino acid sequence of SEQ ID NO: 5, and a LC CDR3 having the amino acid sequence of SEQ ID NO: 39.

In some embodiments, an anti-trypsin 1/trypsin 2 antibody of the present disclosure comprises a HC CDR1, a HC CDR2, and a HC CDR3, which collectively contains no more than 5 amino acid variations (e.g., no more than 5, 4, 3, 2, or I amino acid variation) as compared with the HC CDR1 having the amino acid sequence of SEQ ID NO: 40, HC CDR2 having the amino acid sequence of SEQ ID NO: 41, and HC CDR3 having the amino acid sequence of SEQ ID NO: 42. Alternatively or in addition, the anti-trypsin 1/trypsin 2 antibody of the present disclosure comprises a LC CDR1, a LC CDR2, and a LC CDR3, which collectively contains no more than 5 amino acid variations (e.g., no more than 5, 4, 3, 2 or 1 amino acid variation) as compared with the LC CDR1 having the amino acid sequence of SEQ ID NO: 4, LC CDR2 having the amino acid sequence of SEQ ID NO: 5, and LC CDR3 having the amino acid sequence of SEQ ID NO: 39.

In some embodiments, an anti-trypsin 1/trypsin 2 antibody of the present disclosure comprises a HC CDR1, a HC CDR2, and a HC CDR3 that collectively are at least 80% (e.g., at least 80%, at least 85%, at least 90%, at least 91%, at least 92%, at least 93%, at least 94%, at least 95%, at least 96%, at least 97%, at least 98%, or at least 99%) identical to the HC CDR1 having the amino acid sequence of SEQ ID NO: 40, HC CDR2 having the amino acid sequence of SEQ ID NO: 41, and HC CDR3 having the amino acid sequence of SEQ ID NO: 42. Alternatively or in addition, the anti-trypsin 1/trypsin 2 antibody of the present disclosure comprises a LC CDR1, a LC CDR2, and a LC CDR3 that collectively are at least 80% (e.g., at least 80%, at least 85%, at least 90%, at least 91%, at least 92%, at least 93%, at least 94%, at least 95%, at least 96%, at least 97%, at least 98%, or at least 99%) identical to the to the LC CDR1 having the amino acid sequence of SEQ ID NO: 4, LC CDR2 having the amino acid sequence of SEQ ID NO: 5, and LC CDR3 having the amino acid sequence of SEQ ID NO: 39.

In some embodiments, an anti-trypsin 1/trypsin 2 antibody of the present disclosure comprises: a HC CDR1 having no more than 3 amino acid variations (e.g., no more than 3, 2, or 1 amino acid variation) as compared with the HC CDR1 having the amino acid sequence of SEQ ID NO: 40; a HC CDR2 having no more than 3 amino acid variations (e.g., no more than 3, 2, or I amino acid variation) as compared with the HC CDR2 having the amino acid sequence of SEQ ID NO: 41; and/or a HC CDR3 having no more than 3 amino acid variations (e.g., no more than 3, 2, or 1 amino acid variation) as compared with the HC CDR3 having the amino acid sequence of SEQ ID NO: 42. Alternatively or in addition, the anti-trypsin 1/trypsin 2 antibody of the present disclosure comprises: a LC CDR1 having no more than 3 amino acid variations (e.g., no more than 3, 2, or I amino acid variation) as compared with the LC CDR1 having the amino acid sequence of SEQ ID NO: 4; a LC CDR2 having no more than 3 amino acid variations (e.g., no more than 3, 2, or 1 amino acid variation) as compared with the LC CDR2 having the amino acid sequence of SEQ ID NO: 5; and/or a LC CDR3 having no more than 3 amino acid variations (e.g., no more than 3, 2, or I amino acid variation) as compared with the LC CDR3 having the amino acid sequence of SEQ ID NO: 39.

In some embodiments, an anti-trypsin 1/trypsin 2 antibody of the present disclosure comprises: a HC CDR1 that is at least 80% (e.g., at least 80%, at least 85%, at least 90%, at least 91%, at least 92%, at least 93%, at least 94%, at least 95%, at least 96%, at least 97%, at least 98%, or at least 99%) identical to the HC CDR1 having the amino acid sequence of SEQ ID NO: 40; a HC CDR2 that is at least 80% (e.g., at least 80%, at least 85%, at least 90%, at least 91%, at least 92%, at least 93%, at least 94%, at least 95%, at least 96%, at least 97%, at least 98%, or at least 99%) identical to the HC CDR2 having the amino acid sequence SEQ ID NO: 41; and/or a HC CDR3 that is at least 80% (e.g., at least 80%, at least 85%, at least 90%, at least 91%, at least 92%, at least 93%, at least 94%, at least 95%, at least 96%, at least 97%, at least 98%, or at least 99%) identical to the HC CDR3 having the amino acid sequence of SEQ ID NO: 42. Alternatively or in addition, the anti-trypsin 1/trypsin 2 antibody of the present disclosure comprises: a LC CDR1 that is at least 80% (e.g., at least 80%, at least 85%, at least 90%, at least 91%, at least 92%, at least 93%, at least 94%, at least 95%, at least 96%, at least 97%, at least 98%, or at least 99%) identical to the LC CDR1 having the amino acid sequence of SEQ ID NO: 4; a LC CDR2 that is at least 80% (e.g., at least 80%, at least 85%, at least 90%, at least 91%, at least 92%, at least 93%, at least 94%, at least 95%, at least 96%, at least 97%, at least 98%, or at least 99%) identical to the LC CDR2 having the amino acid sequence of SEQ ID NO: 5; and/or a LC CDR3 that is at least 80% (e.g., at least 80%, at least 85%, at least 90%, at least 91%, at least 92%, at least 93%, at least 94%, at least 95%, at least 96%, at least 97%, at least 98%, or at least 99%) identical to the LC CDR3 having the amino acid sequence of SEQ ID NO: 39.

In some embodiments, an anti-trypsin 1/trypsin 2 antibody of the present disclosure comprises a VH comprising the amino acid sequence of SEQ ID NO: 75. Alternatively or in addition, the anti-trypsin 1/trypsin 2 antibody of the present disclosure comprises a VL comprising the amino acid sequence of SEQ ID NO: 73.

In some embodiments, an anti-trypsin 1/trypsin 2 antibody of the present disclosure comprises a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 75. Alternatively or in addition, the anti-trypsin 1/trypsin 2 antibody of the present disclosure comprises a VL containing no more than 20 amino acid variations (e.g., no more than 20, 19, 18, 17, 16, 15, 14, 13, 12, 11, 9, 8, 7, 6, 5, 4, 3, 2, or I amino acid variation) as compared with the V L as set forth in SEQ ID NO: 73. In some embodiments, the number of amino acid variations (e.g., no more than 20, 19, 18, 17, 16, 15, 14, 13, 12, 11, 10, 9, 8, 7, 6, 5, 4, 3, 2, or I amino acid variation) may occur within a VH of SEQ ID NO: 75 and/or a VL of SEQ ID NO: 73 excluding any of the CDR sequences therein. In some embodiments, an anti-trypsin 1/trypsin 2 antibodies provided herein comprise a heavy chain variable sequence that comprises a framework sequence that that contains no more than 20, 19, 18, 17, 16, 15, 14, 13, 12, 11, 10, 9, 8, 7, 6, 5, 4, 3, 2, or I amino acid variation to the framework sequence of a VH of SEQ ID NO: 75, and/or a light chain variable sequence that comprises a framework sequence that that contains no more than 20, 19, 18, 17, 16, 15, 14, 13, 12, 11, 10, 9, 8, 7, 6, 5, 4, 3, 2, or 1 amino acid variation to the framework sequence of a VL of SEQ ID NO: 73.

In some embodiments, an anti-trypsin 1/trypsin 2 antibody of the present disclosure comprises a VH comprising an amino acid sequence that is at least 80% (e.g., at least 80%, at least 85%, at least 90%, at least 91%, at least 92%, at least 93%, at least 94%, at least 95%, at least 96%, at least 97%, at least 98%, or at least 99%) identical to the VH as set forth in SEQ ID NO: 75. Alternatively or in addition, the anti-trypsin 1/trypsin 2 antibody of the present disclosure comprises a VL comprising an amino acid sequence that is at least 80% (e.g., at least 80%, at least 85%, at least 90%, at least 91%, at least 92%, at least 93%, at least 94%, at least 95%, at least 96%, at least 97%, at least 98%, or at least 99%) identical to the VL as set forth in SEQ ID NO: 73. In some embodiments, the degree of sequence variation (e.g., at least 75%, at least 80%, at least 85%, at least 90%, at least 91%, at least 92%, at least 93%, at least 94%, at least 95%, at least 96%, at least 97%, at least 98%, or at least 99%) may occur within a VH of SEQ ID NO: 75, and/or a VL of SEQ ID NO: 73 excluding any of the CDR sequences therein. In some embodiments, an anti-trypsin 1/trypsin 2 antibody provided herein comprise a heavy chain variable sequence that is at least 75%, at least 80%, at least 85%, at least 90%, at least 91%, at least 92%, at least 93%, at least 94%, at least 95%, at least 96%, at least 97%, at least 98%, or at least 99% identical to the framework sequence of a VH of SEQ ID NO: 75, and/or a light chain variable sequence that comprises a framework sequence that is at least 75%, at least 80%, at least 85%, at least 90%, at least 91%, at least 92%, at least 93%, at least 94%, at least 95%, at least 96%, at least 97%, at least 98%, or at least 99% identical to the framework sequence of a VL of SEQ ID NO: 73.

In some embodiments, an anti-trypsin 1/trypsin 2 antibody of the present disclosure comprises a HC CDR1, HC CDR2 and HC CDR3 of a heavy chain variable domain having the amino acid sequence of SEQ ID NO: 76. Alternatively or in addition, the anti-trypsin 1/trypsin 2 antibody of the present disclosure comprises a LC CDR1, LC CDR2 and LC CDR3 of a light chain variable domain having the amino acid sequence of SEQ ID NO: 73.

In some embodiments, an anti-trypsin 1/trypsin 2 antibody of the present disclosure comprises a HC CDR1 having the amino acid sequence of SEQ ID NO: 40, a HC CDR2 having the amino acid sequence of SEQ ID NO: 41, a HC CDR3 having the amino acid sequence of SEQ ID NO: 43, a LC CDR1 having the amino acid sequence of SEQ ID NO: 4, a LC CDR2 having the amino acid sequence of SEQ ID NO: 5, and a LC CDR3 having the amino acid sequence of SEQ ID NO: 39.

In some embodiments, an anti-trypsin 1/trypsin 2 antibody of the present disclosure comprises a HC CDR1, a HC CDR2, and a HC CDR3, which collectively contains no more than 5 amino acid variations (e.g., no more than 5, 4, 3, 2, or 1 amino acid variation) as compared with the HC CDR1 having the amino acid sequence of SEQ ID NO: 40, HC CDR2 having the amino acid sequence of SEQ ID NO: 41, and HC CDR3 having the amino acid sequence of SEQ ID NO: 43. Alternatively or in addition, the anti-trypsin 1/trypsin 2 antibody of the present disclosure comprises a LC CDR1, a LC CDR2, and a LC CDR3, which collectively contains no more than 5 amino acid variations (e.g., no more than 5, 4, 3, 2 or 1 amino acid variation) as compared with the LC CDR1 having the amino acid sequence of SEQ ID NO: 4, LC CDR2 having the amino acid sequence of SEQ ID NO: 5, and LC CDR3 having the amino acid sequence of SEQ ID NO: 39.

In some embodiments, an anti-trypsin 1/trypsin 2 antibody of the present disclosure comprises a HC CDR1, a HC CDR2, and a HC CDR3 that collectively are at least 80% (e.g., at least 80%, at least 85%, at least 90%, at least 91%, at least 92%, at least 93%, at least 94%, at least 95%, at least 96%, at least 97%, at least 98%, or at least 99%) identical to the HC CDR1 having the amino acid sequence of SEQ ID NO: 40, HC CDR2 having the amino acid sequence of SEQ ID NO: 41, and HC CDR3 having the amino acid sequence of SEQ ID NO: 43. Alternatively or in addition, the anti-trypsin 1/trypsin 2 antibody of the present disclosure comprises a LC CDR1, a LC CDR2, and a LC CDR3 that collectively are at least 80% (e.g., at least 80%, at least 85%, at least 90%, at least 91%, at least 92%, at least 93%, at least 94%, at least 95%, at least 96%, at least 97%, at least 98%, or at least 99%) identical to the to the LC CDR1 having the amino acid sequence of SEQ ID NO: 4, LC CDR2 having the amino acid sequence of SEQ ID NO: 5, and LC CDR3 having the amino acid sequence of SEQ ID NO: 39.

In some embodiments, an anti-trypsin 1/trypsin 2 antibody of the present disclosure comprises: a HC CDR1 having no more than 3 amino acid variations (e.g., no more than 3, 2, or 1 amino acid variation) as compared with the HC CDR1 having the amino acid sequence of SEQ ID NO: 40; a HC CDR2 having no more than 3 amino acid variations (e.g., no more than 3, 2, or I amino acid variation) as compared with the HC CDR2 having the amino acid sequence of SEQ ID NO: 41; and/or a HC CDR3 having no more than 3 amino acid variations (e.g., no more than 3, 2, or 1 amino acid variation) as compared with the HC CDR3 having the amino acid sequence of SEQ ID NO: 43. Alternatively or in addition, the anti-trypsin 1/trypsin 2 antibody of the present disclosure comprises: a LC CDR1 having no more than 3 amino acid variations (e.g., no more than 3, 2, or 1 amino acid variation) as compared with the LC CDR1 having the amino acid sequence of SEQ ID NO: 4; a LC CDR2 having no more than 3 amino acid variations (e.g., no more than 3, 2, or 1 amino acid variation) as compared with the LC CDR2 having the amino acid sequence of SEQ ID NO: 5; and/or a LC CDR3 having no more than 3 amino acid variations (e.g., no more than 3, 2, or I amino acid variation) as compared with the LC CDR3 having the amino acid sequence of SEQ ID NO: 39.

In some embodiments, an anti-trypsin 1/trypsin 2 antibody of the present disclosure comprises: a HC CDR1 that is at least 80% (e.g., at least 80%, at least 85%, at least 90%, at least 91% e, at least 92%, at least 93%, at least 94%, at least 95%, at least 96%, at least 97%, at least 98%, or at least 99%) identical to the HC CDR1 having the amino acid sequence of SEQ ID NO: 40; a HC CDR2 that is at least 80% (e.g., at least 80%, at least 85%, at least 90%, at least 91%, at least 92%, at least 93%, at least 94%, at least 95%, at least 96%, at least 97%, at least 98%, or at least 99%) identical to the HC CDR2 having the amino acid sequence SEQ ID NO: 41; and/or a HC CDR3 that is at least 80% (e.g., at least 80%, at least 85%, at least 90%, at least 91%, at least 92%, at least 93%, at least 94%, at least 95%, at least 96%, at least 97%, at least 98%, or at least 99%) identical to the HC CDR3 having the amino acid sequence of SEQ ID NO: 43. Alternatively or in addition, the anti-trypsin 1/trypsin 2 antibody of the present disclosure comprises: a LC CDR11 that is at least 80% (e.g., at least 80%, at least 85%, at least 90%, at least 91%, at least 92%, at least 93%, at least 94%, at least 95%, at least 96%, at least 97%, at least 98%, or at least 99%) identical to the LC CDR1 having the amino acid sequence of SEQ ID NO: 4; a LC CDR2 that is at least 80% (e.g., at least 80%, at least 85%, at least 90%, at least 91%, at least 92%, at least 93%, at least 94%, at least 95%, at least 96%, at least 97%, at least 98%, or at least 99%) identical to the LC CDR2 having the amino acid sequence of SEQ ID NO: 5; and/or a LC CDR3 that is at least 80% (e.g., at least 80%, at least 85%, at least 90%, at least 91%, at least 92%, at least 93%, at least 94%, at least 95%, at least 96%, at least 97%, at least 98%, or at least 99%) identical to the LC CDR3 having the amino acid sequence of SEQ ID NO: 39.

In some embodiments, an anti-trypsin 1/trypsin 2 antibody of the present disclosure comprises a VH comprising the amino acid sequence of SEQ ID NO: 76. Alternatively or in addition, the anti-trypsin 1/trypsin 2 antibody of the present disclosure comprises a VL comprising the amino acid sequence of SEQ ID NO: 73.

In some embodiments, an anti-trypsin 1/trypsin 2 antibody of the present disclosure comprises a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 76. Alternatively or in addition, the anti-trypsin 1/trypsin 2 antibody of the present disclosure comprises a VL containing no more than 20 amino acid variations (e.g., no more than 20, 19, 18, 17, 16, 15, 14, 13, 12, 11, 9, 8, 7, 6, 5, 4, 3, 2, or I amino acid variation) as compared with the VL as set forth in SEQ ID NO: 73. In some embodiments, the number of amino acid variations (e.g., no more than 20, 19, 18, 17, 16, 15, 14, 13, 12, 11, 10, 9, 8, 7, 6, 5, 4, 3, 2, or I amino acid variation) may occur within a V H of SEQ ID NO: 76 and/or a V L of SEQ ID NO: 73 excluding any of the CDR sequences therein. In some embodiments, an anti-trypsin 1/trypsin 2 antibodies provided herein comprise a heavy chain variable sequence that comprises a framework sequence that that contains no more than 20, 19, 18, 17, 16, 15, 14, 13, 12, 11, 10, 9, 8, 7, 6, 5, 4, 3, 2, or I amino acid variation to the framework sequence of a VHT of SEQ ID NO: 76, and/or a light chain variable sequence that comprises a framework sequence that that contains no more than 20, 19, 18, 17, 16, 15, 14, 13, 12, 11, 10, 9, 8, 7, 6, 5, 4, 3, 2, or 1 amino acid variation to the framework sequence of a VL of SEQ ID NO: 73.

In some embodiments, an anti-trypsin 1/trypsin 2 antibody of the present disclosure comprises a VH comprising an amino acid sequence that is at least 80% (e.g., at least 80%, at least 85%, at least 90%, at least 91%, at least 92%, at least 93%, at least 94%, at least 95%, at least 96%, at least 97%, at least 98%, or at least 99%) identical to the VH as set forth in SEQ ID NO: 76. Alternatively or in addition, the anti-trypsin 1/trypsin 2 antibody of the present disclosure comprises a VL comprising an amino acid sequence that is at least 80% (e.g., at least 80%, at least 85%, at least 90%, at least 91%, at least 92%, at least 93%, at least 94%, at least 95%, at least 96%, at least 97%, at least 98%, or at least 99%) identical to the VL as set forth in SEQ ID NO: 73. In some embodiments, the degree of sequence variation (e.g., at least 75%, at least 80%, at least 85%, at least 90%, at least 91%, at least 92%, at least 93%, at least 94%, at least 95%, at least 96%, at least 97%, at least 98%, or at least 99%) may occur within a VH of SEQ ID NO: 76, and/or a VL of SEQ ID NO: 73 excluding any of the CDR sequences therein. In some embodiments, an anti-trypsin 1/trypsin 2 antibody provided herein comprise a heavy chain variable sequence that is at least 75%, at least 80%, at least 85%, at least 90%, at least 91%, at least 92%, at least 93%, at least 94%, at least 95%, at least 96%, at least 97%, at least 98%, or at least 99% identical to the framework sequence of a VH of SEQ ID NO: 76, and/or a light chain variable sequence that comprises a framework sequence that is at least 75%, at least 80%, at least 85%, at least 90%, at least 91%, at least 92%, at least 93%, at least 94%, at least 95%, at least 96%, at least 97%, at least 98%, or at least 99% identical to the framework sequence of a VL of SEQ ID NO: 73.

In some embodiments, an anti-trypsin 1/trypsin 2 antibody of the present disclosure comprises a HC CDR1, HC CDR2 and HC CDR3 of a heavy chain variable domain having the amino acid sequence of SEQ ID NO: 77. Alternatively or in addition, the anti-trypsin 1/trypsin 2 antibody of the present disclosure comprises a LC CDR1, LC CDR2 and LC CDR3 of a light chain variable domain having the amino acid sequence of SEQ ID NO: 78.

In some embodiments, an anti-trypsin 1/trypsin 2 antibody of the present disclosure comprises a HC CDR1 having the amino acid sequence of SEQ ID NO: 40, a HC CDR2 having the amino acid sequence of SEQ ID NO: 41, a HC CDR3 having the amino acid sequence of SEQ ID NO: 44, a LC CDR1 having the amino acid sequence of SEQ ID NO: 4, a LC CDR2 having the amino acid sequence of SEQ ID NO: 5, and a LC CDR3 having the amino acid sequence of SEQ ID NO: 45.

In some embodiments, an anti-trypsin 1/trypsin 2 antibody of the present disclosure comprises a HC CDR1, a HC CDR2, and a HC CDR3, which collectively contains no more than 5 amino acid variations (e.g., no more than 5, 4, 3, 2, or I amino acid variation) as compared with the HC CDR1 having the amino acid sequence of SEQ ID NO: 40, HC CDR2 having the amino acid sequence of SEQ ID NO: 41, and HC CDR3 having the amino acid sequence of SEQ ID NO: 44. Alternatively or in addition, the anti-trypsin 1/trypsin 2 antibody of the present disclosure comprises a LC CDR1, a LC CDR2, and a LC CDR3, which collectively contains no more than 5 amino acid variations (e.g., no more than 5, 4, 3, 2 or 1 amino acid variation) as compared with the LC CDR1 having the amino acid sequence of SEQ ID NO: 4, LC CDR2 having the amino acid sequence of SEQ ID NO: 5, and LC CDR3 having the amino acid sequence of SEQ ID NO: 45.

In some embodiments, an anti-trypsin 1/trypsin 2 antibody of the present disclosure comprises a HC CDR1, a HC CDR2, and a HC CDR3 that collectively are at least 80% (e.g., at least 80%, at least 85%, at least 90%, at least 91%, at least 92%, at least 93%, at least 94%, at least 95%, at least 96%, at least 97%, at least 98%, or at least 99%) identical to the HC CDR1 having the amino acid sequence of SEQ ID NO: 40, HC CDR2 having the amino acid sequence of SEQ ID NO: 41, and HC CDR3 having the amino acid sequence of SEQ ID NO: 44. Alternatively or in addition, the anti-trypsin 1/trypsin 2 antibody of the present disclosure comprises a LC CDR1, a LC CDR2, and a LC CDR3 that collectively are at least 80% (e.g., at least 80%, at least 85%, at least 90%, at least 91%, at least 92%, at least 93%, at least 94%, at least 95%, at least 96%, at least 97%, at least 98%, or at least 99%) identical to the to the LC CDR1 having the amino acid sequence of SEQ ID NO: 4, LC CDR2 having the amino acid sequence of SEQ ID NO: 5, and LC CDR3 having the amino acid sequence of SEQ ID NO: 45.

In some embodiments, an anti-trypsin 1/trypsin 2 antibody of the present disclosure comprises: a HC CDR1 having no more than 3 amino acid variations (e.g., no more than 3, 2, or 1 amino acid variation) as compared with the HC CDR1 having the amino acid sequence of SEQ ID NO: 40; a HC CDR2 having no more than 3 amino acid variations (e.g., no more than 3, 2, or I amino acid variation) as compared with the HC CDR2 having the amino acid sequence of SEQ ID NO: 41; and/or a HC CDR3 having no more than 3 amino acid variations (e.g., no more than 3, 2, or 1 amino acid variation) as compared with the HC CDR3 having the amino acid sequence of SEQ ID NO: 44. Alternatively or in addition, the anti-trypsin 1/trypsin 2 antibody of the present disclosure comprises: a LC CDR1 having no more than 3 amino acid variations (e.g., no more than 3, 2, or 1 amino acid variation) as compared with the LC CDR1 having the amino acid sequence of SEQ ID NO: 4; a LC CDR2 having no more than 3 amino acid variations (e.g., no more than 3, 2, or 1 amino acid variation) as compared with the LC CDR2 having the amino acid sequence of SEQ ID NO: 5; and/or a LC CDR3 having no more than 3 amino acid variations (e.g., no more than 3, 2, or 1 amino acid variation) as compared with the LC CDR3 having the amino acid sequence of SEQ ID NO: 45.

In some embodiments, an anti-trypsin 1/trypsin 2 antibody of the present disclosure comprises: a HC CDR1 that is at least 80% (e.g., at least 80%, at least 85%, at least 90%, at least 91%, at least 92%, at least 93%, at least 94%, at least 95%, at least 96%, at least 97%, at least 98%, or at least 99%) identical to the HC CDR1 having the amino acid sequence of SEQ ID NO: 40; a HC CDR2 that is at least 80% (e.g., at least 80%, at least 85%, at least 90%, at least 91%, at least 92%, at least 93%, at least 94%, at least 95%, at least 96%, at least 97%, at least 98%, or at least 99%) identical to the HC CDR2 having the amino acid sequence SEQ ID NO: 41; and/or a HC CDR3 that is at least 80% (e.g., at least 80%, at least 85%, at least 90%, at least 91%, at least 92%, at least 93%, at least 94%, at least 95%, at least 96%, at least 97%, at least 98%, or at least 99%) identical to the HC CDR3 having the amino acid sequence of SEQ ID NO: 44. Alternatively or in addition, the anti-trypsin 1/trypsin 2 antibody of the present disclosure comprises: a LC CDR1 that is at least 80% (e.g., at least 80%, at least 85%, at least 90%, at least 91%, at least 92%, at least 93%, at least 94%, at least 95%, at least 96%, at least 97%, at least 98%, or at least 99%) identical to the LC CDR1 having the amino acid sequence of SEQ ID NO: 4; a LC CDR2 that is at least 80% (e.g., at least 80%, at least 85%, at least 90%, at least 91%, at least 92%, at least 93%, at least 94%, at least 95%, at least 96%, at least 97%, at least 98%, or at least 99%) identical to the LC CDR2 having the amino acid sequence of SEQ ID NO: 5; and/or a LC CDR3 that is at least 80% (e.g., at least 80%, at least 85%, at least 90%, at least 91%, at least 92%, at least 93%, at least 94%, at least 95%, at least 96%, at least 97%, at least 98%, or at least 99%) identical to the LC CDR3 having the amino acid sequence of SEQ ID NO: 45.

In some embodiments, an anti-trypsin 1/trypsin 2 antibody of the present disclosure comprises a VH comprising the amino acid sequence of SEQ ID NO: 77. Alternatively or in addition, the anti-trypsin 1/trypsin 2 antibody of the present disclosure comprises a VL comprising the amino acid sequence of SEQ ID NO: 78.

In some embodiments, an anti-trypsin 1/trypsin 2 antibody of the present disclosure comprises a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 77. Alternatively or in addition, the anti-trypsin 1/trypsin 2 antibody of the present disclosure comprises a VL containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the VL as set forth in SEQ ID NO: 78. In some embodiments, the number of amino acid variations (e.g., no more than 20, 19, 18, 17, 16, 15, 14, 13, 12, 11, 10, 9, 8, 7, 6, 5, 4, 3, 2, or I amino acid variation) may occur within a VH of SEQ ID NO: 77 and/or a VL of SEQ ID NO: 78 excluding any of the CDR sequences therein, In some embodiments, an anti-trypsin 1/trypsin 2 antibodies provided herein comprise a heavy chain variable sequence that comprises a framework sequence that that contains no more than 20, 19, 18, 17, 16, 15, 14, 13, 12, 11, 10, 9, 8, 7, 6, 5, 4, 3, 2, or 1 amino acid variation to the framework sequence of a VH of SEQ ID NO: 77, and/or a light chain variable sequence that comprises a framework sequence that that contains no more than 20, 19, 18, 17, 16, 15, 14, 13, 12, 11, 10, 9, 8, 7, 6, 5, 4, 3, 2, or 1 amino acid variation to the framework sequence of a VL of SEQ ID NO: 78.

In some embodiments, an anti-trypsin 1/trypsin 2 antibody of the present disclosure comprises a VH comprising an amino acid sequence that is at least 80% (e.g., at least 80%, at least 85%, at least 90%, at least 91%, at least 92%, at least 93%, at least 94%, at least 95%, at least 96%, at least 97%, at least 98%, or at least 99%) identical to the VH as set forth in SEQ ID NO: 77, Alternatively or in addition, the anti-trypsin 1/trypsin 2 antibody of the present disclosure comprises a VL comprising an amino acid sequence that is at least 80% (e.g., at least 80%, at least 85%, at least 90%, at least 91%, at least 92%, at least 93%, at least 94%, at least 95%, at least 96%, at least 97%, at least 98%, or at least 99%) identical to the VL as set forth in SEQ ID NO: 78. In some embodiments, the degree of sequence variation (e.g., at least 75%, at least 80%, at least 85%, at least 90%, at least 91%, at least 92%, at least 93%, at least 94%, at least 95%, at least 96%, at least 97%, at least 98%, or at least 99%) may occur within a VH of SEQ ID NO: 77, and/or a VL of SEQ ID NO: 78 excluding any of the CDR sequences therein. In some embodiments, an anti-trypsin 1/trypsin 2 antibody provided herein comprise a heavy chain variable sequence that is at least 75%, at least 80%, at least 85%, at least 90%, at least 91%, at least 92%, at least 93%, at least 94%, at least 95%, at least 96%, at least 97%, at least 98%, or at least 99% identical to the framework sequence of a VH of SEQ ID NO: 77, and/or a light chain variable sequence that comprises a framework sequence that is at least 75%, at least 80%, at least 85%, at least 90%, at least 91%, at least 92%, at least 93%, at least 94%, at least 95%, at least 96%, at least 97%, at least 98%, or at least 99% identical to the framework sequence of a V L of SEQ ID NO: 78.

In some embodiments, an anti-trypsin 1/trypsin 2 antibody of the present disclosure comprises a HC CDR1, HC CDR2 and HC CDR3 of a heavy chain variable domain having the amino acid sequence of SEQ ID NO: 79. Alternatively or in addition, the anti-trypsin 1/trypsin 2 antibody of the present disclosure comprises a LC CDR1, LC CDR2 and LC CDR3 of a light chain variable domain having the amino acid sequence of SEQ ID NO: 80.

In some embodiments, an anti-trypsin 1/trypsin 2 antibody of the present disclosure comprises a HC CDR1 having the amino acid sequence of SEQ ID NO: 40, a HC CDR2 having the amino acid sequence of SEQ ID NO: 46, a HC CDR3 having the amino acid sequence of SEQ ID NO: 47, a LC CDR1 having the amino acid sequence of SEQ ID NO: 4, a LC CDR2 having the amino acid sequence of SEQ ID NO: 5, and a LC CDR3 having the amino acid sequence of SEQ ID NO: 48.

In some embodiments, an anti-trypsin 1/trypsin 2 antibody of the present disclosure comprises a HC CDR1, a HC CDR2, and a HC CDR3, which collectively contains no more than 5 amino acid variations (e.g., no more than 5, 4, 3, 2, or I amino acid variation) as compared with the HC CDR1 having the amino acid sequence of SEQ ID NO: 40, HC CDR2 having the amino acid sequence of SEQ ID NO: 46, and HC CDR3 having the amino acid sequence of SEQ ID NO: 47. Alternatively or in addition, the anti-trypsin 1/trypsin 2 antibody of the present disclosure comprises a LC CDR1, a LC CDR2, and a LC CDR3, which collectively contains no more than 5 amino acid variations (e.g., no more than 5, 4, 3, 2 or 1 amino acid variation) as compared with the LC CDR1 having the amino acid sequence of SEQ ID NO: 4, LC CDR2 having the amino acid sequence of SEQ ID NO: 5, and LC CDR3 having the amino acid sequence of SEQ ID NO: 48.

In some embodiments, an anti-trypsin 1/trypsin 2 antibody of the present disclosure comprises a HC CDR1, a HC CDR2, and a HC CDR3 that collectively are at least 80% (e.g., at least 80%, at least 85%, at least 90%, at least 91%, at least 92%, at least 93%, at least 94%, at least 95%, at least 96%, at least 97%, at least 98%, or at least 99%) identical to the HC CDR1 having the amino acid sequence of SEQ ID NO: 40, HC CDR2 having the amino acid sequence of SEQ ID NO: 46, and HC CDR3 having the amino acid sequence of SEQ ID NO: 47. Alternatively or in addition, the anti-trypsin 1/trypsin 2 antibody of the present disclosure comprises a LC CDR1, a LC CDR2, and a LC CDR3 that collectively are at least 80% (e.g., at least 80%, at least 85%, at least 90%, at least 91%, at least 92%, at least 93%, at least 94%, at least 95%, at least 96%, at least 97%, at least 98%, or at least 99%) identical to the to the LC CDR1 having the amino acid sequence of SEQ ID NO: 4, LC CDR2 having the amino acid sequence of SEQ ID NO: 5, and LC CDR3 having the amino acid sequence of SEQ ID NO: 48.

In some embodiments, an anti-trypsin 1/trypsin 2 antibody of the present disclosure comprises: a HC CDR1 having no more than 3 amino acid variations (e.g., no more than 3, 2, or 1 amino acid variation) as compared with the HC CDR1 having the amino acid sequence of SEQ ID NO: 40; a HC CDR2 having no more than 3 amino acid variations (e.g., no more than 3, 2, or I amino acid variation) as compared with the HC CDR2 having the amino acid sequence of SEQ ID NO: 46; and/or a HC CDR3 having no more than 3 amino acid variations (e.g., no more than 3, 2, or 1 amino acid variation) as compared with the HC CDR3 having the amino acid sequence of SEQ ID NO: 47. Alternatively or in addition, the anti-trypsin 1/trypsin 2 antibody of the present disclosure comprises: a LC CDR1 having no more than 3 amino acid variations (e.g., no more than 3, 2, or I amino acid variation) as compared with the LC CDR1 having the amino acid sequence of SEQ ID NO: 4; a LC CDR2 having no more than 3 amino acid variations (e.g., no more than 3, 2, or 1 amino acid variation) as compared with the LC CDR2 having the amino acid sequence of SEQ ID NO: 5; and/or a LC CDR3 having no more than 3 amino acid variations (e.g., no more than 3, 2, or 1 amino acid variation) as compared with the LC CDR3 having the amino acid sequence of SEQ ID NO: 48.

In some embodiments, an anti-trypsin 1/trypsin 2 antibody of the present disclosure comprises: a HC CDR1 that is at least 80% (e.g., at least 80%, at least 85%, at least 90%, at least 91%, at least 92%, at least 93%, at least 94%, at least 95%, at least 96%, at least 97%, at least 98%, or at least 99%) identical to the HC CDR1 having the amino acid sequence of SEQ ID NO: 40; a HC CDR2 that is at least 80% (e.g., at least 80%, at least 85%, at least 90%, at least 91%, at least 92%, at least 93%, at least 94%, at least 95%, at least 96%, at least 97%, at least 98%, or at least 99%) identical to the HC CDR2 having the amino acid sequence SEQ ID NO: 46; and/or a HC CDR3 that is at least 80% (e.g., at least 80%, at least 85%, at least 90%, at least 91%, at least 92%, at least 93%, at least 94%, at least 95%, at least 96%, at least 97%, at least 98%, or at least 99%) identical to the HC CDR3 having the amino acid sequence of SEQ ID NO: 47. Alternatively or in addition, the anti-trypsin 1/trypsin 2 antibody of the present disclosure comprises: a LC CDR1 that is at least 80% (e.g., at least 80%, at least 85%, at least 90%, at least 91%, at least 92%, at least 93%, at least 94%, at least 95%, at least 96%, at least 97%, at least 98%, or at least 99%) identical to the LC CDR1 having the amino acid sequence of SEQ ID NO: 4; a LC CDR2 that is at least 80% (e.g., at least 80%, at least 85%, at least 90%, at least 91%, at least 92%, at least 93%, at least 94%, at least 95%, at least 96%, at least 97%, at least 98%, or at least 99%) identical to the LC CDR2 having the amino acid sequence of SEQ ID NO: 5; and/or a LC CDR3 that is at least 80% (e.g., at least 80%, at least 85%, at least 90%, at least 91%, at least 92%, at least 93%, at least 94%, at least 95%, at least 96%, at least 97%, at least 98%, or at least 99%) identical to the LC CDR3 having the amino acid sequence of SEQ ID NO: 48.

In some embodiments, an anti-trypsin 1/trypsin 2 antibody of the present disclosure comprises a VH comprising the amino acid sequence of SEQ ID NO: 79. Alternatively or in addition, the anti-trypsin 1/trypsin 2 antibody of the present disclosure comprises a VL comprising the amino acid sequence of SEQ ID NO: 80.

In some embodiments, an anti-trypsin 1/trypsin 2 antibody of the present disclosure comprises a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 79. Alternatively or in addition, the anti-trypsin 1/trypsin 2 antibody of the present disclosure comprises a VL containing no more than 20 amino acid variations (e.g., no more than 20, 19, 18, 17, 16, 15, 14, 13, 12, 11, 9, 8, 7, 6, 5, 4, 3, 2, or I amino acid variation) as compared with the V L as set forth in SEQ ID NO: 80. In some embodiments, the number of amino acid variations (e.g., no more than 20, 19, 18, 17, 16, 15, 14, 13, 12, 11, 10, 9, 8, 7, 6, 5, 4, 3, 2, or I amino acid variation) may occur within a VH of SEQ ID NO: 79 and/or a VL of SEQ ID NO: 80 excluding any of the CDR sequences therein. In some embodiments, an anti-trypsin 1/trypsin 2 antibodies provided herein comprise a heavy chain variable sequence that comprises a framework sequence that that contains no more than 20, 19, 18, 17, 16, 15, 14, 13, 12, 11, 10, 9, 8, 7, 6, 5, 4, 3, 2, or I amino acid variation to the framework sequence of a VH of SEQ ID NO: 79, and/or a light chain variable sequence that comprises a framework sequence that that contains no more than 20, 19, 18, 17, 16, 15, 14, 13, 12, 11, 10, 9, 8, 7, 6, 5, 4, 3, 2, or 1 amino acid variation to the framework sequence of a VL of SEQ ID NO: 80.

In some embodiments, an anti-trypsin 1/trypsin 2 antibody of the present disclosure comprises a VH comprising an amino acid sequence that is at least 80% (e.g., at least 80%, at least 85%, at least 90%, at least 91%, at least 92%, at least 93%, at least 94%, at least 95%, at least 96%, at least 97%, at least 98%, or at least 99%) identical to the VH as set forth in SEQ ID NO: 79. Alternatively or in addition, the anti-trypsin 1/trypsin 2 antibody of the present disclosure comprises a VL comprising an amino acid sequence that is at least 80% (e.g., at least 80%, at least 85%, at least 90%, at least 91%, at least 92%, at least 93%, at least 94%, at least 95%, at least 96%, at least 97%, at least 98%, or at least 99%) identical to the VL as set forth in SEQ ID NO: 80. In some embodiments, the degree of sequence variation (e.g., at least 75%, at least 80%, at least 85%, at least 90%, at least 91%, at least 92%, at least 93%, at least 94%, at least 95%, at least 96%, at least 97%, at least 98%, or at least 99%) may occur within a VH of SEQ ID NO: 79, and/or a VL of SEQ ID NO: 80 excluding any of the CDR sequences therein. In some embodiments, an anti-trypsin 1/trypsin 2 antibody provided herein comprise a heavy chain variable sequence that is at least 75%, at least 80%, at least 85%, at least 90%, at least 91%, at least 92%, at least 93%, at least 94%, at least 95%, at least 96%, at least 97%, at least 98%, or at least 99% identical to the framework sequence of a VH of SEQ ID NO: 79, and/or a light chain variable sequence that comprises a framework sequence that is at least 75%, at least 80%, at least 85%, at least 90%, at least 91%, at least 92%, at least 93%, at least 94%, at least 95%, at least 96%, at least 97%, at least 98%, or at least 99% identical to the framework sequence of a VL of SEQ ID NO: 80.

In some embodiments, an anti-trypsin 1/trypsin 2 antibody of the present disclosure comprises a HC CDR1, HC CDR2 and HC CDR3 of a heavy chain variable domain having the amino acid sequence of SEQ ID NO: 81. Alternatively or in addition, the anti-anti-trypsin 1/trypsin 2 antibody of the present disclosure comprises a LC CDR1, LC CDR2 and LC CDR3 of a light chain variable domain having the amino acid sequence of SEQ ID NO: 82.

In some embodiments, an anti-trypsin 1/trypsin 2 antibody of the present disclosure comprises a HC CDR1 having the amino acid sequence of SEQ ID NO: 49, a HC CDR2 having the amino acid sequence of SEQ ID NO: 50, a HC CDR3 having the amino acid sequence of SEQ ID NO: 51, a LC CDR1 having the amino acid sequence of SEQ ID NO: 4, a LC CDR2 having the amino acid sequence of SEQ ID NO: 5, and a LC CDR3 having the amino acid sequence of SEQ ID NO: 52.

In some embodiments, an anti-trypsin 1/trypsin 2 antibody of the present disclosure comprises a HC CDR1, a HC CDR2, and a HC CDR3, which collectively contains no more than 5 amino acid variations (e.g., no more than 5, 4, 3, 2, or 1 amino acid variation) as compared with the HC CDR1 having the amino acid sequence of SEQ ID NO: 49, HC CDR2 having the amino acid sequence of SEQ ID NO: 50, and HC CDR3 having the amino acid sequence of SEQ ID NO: 51. Alternatively or in addition, the anti-trypsin 1/trypsin 2 antibody of the present disclosure comprises a LC CDR1, a LC CDR2, and a LC CDR3, which collectively contains no more than 5 amino acid variations (e.g., no more than 5, 4, 3, 2 or 1 amino acid variation) as compared with the LC CDR1 having the amino acid sequence of SEQ ID NO: 4, LC CDR2 having the amino acid sequence of SEQ ID NO: 5, and LC CDR3 having the amino acid sequence of SEQ ID NO: 52.

In some embodiments, an anti-trypsin 1/trypsin 2 antibody of the present disclosure comprises a HC CDR1, a HC CDR2, and a HC CDR3 that collectively are at least 80% (e.g., at least 80%, at least 85%, at least 90%, at least 91%, at least 92%, at least 93%, at least 94%, at least 95%, at least 96%, at least 97%, at least 98%, or at least 99%) identical to the HC CDR1 having the amino acid sequence of SEQ ID NO: 49, HC CDR2 having the amino acid sequence of SEQ ID NO: 50, and HC CDR3 having the amino acid sequence of SEQ ID NO: 51. Alternatively or in addition, the anti-trypsin 1/trypsin 2 antibody of the present disclosure comprises a LC CDR1, a LC CDR2, and a LC CDR3 that collectively are at least 80% (e.g., at least 80%, at least 85%, at least 90%, at least 91%, at least 92%, at least 93%, at least 94%, at least 95%, at least 96%, at least 97%, at least 98%, or at least 99%) identical to the to the LC CDR1 having the amino acid sequence of SEQ ID NO: 4, LC CDR2 having the amino acid sequence of SEQ ID NO: 5, and LC CDR3 having the amino acid sequence of SEQ ID NO: 52.

In some embodiments, an anti-trypsin 1/trypsin 2 antibody of the present disclosure comprises: a HC CDR1 having no more than 3 amino acid variations (e.g., no more than 3, 2, or 1 amino acid variation) as compared with the HC CDR1 having the amino acid sequence of SEQ ID NO: 49; a HC CDR2 having no more than 3 amino acid variations (e.g., no more than 3, 2, or I amino acid variation) as compared with the HC CDR2 having the amino acid sequence of SEQ ID NO: 50; and/or a HC CDR3 having no more than 3 amino acid variations (e.g., no more than 3, 2, or 1 amino acid variation) as compared with the HC CDR3 having the amino acid sequence of SEQ ID NO: 51. Alternatively or in addition, the anti-trypsin 1/trypsin 2 antibody of the present disclosure comprises: a LC CDR1 having no more than 3 amino acid variations (e.g., no more than 3, 2, or 1 amino acid variation) as compared with the LC CDR1 having the amino acid sequence of SEQ ID NO: 4; a LC CDR2 having no more than 3 amino acid variations (e.g., no more than 3, 2, or I amino acid variation) as compared with the LC CDR2 having the amino acid sequence of SEQ ID NO: 5; and/or a LC CDR3 having no more than 3 amino acid variations (e.g., no more than 3, 2, or I amino acid variation) as compared with the LC CDR3 having the amino acid sequence of SEQ ID NO: 52.

In some embodiments, an anti-trypsin 1/trypsin 2 antibody of the present disclosure comprises: a HC CDR1 that is at least 80% (e.g., at least 80%, at least 85%, at least 90%, at least 91%, at least 92%, at least 93%, at least 94%, at least 95%, at least 96%, at least 97%, at least 98%, or at least 99%) identical to the HC CDR1 having the amino acid sequence of SEQ ID NO: 49; a HC CDR2 that is at least 80% (e.g., at least 80%, at least 85%, at least 90%, at least 91%, at least 92%, at least 93%, at least 94%, at least 95%, at least 96%, at least 97%, at least 98%, or at least 99%) identical to the HC CDR2 having the amino acid sequence SEQ ID NO: 50; and/or a HC CDR3 that is at least 80% (e.g., at least 80%, at least 85%, at least 90%, at least 91%, at least 92%, at least 93%, at least 94%, at least 95%, at least 96%, at least 97%, at least 98%, or at least 99%) identical to the HC CDR3 having the amino acid sequence of SEQ ID NO: 51. Alternatively or in addition, the anti-trypsin 1/trypsin 2 antibody of the present disclosure comprises: a LC CDR1 that is at least 80% (e.g., at least 80%, at least 85%, at least 90%, at least 91%, at least 92%, at least 93%, at least 94%, at least 95%, at least 96%, at least 97%, at least 98%, or at least 99%) identical to the LC CDR1 having the amino acid sequence of SEQ ID NO: 4; a LC CDR2 that is at least 80% (e.g., at least 80%, at least 85%, at least 90%, at least 91%, at least 92%, at least 93%, at least 94%, at least 95%, at least 96%, at least 97%, at least 98%, or at least 99%) identical to the LC CDR2 having the amino acid sequence of SEQ ID NO: 5; and/or a LC CDR3 that is at least 80% (e.g., at least 80%, at least 85%, at least 90%, at least 91%, at least 92%, at least 93%, at least 94%, at least 95%, at least 96%, at least 97%, at least 98%, or at least 99%) identical to the LC CDR3 having the amino acid sequence of SEQ ID NO: 52.

In some embodiments, an anti-trypsin 1/trypsin 2 antibody of the present disclosure comprises a VHI comprising the amino acid sequence of SEQ ID NO: 81. Alternatively or in addition, the anti-trypsin 1/trypsin 2 antibody of the present disclosure comprises a VL comprising the amino acid sequence of SEQ ID NO: 82.

In some embodiments, an anti-trypsin 1/trypsin 2 antibody of the present disclosure comprises a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 81. Alternatively or in addition, the anti-trypsin 1/trypsin 2 antibody of the present disclosure comprises a VL containing no more than 20 amino acid variations (e.g., no more than 20, 19, 18, 17, 16, 15, 14, 13, 12, 11, 9, 8, 7, 6, 5, 4, 3, 2, or I amino acid variation) as compared with the VL as set forth in SEQ ID NO: 82. In some embodiments, the number of amino acid variations (e.g., no more than 20, 19, 18, 17, 16, 15, 14, 13, 12, 11, 10, 9, 8, 7, 6, 5, 4, 3, 2, or I amino acid variation) may occur within a VH of SEQ ID NO: 81 and/or a VL of SEQ ID NO: 82 excluding any of the CDR sequences therein, In some embodiments, an anti-trypsin 1/trypsin 2 antibodies provided herein comprise a heavy chain variable sequence that comprises a framework sequence that that contains no more than 20, 19, 18, 17, 16, 15, 14, 13, 12, 11, 10, 9, 8, 7, 6, 5, 4, 3, 2, or I amino acid variation to the framework sequence of a VH of SEQ ID NO: 81, and/or a light chain variable sequence that comprises a framework sequence that that contains no more than 20, 19, 18, 17, 16, 15, 14, 13, 12, 11, 10, 9, 8, 7, 6, 5, 4, 3, 2, or 1 amino acid variation to the framework sequence of a VL of SEQ ID NO: 82.

In some embodiments, an anti-trypsin 1/trypsin 2 antibody of the present disclosure comprises a VH comprising an amino acid sequence that is at least 80% (e.g., at least 80%, at least 85%, at least 90%, at least 91%, at least 92%, at least 93%, at least 94%, at least 95%, at least 96%, at least 97%, at least 98%, or at least 99%) identical to the VH as set forth in SEQ ID NO: 81. Alternatively or in addition, the anti-trypsin 1/trypsin 2 antibody of the present disclosure comprises a VL comprising an amino acid sequence that is at least 80% (e.g., at least 80%, at least 85%, at least 90%, at least 91%, at least 92%, at least 93%, at least 94%, at least 95%, at least 96%, at least 97%, at least 98%, or at least 99%) identical to the VL as set forth in SEQ ID NO: 82. In some embodiments, the degree of sequence variation (e.g., at least 75%, at least 80%, at least 85%, at least 90%, at least 91%, at least 92%, at least 93%, at least 94%, at least 95%, at least 96%, at least 97%, at least 98%, or at least 99%) may occur within a VH of SEQ ID NO: 81, and/or a VL of SEQ ID NO: 82 excluding any of the CDR sequences therein. In some embodiments, an anti-trypsin 1/trypsin 2 antibody provided herein comprise a heavy chain variable sequence that is at least 75%, at least 80%, at least 85%, at least 90%, at least 91%, at least 92%, at least 93%, at least 94%, at least 95%, at least 96%, at least 97%, at least 98%, or at least 99% identical to the framework sequence of a VH of SEQ ID NO: 81, and/or a light chain variable sequence that comprises a framework sequence that is at least 75%, at least 80%, at least 85%, at least 90%, at least 91%, at least 92%, at least 93%, at least 94%, at least 95%, at least 96%, at least 97%, at least 98%, or at least 99% identical to the framework sequence of a VL of SEQ ID NO: 82.

In some embodiments, an anti-trypsin 1/trypsin 2 antibody of the present disclosure comprises a HC CDR1, HC CDR2 and HC CDR3 of a heavy chain variable domain having the amino acid sequence of SEQ ID NO: 83. Alternatively or in addition, the anti-trypsin 1/trypsin 2 antibody of the present disclosure comprises a LC CDR1, LC CDR2 and LC CDR3 of a light chain variable domain having the amino acid sequence of SEQ ID NO: 70.

In some embodiments, an anti-trypsin 1/trypsin 2 antibody of the present disclosure comprises a HC CDR1 having the amino acid sequence of SEQ ID NO: 27, a HC CDR2 having the amino acid sequence of SEQ ID NO: 28, a HC CDR3 having the amino acid sequence of SEQ ID NO: 53, a LC CDR1 having the amino acid sequence of SEQ ID NO: 30, a LC CDR2 having the amino acid sequence of SEQ ID NO: 31, and a LC CDR3 having the amino acid sequence of SEQ ID NO: 32.

In some embodiments, an anti-trypsin 1/trypsin 2 antibody of the present disclosure comprises a HC CDR1, a HC CDR2, and a HC CDR3, which collectively contains no more than 5 amino acid variations (e.g., no more than 5, 4, 3, 2, or I amino acid variation) as compared with the HC CDR1 having the amino acid sequence of SEQ ID NO: 27, HC CDR2 having the amino acid sequence of SEQ ID NO: 28, and HC CDR3 having the amino acid sequence of SEQ ID NO: 53. Alternatively or in addition, the anti-trypsin 1/trypsin 2 antibody of the present disclosure comprises a LC CDR1, a LC CDR2, and a LC CDR3, which collectively contains no more than 5 amino acid variations (e.g., no more than 5, 4, 3, 2 or 1 amino acid variation) as compared with the LC CDR1 having the amino acid sequence of SEQ ID NO: 30, LC CDR2 having the amino acid sequence of SEQ ID NO: 31, and LC CDR3 having the amino acid sequence of SEQ ID NO: 32.

In some embodiments, an anti-trypsin 1/trypsin 2 antibody of the present disclosure comprises a HC CDR1, a HC CDR2, and a HC CDR3 that collectively are at least 80% (e.g., at least 80%, at least 85%, at least 90%, at least 91%, at least 92%, at least 93%, at least 94%, at least 95%, at least 96%, at least 97%, at least 98%, or at least 99%) identical to the HC CDR1 having the amino acid sequence of SEQ ID NO: 27, HC CDR2 having the amino acid sequence of SEQ ID NO: 28, and HC CDR3 having the amino acid sequence of SEQ ID NO: 53. Alternatively or in addition, the anti-trypsin 1/trypsin 2 antibody of the present disclosure comprises a LC CDR1, a LC CDR2, and a LC CDR3 that collectively are at least 80% (e.g., at least 80%, at least 85%, at least 90%, at least 91%, at least 92%, at least 93%, at least 94%, at least 95%, at least 96%, at least 97%, at least 98%, or at least 99%) identical to the to the LC CDR1 having the amino acid sequence of SEQ ID NO: 30, LC CDR2 having the amino acid sequence of SEQ ID NO: 31, and LC CDR3 having the amino acid sequence of SEQ ID NO: 32.

In some embodiments, an anti-trypsin 1/trypsin 2 antibody of the present disclosure comprises: a HC CDR1 having no more than 3 amino acid variations (e.g., no more than 3, 2, or 1 amino acid variation) as compared with the HC CDR1 having the amino acid sequence of SEQ ID NO: 27; a HC CDR2 having no more than 3 amino acid variations (e.g., no more than 3, 2, or I amino acid variation) as compared with the HC CDR2 having the amino acid sequence of SEQ ID NO: 28; and/or a HC CDR3 having no more than 3 amino acid variations (e.g., no more than 3, 2, or 1 amino acid variation) as compared with the HC CDR3 having the amino acid sequence of SEQ ID NO: 53. Alternatively or in addition, the anti-trypsin 1/trypsin 2 antibody of the present disclosure comprises: a LC CDR1 having no more than 3 amino acid variations (e.g., no more than 3, 2, or I amino acid variation) as compared with the LC CDR1 having the amino acid sequence of SEQ ID NO: 30; a LC CDR2 having no more than 3 amino acid variations (e.g., no more than 3, 2, or I amino acid variation) as compared with the LC CDR2 having the amino acid sequence of SEQ ID NO: 31; and/or a LC CDR3 having no more than 3 amino acid variations (e.g., no more than 3, 2, or 1 amino acid variation) as compared with the LC CDR3 having the amino acid sequence of SEQ ID NO: 32.

In some embodiments, an anti-trypsin 1/trypsin 2 antibody of the present disclosure comprises: a HC CDR1 that is at least 80% (e.g., at least 80%, at least 85%, at least 90%, at least 91%, at least 92%, at least 93%, at least 94%, at least 95%, at least 96%, at least 97%, at least 98%, or at least 99%) identical to the HC CDR1 having the amino acid sequence of SEQ ID NO: 27; a HC CDR2 that is at least 80% (e.g., at least 80%, at least 85%, at least 90%, at least 91%, at least 92%, at least 93%, at least 94%, at least 95%, at least 96%, at least 97%, at least 98%, or at least 99%) identical to the HC CDR2 having the amino acid sequence SEQ ID NO: 28; and/or a HC CDR3 that is at least 80% (e.g., at least 80%, at least 85%, at least 90%, at least 91%, at least 92%, at least 93%, at least 94%, at least 95%, at least 96%, at least 97%, at least 98%, or at least 99%) identical to the HC CDR3 having the amino acid sequence of SEQ ID NO: 53. Alternatively or in addition, the anti-trypsin 1/trypsin 2 antibody of the present disclosure comprises: a LC CDR1 that is at least 80% (e.g., at least 80%, at least 85%, at least 90%, at least 91%, at least 92%, at least 93%, at least 94%, at least 95%, at least 96%, at least 97%, at least 98%, or at least 99%) identical to the LC CDR1 having the amino acid sequence of SEQ ID NO: 30; a LC CDR2 that is at least 80% (e.g., at least 80%, at least 85%, at least 90%, at least 91%, at least 92%, at least 93%, at least 94%, at least 95%, at least 96%, at least 97%, at least 98%, or at least 99%) identical to the LC CDR2 having the amino acid sequence of SEQ ID NO: 31; and/or a LC CDR3 that is at least 80% (e.g., at least 80%, at least 85%, at least 90%, at least 91%, at least 92%, at least 93%, at least 94%, at least 95%, at least 96%, at least 97%, at least 98%, or at least 99%) identical to the LC CDR3 having the amino acid sequence of SEQ ID NO: 32.

In some embodiments, an anti-trypsin 1/trypsin 2 antibody of the present disclosure comprises a VH comprising the amino acid sequence of SEQ ID NO: 83. Alternatively or in addition, the anti-trypsin 1/trypsin 2 antibody of the present disclosure comprises a VL comprising the amino acid sequence of SEQ ID NO: 70.

In some embodiments, an anti-trypsin 1/trypsin 2 antibody of the present disclosure comprises a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 83. Alternatively or in addition, the anti-trypsin 1/trypsin 2 antibody of the present disclosure comprises a VL containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the VL as set forth in SEQ ID NO: 70. In some embodiments, the number of amino acid variations (e.g., no more than 20, 19, 18, 17, 16, 15, 14, 13, 12, 11, 10, 9, 8, 7, 6, 5, 4, 3, 2, or 1 amino acid variation) may occur within a VH of SEQ ID NO: 83 and/or a VL of SEQ ID NO: 70 excluding any of the CDR sequences therein. In some embodiments, an anti-trypsin 1/trypsin 2 antibodies provided herein comprise a heavy chain variable sequence that comprises a framework sequence that that contains no more than 20, 19, 18, 17, 16, 15, 14, 13, 12, 11, 10, 9, 8, 7, 6, 5, 4, 3, 2, or 1 amino acid variation to the framework sequence of a VH of SEQ ID NO: 83, and/or a light chain variable sequence that comprises a framework sequence that that contains no more than 20, 19, 18, 17, 16, 15, 14, 13, 12, 11, 10, 9, 8, 7, 6, 5, 4, 3, 2, or 1 amino acid variation to the framework sequence of a VL of SEQ ID NO: 70.

In some embodiments, an anti-trypsin 1/trypsin 2 antibody of the present disclosure comprises a VH comprising an amino acid sequence that is at least 80% (e.g., at least 80%, at least 85%, at least 90%, at least 91%, at least 92%, at least 93%, at least 94%, at least 95%, at least 96%, at least 97%, at least 98%, or at least 99%) identical to the VH as set forth in SEQ ID NO: 83. Alternatively or in addition, the anti-trypsin 1/trypsin 2 antibody of the present disclosure comprises a VL comprising an amino acid sequence that is at least 80% (e.g., at least 80%, at least 85%, at least 90%, at least 91%, at least 92%, at least 93%, at least 94%, at least 95%, at least 96%, at least 97%, at least 98%, or at least 99%) identical to the VL as set forth in SEQ ID NO: 70. In some embodiments, the degree of sequence variation (e.g., at least 75%, at least 80%, at least 85%, at least 90%, at least 91%, at least 92%, at least 93%, at least 94%, at least 95%, at least 96%, at least 97%, at least 98%, or at least 99%) may occur within a VH of SEQ ID NO: 83, and/or a VL of SEQ ID NO: 70 excluding any of the CDR sequences therein. In some embodiments, an anti-trypsin 1/trypsin 2 antibody provided herein comprise a heavy chain variable sequence that is at least 75%, at least 80%, at least 85%, at least 90%, at least 91%, at least 92%, at least 93%, at least 94%, at least 95%, at least 96%, at least 97%, at least 98%, or at least 99% identical to the framework sequence of a VH of SEQ ID NO: 83, and/or a light chain variable sequence that comprises a framework sequence that is at least 75%, at least 80%, at least 85%, at least 90%, at least 91%, at least 92%, at least 93%, at least 94%, at least 95%, at least 96%, at least 97%, at least 98%, or at least 99% identical to the framework sequence of a VL of SEQ ID NO: 70.

In some embodiments, an anti-trypsin 1/trypsin 2 antibody of the present disclosure comprises a HC CDR1, HC CDR2 and HC CDR3 of a heavy chain variable domain having the amino acid sequence of SEQ ID NO: 84. Alternatively or in addition, the anti-trypsin 1/trypsin 2 antibody of the present disclosure comprises a LC CDR1, LC CDR2 and LC CDR3 of a light chain variable domain having the amino acid sequence of SEQ ID NO: 70.

In some embodiments, an anti-trypsin 1/trypsin 2 antibody of the present disclosure comprises a HC CDR1 having the amino acid sequence of SEQ ID NO: 54, a HC CDR2 having the amino acid sequence of SEQ ID NO: 55, a HC CDR3 having the amino acid sequence of SEQ ID NO: 56, a LC CDR1 having the amino acid sequence of SEQ ID NO: 30, a LC CDR2 having the amino acid sequence of SEQ ID NO: 31, and a LC CDR3 having the amino acid sequence of SEQ ID NO: 32.

In some embodiments, an anti-trypsin 1/trypsin 2 antibody of the present disclosure comprises a HC CDR1, a HC CDR2, and a HC CDR3, which collectively contains no more than 5 amino acid variations (e.g., no more than 5, 4, 3, 2, or I amino acid variation) as compared with the HC CDR1 having the amino acid sequence of SEQ ID NO: 54, HC CDR2 having the amino acid sequence of SEQ ID NO: 55, and HC CDR3 having the amino acid sequence of SEQ ID NO: 56. Alternatively or in addition, the anti-trypsin 1/trypsin 2 antibody of the present disclosure comprises a LC CDR1, a LC CDR2, and a LC CDR3, which collectively contains no more than 5 amino acid variations (e.g., no more than 5, 4, 3, 2 or 1 amino acid variation) as compared with the LC CDR1 having the amino acid sequence of SEQ ID NO: 30, LC CDR2 having the amino acid sequence of SEQ ID NO: 31, and LC CDR3 having the amino acid sequence of SEQ ID NO: 32.

In some embodiments, an anti-trypsin 1/trypsin 2 antibody of the present disclosure comprises a HC CDR1, a HC CDR2, and a HC CDR3 that collectively are at least 80% (e.g., at least 80%, at least 85%, at least 90%, at least 91%, at least 92%, at least 93%, at least 94%, at least 95%, at least 96%, at least 97%, at least 98%, or at least 99%) identical to the HC CDR1 having the amino acid sequence of SEQ ID NO: 54, HC CDR2 having the amino acid sequence of SEQ ID NO: 55, and HC CDR3 having the amino acid sequence of SEQ ID NO: 56. Alternatively or in addition, the anti-trypsin 1/trypsin 2 antibody of the present disclosure comprises a LC CDR1, a LC CDR2, and a LC CDR3 that collectively are at least 80% (e.g., at least 80%, at least 85%, at least 90%, at least 91%, at least 92%, at least 93%, at least 94%, at least 95%, at least 96%, at least 97%, at least 98%, or at least 99%) identical to the to the LC CDR1 having the amino acid sequence of SEQ ID NO: 30, LC CDR2 having the amino acid sequence of SEQ ID NO: 31, and LC CDR3 having the amino acid sequence of SEQ ID NO: 32.

In some embodiments, an anti-trypsin 1/trypsin 2 antibody of the present disclosure comprises: a HC CDR1 having no more than 3 amino acid variations (e.g., no more than 3, 2, or 1 amino acid variation) as compared with the HC CDR1 having the amino acid sequence of SEQ ID NO: 54; a HC CDR2 having no more than 3 amino acid variations (e.g., no more than 3, 2, or I amino acid variation) as compared with the HC CDR2 having the amino acid sequence of SEQ ID NO: 55; and/or a HC CDR3 having no more than 3 amino acid variations (e.g., no more than 3, 2, or 1 amino acid variation) as compared with the HC CDR3 having the amino acid sequence of SEQ ID NO: 56. Alternatively or in addition, the anti-trypsin 1/trypsin 2 antibody of the present disclosure comprises: a LC CDR1 having no more than 3 amino acid variations (e.g., no more than 3, 2, or 1 amino acid variation) as compared with the LC CDR1 having the amino acid sequence of SEQ ID NO: 30; a LC CDR2 having no more than 3 amino acid variations (e.g., no more than 3, 2, or 1 amino acid variation) as compared with the LC CDR2 having the amino acid sequence of SEQ ID NO: 31; and/or a LC CDR3 having no more than 3 amino acid variations (e.g., no more than 3, 2, or 1 amino acid variation) as compared with the LC CDR3 having the amino acid sequence of SEQ ID NO: 32.

In some embodiments, an anti-trypsin 1/trypsin 2 antibody of the present disclosure comprises: a HC CDR1 that is at least 80% (e.g., at least 80%, at least 85%, at least 90%, at least 91%, at least 92%, at least 93%, at least 94%, at least 95%, at least 96%, at least 97%, at least 98%, or at least 99%) identical to the HC CDR1 having the amino acid sequence of SEQ ID NO: 54; a HC CDR2 that is at least 80% (e.g., at least 80%, at least 85%, at least 90%, at least 91%, at least 92%, at least 93%, at least 94%, at least 95%, at least 96%, at least 97%, at least 98%, or at least 99%) identical to the HC CDR2 having the amino acid sequence SEQ ID NO: 55; and/or a HC CDR3 that is at least 80% (e.g., at least 80%, at least 85%, at least 90%, at least 91%, at least 92%, at least 93%, at least 94%, at least 95%, at least 96%, at least 97%, at least 98%, or at least 99%) identical to the HC CDR3 having the amino acid sequence of SEQ ID NO: 56. Alternatively or in addition, the anti-trypsin 1/trypsin 2 antibody of the present disclosure comprises: a LC CDR1 that is at least 80% (e.g., at least 80%, at least 85%, at least 90%, at least 91%, at least 92%, at least 93%, at least 94%, at least 95%, at least 96%, at least 97%, at least 98%, or at least 99%) identical to the LC CDR1 having the amino acid sequence of SEQ ID NO: 30; a LC CDR2 that is at least 80% (e.g., at least 80%, at least 85%, at least 90%, at least 91%, at least 92%, at least 93%, at least 94%, at least 95%, at least 96%, at least 97%, at least 98%, or at least 99%) identical to the LC CDR2 having the amino acid sequence of SEQ ID NO: 31; and/or a LC CDR3 that is at least 80% (e.g., at least 80%, at least 85%, at least 90%, at least 91%, at least 92%, at least 93%, at least 94%, at least 95%, at least 96%, at least 97%, at least 98%, or at least 99%) identical to the LC CDR3 having the amino acid sequence of SEQ ID NO: 32.

In some embodiments, an anti-trypsin 1/trypsin 2 antibody of the present disclosure comprises a VH comprising the amino acid sequence of SEQ ID NO: 84. Alternatively or in addition, the anti-trypsin 1/trypsin 2 antibody of the present disclosure comprises a VL comprising the amino acid sequence of SEQ ID NO: 70.

In some embodiments, an anti-trypsin 1/trypsin 2 antibody of the present disclosure comprises a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 84. Alternatively or in addition, the anti-trypsin 1/trypsin 2 antibody of the present disclosure comprises a VL containing no more than 20 amino acid variations (e.g., no more than 20, 19, 18, 17, 16, 15, 14, 13, 12, 11, 9, 8, 7, 6, 5, 4, 3, 2, or I amino acid variation) as compared with the VL as set forth in SEQ ID NO: 70. In some embodiments, the number of amino acid variations (e.g., no more than 20, 19, 18, 17, 16, 15, 14, 13, 12, 11, 10, 9, 8, 7, 6, 5, 4, 3, 2, or 1 amino acid variation) may occur within a VH of SEQ ID NO: 84 and/or a VL of SEQ ID NO: 70 excluding any of the CDR sequences therein. In some embodiments, an anti-trypsin 1/trypsin 2 antibodies provided herein comprise a heavy chain variable sequence that comprises a framework sequence that that contains no more than 20, 19, 18, 17, 16, 15, 14, 13, 12, 11, 10, 9, 8, 7, 6, 5, 4, 3, 2, or I amino acid variation to the framework sequence of a VH of SEQ ID NO: 84, and/or a light chain variable sequence that comprises a framework sequence that that contains no more than 20, 19, 18, 17, 16, 15, 14, 13, 12, 11, 10, 9, 8, 7, 6, 5, 4, 3, 2, or 1 amino acid variation to the framework sequence of a VL of SEQ ID NO: 70.

In some embodiments, an anti-trypsin 1/trypsin 2 antibody of the present disclosure comprises a VH comprising an amino acid sequence that is at least 80% (e.g., at least 80%, at least 85%, at least 90%, at least 91%, at least 92%, at least 93%, at least 94%, at least 95%, at least 96%, at least 97%, at least 98%, or at least 99%) identical to the VH as set forth in SEQ ID NO: 84. Alternatively or in addition, the anti-trypsin 1/trypsin 2 antibody of the present disclosure comprises a VL comprising an amino acid sequence that is at least 80% (e.g., at least 80%, at least 85%, at least 90%, at least 91%, at least 92%, at least 93%, at least 94%, at least 95%, at least 96%, at least 97%, at least 98%, or at least 99%) identical to the VL as set forth in SEQ ID NO: 70. In some embodiments, the degree of sequence variation (e.g., at least 75%, at least 80%, at least 85%, at least 90%, at least 91%, at least 92%, at least 93%, at least 94%, at least 95%, at least 96%, at least 97%, at least 98%, or at least 99%) may occur within a VH of SEQ ID NO: 84, and/or a VL of SEQ ID NO: 70 excluding any of the CDR sequences therein. In some embodiments, an anti-trypsin 1/trypsin 2 antibody provided herein comprise a heavy chain variable sequence that is at least 75%, at least 80%, at least 85%, at least 90%, at least 91%, at least 92%, at least 93%, at least 94%, at least 95%, at least 96%, at least 97%, at least 98%, or at least 99% identical to the framework sequence of a VH of SEQ ID NO: 84, and/or a light chain variable sequence that comprises a framework sequence that is at least 75%, at least 80%, at least 85%, at least 90%, at least 91%, at least 92%, at least 93%, at least 94%, at least 95%, at least 96%, at least 97%, at least 98%, or at least 99% identical to the framework sequence of a VL of SEQ ID NO: 70.

The antibodies described herein can be in any antibody form, including, but not limited to, intact (i.e., full-length) antibodies, antigen-binding fragments thereof (such as Fab, F(ab′), F(ab′)2, Fv), single chain antibodies, bi-specific antibodies, or nanobodies. In some embodiments, the anti-trypsin 1/trypsin 2 antibody described herein is a scFv. In some embodiments, the anti-trypsin 1/trypsin 2 antibody described herein is a scFv-Fab (e.g., scFv fused to a portion of a constant region).

In some embodiments, an anti-trypsin 1/trypsin 2 antibody of the present disclosure is a chimeric antibody, which can include a heavy constant region and a light constant region from a human antibody. Chimeric antibodies refer to antibodies having a variable region or part of variable region from a first species and a constant region from a second species. Typically, in these chimeric antibodies, the variable region of both light and heavy chains mimics the variable regions of antibodies derived from one species of mammals (e.g., a non-human mammal such as mouse, rabbit, and rat), while the constant portions are homologous to the sequences in antibodies derived from another mammal such as human. In some embodiments, amino acid modifications can be made in the variable region and/or the constant region.

In some embodiments, an antibody of the present disclosure comprises a VL domain and/or VH domain of any one of the anti-trypsin 1/trypsin 2 antibodies selected from Tables 1a and 1b, and comprises a constant region comprising the amino acid sequences of the constant regions of an IgG, IgE, IgM, IgD, IgA or IgY immunoglobulin molecule, any class (e.g., IgG1, IgG2, IgG3, IgG4, IgA1 and IgA2), or any subclass (e.g., IgG2a and IgG2b) of immunoglobulin molecule. Non-limiting examples of human constant regions are described in the art, e.g., see Kabat E A et al., (1991) supra.

In some embodiments, the light chain of any of the anti-trypsin 1/trypsin 2 antibodies described herein may further comprise a light chain constant region (CL), which can be any CL known in the art. In some examples, the CL is a kappa light chain. In other examples, the CL is a lambda light chain. In some embodiments, the CL is a kappa light chain. In some embodiments, an antibody of the present disclosure comprises a VL domain and/or VH domain of any one of the anti-trypsin 1/trypsin 2 antibodies selected from Tables 1a and 1b, and further comprises a kappa light chain constant domain.

Other antibody heavy and light chain constant regions are well known in the art, e.g., those provided in the IMGT database or at VBASE2, both of which are incorporated by reference herein.

In some embodiments, conservative mutations can be introduced into antibody sequences (e.g., CDRs or framework sequences) at positions where the residues are not likely to be involved in interacting with a target antigen (e.g., human or non-human primate trypsin 1 and/or human or non-human primate trypsin 2), for example, as determined based on a crystal structure. In some embodiments, one, two or more mutations (e.g., amino acid substitutions) are introduced into the Fc region of an anti-trypsin 1/trypsin 2 antibody described herein (e.g., in a CH2 domain (residues 231-340 of human IgG1) and/or CH3 domain (residues 341-447 of human IgG1) and/or the hinge region, with numbering according to the Kabat numbering system (e.g., the EU index in Kabat)) to alter one or more functional properties of the antibody, such as serum half-life, complement fixation, Fc receptor binding and/or antigen-dependent cellular cytotoxicity.

In some embodiments, one, two or more mutations (e.g., amino acid substitutions) are introduced into the hinge region of the Fc region (CH1 domain) such that the number of cysteine residues in the hinge region are altered (e.g., increased or decreased) as described in, e.g., U.S. Pat. No. 5,677,425. The number of cysteine residues in the hinge region of the CH1 domain can be altered to, e.g., facilitate assembly of the light and heavy chains, or to alter (e.g., increase or decrease) the stability of the antibody or to facilitate linker conjugation.

In some embodiments, one, two or more mutations (e.g., amino acid substitutions) are introduced into the Fc region of an antibody described herein (e.g., in a CH2 domain (residues 231-340 of human IgG1) and/or CH3 domain (residues 341-447 of human IgG1) and/or the hinge region, with numbering according to the Kabat numbering system (e.g., the EU index in Kabat)) to increase or decrease the affinity of the antibody for an Fc receptor (e.g., an activated Fc receptor) on the surface of an effector cell. Mutations in the Fc region of an antibody that decrease or increase the affinity of an antibody for an Fc receptor and techniques for introducing such mutations into the Fc receptor or fragment thereof are known to one of skill in the art. Examples of mutations in the Fc receptor of an antibody that can be made to alter the affinity of the antibody for an Fc receptor are described in, e.g., Smith P et al., (2012) PNAS 109: 6181-6186, U.S. Pat. No. 6,737,056, and International Publication Nos. WO 02/060919; WO 98/23289; and WO 97/34631, which are incorporated herein by reference.

In some embodiments, one, two or more amino acid mutations (i.e., substitutions, insertions or deletions) are introduced into an IgG constant domain, or FcRn-binding fragment thereof (preferably an Fc or hinge-Fc domain fragment) to alter (e.g., decrease or increase) half-life of the antibody in vivo. See, e.g., International Publication Nos. WO 02/060919; WO 98/23289; and WO 97/34631; and U.S. Pat. Nos. 5,869,046, 6,121,022, 6,277,375 and 6,165,745 for examples of mutations that will alter (e.g., decrease or increase) the half-life of an antibody in vivo.

In some embodiments, one, two or more amino acid mutations (i.e., substitutions, insertions or deletions) are introduced into an IgG constant domain, or FcRn-binding fragment thereof (preferably an Fc or hinge-Fc domain fragment) to decrease the half-life of the anti-trypsin 1/trypsin 2 antibody in vivo. In some embodiments, one, two or more amino acid mutations (i.e., substitutions, insertions or deletions) are introduced into an IgG constant domain, or FcRn-binding fragment thereof (preferably an Fc or hinge-Fc domain fragment) to increase the half-life of the antibody in vivo. In some embodiments, the antibodies can have one or more amino acid mutations (e.g., substitutions) in the second constant (CH2) domain (residues 231-340 of human IgG1) and/or the third constant (CH3) domain (residues 341-447 of human IgG1), with numbering according to the EU index in Kabat (Kabat E A et al., (1991) supra). In some embodiments, the constant region of the IgG1 of an antibody described herein comprises a methionine (M) to tyrosine (Y) substitution in position 252, a serine (S) to threonine (T) substitution in position 254, and a threonine (T) to glutamic acid (E) substitution in position 256, numbered according to the EU index as in Kabat. See U.S. Pat. No. 7,658,921, which is incorporated herein by reference. This type of mutant IgG, referred to as “YTE mutant” has been shown to display fourfold increased half-life as compared to wild-type versions of the same antibody (see Dall'Acqua W F et al., (2006) J Biol Chem 281: 23514-24). In some embodiments, an antibody comprises an IgG constant domain comprising one, two, three or more amino acid substitutions of amino acid residues at positions 251-257, 285-290, 308-314, 385-389, and 428-436, numbered according to the EU index as in Kabat.

In some embodiments, an antibody comprises an Fc region that has been engineered for half-life extension purposes, e.g., by introducing M428L and/or N434A substitutions. Non-limiting examples of such Fc variants effecting half-life in circulation are provided in Saunders K O, Conceptual Approaches to Modulating Antibody Effector Functions and Circulation Half-Life, Front Immunol. 2019; 10: 1296, the contents of which are incorporated herein by reference.

In some embodiments, one, two or more amino acid substitutions are introduced into an IgG constant domain Fc region to alter the effector function(s) of the anti-trypsin 1/trypsin 2 antibody, e.g., by introducing Leu234Ala and Leu235Ala mutations (commonly called LALA mutations). The effector ligand to which affinity is altered can be, for example, an Fc receptor or the C1 component of complement. This approach is described in further detail in U.S. Pat. Nos. 5,624,821 and 5,648,260. In some embodiments, the deletion or inactivation (through point mutations or other means) of a constant region domain can reduce Fc receptor binding of the circulating antibody thereby increasing tumor localization. See, e.g., U.S. Pat. Nos. 5,585,097 and 8,591,886 for a description of mutations that delete or inactivate the constant domain and thereby increase tumor localization. In some embodiments, one or more amino acid substitutions may be introduced into the Fc region of an antibody described herein to remove potential glycosylation sites on Fc region, which may reduce Fc receptor binding (see, e.g., Shields R L et al., (2001) J Biol Chem 276: 6591-604).

In some embodiments, one or more amino in the constant region of an anti-trypsin 1/trypsin 2 antibody described herein can be replaced with a different amino acid residue such that the antibody has altered C1q binding and/or reduced or abolished complement dependent cytotoxicity (CDC). This approach is described in further detail in U.S. Pat. No. 6,194,551 (Idusogie et al). In some embodiments, one or more amino acid residues in the N-terminal region of the CH2 domain of an antibody described herein are altered to thereby alter the ability of the antibody to fix complement. This approach is described further in International Publication No. WO 94/29351. In some embodiments, the Fc region of an antibody described herein is modified to increase the ability of the antibody to mediate antibody dependent cellular cytotoxicity (ADCC) and/or to increase the affinity of the antibody for an Fcγ receptor. This approach is described further in International Publication No. WO 00/42072.

In some embodiments, an antibody comprises an Fc variant comprising amino acid substitutions L234A, L235E, and P329G, wherein numbering is according to the EU index. In some embodiments, the antibody comprising the Fc variant exhibits reduced affinity to one or more or each of FcγRJ, FcγRIIA, FcγRIIIA, and C1q as compared to an antibody comprising the wild-type human Fc region. Examples of such Fc variants are provided in International Patent Application Publication No.: WO 2021/055669 entitled, FC VARIANTS WITH REDUCED EFFECTOR FUNCTION, published on Mar. 25, 2021; and US Patent Application Publication No.: US 2021-0087271 entitled, FC VARIANTS WITH REDUCED EFFECTOR FUNCTION, published on Mar. 25, 2021, the contents of which are incorporated herein by reference.

In some embodiments, the heavy and/or light chain variable domain(s) sequence(s) of the antibodies provided herein can be used to generate, for example, CDR-grafted, chimeric, humanized, or composite human antibodies or antigen-binding fragments, as described elsewhere herein. As understood by one of ordinary skill in the art, any variant, CDR-grafted, chimeric, humanized, or composite antibodies derived from any of the antibodies provided herein may be useful in the compositions and methods described herein and will maintain the ability to specifically bind trypsin 1 and trypsin 2, such that the variant, CDR-grafted, chimeric, humanized, or composite antibody has at least 50%, at least 60%, at least 70%, at least 80%, at least 90%, at least 95% or more binding to trypsin 1 and trypsin 2 relative to the original antibody from which it is derived.

In some embodiments, the antibodies provided herein comprise mutations that confer desirable properties to the antibodies. For example, to avoid potential complications due to Fab-arm exchange, which is known to occur with native IgG4 mAbs, the antibodies provided herein may comprise a stabilizing ‘Adair’ mutation (Angal S., et al., “A single amino acid substitution abolishes the heterogeneity of chimeric mouse/human (IgG4) antibody,” Mol Immunol 30, 105-108; 1993), where serine 228 (EU numbering; residue 241 Kabat numbering) is converted to proline resulting in an IgG1-like hinge sequence. Accordingly, any of the antibodies may include a stabilizing ‘Adair’ mutation.

In some embodiments, an antibody is modified, e.g., modified via glycosylation, phosphorylation, sumoylation, and/or methylation. In some embodiments, an antibody is a glycosylated antibody, which is conjugated to one or more sugar or carbohydrate molecules. In some embodiments, the one or more sugar or carbohydrate molecule are conjugated to the antibody via N-glycosylation, O-glycosylation, C-glycosylation, glypiation (GPI anchor attachment), and/or phosphoglycosylation. In some embodiments, the one or more sugar or carbohydrate molecules are monosaccharides, disaccharides, oligosaccharides, or glycans. In some embodiments, the one or more sugar or carbohydrate molecule is a branched oligosaccharide or a branched glycan. In some embodiments, the one or more sugar or carbohydrate molecule includes a mannose unit, a glucose unit, an N-acetylglucosamine unit, an N-acetylgalactosamine unit, a galactose unit, a fucose unit, or a phospholipid unit. In some embodiments, there are about 1-10, about 1-5, about 5-10, about 1-4, about 1-3, or about 2 sugar molecules. In some embodiments, a glycosylated antibody is fully or partially glycosylated. In some embodiments, an antibody is glycosylated by chemical reactions or by enzymatic means. In some embodiments, an antibody is glycosylated in vitro or inside a cell, which may optionally be deficient in an enzyme in the N- or O-glycosylation pathway, e.g. a glycosyltransferase. In some embodiments, an antibody is functionalized with sugar or carbohydrate molecules as described in International Patent Application Publication WO2014065661, published on May 1, 2014, entitled, “Modified antibody, antibody-conjugate and process for the preparation thereof”.

In some embodiments, any one of the anti-trypsin 1/trypsin 2 antibodies described herein may comprise a signal peptide in the heavy and/or light chain sequence (e.g., a N-terminal signal peptide). In some embodiments, the anti-trypsin 1/trypsin 2 antibody described herein comprises any one of the VH and VL sequences, any one of the IgG heavy chain and light chain sequences, or any one of the F(ab′) heavy chain and light chain sequences described herein, and further comprises a signal peptide (e.g., a N-terminal signal peptide).

In some embodiments, an antibody provided here is a multi-specific antibody (e.g., a bispecific antibody). For example, in some embodiments, one or more anti-trypsin 1/trypsin 2 antibodies may be combined with one or more different anti-trypsin 1/trypsin 2 antibody to produce a multi-specific or a bispecific anti-trypsin 1/trypsin 2 antibody. For example, one or more anti-trypsin 1/trypsin 2 antibody as described herein (Table 1a and Table 1b), can be combined with one or more different anti-trypsin 1/trypsin 2 antibody described herein (Table 1a and Table 1b) to produce a multi-specific antibody.

In some embodiments, one or more anti-trypsin 1/trypsin 2 antibodies may be combined with any other appropriate therapeutic antibodies to produce a multi-specific or a bispecific anti-trypsin 1/trypsin 2/additional target antibody. For example, an anti-trypsin 1/trypsin 2 antibody as described herein (Table 1a and Table 1b), can be combined with any appropriate antibody to produce a bispecific antibody. Such additional therapeutic antibody may be, for example, an anti-intercellular adhesion molecule-1 (ICAM-1) antibody (see, e.g., Werner et al., Specific Therapy for Local and Systemic Complications of Acute Pancreatitis With Monoclonal Antibodies Against ICAM-1, Ann Surg. 1999 June; 229(6): 834)).

### III. Preparation of the Anti-Trypsin 1/Trypsin 2 Antibodies

Antibodies described herein can be made by any method known in the art. See, for example, Harlow and Lane, (1998) Antibodies: A Laboratory Manual, Cold Spring Harbor Laboratory, New York.

In some embodiments, antibodies specific to a target antigen (e.g., trypsin 1 and/or trypsin 2) can be made by the conventional hybridoma technology. The full-length target antigen or a fragment thereof, optionally coupled to a carrier protein such as KLH, can be used to immunize a host animal for generating antibodies binding to that antigen. The route and schedule of immunization of the host animal are generally in keeping with established and conventional techniques for antibody stimulation and production, as further described herein. General techniques for production of mouse, humanized, and human antibodies are known in the art and are described herein. It is contemplated that any mammalian subject including humans or antibody producing cells therefrom can be manipulated to serve as the basis for production of mammalian, including human hybridoma cell lines. Typically, the host animal is inoculated intraperitoneally, intramuscularly, orally, subcutaneously, intraplantar, and/or intradermally with an amount of immunogen, including as described herein.

If desired, an antibody (monoclonal or polyclonal) of interest (e.g., produced by a hybridoma) may be sequenced and the polynucleotide sequence may then be cloned into a vector for expression or propagation. The sequence encoding the antibody of interest may be maintained in vector in a host cell and the host cell can then be expanded and frozen for future use. In an alternative, the polynucleotide sequence may be used for genetic manipulation to “humanize” the antibody or to improve the affinity (affinity maturation), or other characteristics of the antibody. For example, the constant region may be engineered to more resemble human constant regions to avoid immune response if the antibody is used in clinical trials and treatments in humans. It may be desirable to genetically manipulate the antibody sequence to obtain greater affinity to the target antigen and greater efficacy. It will be apparent to one of skill in the art that one or more polynucleotide changes can be made to the antibody and still maintain its binding specificity to the target antigen.

In other embodiments, fully human antibodies can be obtained by using commercially available mice that have been engineered to express specific human immunoglobulin proteins. Transgenic animals that are designed to produce a more desirable (e.g., fully human antibodies) or more robust immune response may also be used for generation of humanized or human antibodies. Examples of such technology are Xenomouse® from Amgen, Inc. (Fremont, CA) and HuMAb-Mouse® and TC Mouse™ from Medarex, Inc. (Princeton, NJ) or H2L2 mice from Harbour Antibodies BV (Holland). In another alternative, antibodies may be made recombinantly by phage display or yeast technology. See, for example, U.S. Pat. Nos. 5,565,332; 5,580,717; 5,733,743; and 6,265,150; and Winter et al., (1994) Annu. Rev. Immunol. 12:433-455. Alternatively, the phage display technology (McCafferty et al., (1990) Nature 348:552-553) can be used to produce human antibodies and antibody fragments in vitro, from immunoglobulin variable (V) domain gene repertoires from unimmunized donors.

Antigen-binding fragments of an intact antibody (full-length antibody) can be prepared via routine methods. For example, F(ab′)2 fragments can be produced by pepsin digestion of an antibody molecule, and Fab fragments that can be generated by reducing the disulfide bridges of F(ab′)2 fragments. Genetically engineered antibodies, such as humanized antibodies, chimeric antibodies, single-chain antibodies, and bi-specific antibodies, can be produced via, e.g., conventional recombinant technology. In one example, DNA encoding a monoclonal antibodies specific to a target antigen can be readily isolated and sequenced using conventional procedures (e.g., by using oligonucleotide probes that are capable of binding specifically to genes encoding the heavy and light chains of the monoclonal antibodies). The hybridoma cells serve as a preferred source of such DNA. Once isolated, the DNA may be placed into one or more expression vectors, which are then transfected into host cells such as E. coli cells, simian COS cells, Chinese hamster ovary (CHO) cells, human HEK293 cells, or myeloma cells that do not otherwise produce immunoglobulin protein, to obtain the synthesis of monoclonal antibodies in the recombinant host cells. See, e.g., PCT Publication No. WO 87/04462. The DNA can then be modified, for example, by substituting the coding sequence for human heavy and light chain constant domains in place of the homologous murine sequences, Morrison et al., (1984) Proc. Nat. Acad. Sci. 81:6851, or by covalently joining to the immunoglobulin coding sequence all or part of the coding sequence for a non-immunoglobulin polypeptide. In that manner, genetically engineered antibodies, such as “chimeric” or “hybrid” antibodies; can be prepared that have the binding specificity of a target antigen.

A single-chain antibody can be prepared via recombinant technology by linking a nucleotide sequence coding for a heavy chain variable region and a nucleotide sequence coding for a light chain variable region. Preferably, a flexible linker is incorporated between the two variable regions.

Antibodies obtained following a method known in the art and described herein can be characterized using methods well known in the art. For example, one method is to identify the epitope to which the antigen binds, or “epitope mapping.” There are many methods known in the art for mapping and characterizing the location of epitopes on proteins, including solving the crystal structure of an antibody-antigen complex, competition assays, gene fragment expression assays, and synthetic peptide-based assays, as described, for example, in Chapter 11 of Harlow and Lane, Using Antibodies, a Laboratory Manual, Cold Spring Harbor Laboratory Press, Cold Spring Harbor, N.Y., 1999. In one example, epitope mapping can be accomplished use H/D-Ex (hydrogen deuterium exchange) coupled with proteolysis and mass spectrometry. In an additional example, epitope mapping can be used to determine the sequence to which an antibody binds. The epitope can be a linear epitope, i.e., contained in a single stretch of amino acids, or a conformational epitope formed by a three-dimensional interaction of amino acids that may not necessarily be contained in a single stretch (primary structure linear sequence). Peptides of varying lengths (e.g., at least 4-6 amino acids long) can be isolated or synthesized (e.g., recombinantly) and used for binding assays with an antibody. In another example, the epitope to which the antibody binds can be determined in a systematic screening by using overlapping peptides derived from the target antigen sequence and determining binding by the antibody. According to the gene fragment expression assays, the open reading frame encoding the target antigen is fragmented either randomly or by specific genetic constructions and the reactivity of the expressed fragments of the antigen with the antibody to be tested is determined. The gene fragments may, for example, be produced by PCR and then transcribed and translated into protein in vitro, in the presence of radioactive amino acids. The binding of the antibody to the radioactively labeled antigen fragments is then determined by immunoprecipitation and gel electrophoresis. Certain epitopes can also be identified by using large libraries of random peptide sequences displayed on the surface of phage particles (phage libraries). Alternatively, a defined library of overlapping peptide fragments can be tested for binding to the test antibody in simple binding assays. In an additional example, mutagenesis of an antigen binding domain, domain swapping experiments and alanine scanning mutagenesis can be performed to identify residues required, sufficient, and/or necessary for epitope binding. Alternatively, competition assays can be performed using other antibodies known to bind to the same antigen to determine whether an antibody binds to the same epitope as the other antibodies. Competition assays are well known to those of skill in the art.

In some examples, an antibody described herein is prepared by recombinant technology as exemplified below. Nucleic acids encoding the heavy and light chain of an antibody as described herein can be cloned into one expression vector, each nucleotide sequence being in operable linkage to a suitable promoter. In one example, each of the nucleotide sequences encoding the heavy chain and light chain is in operable linkage to a distinct promoter. Alternatively, the nucleotide sequences encoding the heavy chain and the light chain can be in operable linkage with a single promoter, such that both heavy and light chains are expressed from the same promoter. When necessary, an internal ribosomal entry site (IRES) can be inserted between the heavy chain and light chain encoding sequences.

In some examples, the nucleotide sequences encoding the two chains of the antibody are cloned into two vectors, which can be introduced into the same or different cells. When the two chains are expressed in different cells, each of them can be isolated from the host cells expressing such and the isolated heavy chains and light chains can be mixed and incubated under suitable conditions allowing for the formation of the antibody.

Generally, a nucleic acid sequence encoding one or all chains of an antibody can be cloned into a suitable expression vector in operable linkage with a suitable promoter using methods known in the art. For example, the nucleotide sequence and vector can be contacted, under suitable conditions, with a restriction enzyme to create complementary ends on each molecule that can pair with each other and be joined together with a ligase. Alternatively, synthetic nucleic acid linkers can be ligated to the termini of a gene. These synthetic linkers contain nucleic acid sequences that correspond to a particular restriction site in the vector. The selection of expression vectors/promoter would depend on the type of host cells for use in producing the antibodies.

A variety of promoters can be used for expression of the antibodies described herein, including, but not limited to, cytomegalovirus (CMV) intermediate early promoter, a viral LTR such as the Rous sarcoma virus LTR, HIV-LTR, HTLV-1 LTR, the simian virus 40 (SV40) early promoter, E. coli lac UV promoter, and the herpes simplex tk virus promoter.

Regulatable promoters can also be used. Such regulatable promoters include those using the lac repressor from E. coli as a transcription modulator to regulate transcription from lac operator bearing mammalian cell promoters (Brown, M. et al., Cell, 49:603-612 (1987)), those using the tetracycline repressor (tetR) (Gossen, M., and Bujard, H., Proc. Natl. Acad. Sci. USA 89:5547-555115 (1992); Yao, F. et al., Human Gene Therapy, 9:1939-1950 (1998); Shockelt, P., et al., Proc. Natl. Acad. Sci. USA, 92:6522-6526 (1995)). Other systems include FK506 dimer, VP16 or p65 using astradiol, RU486, diphenol murislerone, or rapamycin. Inducible systems are available from Invitrogen, Clontech and Ariad, among others.

Regulatable promoters that include a repressor with the operon can be used. In one embodiment, the lac repressor from E. coli can function as a transcriptional modulator to regulate transcription from lac operator-bearing mammalian cell promoters [[M. Brown et al., Cell, 49:603-612 (1987)]]; Gossen and Bujard (1992); [[M. Gossen et al., Natl. Acad. Sci. USA, 89:5547-5551(1992)]] combined the tetracycline repressor (tetR) with the transcription activator (VP 16) to create a tetR-mammalian cell transcription activator fusion protein, tTa (tetR-VP 16), with the tetO bearing minimal promoter derived from the human cytomegalovirus (hCMV) promoter to create a tetR-tet operator system to control gene expression in mammalian cells. In one embodiment, a tetracycline inducible switch is used. The tetracycline repressor (tetR) alone, rather than the tetR-mammalian cell transcription factor fusion derivatives can function as potent trans-modulator to regulate gene expression in mammalian cells when the tetracycline operator is properly positioned downstream for the TATA element of the CMVIE promoter (Yao et al., Human Gene Therapy). One particular advantage of this tetracycline inducible switch is that it does not require the use of a tetracycline repressor-mammalian cells transactivator or repressor fusion protein, which in some instances can be toxic to cells (Gossen 5 et al., Natl. Acad. Sci. USA, 89:5547-5551 (1992); Shockett et al., Proc. Natl. Acad. Sci. USA, 92:6522-6526 (1995)), to achieve its regulatable effects.

Additionally, the vector can contain, for example, some or all of the following: a selectable marker gene, such as the neomycin gene for selection of stable or transient transfectants in mammalian cells; enhancer/promoter sequences from the immediate early gene of human CMV for high levels of transcription; transcription termination and RNA processing signals from SV40 for mRNA stability; SV40 polyoma origins of replication and ColE1 for proper episomal replication; internal ribosome binding sites (IRESes), versatile multiple cloning sites; and T7 and SP6 RNA promoters for in vitro transcription of sense and antisense RNA. Suitable vectors and methods for producing vectors containing transgenes are well known and available in the art. Examples of polyadenylation signals useful to practice the methods described herein include, but are not limited to, human collagen I polyadenylation signal, human collagen II polyadenylation signal, and SV40 polyadenylation signal.

One or more vectors (e.g., expression vectors) comprising nucleic acids encoding any of the antibodies (e.g., the nucleic acid coding sequence listed in Table 3) may be introduced into suitable host cells for producing the antibodies. Non-limiting examples of the host cells include Chinese hamster ovary (CHO) cells, dhfr− CHO cell, human embryonic kidney (HEK)-293 cells, verda reno (VERO) cells, nonsecreting null (NSO) cells, human embryonic retinal (PER.C6) cells, Sp2/0 cells, baby hamster kidney (BHK) cells, Madin-Darby Canine Kidney (MDCK) cells, Madin-Darby Bovine Kidney (MDBK) cells, and monkey kidney CV1 line transformed by SV40 (COS) cells. In some embodiments, the host cell expressing the antibodies described herein are CHO cells. The host cells can be cultured under suitable conditions for expression of the antibody or any polypeptide chain thereof. Such antibodies or polypeptide chains thereof can be recovered by the cultured cells (e.g., from the cells or the culture supernatant) via a conventional method, e.g., affinity purification. If necessary, polypeptide chains of the antibody can be incubated under suitable conditions for a suitable period of time allowing for production of the antibody. In some embodiments, the host cell comprises the nucleic acid encoding the heavy chain of the antibody described herein. In some embodiments, the host cell comprises the nucleic acid encoding the light chain of the antibody described herein. In some embodiments, the host cell comprises the nucleic acid encoding the heavy chain and the nucleic acid encoding the light chain.

In some embodiments, methods for preparing an antibody described herein involve a recombinant expression vector that encodes both the heavy chain and the light chain of an antibody described herein, as also described herein. The recombinant expression vector can be introduced into a suitable host cell (e.g., a dhfr− CHO cell) by a conventional method, e.g., calcium phosphate mediated transfection. Positive transformant host cells can be selected and cultured under suitable conditions allowing for the expression of the two polypeptide chains that form the antibody, which can be recovered from the cells or from the culture medium. When necessary, the two chains recovered from the host cells can be incubated under suitable conditions allowing for the formation of the antibody.

In one example, two recombinant expression vectors are provided, one encoding the heavy chain of the antibody and the other encoding the light chain of the antibody. Both of the two recombinant expression vectors can be introduced into a suitable host cell (e.g., dhfr− CHO cell) by a conventional method, e.g., calcium phosphate-mediated transfection.

Alternatively, each of the expression vectors can be introduced into a suitable host cells. Positive transformants can be selected and cultured under suitable conditions allowing for the expression of the polypeptide chains of the antibody. When the two expression vectors are introduced into the same host cells, the antibody produced therein can be recovered from the host cells or from the culture medium. If necessary, the polypeptide chains can be recovered from the host cells or from the culture medium and then incubated under suitable conditions allowing for formation of the antibody. When the two expression vectors are introduced into different host cells, each of them can be recovered from the corresponding host cells or from the corresponding culture media. The two polypeptide chains can then be incubated under suitable conditions for formation of the antibody.

Standard molecular biology techniques are used to prepare the recombinant expression vector, transfect the host cells, select for transformants, culture the host cells and recovery of the antibodies from the culture medium. For example, some antibodies can be isolated by affinity chromatography with a Protein A or Protein G coupled matrix.

Any of the nucleic acids encoding the heavy chain, the light chain, or both of an antibody as described herein (e.g., as provided in Table 3), vectors (e.g., expression vectors) containing such; and host cells comprising the vectors are within the scope of the present disclosure.

TABLE 3
Nucleic acids Sequences encoding the VH/VL of anti-trypsin 1/trypsin 2
antibodies listed in Table 1a

| | | | SEQ ID |
|---|---|---|---|
| Antibody | | Nucleic Acid Sequences | NO |
| Trypsin 1/Trypsin | VH | GAGGTGCAGCTGCTGGAAAGCGGAGGAGGCCTGGTGCAACC | 105 |
| 2-Dual-L1-Ab1 | | CGGCGGTTCGCTGAGGCTGAGCTGTGCCGCCTCTGGTTTCA | |
| | | CCTTTTCAAGCTACGCGATGAGCTGGGTCAGACAGGCCCCG | |
| | | GGAAAGGGATTGGAATGGGTGTCCGCTATCTCCGCTTCCGG | |
| | | AGGGTCAACCTACTACGCCGACTCCGTGAAGGGACGGTTCA | |
| | | CCATCTCGCGCGACAACTCCAAGAACACCCTTTACCTCCAA | |
| | | ATGAACTCCCTGAGGGCCGAGGACACTGCAGTCTACTACTG | |
| | | CGCGAGAGGCTCACCTATTGGCTACTCCTATGCCCGCGTGT | |
| | | CGTACTACTACTACATGGATGTCTGGGGAAAGGGGACTACC | |
| | | GTGACTGTCTCCTCG | |
| | VL | GACATCCAAATGACTCAGTCCCCGTCCTCCGTGTCTGCCTC | 106 |
| | | CGTGGGAGATAGAGTGACCATCACTTGTCGGGCCTCCCAAG | |
| | | GCATTTCACGCTGGCTGGCCTGGTACCAGCAGAAACCAGGG | |
| | | AAGGCACCCAAGCTCCTGATCTACGCCGCGTCGTCACTGCA | |
| | | ATCCGGAGTCCCTTCGCGGTTCTCCGGTTCGGGATCCGGAA | |
| | | CCGATTTCACCCTTACCATTAGCAGCCTGCAGCCTGAGGAC | |
| | | TTCGCTACCTACTACTGCCAGCAGGGGAACAGCTTTCCGCT | |
| | | CACCTTCGGCGGAGGCACTAAGGTCGAAATCAAG | |
| Trypsin 1/Trypsin | VH | GAGGTGCAGCTGCTGGAATCCGGGGGCGGACTGGTGCAGCC | 107 |
| 2-Dual-L1-Ab2 | | CGGCGGCTCCCTGAGGCTTTCATGTGCCGCCTCCGGGTTTA | |
| | | CCTTCGGCGACTACGCCATGTGGTGGGTCAGACAGGCGCCG | |
| | | GGAAAGGGACTGGAATGGGTGTCCGCCATTTCCGCCTCGGG | |
| | | CGACGTGACCTACTACGCGGATTCCGTGAAGGGTCGCTTCA | |
| | | CTATCTCTCGGGACAACAGCAAAAACACCCTCTACTTGCAA | |
| | | ATGAACTCACTGCGCGCTGAGGACACCGCAGTGTATTACTG | |
| | | CGCCCGGGGAAGCCTCATCGGATACAGCTATGCCCGGGTGT | |
| | | CGTACTACTACTACTCCGATGTCTGGGGAAAGGGTACCACT | |
| | | GTCACTGTGTCGAGC | |
| | VL | GACATCCAAATGACCCAGTCGCCCTCATCCGTGAGCGCCTC | 108 |
| | | TGTGGGCGACAGAGTGACCATCACGTGTCGGGCCAGCCAGG | |
| | | GCATTTCGCGGTGGCTCGCATGGTATCAGCAGAAGCCTGGA | |
| | | AAAGCCCCAAAGCTGCTGATCTACGCCGCTAGCAGCCTTCA | |
| | | GTCCGGAGTGCCGTCCCGCTTCTCCGGGTCGGGATCCGGGA | |
| | | CTGATTTCACCCTGACCATTTCCTCATTGCAACCCGAGGAC | |
| | | TTCGCGACCTACTACTGCCAACAGGGCTGGTCCTTCCCGCT | |
| | | GACTTTTGGTGGCGGAACTAAGGTCGAAATCAAG | |
| Trypsin 1/Trypsin | VH | CAAGTGCAGCTGCAGGAATCAGGTCCTGGGCTGGTCAAGCC | 109 |
| 2-Dual-L2-Ab1 | | CAGCGAGACTCTGTCTCTCACTTGCACGGTGTCCGGTGGCA | |
| | | GCATCTCGTCCTACTATTGGTCGTGGATTAGGCAACCAGCC | |
| | | GGAAAGGGACTGGAATGGATCGGCCGGATCTACACTTCGGG | |
| | | GAGCACCAACTACAACCCGAGCCTCAAGTCACGCGTCACCA | |
| | | TGTCCGTGGACACCTCCAAGAACCAGTTCTCCCTGAAACTT | |
| | | TCGTCCGTGACTGCCGCTGATACCGCGGTGTACTACTGCGC | |
| | | ACGGGACCTGACCAACTACGGCTCCGGAAAGTCCATTAGAT | |
| | | TCTACGGAATGGACGTCTGGGGCCAGGGAACCACTGTGACC | |
| | | GTGTCCAGC | |
| | VL | GATATTCAAATGACCCAGTCACCGTCCTCCCTGTCCGCAAG | 110 |
| | | CGTCGGTGACCGCGTGACTATTACGTGTCAAGCCAGCCAGG | |
| | | ACATCTCCAACTACCTGAACTGGTATCAGCAGAAGCCTGGA | |
| | | AAGGCGCCAAAGCTGCTCATCTACGATGCCTCTAATCTGGA | |
| | | AACCGGAGTGCCCTCGAGATTCAGCGGATCAGGATCCGGGA | |
| | | CCGACTTTACCTTCACCATCTCGTCCCTGCAACCGGAAGAT | |
| | | ATTGCTACTTACTACTGCCAGCAGGACGACAACTTCATCAC | |
| | | TTTCGGCGGCGGCACCAAAGTGGAGATCAAG | |
| Trypsin 1/Trypsin | VH | CAAGTGCAGCTTCAGGAGTCCGGCCCAGGACTGGTCAAGCC | 111 |
| 2-Dual-L2-Ab2 | | GAGCGAAACTTTGTCCCTGACTTGCACGGTGTCCGGCGGAT | |
| | | CGATCAGCAGCAATTACTGGCATTGGATTCGGCAGCCGGCC | |
| | | GGGAAGGGACTCGAATGGATCGGCAGGATTCACACCTCCGG | |
| | | CTCCACCTTTTACAACCCTTCACTGAAATCGCGCGTGACCA | |
| | | TGTCCGTGGATACCTCCAAGAACCAGTTCAGCCTGAAGCTC | |
| | | TCCTCGGTGACCGCTGCCGACACTGCGGTGTACTATTGCGC | |
| | | AAGAGACATCACCAACTACGGTTCGGGAAAGTCAATCCGGT | |
| | | TCTACCCCATGGACGTCTGGGGTCAAGGGACTACAGTGACC | |
| | | GTGTCCTCT | |
| | VL | GATATCCAAATGACCCAGTCGCCCTCATCCCTGTCCGCCTC | 112 |
| | | CGTGGGTGATCGCGTGACCATTACTTGCCAAGCCAGCCAGG | |
| | | ACATTTCGAACTACCTGAACTGGTATCAGCAGAAGCCTGGC | |
| | | AAAGCCCCGAAGCTTCTGATCTACGACGCTTCAAACCTGGA | |
| | | AACCGGAGTGCCATCCCGGTTCTCCGGATCCGGGTCGGGAA | |
| | | CCGACTTCACCTTCACAATCAGCAGCCTCCAGCCGGAAGAT | |
| | | ATCGCGACTTACTACTGCCAACAAGACGACACGTTTATCAC | |
| | | CTTCGGGGGCGGCACTAAGGTCGAGATTAAG | |
| Trypsin 1/Trypsin | VH | CAAGTGCAGCTGCAAGAGAGCGGTCCGGGGCTGGTCAAGCC | 113 |
| 2-Dual-L2-Ab3 | | CTCGGAAACTCTCTCACTGACCTGTACCGTGTCCGGCGGCT | |
| | | CGATTTCCTCCCACTATTGGCATTGGATCAGGCAGCCAGCC | |
| | | GGGAAGGGCTTGGAATGGATCGGACGGATTGCTACCAGCGG | |
| | | TTCAACGTACTACAACCCTAGCCTGAAGTCCAGAGTGACAA | |
| | | TGTCCGTGGACACCTCCAAGAACCAGTTCTCTCTGAAACTC | |
| | | TCCTCCGTGACCGCAGCCGATACTGCGGTGTACTACTGCGC | |
| | | CCGGGACCTTACCAACTACGGCATCACTAAGAGCATCCGCT | |
| | | TCTACGGAATGGACGTCTGGGGACAGGGAACCACCGTGACT | |
| | | GTGTCGTCC | |
| | VL | GACATCCAAATGACTCAGTCCCCATCAAGCCTGAGCGCCTC | 114 |
| | | CGTGGGGGATCGCGTGACCATTACGTGTCAGGCGTCCCAGG | |
| | | ACATTAGCAACTACCTGAACTGGTACCAGCAGAAGCCTGGG | |
| | | AAGGCCCCGAAGCTCCTTATCTACGATGCCTCGAATCTGGA | |
| | | AACTGGAGTGCCGTCACGGTTCTCGGGATCCGGCTCCGGCA | |
| | | CCGACTTCACCTTTACCATTTCGTCCCTGCAACCCGAGGAC | |
| | | ATCGCTACCTACTATTGCCAACAAGTGGACAACTTCATCAC | |
| | | CTTCGGTGGCGGAACTAAGGTCGAAATCAAA | |
| Trypsin 1/Trypsin | VH | CAAGTGCAACTGCAGGAATCCGGACCGGGGCTTGTGAAGCC | 115 |
| 2-Dual-L2-Ab4 | | CTCCGAGACTCTGTCACTGACTTGCACTGTCTCGGGTGGCA | |
| | | GCATCTCCTCCCACTACTGGCATTGGATCAGACAGCCAGCC | |
| | | GGAAAGGGACTGGAATGGATTGGCAGGATCCACACCTCGGG | |
| | | TTCCACCTTCTACAACCCTAGCCTCAAGTCCCGCGTGACGA | |
| | | TGAGCGTGGACACCTCTAAAAACCAGTTCAGCCTCAAGTTG | |
| | | TCCTCCGTGACCGCTGCGGATACTGCCGTGTACTATTGTGC | |
| | | CCGGGACCTGACCAACTACGGCTGGTCCAAGTCGATTCGGT | |
| | | TTTACGGAATGGACGTCTGGGGCCAGGGGACAACCGTGACC | |
| | | GTGTCATCG | |
| | VL | GACATTCAAATGACCCAGAGCCCATCCTCACTGTCGGCGTC | 116 |
| | | CGTGGGGGATCGGGTCACTATCACCTGTCAAGCCAGCCAGG | |
| | | ACATCAGCAACTACCTCAACTGGTACCAGCAGAAGCCCGGG | |
| | | AAGGCTCCTAAGCTGCTTATCTATGACGCCTCCAATCTGGA | |
| | | AACCGGAGTGCCGTCACGCTTCTCGGGATCCGGCTCCGGTA | |
| | | CTGACTTTACCTTCACCATCTCGTCCCTGCAACCGGAAGAT | |
| | | ATTGCCACCTACTACTGCCAGCAGACCGACAACTTCATTAC | |
| | | TTTCGGCGGCGGAACGAAAGTGGAGATCAAG | |
| Trypsin 1/Trypsin | VH | CAAGTGCAGCTGGTGCAGTCCGGTGCCGAAGTCAAGAAGCC | 117 |
| 2-Dual-L3-Ab1 | | AGGAGCCAGCGTGAAAGTGTCCTGCAAGGCCTCCGGCTATA | |
| | | CCTTCACTGGGTACTACATGCACTGGGTCCGCCAAGCACCT | |
| | | GGCCAGGGACTCGAATGGATGGGCTCCATTAACCCGTCGAG | |
| | | CGGAGGAACCAACTACGCGCAGAAGTTCCAGGGCAGAGTGA | |
| | | CCATGACTAGGGACACCTCGATCTCCACGGCCTACATGGAG | |
| | | CTGTCACGGCTGCGGTCGGATGACACTGCCGTGTACTACTG | |
| | | CGCGAGAGTGGGAGGAGCTGCTGCCGCTGGAAAGGCATACA | |
| | | CCGCCTCCGCGTTCGATATCTGGGGACAGGGGACCATGGTC | |
| | | ACTGTGTCCAGC | |
| | VL | GAAATCGTGATGACCCAATCCCCGGCCACTCTGTCGGTGTC | 118 |
| | | ACCAGGAGAGCGGGCCACCTTGAGCTGCCGCGCTAGCCAGT | |
| | | CCGTGGGCTCCAACCTCGCGTGGTACCAGCAGAAGCCCGGA | |
| | | CAGGCACCTCGCCTCCTGATCTACGGTGCCTCCACTCGGGC | |
| | | TACCGGAATCCCTGCGAGATTCTCAGGGTCCGGTTCCGGCA | |
| | | CCGAATTCACCCTGACGATCTCGAGCCTGCAGTCCGAGGAC | |
| | | TTCGCCGTGTACTACTGTCAACAGTTCCACAACTGGCCCCC | |
| | | GCTGACTTTTGGAGGAGGGACCAAGGTCGAGATTAAG | |
| Trypsin 1/Trypsin | VH | CAAGTGCAGCTCGTGCAGTCCGGGGCAGAAGTCAAGAAGCC | 119 |
| 2-Dual-L3-Ab2 | | GGGTGCCAGCGTGAAAGTGTCCTGCAAAGCCAGCGGTTACA | |
| | | CTTTCACCGACCACTACATGCATTGGGTCCGCCAGGCGCCC | |
| | | GGACAGGGACTGGAGTGGATGGGCGCTATTAACCCTTCATC | |
| | | CGGCGGAACGGCCTATGCCCAAAAGTTCCAGGGGAGAGTGA | |
| | | CCATGACCCGGGACACCTCAATCTCCACCGCCTACATGGAA | |
| | | CTGTCGAGGCTGCGGAGCGACGATACTGCGGTGTACTACTG | |
| | | TGCACGCGTGGGCGGCGCTGCCCGGGTCGGAAAGGCCTACA | |
| | | CCGCCTCGGCGTTTGATATCTGGGGACAGGGAACTATGGTC | |
| | | ACCGTGTCCTCC | |
| | VL | GAAATCGTGATGACCCAATCCCCGGCCACTCTGTCGGTGTC | 120 |
| | | ACCAGGAGAGCGGGCCACCTTGAGCTGCCGCGCTAGCCAGT | |
| | | CCGTGGGCTCCAACCTCGCGTGGTACCAGCAGAAGCCCGGA | |
| | | CAGGCACCTCGCCTCCTGATCTACGGTGCCTCCACTCGGGC | |
| | | TACCGGAATCCCTGCGAGATTCTCAGGGTCCGGTTCCGGCA | |
| | | CCGAATTCACCCTGACGATCTCGAGCCTGCAGTCCGAGGAC | |
| | | TTCGCCGTGTACTACTGTCAACAGTTCCACAACTGGCCCCC | |
| | | GCTGACTTTTGGAGGAGGGACCAAGGTCGAGATTAAG | |
| Trypsin 1/Trypsin | VH | CAAGTGCAGCTGGTGCAATCCGGCGCCGAGGTCAAGAAGCC | 121 |
| 2-Dual-L4-Ab1 | | TGGAGCCTCCGTGAAAGTGTCCTGCAAGGCCTCGGGTTACA | |
| | | CTTTCACCTCATACGGGATTTCATGGGTCAGACAGGCACCG | |
| | | GGACAGGGACTCGAATGGATGGGCTGGATCAGCGCCTACTC | |
| | | GGGCGACACCAACTACGCCCAGAAGCTGCAGGGTCGGGTCA | |
| | | CAATGACCACCGACACCAGCACGTCCACCGCGTACATGGAA | |
| | | CTTAGGTCCCTGCGGTCCGATGACACTGCTGTGTACTACTG | |
| | | TGCGCGCGGAGATGAGGTGGTCGGAAAGATGACCTACTATT | |
| | | ACGGGATGGACGTCTGGGGCCAGGGAACTACCGTGACTGTG | |
| | | TCGAGC | |
| | VL | GACATTCAAATGACTCAGTCCCCGTCCTCCGTGTCGGCTTC | 122 |
| | | CGTGGGCGACAGGGTCACGATCACCTGTCGCGCCTCCCAAG | |
| | | GGATCAGCAGATGGCTGGCCTGGTACCAGCAGAAGCCAGGG | |
| | | AAAGCCCCCAAGCTGCTTATCTATGCCGCGAGCTCACTGCA | |
| | | GTCCGGAGTGCCTAGCCGGTTCTCCGGCTCCGGATCAGGAA | |
| | | CCGACTTTACCCTCACCATCTCGAGCCTGCAGCCCGAAGAT | |
| | | TTCGCCACTTACTACTGCCAACAAGCAAACTCGTTCCCGAT | |
| | | CACTTTCGGTGGAGGCACCAAGGTCGAGATTAAG | |
| Trypsin 1/Trypsin | VH | CAAGTGCAACTGGTGCAGTCCGGTGCCGAGGTCAAGAAGCC | 123 |
| 2-Dual-L4-Ab2 | | CGGAGCGTCCGTGAAAGTGTCCTGCAAGGCCTCGGGATACA | |
| | | CTTTCACCGACTACGCGATTAGCTGGGTCAGACAGGCCCCG | |
| | | GGACAGGGCCTGGAGTGGATGGGCTGGATCTCCGCATCAAC | |
| | | CGCCGACACTAACTACGCCCAGAAGCTCCAGGGTCGCGTGA | |
| | | CTATGACCACCGACACAAGCACTTCCACCGCCTACATGGAA | |
| | | CTGCGGTCCCTTCGGAGCGACGATACCGCTGTGTACTATTG | |
| | | TGCCCGCGGCGACGAAGTGGTCGGAAAGATGACCTACTACT | |
| | | ACGGGATGGATGTCTGGGGCCAGGGGACCACCGTGACGGTG | |
| | | TCATCG | |
| | VL | GACATTCAAATGACTCAGTCCCCGTCCTCCGTGTCGGCTTC | 124 |
| | | CGTGGGCGACAGGGTCACGATCACCTGTCGCGCCTCCCAAG | |
| | | GGATCAGCAGATGGCTGGCCTGGTACCAGCAGAAGCCAGGG | |
| | | AAAGCCCCCAAGCTGCTTATCTATGCCGCGAGCTCACTGCA | |
| | | GTCCGGAGTGCCTAGCCGGTTCTCCGGCTCCGGATCAGGAA | |
| | | CCGACTTTACCCTCACCATCTCGAGCCTGCAGCCCGAAGAT | |
| | | TTCGCCACTTACTACTGCCAACAAGCAAACTCGTTCCCGAT | |
| | | CACTTTCGGTGGAGGCACCAAGGTCGAGATTAAG | |
| Trypsin 1/Trypsin | VH | CAAGTGCAGCTCGTGCAGTCGGGCGCTGAAGTGAAAAAGCC | 125 |
| 2-Dual-L4-Ab3 | | CGGAGCCTCCGTGAAAGTGTCCTGCAAGGCCTCCGGGTACA | |
| | | CTTTCACCGACTACGCAATCAGCTGGGTCAGACAGGCCCCG | |
| | | GGCCAGGGTCTTGAGTGGATGGGTTGGATCTCCGCCTCAAC | |
| | | CGCCGACACCAACTACGCGCAAAAGCTGCAGGGAAGGGTCA | |
| | | CCATGACCACCGACACGTCCACATCAACTGCGTACATGGAG | |
| | | CTGCGCTCGCTGCGGAGCGATGACACCGCCGTGTACTATTG | |
| | | TGCCCGGGGCGATGAAGTGGTCGGAAAGCGCACTTACTACA | |
| | | TTGGCATGGACGTCTGGGGACAGGGGACTACCGTGACCGTC | |
| | | AGCTCC | |
| | VL | GACATTCAAATGACTCAGTCCCCGTCCTCCGTGTCGGCTTC | 126 |
| | | CGTGGGCGACAGGGTCACGATCACCTGTCGCGCCTCCCAAG | |
| | | GGATCAGCAGATGGCTGGCCTGGTACCAGCAGAAGCCAGGG | |
| | | AAAGCCCCCAAGCTGCTTATCTATGCCGCGAGCTCACTGCA | |
| | | GTCCGGAGTGCCTAGCCGGTTCTCCGGCTCCGGATCAGGAA | |
| | | CCGACTTTACCCTCACCATCTCGAGCCTGCAGCCCGAAGAT | |
| | | TTCGCCACTTACTACTGCCAACAAGCAAACTCGTTCCCGAT | |
| | | CACTTTCGGTGGAGGCACCAAGGTCGAGATTAAG | |
| Trypsin 1/Trypsin | VH | CAAGTGCAGCTCGTGCAGTCCGGCGCCGAAGTCAAGAAGCC | 127 |
| 2-Dual-L4-Ab4 | | CGGTGCATCAGTGAAAGTCAGCTGCAAGGCCTCAGGGTACA | |
| | | CATTCACGGACTACGCGATTAGCTGGGTCAGACAGGCTCCG | |
| | | GGCCAAGGTCTTGAGTGGATGGGCTGGATCTCGGCCTCCAC | |
| | | CGCCGATACCAACTACGCCCAGAAGCTGCAGGGCCGCGTGA | |
| | | CCATGACTACCGATACCTCCACCTCGACTGCCTACATGGAG | |
| | | CTGCGGTCCCTGCGGAGCGACGACACTGCCGTGTATTACTG | |
| | | TGCGAGGGGAACCGAAGTCGTGGGAAAGATGACCGTGTACC | |
| | | TGGGGATGGACGTCTGGGGACAGGGAACCACTGTGACTGTG | |
| | | TCCTCC | |
| | VL | GACATTCAAATGACTCAGTCCCCGTCCTCCGTGTCGGCTTC | 128 |
| | | CGTGGGCGACAGGGTCACGATCACCTGTCGCGCCTCCCAAG | |
| | | GGATCAGCAGATGGCTGGCCTGGTACCAGCAGAAGCCAGGG | |
| | | AAAGCCCCCAAGCTGCTTATCTATGCCGCGAGCTCACTGCA | |
| | | GTCCGGAGTGCCTAGCCGGTTCTCCGGCTCCGGATCAGGAA | |
| | | CCGACTTTACCCTCACCATCTCGAGCCTGCAGCCCGAAGAT | |
| | | TTCGCCACTTACTACTGCCAACAAGCAAACTCGTTCCCGAT | |
| | | CACTTTCGGTGGAGGCACCAAGGTCGAGATTAAG | |
| Trypsin 1/Trypsin | VH | CAAGTGCAACTCGTGCAGTCCGGAGCAGAAGTCAAGAAGCC | 129 |
| 2-Dual-L4-Ab5 | | CGGGGCTTCCGTGAAAGTGTCCTGCAAGGCCTCCGGCTACA | |
| | | CCTTCACTGACTACGCGATTTCCTGGGTCCGCCAGGCCCCG | |
| | | GGCCAGGGTCTGGAGTGGATGGGCTGGATCAGCGCCTCGAC | |
| | | CGCCGACACTAACTATGCCCAAAAGCTGCAGGGTCGCGTGA | |
| | | CAATGACCACTGACACCTCCACGAGCACCGCCTACATGGAG | |
| | | CTTAGGTCACTGCGGAGCGATGACACCGCCGTGTACTACTG | |
| | | TGCGAGACAGGATGAAGTCGTGGGGAAGATGACCTACTACA | |
| | | TCGGAACCGACGTCTGGGGACAGGGAACTACTGTGACCGTG | |
| | | TCGTCA | |
| | VL | GACATTCAAATGACCCAGAGCCCCTCGTCCGTGTCTGCCTC | 130 |
| | | GGTCGGCGACCGCGTGACTATCACTTGCCGGGCTAGCCAGG | |
| | | GAATCTCCAGATGGCTTGCCTGGTATCAGCAGAAGCCTGGG | |
| | | AAGGCCCCAAAACTGTTGATCTACGCCGCCTCATCCCTCCA | |
| | | ATCCGGAGTGCCGTCAAGGTTCTCCGGAAGCGGAAGCGGCA | |
| | | CTGATTTTACGCTGACCATTTCCTCCCTGCAACCCGAGGAC | |
| | | TTCGCGACCTACTACTGTCAGCAAGCAAACTCGTTCCCGCT | |
| | | GACCTTCGGTGGCGGGACCAAGGTCGAAATCAAG | |
| Trypsin 1/Trypsin | VH | CAAGTGCAACTGGTGCAGTCGGGTGCCGAAGTCAAGAAGCC | 131 |
| 2-Dual-L4-Ab6 | | CGGGGCGTCCGTGAAAGTGTCCTGCAAGGCCTCCGGCTACA | |
| | | CCTTCACCGATTACGCCATTAGCTGGGTCCGCCAGGCCCCG | |
| | | GGACAGGGCCTGGAGTGGATGGGATGGATCTCGGCTGGAAC | |
| | | GGCCGACACTAACTACGCCCAGAAGCTCCAGGGCAGAGTGA | |
| | | CCATGACCACCGACACTTCCACTTCAACCGCCTACATGGAA | |
| | | CTTCGGTCCCTGAGGTCCGACGATACAGCGGTGTACTACTG | |
| | | TGCACGGGGCGACGAGGTCGTGGGGAAGATGACCTACTACT | |
| | | ATGATATGGACGTCTGGGGACAGGGTACCACCGTGACTGTG | |
| | | TCCAGC | |
| | VL | GACATTCAAATGATCCAGTCGCCTAGCTCCGTGTCCGCCTC | 132 |
| | | CGTGGGGGACCGCGTGACTATTACCTGTCGGGCCTCACAAG | |
| | | GCATCTCCAGATGGCTTGCCTGGTACCAGCAGAAGCCCGGA | |
| | | AAGGCCCCGAAACTCCTGATCTATGCGGCTAGCAGCCTGCA | |
| | | GTCCGGAGTGCCCTCCCGGTTCTCCGGCTCGGGATCCGGGA | |
| | | CCGATTTCACCCTGACTATTTCGAGCCTGCAGCCAGAGGAC | |
| | | TTCGCAACCTACTACTGCCAACAGGCCTCATCTTTCCCGAT | |
| | | CACCTTTGGTGGCGGAACTAAGGTCGAAATCAAG | |
| Trypsin 1/Trypsin | VH | CAAGTGCAACTGGTGCAGTCCGGCGCAGAGGTCAAGAAGCC | 133 |
| 2-Dual-L4-Ab7 | | CGGAGCCTCCGTGAAAGTGTCGTGCAAAGCCAGCGGATACA | |
| | | CTTTCGACGACTACGGGATCTCATGGGTCAGACAGGCGCCG | |
| | | GGACAGGGCCTGGAGTGGATGGGTTGGATTAGCGCCACCGT | |
| | | GGCTGACACTAACTACGCCCAGAAGCTCCAGGGCCGCGTGA | |
| | | CCATGACGACCGATACCAGCACTTCCACCGCCTACATGGAA | |
| | | CTTAGGTCCCTGCGGTCGGATGATACAGCCGTGTACTACTG | |
| | | TGCGCGGGGCGACGAACCTGTCGGGAAGATGACCTACTACT | |
| | | ATGGAATGGACGTCTGGGGACAGGGTACCACCGTGACTGTG | |
| | | TCCTCC | |
| | VL | GATATCCAAATGACCCAGTCCCCAAGCTCCGTGTCGGCCTC | 134 |
| | | CGTGGGAGACAGAGTGACCATCACTTGTCGGGCCAGCCAGG | |
| | | GCATTTCCCGCTGGTTGGCTTGGTATCAGCAGAAGCCCGGG | |
| | | AAGGCCCCGAAGCTGCTCATCTACGCGGCCAGCTCACTGCA | |
| | | GTCGGGAGTCCCGTCAAGGTTCTCTGGATCCGGATCGGGCA | |
| | | CTGACTTCACCCTGACGATTTCCTCCCTGCAACCCGAGGAC | |
| | | TTTGCAACTTACTACTGCCAACAGGCCAACAGCCTTCCTAT | |
| | | CACCTTCGGTGGCGGGACCAAAGTGGAAATCAAG | |
| Trypsin 1/Trypsin | VH | CAAGTGCAGCTGGTGCAGTCAGGGGCAGAAGTCAAGAAGCC | 135 |
| 2-Dual-L5-Ab1 | | TGGAGCCAGCGTGAAAGTGTCCTGCAAGGCCTCGGGTTATA | |
| | | CTTTCACCGGGTACTACATGCATTGGGTCAGACAGGCCCCA | |
| | | GGACAGGGACTGGAGTGGATGGGCTCGATCAACCCGAGCTC | |
| | | AGGAGGCACCAACTACGCGCAGAAGTTTCAGGGCCGCGTCA | |
| | | CCATGACGCGCGACACCTCCATCTCCACTGCCTACATGGAG | |
| | | CTCTCCCGGCTGAGAAGCGACGATACTGCGGTGTACTACTG | |
| | | CGCTAGGGACCCCGTGCTGCTGCTTCGGCAACAGCGGATTC | |
| | | TCCACGAATCGGCCTTCGATATCTGGGGACAAGGCACCACT | |
| | | GTGACCGTGTCCTCC | |
| | VL | GAAATCGTGATGACCCAATCCCCGGCCACTCTGTCGGTGTC | 136 |
| | | ACCAGGAGAGCGGGCCACCTTGAGCTGCCGCGCTAGCCAGT | |
| | | CCGTGGGCTCCAACCTCGCGTGGTACCAGCAGAAGCCCGGA | |
| | | CAGGCACCTCGCCTCCTGATCTACGGTGCCTCCACTCGGGC | |
| | | TACCGGAATCCCTGCGAGATTCTCAGGGTCCGGTTCCGGCA | |
| | | CCGAATTCACCCTGACGATCTCGAGCCTGCAGTCCGAGGAC | |
| | | TTCGCCGTGTACTACTGTCAACAGTTCCACAACTGGCCCCC | |
| | | GCTGACTTTTGGAGGAGGGACCAAGGTCGAGATTAAG | |
| Trypsin 1/Trypsin | VH | CAGGTCCAACTCGTGCAGTCGGGAGCCGAAGTCAAGAAGCC | 137 |
| 2-Dual-L5-Ab2 | | CGGAGCATCCGTGAAAGTGTCCTGCAAGGCGTCCGGCTACA | |
| | | CCTTTGAGGGGCACTATATGCACTGGGTCAGACAGGCCCCA | |
| | | GGTCAAGGCCTCGAATGGATGGGGGCCATCAACCCGTACTC | |
| | | AGGACAGACCAACTACGCGCAGAAGTTCCAGGGTCGCGTGA | |
| | | CGATGACCCGGGACACTAGCATCTCGACCGCCTACATGGAA | |
| | | CTGTCCAGATTGCGCTCCGATGACACTGCCGTGTACTACTG | |
| | | TGCTCGGGATTTTGTGCTTCTGCTGAGGCAGAGCCGGATCC | |
| | | TGCATGAGAGCGCCTTCGACATTTGGGGCCAGGGAACTACC | |
| | | GTGACCGTGTCCAGC | |
| | VL | GAAATCGTGATGACCCAATCCCCGGCCACTCTGTCGGTGTC | 138 |
| | | ACCAGGAGAGCGGGCCACCTTGAGCTGCCGCGCTAGCCAGT | |
| | | CCGTGGGCTCCAACCTCGCGTGGTACCAGCAGAAGCCCGGA | |
| | | CAGGCACCTCGCCTCCTGATCTACGGTGCCTCCACTCGGGC | |
| | | TACCGGAATCCCTGCGAGATTCTCAGGGTCCGGTTCCGGCA | |
| | | CCGAATTCACCCTGACGATCTCGAGCCTGCAGTCCGAGGAC | |
| | | TTCGCCGTGTACTACTGTCAACAGTTCCACAACTGGCCCCC | |
| | | GCTGACTTTTGGAGGAGGGACCAAGGTCGAGATTAAG | |

In some embodiments, the present disclosure provides an isolated nucleic acid comprising a sequence at least 60% (e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 105, and/or a sequence at least 60% (e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 106. In some embodiments, the present disclosure provides an expression vector comprising: an isolated nucleic acid comprising a sequence at least 60% (e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 105, and/or a sequence at least 60% (e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 106.

In some embodiments, an anti-trypsin a/trypsin 2 antibody described herein is produced by expressing (i) a nucleic acid at least 60% (e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 105, and/or (ii) a nucleic acid at least 60% (e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 106.

In some embodiments, the present disclosure provides an isolated nucleic acid comprising a sequence at least 60% (e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 107, and/or a sequence at least 60% (e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 108. In some embodiments, the present disclosure provides an expression vector comprising: an isolated nucleic acid comprising a sequence at least 60% (e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 107, and/or a sequence at least 60% (e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 108.

In some embodiments, an anti-trypsin a/trypsin 2 antibody described herein is produced by expressing (i) a nucleic acid at least 60% (e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 107, and/or (ii) a nucleic acid at least 60% (e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 108.

In some embodiments, the present disclosure provides an isolated nucleic acid comprising a sequence at least 60% (e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 109, and/or a sequence at least 60% (e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 110. In some embodiments, the present disclosure provides an expression vector comprising: an isolated nucleic acid comprising a sequence at least 60% (e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 109, and/or a sequence at least 60% (e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 110.

In some embodiments, an anti-trypsin a/trypsin 2 antibody described herein is produced by expressing (i) a nucleic acid at least 60% (e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 109, and/or (ii) a nucleic acid at least 60% (e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 110.

In some embodiments, the present disclosure provides an isolated nucleic acid comprising a sequence at least 60% (e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 111, and/or a sequence at least 60% (e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 112. In some embodiments, the present disclosure provides an expression vector comprising: an isolated nucleic acid comprising a sequence at least 60% (e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 111, and/or a sequence at least 60% (e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 112.

In some embodiments, an anti-trypsin a/trypsin 2 antibody described herein is produced by expressing (i) a nucleic acid at least 60% (e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 111, and/or (ii) a nucleic acid at least 60% (e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 112.

In some embodiments, the present disclosure provides an isolated nucleic acid comprising a sequence at least 60% (e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 113, and/or a sequence at least 60% (e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 114. In some embodiments, the present disclosure provides an expression vector comprising: an isolated nucleic acid comprising a sequence at least 60% (e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 113, and/or a sequence at least 60% (e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 114.

In some embodiments, an anti-trypsin a/trypsin 2 antibody described herein is produced by expressing (i) a nucleic acid at least 60% (e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 113, and/or (ii) a nucleic acid at least 60% (e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 114.

In some embodiments, the present disclosure provides an isolated nucleic acid comprising a sequence at least 60% (e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 115, and/or a sequence at least 60% (e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 116. In some embodiments, the present disclosure provides an expression vector comprising: an isolated nucleic acid comprising a sequence at least 60% (e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 115, and/or a sequence at least 60% (e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 116.

In some embodiments, an anti-trypsin a/trypsin 2 antibody described herein is produced by expressing (i) a nucleic acid at least 60% (e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 115, and/or (ii) a nucleic acid at least 60% (e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 116.

In some embodiments, the present disclosure provides an isolated nucleic acid comprising a sequence at least 60% (e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 117, and/or a sequence at least 60% (e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 118. In some embodiments, the present disclosure provides an expression vector comprising: an isolated nucleic acid comprising a sequence at least 60% (e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 117, and/or a sequence at least 60% (e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 118.

In some embodiments, an anti-trypsin a/trypsin 2 antibody described herein is produced by expressing (i) a nucleic acid at least 60% (e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 117, and/or (ii) a nucleic acid at least 60% (e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 118.

In some embodiments, the present disclosure provides an isolated nucleic acid comprising a sequence at least 60% (e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 119, and/or a sequence at least 60% (e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 120. In some embodiments, the present disclosure provides an expression vector comprising: an isolated nucleic acid comprising a sequence at least 60% (e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 119, and/or a sequence at least 60% (e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 120.

In some embodiments, an anti-trypsin a/trypsin 2 antibody described herein is produced by expressing (i) a nucleic acid at least 60% (e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 119, and/or (ii) a nucleic acid at least 60% (e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 120.

In some embodiments, the present disclosure provides an isolated nucleic acid comprising a sequence at least 60% (e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 121, and/or a sequence at least 60% (e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 122. In some embodiments, the present disclosure provides an expression vector comprising: an isolated nucleic acid comprising a sequence at least 60% (e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 121, and/or a sequence at least 60% (e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 122.

In some embodiments, an anti-trypsin a/trypsin 2 antibody described herein is produced by expressing (i) a nucleic acid at least 60% (e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 121, and/or (ii) a nucleic acid at least 60% (e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 122.

In some embodiments, the present disclosure provides an isolated nucleic acid comprising a sequence at least 60% (e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 123, and/or a sequence at least 60% (e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 124. In some embodiments, the present disclosure provides an expression vector comprising: an isolated nucleic acid comprising a sequence at least 60% (e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 123, and/or a sequence at least 60% (e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 124.

In some embodiments, an anti-trypsin a/trypsin 2 antibody described herein is produced by expressing (i) a nucleic acid at least 60% (e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 123, and/or (ii) a nucleic acid at least 60% (e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 124.

In some embodiments, the present disclosure provides an isolated nucleic acid comprising a sequence at least 60% (e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 125, and/or a sequence at least 60% (e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 126. In some embodiments, the present disclosure provides an expression vector comprising: an isolated nucleic acid comprising a sequence at least 60% (e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 125, and/or a sequence at least 60% (e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 126.

In some embodiments, an anti-trypsin a/trypsin 2 antibody described herein is produced by expressing (i) a nucleic acid at least 60% (e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 125, and/or (ii) a nucleic acid at least 60% (e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 126.

In some embodiments, the present disclosure provides an isolated nucleic acid comprising a sequence at least 60% (e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 127, and/or a sequence at least 60% (e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 128. In some embodiments, the present disclosure provides an expression vector comprising: an isolated nucleic acid comprising a sequence at least 60% (e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 127, and/or a sequence at least 60% (e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 128.

In some embodiments, an anti-trypsin a/trypsin 2 antibody described herein is produced by expressing (i) a nucleic acid at least 60% (e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 127, and/or (ii) a nucleic acid at least 60% (e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 128.

In some embodiments, the present disclosure provides an isolated nucleic acid comprising a sequence at least 60% (e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 129, and/or a sequence at least 60% (e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 130. In some embodiments, the present disclosure provides an expression vector comprising: an isolated nucleic acid comprising a sequence at least 60% (e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 129, and/or a sequence at least 60% (e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 130.

In some embodiments, an anti-trypsin a/trypsin 2 antibody described herein is produced by expressing (i) a nucleic acid at least 60% (e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 129, and/or (ii) a nucleic acid at least 60% (e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 130.

In some embodiments, the present disclosure provides an isolated nucleic acid comprising a sequence at least 60% (e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 131, and/or a sequence at least 60% (e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 132. In some embodiments, the present disclosure provides an expression vector comprising: an isolated nucleic acid comprising a sequence at least 60% (e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 131, and/or a sequence at least 60% (e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 132.

In some embodiments, an anti-trypsin a/trypsin 2 antibody described herein is produced by expressing (i) a nucleic acid at least 60% (e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 131, and/or (ii) a nucleic acid at least 60% (e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 132.

In some embodiments, the present disclosure provides an isolated nucleic acid comprising a sequence at least 60% (e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 133, and/or a sequence at least 60% (e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 134. In some embodiments, the present disclosure provides an expression vector comprising: an isolated nucleic acid comprising a sequence at least 60% (e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 133, and/or a sequence at least 60% (e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 134.

In some embodiments, an anti-trypsin a/trypsin 2 antibody described herein is produced by expressing (i) a nucleic acid at least 60% (e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 133, and/or (ii) a nucleic acid at least 60% (e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 134.

In some embodiments, the present disclosure provides an isolated nucleic acid comprising a sequence at least 60% (e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 135, and/or a sequence at least 60% (e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 136. In some embodiments, the present disclosure provides an expression vector comprising: an isolated nucleic acid comprising a sequence at least 60% (e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 135, and/or a sequence at least 60% (e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 136.

In some embodiments, an anti-trypsin a/trypsin 2 antibody described herein is produced by expressing (i) a nucleic acid at least 60% (e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 135, and/or (ii) a nucleic acid at least 60% (e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 136.

In some embodiments, the present disclosure provides an isolated nucleic acid comprising a sequence at least 60% (e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 137, and/or a sequence at least 60% (e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 138. In some embodiments, the present disclosure provides an expression vector comprising: an isolated nucleic acid comprising a sequence at least 60% (e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 137, and/or a sequence at least 60% (e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 138.

In some embodiments, an anti-trypsin a/trypsin 2 antibody described herein is produced by expressing (i) a nucleic acid at least 60% (e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 137, and/or (ii) a nucleic acid at least 60% (e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 138.

Accordingly, the present disclosure provides methods for producing the antibody, the methods comprising culturing the recombinant cells under conditions suitable for expression of the antibody from the expression vector by the recombinant cell. Recombinant cells expressing the antibody can be cultured in any suitable condition known in the art. In some embodiments, the method further comprising isolating the antibody from the culture media in which the cell or cells were cultured using any suitable known methods in the art.

### IV. Pharmaceutical Composition

The antibodies, as well as the encoding nucleic acids or nucleic acid sets, vectors comprising such, or host cells comprising the vectors, as described herein can be mixed with a pharmaceutically acceptable carrier (excipient) to form a pharmaceutical composition for use in treating a target disease. “Acceptable” means that the carrier must be compatible with the active ingredient of the composition (and preferably, capable of stabilizing the active ingredient) and not deleterious to the subject to be treated. Pharmaceutically acceptable excipients (carriers) including buffers, which are well known in the art. See, e.g., Remington: The Science and Practice of Pharmacy 20th Ed. (2000) Lippincott Williams and Wilkins, Ed. K. E. Hoover.

In some embodiments, an antibody (e.g., an anti-trypsin 1/trypsin 2 antibody) containing pharmaceutical composition disclosed herein may further comprise a suitable buffer agent. A buffer agent is a weak acid or base used to maintain the pH of a solution near a chosen value after the addition of another acid or base. In some examples, the buffer agent disclosed herein can be a buffer agent capable of maintaining physiological pH despite changes in carbon dioxide concentration (produced by cellular respiration). Exemplary buffer agents include, but are not limited to a HEPES (4-(2-hydroxyethyl)-1-piperazineethanesulfonic acid) buffer, Dulbecco's phosphate-buffered saline (DPBS) buffer, or Phosphate-buffered Saline (PBS) buffer. Such buffers may comprise disodium hydrogen phosphate and sodium chloride, or potassium dihydrogen phosphate and potassium chloride.

The pharmaceutical composition described herein comprises one or more suitable salts. A salt is an ionic compound that can be formed by the neutralization reaction of an acid and a base. (Skoog, D. A; West, D. M.; Holler, J. F.; Crouch, S. R. (2004). “chapters 14-16”. Fundamentals of Analytical Chemistry (8th ed.)). Salts are composed of related numbers of cations (positively charged ions) and anions (negative ions) so that the product is electrically neutral (without a net charge).

In some embodiments, the pharmaceutical compositions can comprise pharmaceutically acceptable carriers, excipients, or stabilizers in the form of lyophilized formulations or aqueous solutions. (Remington: The Science and Practice of Pharmacy 20th Ed. (2000) Lippincott Williams and Wilkins, Ed. K. E. Hoover). In some embodiments, the pharmaceutical composition can be formulated for intravenous injection. In some embodiments, the pharmaceutical composition can be formulated for subcutaneous injection.

The pharmaceutical compositions to be used for in vivo administration must be sterile. This is readily accomplished by, for example, filtration through sterile filtration membranes. Therapeutic antibody compositions are generally placed into a container having a sterile access port, for example, an intravenous or subcutaneous solution bag or vial having a stopper pierceable by a hypodermic injection needle.

### V. Methods of Use

In certain aspects, the disclosure provides methods and related compositions for treating conditions associated with trypsin 1/trypsin 2 activation related dysregulation, e.g., pancreatitis. Accordingly, aspects of the disclosure relate to methods and compositions (e.g., anti-trypsin 1/trypsin 2 dual inhibitor antibodies) useful for inhibiting trypsin 1/trypsin 2 protease activity for treating pancreatitis (e.g., hereditary pancreatitis, acute pancreatitis, chronic pancreatitis, etc.).

In some embodiments, the present disclosure contemplates using any inhibitor antibody for trypsin 1 and/or trypsin 2 for treating pancreatitis. In some embodiments, methods provided herein relate to treating pancreatitis by administering to a subject in need thereof an effective amount of a trypsin 1 and/or 2 inhibitor antibody, wherein the trypsin 1 and/or 2 inhibitor antibody binds to an active trypsin 1 and/or 2 and inhibits protease activity of the active trypsin.

In some embodiments, the present disclosure provides methods of inhibiting trypsin 1/trypsin 2 protease activity to treat pancreatitis. In some embodiments, the methods and compositions of the present disclosure inhibit trypsin activity in the pancreas, in acinar cells, in the common bile duct, or in the small intestine.

In some embodiments, the present disclosure provides methods and related compositions for treating a subject having or suspected of having pancreatitis. In some embodiments, the subject is diagnosed with pancreatitis. Methods of diagnosing pancreatitis include, but are not limited to, performing a blood test, abdominal ultrasound, computerized tomography (CT) scan, magnetic resonance imaging (MRI), endoscopic ultrasound, or stool test.

Further aspects of the disclosure relate to methods and compositions (e.g., dual inhibitor anti-trypsin 1/trypsin 2 antibodies) useful for treating hereditary pancreatitis, which is a genetic condition typically characterized by recurrent episodes of inflammation of the pancreas (pancreatitis). Accordingly, in some embodiments, methods of treating subjects having hereditary pancreatitis are provided herein that comprise administering an effective amount of one or more dual inhibitor anti-trypsin 1/trypsin 2 antibodies to the subject. In some embodiments, hereditary pancreatitis presents in a subject as an autosomal dominant disease associated with elevated trypsin activity in the pancreas. In some embodiments, hereditary pancreatitis is caused by pathogenic mutations in certain genes (e.g., PRSS1 (cationic trypsinogen), CTRC (chymotrypsin C) and SPINK1 (serine protease inhibitor Kazal type 1)) that result in increased pancreatic trypsin activity. In some embodiments, increased conversion of trypsinogen to trypsin results from mutations that stimulate autoactivation of trypsin in hereditary pancreatitis. However, in some embodiments, increased conversion of trypsinogen to trypsin occurs because of decreases in or loss of the protective mechanisms of trypsin inhibition (e.g., by pathogenic mutations in SPINK1). In other embodiments, increased conversion of trypsinogen to trypsin occurs because of decreases in trypsinogen degradation activity (e.g., by pathogenic mutations in CTRC2). Non-limiting examples of genes and corresponding mutations or polymorphisms associated with hereditary pancreatitis are shown in Table 4 below. Accordingly, in some embodiments, a subject administered a dual inhibitor anti-Trypsin 1/Trypsin 2 antibody according to methods provided herein has a genetic alteration identified in Table 4.

TABLE 4
Non-limiting examples of genes and corresponding mutations
or polymorphisms associated with hereditary pancreatitis.

| | Gene | Entrez Gene | |
|---|---|---|---|
| Gene Name | Symbol | ID No. | Mutations Associated with HP |
| Cationic | PRSS1 | 5644 | A16V; R122C; R122H; N291; D19A; |
| trypsinogen | | | D21A; G208A; D22G; K23R; R122C; |
| | | | N29T; copy number variants; promoter |
| | | | polymorphisms |
| Serine protease | SPINK1 | 6690 | N34S; IVS3 + 2T > C |
| inhibitor Kazal- | | | |
| type 1 | | | |
| Chymotrypsinogen | CTRC | 11330 | A73T; K247_R254del; |
| C | | | |
| Calcium-sensing | CASR | 846 | L173P; A986S; Exon 7 R990G |
| receptor | | | |
| Carboxypeptidase | CPA1 | 1357 | N256K |
| A | | | |
| Pancreatic lipase | PNLIP | 5406 | P245A; I265R; F300L; S304F; F314L |
| Claudin-2 | CLDN2 | 9075 | rs12008279; rs4409525 |
| Carboxyl ester | CEL | 1056 | CEL-HYB1 allele |
| lipase | | | |
| Anionic | PRSS2 | 5645 | G191R; copy number variants; gene |
| trypsinogen | | | conversion between PRSS1 and PRSS2 |
| Chymotrypsin B1 | CTRB1; | 1504; 440387 | 16.6 kb inversion in CTRB1-CTRB2 |
| and B2 | CTRB2 | | locus |

In some embodiments, a subject administered a dual inhibitor anti-trypsin 1/trypsin 2 antibody according to methods provided herein has one or more symptoms of hereditary pancreatitis. For example, in some embodiments, episodes of pancreatitis can lead to tissue damage and loss of or decrease in pancreatic function. In some embodiments, subjects having hereditary pancreatitis begin to exhibit symptoms in late childhood, which may include manifestations of acute pancreatitis. In some embodiments, such pancreatitis episodes lead to, or are associated with, abdominal pain, fever, nausea, and/or vomiting. In some embodiments, such pancreatitis episodes may last from one to three days or more. In some embodiments, hereditary pancreatitis progresses to multiple recurrent episodes of acute pancreatitis that recur over a period of at least a year (e.g., 1 to 2 years). In some embodiments, this recurrent acute pancreatitis leads to chronic pancreatitis, in which the pancreas is persistently inflamed. In some embodiments, chronic pancreatitis develops by early adulthood (e.g., in the range of 18 to 45 years of age) in a subject. In some embodiments, subjects having hereditary pancreatitis that has progressed to the stage of chronic pancreatitis experience occasional or frequent abdominal, flatulence, and/or bloating. In some embodiments, subjects having hereditary pancreatitis develop abnormal calcium deposits in the pancreas (pancreatic calcifications), which may occur by early adulthood. In some embodiments, hereditary pancreatitis manifests as spontaneous acute pancreatitis with edema, necrosis and/or serum amylase elevation followed by progressive atrophic chronic pancreatitis with acinar cell loss, fibrosis, dilated ducts and adipose replacement at later stages. In some embodiments, treatment with the methods and compositions provided in this application prevents or reduces inflammation of the pancreas. In some embodiments, treatment with the methods and compositions provided in this application prevents or reduces one or more of edema, necrosis and/or serum amylase elevation followed by progressive atrophic chronic pancreatitis with acinar cell loss, fibrosis, dilated ducts, and adipose replacement. In some embodiments, treatment with the methods and compositions contemplated in this application prevents or reduces calcium deposits in the pancreas. In some embodiments, treatment with the methods and compositions contemplated in this application prevents or reduces occasional or frequent abdominal, flatulence, and/or bloating.

Further aspects of the disclosure relate to methods and compositions (e.g., dual inhibitor anti-trypsin 1/trypsin 2 antibodies) useful for treating acute pancreatitis. Accordingly, in some embodiments, methods of treating such subjects having acute pancreatitis are provided herein that comprise administering an effective amount of one or more dual inhibitor anti-trypsin 1/trypsin 2 antibodies to a subject. In some embodiments, the acute pancreatitis may be associated with pain that begins slowly or suddenly in a subject's upper abdomen, which pain may spread to a subject's back. In some embodiments, the pain is mild or severe. In some embodiments, the pain may last for several days (e.g., 5 to 7 days) or more. In some embodiments, subjects having acute pancreatitis may exhibit fever, nausea and/or vomiting, as well as a rapid heartbeat and/or a swollen or tender abdomen. In some embodiments, subjects having acute pancreatitis have elevated diagnostic enzyme levels, such as elevated amylase and/or lipase levels that are above physiologically normal levels, for example as reported, in Matull W R, et al., Biochemical Markers of Acute Pancreatitis, J Clin Pathol. 2006 April; 59(4): 340-344, the contents of which are incorporated herein by reference.

In some embodiments, acute pancreatitis is caused by gallstones, heavy alcohol use, or treatment with certain medicines. Medicines causing acute pancreatitis include, but are not limited to, didanosine, asparaginase, sulindac, sulfasalazine, azathioprine, furosemide, tetracycline, cytarabine, valproic acid, pentavent antimonials, pentamidine, mercaptopurine, steroids, mesalamine, estrogen and preparations thereof, trimethoprim/sulfamethoxazole, interferon alpha-2b, enalapril, opiates, carbamazepine, acetaminophen, pheformin, rifampin, octreotide, lamivudine, hydrochlorothiazide, erythromycin, cisplatin, and cyclopenthiazide. In some embodiments, acute pancreatitis is caused by an infection, such as by viruses or parasites. Infections causing acute pancreatitis include, but are not limited to infections by hepatotropic viruses (e.g. hepatitis A, B, C, D and E), Coxsackie virus, cytomegalovirus (CMV), human immunodeficiency virus (HIV), herpes simplex virus (HSV), mumps virus, varicella-zoster virus, Mycoplasma pneumoniae, leptospirosis, and Ascaris lumbricoides. In some embodiments, acute pancreatitis is caused by an abdominal injury. In some embodiments, acute pancreatitis is caused by pancreatic cancer. In some embodiments, acute pancreatitis is caused by a procedure referred to as endoscopic retrograde cholangiopancreatography (ERCP), which may be performed to treat another condition, e.g., pancreas divisum, removal of bile duct stones, or relief of biliary obstruction. In some embodiments, acute pancreatitis is associated with hereditary pancreatitis. In some embodiments, the acute pancreatitis is recurrent acute pancreatitis.

Further aspects of the disclosure relate to methods and compositions (e.g., dual inhibitor anti-trypsin 1/trypsin 2 antibodies) useful for treating chronic pancreatitis, which is characterized by chronic inflammation and progressive fibrosis of the pancreas, with loss of acinar cell mass. In some embodiments, chronic pancreatitis leads to irreversible morphologic changes, loss of pancreatic function, and increased risk of pancreatic cancer. Accordingly, in some embodiments, methods of treating such subjects having chronic pancreatitis are provided herein that comprise administering an effective amount of one or more anti-trypsin 1/trypsin 2 antibodies to the subject. In some embodiments, a subject having or suspected of having chronic pancreatitis has or reports pain, e.g., in the upper abdomen, which may spread to the subjects back. In some embodiments, the reported pain becomes constant and/or severe and can become worse after eating. In some embodiments, a subject having chronic pancreatitis experiences diarrhea, nausea, and greasy, foul-smelling stools, vomiting, and/or weight loss.

In some embodiments, chronic pancreatitis is caused by gallstones, heavy alcohol use, or treatment with certain medicines. Medicines causing acute pancreatitis include, but are not limited to, didanosine, asparaginase, sulindac, sulfasalazine, azathioprine, furosemide, tetracycline, cytarabine, valproic acid, pentavent antimonials, pentamidine, mercaptopurine, steroids, mesalamine, estrogen and preparations thereof, trimethoprim/sulfamethoxazole, interferon alpha-2b, enalapril, opiates, carbamazepine, acetaminophen, pheformin, rifampin, octreotide, lamivudine, hydrochlorothiazide, erythromycin, cisplatin, and cyclopenthiazide. In some embodiments, chronic pancreatitis is caused by an infection, such as by viruses or parasites. Infections causing acute pancreatitis include, but are not limited to infections by hepatotropic viruses (e.g. hepatitis A, B, C, D and E), Coxsackie virus, cytomegalovirus (CMV), human immunodeficiency virus (HIV), herpes simplex virus (HSV), mumps virus, varicella-zoster virus, Mycoplasma pneumoniae, leptospirosis, and Ascaris lumbricoides. In some embodiments, chronic pancreatitis is caused by an abdominal injury. In some embodiments, chronic pancreatitis is caused by pancreatic cancer. In some embodiments, chronic pancreatitis is caused by a procedure referred to as endoscopic retrograde cholangiopancreatography (ERCP), which may be performed to treat another condition, e.g., pancreas divisum, removal of bile duct stones, or relief of biliary obstruction. In some embodiments, chronic pancreatitis is associated with hereditary pancreatitis.

In some embodiments, a composition may be administered once. In some embodiments, a composition will be administered on multiple occasions. In some embodiments, a composition will be administered daily, biweekly, weekly, bimonthly, monthly, or at any time interval that provide suitable (e.g., maximal) efficacy while minimizing safety risks to the subject. Generally, the efficacy and the treatment and safety risks may be monitored throughout the course of treatment.

In some embodiments, a subject may be administered a composition provided herein (e.g., an anti-trypsin 1/trypsin 2 antibody) at one or more intervals during a set period of time. In some cases, periods of time during which a subject is administered a composition at one or more intervals may be separated by periods of time in which the subject is not administered the composition. In some embodiments, the relative durations of respective periods of time may depend on the subject's response to treatment or severity of disease or both and/or may be determined based on the judgment of a treating physician.

In some embodiments, an antibody can be administered parenterally. For example, a parenterally administered composition may be administered topically, transmucosally, by subcutaneous, intracutaneous, intravenous, intraperitoneal, intratumor, intramuscular, intraarticular, intraarterial, or infusion techniques.

In some embodiments, an antibody (e.g., an anti-trypsin 1/trypsin 2 antibody) is administered intravenously. In some embodiments, an antibody (e.g., an anti-trypsin 1/trypsin 2 antibody) is administered subcutaneously or topically.

For intravenous injection, water soluble antibodies can be administered by the drip method, whereby a pharmaceutical formulation containing the antibody and a physiologically acceptable excipient is infused. Physiologically acceptable excipients may include, for example, 5% dextrose, 0.9% saline. Ringer's solution or other suitable excipients. Other injectable compositions may contain various carriers such as vegetable oils, dimethylactamide, dimethyformamide, ethyl lactate, ethyl carbonate, isopropyl myristate, ethanol, and polyols (glycerol, propylene glycol, liquid polyethylene glycol, and the like). In some cases, preparations, e.g., a sterile formulation of a suitable soluble salt form of the antibody, can be dissolved and administered in a pharmaceutical excipient such as Water-for-Injection, 0.9% saline, or 5% glucose solution.

In one embodiment, an antibody is administered via site-specific or targeted local delivery techniques (e.g., to the pancreas).

An anti-trypsin 1/trypsin 2 antibody and treatment methods involving such as described in the present disclosure may be utilized in combination with other types of therapy for the target disease or disorder disclosed herein. In this context, an antibody composition and a therapeutic agent may be given either simultaneously or sequentially. Such therapies can be administered simultaneously or sequentially (in any order) with the treatment according to the present disclosure.

Accordingly, aspects of the disclosure related to methods and compositions (e.g., anti-trypsin 1/trypsin 2 antibodies). In some embodiments, antibodies as described herein, can be administered as a combination therapy (concomitantly or sequentially, e.g., over time). In some embodiments, the combination therapy comprises administering one or more of the antibodies as described herein (e.g., anti-trypsin 1/trypsin 2 antibodies) and at least one additional therapeutic agent (e.g., one, two, three, four, five, six, or seven therapeutic agents). In some embodiments, one or more of the antibodies as described herein and at least one additional therapeutic agent (e.g., one, two, three, four, five, six, or seven therapeutic agents) are administered together. In some embodiments, one or more of the antibodies as described herein and at least one additional therapeutic agent (e.g., one, two, three, four, five, six, or seven therapeutic agents) are administered separately.

### VI. Kits for Therapeutic and Diagnostic Applications

The present disclosure also provides kits for the applications as disclosed herein. Such kits can include one or more containers comprising an antibody, e.g., any of those described herein.

In some embodiments, the kit can comprise instructions for use in accordance with any of the methods described herein. The included instructions can comprise a description of administration of the antibody to treat, delay the onset, or alleviate a target disease as those described herein. The kit may further comprise a description of selecting an individual suitable for treatment based on identifying whether that individual has the target disease. In still other embodiments, the instructions comprise a description of administering an antibody to an individual at risk of the target disease.

The instructions relating to the use of an antibody described generally include information as to dosage, dosing schedule, and route of administration for the intended treatment. The containers may be unit doses, bulk packages (e.g., multi-dose packages) or sub-unit doses. Instructions supplied in the kits of the invention are typically written instructions on a label or package insert (e.g., a paper sheet included in the kit), but machine-readable instructions (e.g., instructions carried on a magnetic or optical storage disk) are also acceptable.

The label or package insert indicates that the composition is used for treating, delaying the onset and/or alleviating a disease or disorder. Instructions may be provided for practicing any of the methods described herein.

The kits of this invention are in suitable packaging. Suitable packaging includes, but is not limited to, vials, bottles, jars, flexible packaging (e.g., sealed Mylar or plastic bags), and the like.

Also contemplated are packages for use in combination with a specific device, such as an infusion device, such as a minipump. A kit may have a sterile access port (for example the container may be an intravenous solution bag or a vial having a stopper pierceable by a hypodermic injection needle). The container may also have a sterile access port (for example the container may be an intravenous solution bag or a vial having a stopper pierceable by a hypodermic injection needle). At least one active agent in the composition is an antibody as those described herein.

Kits may optionally provide additional components such as buffers and interpretive information. Normally, the kit comprises a container and a label or package insert(s) on or associated with the container. In some embodiments, the invention provides articles of manufacture comprising contents of the kits described above.

Also provided herein are kits for use in detecting the target protein (e.g., trypsin 1 and/or trypsin 2) in a sample. Such a kit may comprise any of the antibodies described herein. In some instances, the antibody can be conjugated with a detectable label as those described herein. As used herein, “conjugated” or “attached” means two entities are associated, preferably with sufficient affinity that the therapeutic/diagnostic benefit of the association between the two entities is realized. The association between the two entities can be either direct or via a linker, such as a polymer linker. Conjugated or attached can include covalent or noncovalent bonding as well as other forms of association, such as entrapment, e.g., of one entity on or within the other, or of either or both entities on or within a third entity, such as a micelle.

Alternatively or in addition, the kit may comprise a secondary antibody capable of binding to an antibody described herein. The kit may further comprise instructions for using the antibody for detecting the target protein (e.g., trypsin 1 and/or trypsin 2).

### EXAMPLES

### Example 1: Generation and Selection of Dual Inhibitor Anti-Trypsin 1/Trypsin 2 Antibodies

Antibody binding kinetic experiments via Biacore were performed to screen for anti-trypsin 1/trypsin 2 antibodies having high affinity to the respective targets. All screening assays were performed at 25 C. The running buffer used was 20 mM HEPES, 300 mM NaCl, 1 mM CaCl₂, 0.01% Tween-20, pH 7.5. Antibodies (1 ug/mL) were captured on Fc2-4 of a Series S ProteinA Sensor Chip (Cytiva) flowing at 10 uL/min for 30 s. Kinetics measurements were made in single-cycle kinetics mode with a series of 4-5 concentrations with top concentration of 10-200 nM and serial dilution 4-fold. Contact time was typically 180-300 s and dissociation time ranged from 300-3600 s. Flow rate of 30 uL/min was typically used. The sensor chip was regenerated with 10 mM glycine, pH 1.5 with a contact time of 30 s flowing at 50 uL/min.

Trypsin protease inhibition assay were performed to evaluate the potency of the anti-trypsin 1/typsin 2 antibodies in inhibiting trypsin 1 or trypsin 2 protease activity respectively. Final concentrations of 0.5 nM of human PRSS1 (R&D Systems), PRSS2 (R&D Systems) and Native Trypsin (abcam) were prepared in assay buffer (1×HEPES buffered Saline, 1 mM CaCl₂, 0.05% Tween 20, pH 7.4). Both PRSS1 and PRSS2 were activated overnight with 20 units of enterokinase (New England Biolabs Catalog #P8070). Native Trypsin was already activated. Antibodies were diluted in 1×HEPES buffered saline to testing concentrations and added to enzyme. Enzyme and antibodies were incubated for 10 minutes prior to substrate addition. Substrate is then added (10 uM final concentration of MCA-RPKPVE-Nva-WRK(Dnp)-NH2 fluorogenic substrate (Anaspec). Fluorescence is then measured every 3 minutes for 60 min at room temperature. IC50 was calculated.

The results show that dual inhibitor antibodies described in Table 1a showed binding specificity to both trypsin 1 and typsin 2. The antibodies are potent in inhibiting trypsin 1 and trypsin 2 activities. A summary of results are provided below in Table 5.

TABLE 5
Anti-trypsin 1/trypsin 2 Antibody Affinity and Potency

| | | | IC50 Against | KD via | KD via |
|---|---|---|---|---|---|
| | IC50 Against | IC50 Against | 0.25 nM Native | Biacore | Biacore |
| | 1 nM hTryp-1 | 1 nM hTryp-2 | Trypsin | Trypsin 1 | Trypsin 2 |
| Antibody Name | (nM) | (nM) | (nM) | S to A | S to A |
| Trypsin 1/Trypsin | 63.0 | 79.0 | | 2.54E−09 | 7.63E−10 |
| 2 -Dual-L1-Ab1 | | | | | |
| Trypsin 1/Trypsin | 3.70 | 5.33 | 7.98 | <1.00E−11 | <1.00E−11 |
| 2 -Dual-L1-Ab2 | | | | | |
| Trypsin 1/Trypsin | 46.00 | 79.00 | | 2.61E−09 | 2.2E−09 |
| 2 -Dual-L2-Ab1 | | | | | |
| Trypsin 1/Trypsin | 2.67 | 3.30 | 5.51 | 8.91E−13 | 9.88E−12 |
| 2 -Dual-L2-Ab2 | | | | | |
| Trypsin 1/Trypsin | 2.30 | 2.44 | 4.63 | 1.18E−11 | 2.19E−11 |
| 2 -Dual-L2-Ab3 | | | | | |
| Trypsin 1/Trypsin | 3.71 | 5.60 | 4.97 | <1.00E−11 | 9.04E−12 |
| 2 -Dual-L2-Ab4 | | | | | |
| Trypsin 1/Trypsin | 5.00 | 16.00 | | 1.77E−11 | 1.59E−10 |
| 2 -Dual-L3-Ab1 | | | | | |
| Trypsin 1/Trypsin | 2.75 | 4.29 | 5.95 | 5.62E−12 | 2.92E−11 |
| 2 -Dual-L3-Ab2 | | | | | |
| Trypsin 1/Trypsin | 1.80 | 14.00 | | 8.09E−11 | 5.47E−10 |
| 2 -Dual-L4-Ab1 | | | | | |
| Trypsin 1/Trypsin | 2.07 | 2.03 | 5.51 | <1.00E−11 | 3.72E−11 |
| 2 -Dual-L4-Ab2 | | | | | |
| Trypsin 1/Trypsin | 2.31 | 2.01 | 4.43 | 8.41E−12 | 4.51E−11 |
| 2 -Dual-L4-Ab3 | | | | | |
| Trypsin 1/Trypsin | 2.05 | 2.01 | 5.48 | <1.00E−11 | 2.06E−11 |
| 2 -Dual-L4-Ab4 | | | | | |
| Trypsin 1/Trypsin | 2.4 | 2.7 | 4.8 | 4.65E−12 | 2.74E−11 |
| 2 -Dual-L4-Ab5 | | | | | |
| Trypsin 1/Trypsin | 2.3 | 1.9 | 4.2 | 1.13E−11 | 3.65E−11 |
| 2 -Dual-L4-Ab6 | | | | | |
| Trypsin 1/Trypsin | 1.9 | 2.5 | 4.7 | 8.75E−12 | 4.37E−11 |
| 2 -Dual-L4-Ab7 | | | | | |
| Trypsin 1/Trypsin | 45.0 | 23.0 | | 1.51E−10 | 2.27E−10 |
| 2 -Dual-L5-Ab1 | | | | | |
| Trypsin 1/Trypsin | 5.87 | 6.09 | 9.82 | <1.00E−11 | 6.48E−12 |
| 2 -Dual-L5-Ab2 | | | | | |

Further, the anti-trypsin 1/trypsin 2 antibodies were tested for their off-target effects on proteases other than trypsin 1 and trypsin 2. The following other off target proteases were screened based on activity assays: Chymotrypsin (final concentration of 0.5 ug/mL), KLK5 (final concentration of 6 nM), Factor Xa (final concentration of 0.1 ug/mL), Cathepsin B (final concentration of 1 nM), Cathepsin G (final concentration of 12.5 nM), Pancreatic Elastase (final concentration of 25 nM), Plasmin (final concentration of 2.5 nM). Substrates for protease assays were as follows: 20 uM final concentration of Suc-AAPF-AMC for Chymotrypsin, 250 uM final concentration of Boc-Val-Pro-Arg-AMC for KLK5, 20 uM final concentration of MCA-RPKPVG-Nva1-WRK(Dnp)-NH2 (SEQ ID NO: 139) for Factor Xa, 10 uM final concentration of Z-Leu-Arg-AMC for Cathepsin B, 300 uM final concentration of Suc-AAPF-AMC for Cathepsin G, 300 uM final concentration of MeOSuc-AAPV-AMC (SEQ ID NO: 140) for Pancreatic Elastase and 200 uM of Suc-AFK-AMC for Plasmin. No significant inhibition was observed from the anti-trypsin 1/trypsin 2 antibodies on cleavage activity of chymotrypsin, KLK5, factor Xa, cathepsin B, cathepsin G, elastase, and plasmin.

### Example 2 Anti-Trypsin I/Trypsin 2 Antibodies for Treating Pancreatitis in a Rat Pancreatitis Model

Caerulein-induced pancreatitis model was used to evaluate the efficacy of anti-trypsin 1/trypsin 2 antibody in treating pancreatitis. Three rat surrogate anti-trypsin 1/trypsin 2 antibodies (R1, R2, and R3) were developed for this study. These dual inhibitor antibodies specifically bind to and inhibit trypsin 1 and trypsin 2 protease activity. In this study, antibodies R1, R2, or R3 was administered twice (2×) via intraperitoneal route (IP) in caerulein-induced pancreatitis in male Lewis rats. Body weights were measured prior to randomization on study day −2 and treatment was initiated. The animals were dosed IP 39 hours and 15 hours prior to first caerulein (CRN) injection with control IgG (60 mg/kg), R1 (60 mg/kg), R2 (60 mg/kg), or R3 (60 mg/kg). One positive control group was treated orally (PO) with Camostat (300 mg/kg) 24 hours and 1 hour prior to first CRN injection. Animals were fasted overnight (day −1) prior to disease induction. On study day 0, animals were administered 200 μL of 50 μg/mL caerulein (IP) hourly for a total of 7 doses (at time points 0, 1, 2, 3, 4, 5, and 6 h) to induce pancreatitis. Negative vehicle control were administered saline for IP challenge and treatment. The animals were terminated one hour after the final injection of caerulein for necropsy and tissue collection. Efficacy evaluation was based on pancreas histopathology.

### Acinar Cell Degeneration Count and Score

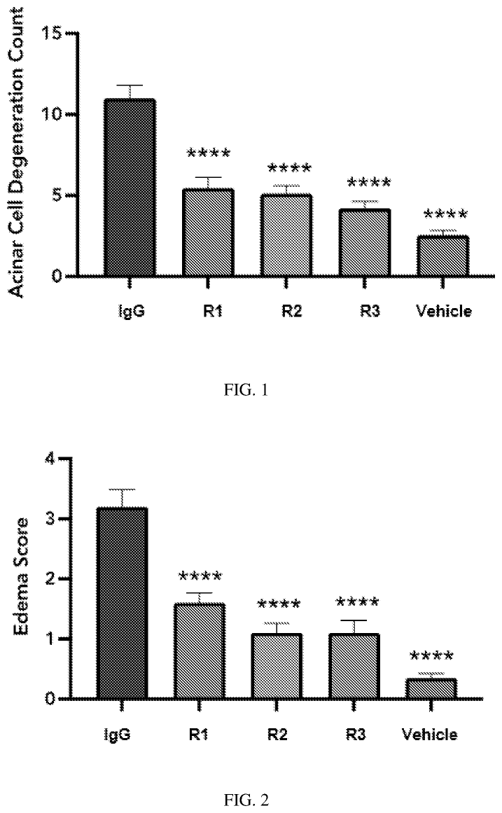

The pancreas was visually divided into 5 subregions, and acinar cells with degeneration or necrosis were counted in the most severely-affected 25 μm×100 μm (400×) fields in each of these subregions using an area on the micrometer that is 10×40 units (each unit is 2.5 μm, so 25 μm×100 μm=2500 μm²), and a mean value across all five fields is calculated for each animal. The results showed that antibodies R1, R2, and R3 were able to reduce Acinar Cell Degeneration significantly compared to animals received IgG control (FIG. 1).

### Edema Score

Edema was scored by a pathologist according to the following criteria: 0=None, 0.5=One to several small foci in adipose tissue around pancreas, 1=One to several large foci in adipose tissue around pancreas, 2=Multifocal mild affecting 10⁻²⁵% of the lobules, 3=Multifocal moderate affecting 26-50% of the lobules, 4=Diffuse marked affecting 51-75% of the lobules, 5=Diffuse severe affecting greater than 75% of the lobules. The results showed that antibodies R1, R2, and R3 were able to reduce edema significantly compared to animals received IgG control (FIG. 2).

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions.

### EQUIVALENTS AND SCOPE

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03. It should be appreciated that embodiments described in this document using an open-ended transitional phrase (e.g., “comprising”) are also contemplated, in alternative embodiments, as “consisting of” and “consisting essentially of” the feature described by the open-ended transitional phrase. For example, if the application describes “a composition comprising A and B,” the application also contemplates the alternative embodiments “a composition consisting of A and B” and “a composition consisting essentially of A and B.”

Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

The recitation of a listing of chemical groups in any definition of a variable herein includes definitions of that variable as any single group or combination of listed groups. The recitation of an embodiment for a variable herein includes that embodiment as any single embodiment or in combination with any other embodiments or portions thereof. The recitation of an embodiment herein includes that embodiment as any single embodiment or in combination with any other embodiments or portions thereof.

What is claimed is:

1. A dual inhibitor antibody that specifically binds to trypsin 1 and trypsin 2, the dual inhibitor antibody comprising
(a) a HC CDR1, HC CDR2 and HC CDR3 of a heavy chain variable domain (VH) having the amino acid sequence of SEQ ID NO: 57, and a LC CDR1, LC CDR2 and LC CDR3 of a light chain variable domain (VL) having the amino acid sequence of SEQ ID NO: 58;
(b) a HC CDR1, HC CDR2 and HC CDR3 of a heavy chain variable domain having the amino acid sequence of SEQ ID NO: 59, and a LC CDR1, LC CDR2 and LC CDR3 of a light chain variable domain having the amino acid sequence of SEQ ID NO: 60;
(c) a HC CDR1, HC CDR2 and HC CDR3 of a heavy chain variable domain having the amino acid sequence of SEQ ID NO: 61, and a LC CDR1, LC CDR2 and LC CDR3 of a light chain variable domain having the amino acid sequence of SEQ ID NO: 62;
(d) a HC CDR1, HC CDR2 and HC CDR3 of a heavy chain variable domain having the amino acid sequence of SEQ ID NO: 63, and a LC CDR1, LC CDR2 and LC CDR3 of a light chain variable domain having the amino acid sequence of SEQ ID NO: 64;
(e) a HC CDR1, HC CDR2 and HC CDR3 of a heavy chain variable domain having the amino acid sequence of SEQ ID NO: 65, and a LC CDR1, LC CDR2 and LC CDR3 of a light chain variable domain having the amino acid sequence of SEQ ID NO: 66;
(f) a HC CDR1, HC CDR2 and HC CDR3 of a heavy chain variable domain having the amino acid sequence of SEQ ID NO: 67, and a LC CDR1, LC CDR2 and LC CDR3 of a light chain variable domain having the amino acid sequence of SEQ ID NO: 68;
(g) a HC CDR1, HC CDR2 and HC CDR3 of a heavy chain variable domain having the amino acid sequence of SEQ ID NO: 69, and a LC CDR1, LC CDR2 and LC CDR3 of a light chain variable domain having the amino acid sequence of SEQ ID NO: 70;
(h) a HC CDR1, HC CDR2 and HC CDR3 of a heavy chain variable domain having the amino acid sequence of SEQ ID NO: 71, and a LC CDR1, LC CDR2 and LC CDR3 of a light chain variable domain having the amino acid sequence of SEQ ID NO: 70;
(i) a HC CDR1, HC CDR2 and HC CDR3 of a heavy chain variable domain having the amino acid sequence of SEQ ID NO: 72, and a LC CDR1, LC CDR2 and LC CDR3 of a light chain variable domain having the amino acid sequence of SEQ ID NO: 73;
(j) a HC CDR1, HC CDR2 and HC CDR3 of a heavy chain variable domain having the amino acid sequence of SEQ ID NO: 74, and a LC CDR1, LC CDR2 and LC CDR3 of a light chain variable domain having the amino acid sequence of SEQ ID NO: 73;
(k) a HC CDR1, HC CDR2 and HC CDR3 of a heavy chain variable domain having the amino acid sequence of SEQ ID NO: 75, and a LC CDR1, LC CDR2 and LC CDR3 of a light chain variable domain having the amino acid sequence of SEQ ID NO: 73;
(l) a HC CDR1, HC CDR2 and HC CDR3 of a heavy chain variable domain having the amino acid sequence of SEQ ID NO: 76, and a LC CDR1, LC CDR2 and LC CDR3 of a light chain variable domain having the amino acid sequence of SEQ ID NO: 73;
(m) a HC CDR1, HC CDR2 and HC CDR3 of a heavy chain variable domain having the amino acid sequence of SEQ ID NO: 77, and a LC CDR1, LC CDR2 and LC CDR3 of a light chain variable domain having the amino acid sequence of SEQ ID NO: 78;
(n) a HC CDR1, HC CDR2 and HC CDR3 of a heavy chain variable domain having the amino acid sequence of SEQ ID NO: 79, and a LC CDR1, LC CDR2 and LC CDR3 of a light chain variable domain having the amino acid sequence of SEQ ID NO: 80;
(o) a HC CDR1, HC CDR2 and HC CDR3 of a heavy chain variable domain having the amino acid sequence of SEQ ID NO: 81, and a LC CDR1, LC CDR2 and LC CDR3 of a light chain variable domain having the amino acid sequence of SEQ ID NO: 82;
(p) a HC CDR1, HC CDR2 and HC CDR3 of a heavy chain variable domain having the amino acid sequence of SEQ ID NO: 83, and a LC CDR1, LC CDR2 and LC CDR3 of a light chain variable domain having the amino acid sequence of SEQ ID NO: 70; or
(q) a HC CDR1, HC CDR2 and HC CDR3 of a heavy chain variable domain having the amino acid sequence of SEQ ID NO: 84, and a LC CDR1, LC CDR2 and LC CDR3 of a light chain variable domain having the amino acid sequence of SEQ ID NO: 70.

2. The dual inhibitor antibody of claim 1, wherein the dual inhibitor antibody comprises:
(a) a HC CDR1 having the amino acid sequence of SEQ ID NO: 1, a HC CDR2 having the amino acid sequence of SEQ ID NO: 2, a HC CDR3 having the amino acid sequence of SEQ ID NO: 3, a LC CDR1 having the amino acid sequence of SEQ ID NO: 4, a LC CDR2 having the amino acid sequence of SEQ ID NO: 5, and a LC CDR3 having the amino acid sequence of SEQ ID NO: 6;
(b) a HC CDR1 having the amino acid sequence of SEQ ID NO: 7, a HC CDR2 having the amino acid sequence of SEQ ID NO: 8, a HC CDR3 having the amino acid sequence of SEQ ID NO: 9, a LC CDR1 having the amino acid sequence of SEQ ID NO: 4, a LC CDR2 having the amino acid sequence of SEQ ID NO: 5, and a LC CDR3 having the amino acid sequence of SEQ ID NO: 10;
(c) a HC CDR1 having the amino acid sequence of SEQ ID NO: 11, a HC CDR2 having the amino acid sequence of SEQ ID NO: 12, a HC CDR3 having the amino acid sequence of SEQ ID NO: 13, a LC CDR1 having the amino acid sequence of SEQ ID NO: 14, a LC CDR2 having the amino acid sequence of SEQ ID NO: 15, and a LC CDR3 having the amino acid sequence of SEQ ID NO: 16;
(d) a HC CDR1 having the amino acid sequence of SEQ ID NO: 17, a HC CDR2 having the amino acid sequence of SEQ ID NO: 18, a HC CDR3 having the amino acid sequence of SEQ ID NO: 19, a LC CDR1 having the amino acid sequence of SEQ ID NO: 14, a LC CDR2 having the amino acid sequence of SEQ ID NO: 15, and a LC CDR3 having the amino acid sequence of SEQ ID NO: 20;
(e) a HC CDR1 having the amino acid sequence of SEQ ID NO: 21, a HC CDR2 having the amino acid sequence of SEQ ID NO: 22, a HC CDR3 having the amino acid sequence of SEQ ID NO: 23, a LC CDR1 having the amino acid sequence of SEQ ID NO: 14, a LC CDR2 having the amino acid sequence of SEQ ID NO: 15, and a LC CDR3 having the amino acid sequence of SEQ ID NO: 24;
(f) a HC CDR1 having the amino acid sequence of SEQ ID NO: 21, a HC CDR2 having the amino acid sequence of SEQ ID NO: 18, a HC CDR3 having the amino acid sequence of SEQ ID NO: 25, a LC CDR1 having the amino acid sequence of SEQ ID NO: 14, a LC CDR2 having the amino acid sequence of SEQ ID NO: 15, and a LC CDR3 having the amino acid sequence of SEQ ID NO: 26;
(g) a HC CDR1 having the amino acid sequence of SEQ ID NO: 27, a HC CDR2 having the amino acid sequence of SEQ ID NO: 28, a HC CDR3 having the amino acid sequence of SEQ ID NO: 29, a LC CDR1 having the amino acid sequence of SEQ ID NO: 30, a LC CDR2 having the amino acid sequence of SEQ ID NO: 31, and a LC CDR3 having the amino acid sequence of SEQ ID NO: 32;
(h) a HC CDR1 having the amino acid sequence of SEQ ID NO: 33, a HC CDR2 having the amino acid sequence of SEQ ID NO: 34, a HC CDR3 having the amino acid sequence of SEQ ID NO: 35, a LC CDR1 having the amino acid sequence of SEQ ID NO: 30, a LC CDR2 having the amino acid sequence of SEQ ID NO: 31, and a LC CDR3 having the amino acid sequence of SEQ ID NO: 32;
(i) a HC CDR1 having the amino acid sequence of SEQ ID NO: 36, a HC CDR2 having the amino acid sequence of SEQ ID NO: 37, a HC CDR3 having the amino acid sequence of SEQ ID NO: 38, a LC CDR1 having the amino acid sequence of SEQ ID NO: 4, a LC CDR2 having the amino acid sequence of SEQ ID NO: 5, and a LC CDR3 having the amino acid sequence of SEQ ID NO: 39;
(j) a HC CDR1 having the amino acid sequence of SEQ ID NO: 40, a HC CDR2 having the amino acid sequence of SEQ ID NO: 41, a HC CDR3 having the amino acid sequence of SEQ ID NO: 38, a LC CDR1 having the amino acid sequence of SEQ ID NO: 4, a LC CDR2 having the amino acid sequence of SEQ ID NO: 5, and a LC CDR3 having the amino acid sequence of SEQ ID NO: 39;
(k) a HC CDR1 having the amino acid sequence of SEQ ID NO: 40, a HC CDR2 having the amino acid sequence of SEQ ID NO: 41, a HC CDR3 having the amino acid sequence of SEQ ID NO: 42, a LC CDR1 having the amino acid sequence of SEQ ID NO: 4, a LC CDR2 having the amino acid sequence of SEQ ID NO: 5, and a LC CDR3 having the amino acid sequence of SEQ ID NO: 39;
(l) a HC CDR1 having the amino acid sequence of SEQ ID NO: 40, a HC CDR2 having the amino acid sequence of SEQ ID NO: 41, a HC CDR3 having the amino acid sequence of SEQ ID NO: 43, a LC CDR1 having the amino acid sequence of SEQ ID NO: 4, a LC CDR2 having the amino acid sequence of SEQ ID NO: 5, and a LC CDR3 having the amino acid sequence of SEQ ID NO: 39;
(m) a HC CDR1 having the amino acid sequence of SEQ ID NO: 40, a HC CDR2 having the amino acid sequence of SEQ ID NO: 41, a HC CDR3 having the amino acid sequence of SEQ ID NO: 44, a LC CDR1 having the amino acid sequence of SEQ ID NO: 4, a LC CDR2 having the amino acid sequence of SEQ ID NO: 5, and a LC CDR3 having the amino acid sequence of SEQ ID NO: 45;
(n) a HC CDR1 having the amino acid sequence of SEQ ID NO: 40, a HC CDR2 having the amino acid sequence of SEQ ID NO: 46, a HC CDR3 having the amino acid sequence of SEQ ID NO: 47, a LC CDR1 having the amino acid sequence of SEQ ID NO: 4, a LC CDR2 having the amino acid sequence of SEQ ID NO: 5, and a LC CDR3 having the amino acid sequence of SEQ ID NO: 48;
(o) a HC CDR1 having the amino acid sequence of SEQ ID NO: 49, a HC CDR2 having the amino acid sequence of SEQ ID NO: 50, a HC CDR3 having the amino acid sequence of SEQ ID NO: 51, a LC CDR1 having the amino acid sequence of SEQ ID NO: 4, a LC CDR2 having the amino acid sequence of SEQ ID NO: 5, and a LC CDR3 having the amino acid sequence of SEQ ID NO: 52;
(p) a HC CDR1 having the amino acid sequence of SEQ ID NO: 27, a HC CDR2 having the amino acid sequence of SEQ ID NO: 28, a HC CDR3 having the amino acid sequence of SEQ ID NO: 53, a LC CDR1 having the amino acid sequence of SEQ ID NO: 30, a LC CDR2 having the amino acid sequence of SEQ ID NO: 31, and a LC CDR3 having the amino acid sequence of SEQ ID NO: 32; or
(q) a HC CDR1 having the amino acid sequence of SEQ ID NO: 54, a HC CDR2 having the amino acid sequence of SEQ ID NO: 55, a HC CDR3 having the amino acid sequence of SEQ ID NO: 56, a LC CDR1 having the amino acid sequence of SEQ ID NO: 30, a LC CDR2 having the amino acid sequence of SEQ ID NO: 31, and a LC CDR3 having the amino acid sequence of SEQ ID NO: 32.

3. The dual inhibitor antibody of claim 1 or 2, the dual inhibitor antibody comprising
(a) a VH comprising the amino acid sequence of SEQ ID NO: 57, and a VL comprising the amino acid sequence of SEQ ID NO: 58;
(b) a VH comprising the amino acid sequence of SEQ ID NO: 59, and a VL comprising the amino acid sequence of SEQ ID NO: 60;
(c) a VH comprising the amino acid sequence of SEQ ID NO: 61, and a VL comprising the amino acid sequence of SEQ ID NO: 62;
(d) a VH comprising the amino acid sequence of SEQ ID NO: 63, and a VL comprising the amino acid sequence of SEQ ID NO: 64;
(e) a VH comprising the amino acid sequence of SEQ ID NO: 65, and a VL comprising the amino acid sequence of SEQ ID NO: 66;
(f) a VH comprising the amino acid sequence of SEQ ID NO: 67, and a VL comprising the amino acid sequence of SEQ ID NO: 68;
(g) a VH comprising the amino acid sequence of SEQ ID NO: 69, and a VL comprising the amino acid sequence of SEQ ID NO: 70;
(h) a VH comprising the amino acid sequence of SEQ ID NO: 71, and a VL comprising the amino acid sequence of SEQ ID NO: 70;
(i) a VH comprising the amino acid sequence of SEQ ID NO: 72, and a VL comprising the amino acid sequence of SEQ ID NO: 73;
(j) a VH comprising the amino acid sequence of SEQ ID NO: 74, and a VL comprising the amino acid sequence of SEQ ID NO: 73;
(k) a VH comprising the amino acid sequence of SEQ ID NO: 75, and a VL comprising the amino acid sequence of SEQ ID NO: 73;
(l) a VH comprising the amino acid sequence of SEQ ID NO: 76, and a VL comprising the amino acid sequence of SEQ ID NO: 73;
(m) a VH comprising the amino acid sequence of SEQ ID NO: 77, and a VL comprising the amino acid sequence of SEQ ID NO: 78;
(n) a VH comprising the amino acid sequence of SEQ ID NO: 79, and a VL comprising the amino acid sequence of SEQ ID NO: 80;
(o) a VH comprising the amino acid sequence of SEQ ID NO: 81, and a VL comprising the amino acid sequence of SEQ ID NO: 82;
(p) a VH comprising the amino acid sequence of SEQ ID NO: 83, and a VL comprising the amino acid sequence of SEQ ID NO: 70; or
(q) a VH comprising the amino acid sequence of SEQ ID NO: 84, and a VL comprising the amino acid sequence of SEQ ID NO: 70.

4. A dual inhibitor antibody comprising
(i) a HC CDR1, HC CDR2, HC CDR3, LC CDR1, LC CDR2, and/or LC CDR3 of any one of the antibodies listed in Table 1a and Table 1b; or
(ii) a VH and/or a VL of any one of the antibodies listed in Table 1a.

5. A composition comprising the dual inhibitor antibody of any one of claims 1-4 and a pharmaceutically acceptable carrier.

6. A nucleic acid encoding the dual inhibitor antibody of any one of claims 1-4.

7. A method of treating pancreatitis, the method comprising administering to a subject in need thereof an effective amount of a dual inhibitor antibody of any one of claims 1-4.

8. The method of claim 7, wherein the subject has hereditary pancreatitis, acute pancreatitis, recurrent acute pancreatitis, or chronic pancreatitis.

9. A method of treating pancreatitis, the method comprising administering to a subject in need thereof an effective amount of at least one antibody that specifically binds to trypsin 1 and/or trypsin 2.

10. The method of claim 9, wherein the at least one antibody specifically binds to trypsin 1.

11. The method of claim 9 or 10, wherein the at least one antibody specifically binds to the active site of trypsin 1.

12. The method of any one of claims 9-11, wherein the at least one antibody inhibits protease activity of trypsin 1.

13. The method of claim 9, wherein the at least one antibody specifically binds to trypsin 2.

14. The method of claim 9 or 13, wherein the at least one antibody specifically binds to the active site of trypsin 2.

15. The method of any one of claims 9 or 13-14, wherein the at least one antibody inhibits protease activity of trypsin 2.

16. The method of claim 9, further comprising administering to the subject at least one antibody that specifically binds to trypsin 1 and trypsin 2 and inhibits their respective protease activities.

17. The method of claim 16, wherein the at least one antibody is a dual inhibitor antibody that specifically binds to trypsin 1 and trypsin 2.

18. The method of any one of claims 9-17, wherein the subject has hereditary pancreatitis, acute pancreatitis, recurrent acute pancreatitis, or chronic pancreatitis.

19. A polypeptide comprising a sequence as set forth in SEQ ID NO: 103 or a fragment thereof suitable for generating an antibody that specifically binds to and inhibits protease activity of trypsin 1, optionally wherein the polypeptide comprises a detectable tag, such as a His-tag.

20. A polypeptide comprising a sequence as set forth in SEQ ID NO: 104 or a fragment thereof suitable for generating an antibody that specifically binds to and inhibits protease activity of trypsin 2, optionally wherein the polypeptide comprises a detectable tag, such as a His-tag.

21. A method of generating a dual inhibitor anti-trypsin 1/trypsin 2 antibody, the method comprising generating an antibody that i) specifically binds to the polypeptide of claim 19 and inhibits protease activity of trypsin 1 and ii) specifically binds to the polypeptide of claim 20 and inhibits protease activity of trypsin 2.
